(19) United States
(12) Patent Application Publication
(10) Pub. No.: US 2023/0001008 A1
(43) Pub. Date: Jan. 5, 2023

(54) COMPOSITIONS AND METHODS FOR TARGETING CELLULAR MOLECULES

(71) Applicant: KUMQUAT BIOSCIENCES INC., San Diego, CA (US)

(72) Inventors: Yi Liu, San Diego, CA (US); Liansheng Li, San Diego, CA (US); Matthew R. Janes, San Diego, CA (US); Karen K. Wong, San Diego, CA (US)

(21) Appl. No.: 17/636,692

(22) PCT Filed: Aug. 20, 2020
(86) PCT No.: PCT/US2020/047278

§ 371 Date: Feb. 18, 2022

Related U.S. Application Data
(60) Provisional application No. 62/889,501, filed on Aug. 20, 2019.

(51) Int. Cl. A61K 47/68; A61K 45/06; A61P 35/00

(52) U.S. Cl. CPC A61K 47/6897; A61K 45/06; A61K 47/6813; A61P 35/00

(57) ABSTRACT

The present disclosure provides isolated polypeptide comprising an antigen binding unit directed to a cellular targete bound by an exogenous molecule. The polypeptides, cells comprising the same are useful for targeting intracellular targets or intracellular portions of a target. The compositions and methods disclosed herein have a range of utilities as therapeutics, diagnostics, research tools.

### CROSS-REFERENCE

This application claims priority to U.S. Ser. No. 62/889,501 filed Aug. 20, 2019, the content of which is incorporated herein in its entirety.

### BACKGROUND

The lack of therapeutic efficacy and the prevalence of side effects of a therapy during development are often due to non-specific or off-target delivery of the underlying therapeutic. Antibodies, as therapeutic or diagnostic agents, rely on their target-binding specificities to carry out their biological functions in vivo. In particular, antibody therapeutics have been shown to (i) target secreted growth factors to reduce tumor angiogenesis (e.g., bevacizumab); (ii) bind cell surface cancer markers to inhibit immune check points and induce stronger immune cell response (e.g., ipilimumab and nivolumab); and (iii) deliver radioisotopes (e.g., ibritumomab tiuxetan) or toxic drugs (e.g., brentuximab vedotin) by interacting with the extracellular domains of target molecules that are preferentially expressed on the disease cells or tissues of interest. In recent years, antibodies have been used in conjunction with immunotherapy, by which immune cells are recruited to cancer tissues via bispecific antibodies (e.g., blinatumomab) or chimeric antigen receptor (CAR) T cells. However, many therapeutic antibodies are not tumor specific as the corresponding cellular antigens are expressed in both cancer and normal tissues, and thus causing undesired side effects including toxicity. Indeed, the difficulty in identifying tumor-unique antigens continues to hamper the development of more efficacious antibody therapeutics.

Another major limitation to the conventional antibody-based therapies is that they are typically restricted to targeting extracellular molecules or extracellular domains of the membrane bound molecules. It is well known that disease formation and progression involve an intricate and temporal activation and downregulation of by many more intracellular molecules. Extracellular targets constitute merely a small portion of the cellular targets that regulate the overall cellular function.

Despite the advent in antibody-based therapeutics and many other cancer therapies, cancer remains the second leading cause of human death. There were close to 10 million deaths from cancer worldwide in 2018 and 17 million new cases were diagnosed. In the United States alone, cancer causes the death of over a half-million people annually, with some 1.7 million new cases diagnosed per year (excluding basal cell and squamous cell skin cancers). Lung, liver, stomach, and bowel are the most common causes of cancer death worldwide, accounting for more than four in ten of all cancer deaths.

### SUMMARY

In view of the foregoing, there remains a considerable need for a new design of therapeutics and diagnostics that can specifically target intracellular or intracellular portions of cancer or other disease targets. There also exists a pressing need for identifying tumor neoantigens that are unique to the tumor microenvironment. The present disclosure addresses these needs, and provides additional advantages applicable for diagnosis, prognosis, and treatment for a wide diversity of diseases.

In one aspect, the disclosure provides an isolated polypeptide comprising an antigen binding unit, wherein the antigen binding unit (a) exhibits specific binding to a cellular target covalently bound by an exogenous molecule (bound target), but (b) lacks specific binding to the cellular target that is not bound to the exogenous molecule (unbound target). In another aspect the disclosure provides an isolated polypeptide comprising an antigen binding unit, wherein the antigen binding unit (a) exhibits specific binding to an intracellular target or an intracellular portion of a target, which target being bound by an exogenous molecule (bound target), but (b) lacks specific binding to the intracellular target or the intracellular portion of the target, which is not bound to the exogenous molecule (unbound target).

In another aspect, the present disclosure provides a multivalent antigen binding unit comprising a first binding domain and a second binding domain, wherein the first binding domain exhibits (a) specific binding to a cellular target covalently bound by an exogenous molecule (bound target), but (b) lacks specific binding to the cellular target that is not bound to the exogenous molecule (unbound target); and the second antigen binding domain comprises a functional unit capable of modulating one or more cellular functions including apoptosis, cell proliferation, cell differentiation, cell migration, cytotoxicity, release or trafficking of intercellular molecules, growth factor, metabolite, chemical compound, or a combination thereof. In a separate but related aspect, the disclosure provides a multivalent antigen binding unit comprising a first and a second binding domain, wherein the first binding domain exhibits (a) specific binding to an intracellular target or an intracellular portion of a target, which target being bound by an exogenous molecule (bound target), but (b) lacks specific binding to the intracellular target or the intracellular portion of the target, which is not bound to the exogenous molecule (unbound target); and the second antigen binding domain comprises a functional unit capable of modulating one or more cellular functions including apoptosis, cell proliferation, cell differentiation, cell migration, cytotoxicity, release or trafficking of intercellular molecules, growth factor, metabolite, chemical compound, or a combination thereof.

The tumor associate polypeptide to which the exogenous molecule binds can be any polypeptide (full length or a fragment thereof) whose expression and/or activity is associated with a tumor or cancerous cell. In some embodiments, the tumor associated polypeptide comprises Ras, EGFR, FGFR, PI3Kinase, BTK, Her2. Tumor associated polypeptides encompass any other tumor associated polypeptides known in the art or disclosed herein. Of particular interest are a K-ras polypeptide having a G to C mutation at residue 12, or N-ras polypeptide having a G to C mutation at the corresponding residue, or H-ras polypeptide having a G to C mutation at the corresponding residue.

The exogenous molecule can be is a modulator that activates or inhibits an activity of a cellular target of interest. In some embodiments, the exogenous molecule is a small molecule that covalently binds to the target. In some embodiment, the exogenous molecule comprises a Ras inhibitor, an EGFR inhibitor, an FGFR inhibitor, a PI3Kinase inhibitor, a BTK inhibitor, a Her2 inhibitor, or inhibitor of any cellular target disclosed herein. In some embodiments, the exogenous molecule is a small molecule capable of covalently binding to and inhibiting an activity of the target. In some embodiment, the exogenous molecule induces formation of an epitope upon covalently binding to said target. In some embodiment, the induced epitope is part of a binding pocket induced by binding of the target to the small molecule. In some embodiments, the induced epitope is representative of a neoantigen. A neoantigen can be unique to the tumor microenvironment and/or can be formed in response to an administration of the exogenous molecule to a cancer subject.

In some embodiment, a subject antigen binding unit comprises a whole antibody or a fragment thereof, including without limitation a Fab, F(ab′)2, a single chain variable fragment (scFv), a variable fragment (Fv), a single-unit antibody (SdAb), a minibody, a diabody, and a camelid antibody. In some embodiment, a subject antigen binding unit binds to a switch unit of K-ras that comprises one or more residues selected from the group consisting of cysteine 12, K16, D69, M72, Y96, and Q99.

In some embodiment, a subject polypeptide further comprises a functional unit that mediates a biological function in addition to the binding capability of the antigen binding unit. Such function unit may mediate apoptosis, cell proliferation, cell differentiation, cell migration, cytotoxicity, release or trafficking of intercellular molecules, growth factor, metabolite, chemical compound and/or a combination thereof. In some embodiments, the functional unit comprises a cytokine, a chemokine, a radioisotope, a fluorophore, or a toxin, or a binding unit exhibits specific binding to an immune cell antigen, a cytokine, a chemokine, a radioisotope, a fluorophore, or a toxin. In some embodiments, the binding of the functional unit to the immune cell antigen modulates an activity of the immune cell selected from the group consisting of: cytokine release; cytotoxicity of the immune cell; proliferation of the immune cell; differentiation, dedifferentiation or transdifferentiation of the immune cell; clonal expansion of the immune cell; trafficking of the immune cell; exhaustion and/or reactivation of the immune cell; and a combination thereof. Where desired, the functional unit comprises a binding unit exhibits specific binding to a cluster of differentiation 3 (CD3) polypeptide expressed on an immune cell. The CD3 polypeptide can an epsilon chain, a delta chain, and/or a gamma chain of CD3.

In some embodiment, a function unit comprises another binding agent capable of specific binding to an antigen distinct from the cellular target. In some embodiments, the antigen is selected from the group consisting of PDL1, TNF beta, CD2, CD3, CD5, CD7, and CD137. In some embodiments, the function unit is capable of binding to an immune cell antigen including without limitation a check point antigen selected from the group consisting of PD1, Siglec-15 (S15), CTLA-4, LAG3, TIM3, TIGIT, OX40, and CD93.

Provide here are multivalent antigen binding units, which can be bivalent, trivalent, tetra-valent or more. Where desired, the first and/or second antigen binding domains in the multivalent antigen binding unit can be conjugated to a label. In some embodiments, the first antigen binding domain exhibits specific binding to a tumor associated polypeptide, and the second antigen binding domain exhibits binding to a cell antigen that mediates one or more of the following selected from cytokine release, cytotoxicity of the immune cell, proliferation of the immune cell, differentiation, dedifferentiation or transdifferentiation of the immune cell, clonal expansion of the immune cell, trafficking of the immune cell, exhaustion and/or reactivation of the immune cell, and a combination thereof, or vice versa. In some embodiments, the first antigen binding domain exhibits specific binding to a tumor associated polypeptide selected from the group consisting of Ras, EGFR, FGFR, PI3Kinase, BTK, and Her2. In some other embodiments, the first antigen binding domain exhibits specific binding to Ras, EGFR, FGFR, PI3Kinase, BTK, and Her2 bound by the exogenous molecule, wherein the exogenous molecule is capable of covalently binding to and inhibiting an activity of Ras, EGFR, FGFR, PI3Kinase, BTK, and Her2, and wherein the second antigen binding domain exhibits specific binding a cell antigen selected from the group consisting of PDL1, TNF beta, CD2, CD3, CD5, CD7, CD137, PD1, Siglec-15 (S15), CTLA-4, LAG3, TIM3, TIGIT, OX40, and CD93, or vice versa. In some embodiments, the first antigen binding domain exhibits specific binding to Ras, EGFR, FGFR, PI3Kinase, BTK, or Her2 bound by a respective covalent inhibitor, and the second antigen binding domain exhibits specific binding to a check point antigen selected from the group consisting of Siglec-15 (S15), PD1, CTLA-4, LAG3, TIM3, TIGIT, OX40, cluster of differentiation 93 (CD93), ADORA2A, cluster of differentiation 276 (CD276), VTCN1, BTLA, IDO1, KIR3DL1, VISTA, cluster of differentiation 244 (CD244), CISH, HPRT1, AAVS1, CCR5, CD160, cluster of differentiation 96 (CD96), cluster of differentiation 355 (CD355), SIGLEC7, SIGLEC9, TNFRSF10A, TNFRSF10B, CASP3, CASP6, CASP7, CASP8, CASP10, FADD, FAS, TGFBRII, TGFBRI, SMAD2, SMAD3, SMAD4, SKI, SKIL, TGIF1, IL10RA, IL10RB, CSK, PAG1, EGLN3, or combinations thereof. In some embodiment, the second antigen binding domain exhibits specific binding to an immune cell antigen expressed by B cells, T cells, NK cells, KHYG cells, and/or hematopoietic stem cells. Ins some embodiments, the second antigen binding domain exhibits specific binding to a CD3 polypeptide, which include without limitation an epsilon chain, a delta chain, and/or a gamma chain of CD3. In some embodiments, the first antigen binding domain exhibits specific binding to Ras bound by a small molecule covalent inhibitor, and the second antigen binding domain exhibits specific binding to an epsilon chain of CD3.

In yet another aspect, the present disclosure provides a chimeric antigen receptor (CAR) or a T cell receptor, comprising a polypeptide (including multivalent antigen binding units) disclosed herein.

In still yet another aspect, the present disclosure provides a modified immune cell comprising one or more chimeric antigen receptors (CARs) or TCRs disclosed herein. In some embodiments, the CAR comprises comprising an antigen binding unit, wherein said binding unit comprises: (a) a first antigen binding domain (i) exhibiting specific binding to a cellular target covalently bound by an exogenous molecule (bound target), but lacks specific binding to the cellular target that is not bound to the exogenous molecule (unbound target), or (ii) exhibiting specific binding to an intracellular target or an intracellular portion of a target, which target being bound by an exogenous molecule (bound target), but lacks specific binding to the intracellular target or the intracellular portion of the target, which is not bound to the exogenous molecule (unbound target); and (b) a second antigen binding domain exhibiting specific binding to an immune cell antigen, and wherein each CAR of said one or more CARs further comprises a transmembrane unit and an intracellular region comprising an immune cell signaling unit.

In some embodiments, a modified immune cell comprising one or more T cell receptors (TCR) comprising an antigen binding unit, wherein said binding unit comprises: (a) a first antigen binding domain (i) exhibiting specific binding to a cellular target covalently bound by an exogenous molecule (bound target), but lacks specific binding to the cellular target that is not bound to the exogenous molecule (unbound target), or (ii) exhibiting specific binding to an intracellular target or an intracellular portion of a target, which target being bound by an exogenous molecule (bound target), but lacks specific binding to the intracellular target or the intracellular portion of the target, which is not bound to the exogenous molecule (unbound target); and (b) a second antigen binding domain exhibiting specific binding to an immune cell antigen, and wherein each TCR of said one or more TCRs further comprises a transmembrane unit and an intracellular region comprising an immune cell signaling unit. In some embodiments, the immune cell signaling unit of the receptor polypeptide comprises a primary signaling unit comprising an immunoreceptor tyrosine-based activation motif (ITAM). In some embodiments, the immune cell signaling unit comprises a primary signaling unit of a protein selected from the group consisting of: an Fcγ receptor (FcγR), an Fcε receptor (FcεR), an Fcα receptor (FcαR), neonatal Fc receptor (FcRn), CD3, CD3 ζ, CD3 γ, CD3 δ, CD3 ε, CD4, CD5, CD8, CD21, CD22, CD28, CD32, CD40L (CD154), CD45, CD66d, CD79a, CD79b, CD80, CD86, CD278 (also known as ICOS), CD247 ζ, CD247 η, DAP10, DAP12, FYN, LAT, Lck, MAPK, MHC complex, NFAT, NF-κB, PLC-γ, iC3b, C3dg, C3d, and Zap70. Where desired, the primary signaling unit comprises a CD3 ζ signaling unit, or an ITAM) of CD3 ζ. In some embodiment, the immune cell signaling unit comprises a co-stimulatory unit. Non-limiting co-stimulatory unit comprises a signaling unit of a MHC class I molecule, a TNF receptor protein, an immunoglobulin-like protein, a cytokine receptor, an integrin, a signaling lymphocytic activation molecule (SLAM protein), an activating NK cell receptor, or a Toll ligand receptor. Other suitable co-stimulatory unit comprises a signaling unit of a molecule selected from the group consisting of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8 α, CD8 β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6.

In some embodiment, a subject modified immune cell comprises an enhancer moiety capable of enhancing one or more activities of said engineered immune cell. Encompassed are enhancer moieties selected from the group consisting of IL-2, IL-3, IL-4, IL-6, IL-7, IL-10, IL-11, IL-12, IL-15, IL-17, IL-18, IL-21, IL-23, PD-1, PD-L1, CD122, CSF1R, CTAL-4, TIM-3, TGFRbeta, receptors for the same, functional fragments thereof, functional variants thereof, and combinations thereof. In some embodiments, expression or activity of an endogenous TCR is reduced in a subject modified immune cell. In some embodiments, a subject modified immune cell comprises an inducible cell death moiety, which inducible cell death moiety effects suicide of said modified immune cell upon contact with a cell death activator. Where desired, an inducible cell death moiety is selected from the group consisting of rapaCasp9, iCasp9, HSV-TK, ΔCD20, mTMPK, ΔCD19, RQR8, and EGFRt. Where desired, a suitable inducible cell death moiety can be HSV-TK, and the cell death activator is GCV. Where further desired, a suitable inducible cell death moiety can be iCasp9, and the cell death activator is AP1903.

Also provided in the present disclosure is a method of treating cancer in a subject in need thereof comprising: administering to the subject a subject polypeptide disclosed herein. In some embodiment the polypeptide is a multivalent antigen binding unit disclosed herein. In some embodiment, the subject has been exposed to the exogenous molecule, e.g., a covalent inhibitor of a target disclosed herein.

Further provided in the present disclosure is a cell therapy, comprising administering to a subject in need thereof a population of cells comprising a subject modified immune cell as disclosed herein, wherein the subject has been exposed to the covalent inhibitor specific for the target.

Provided also is a method of targeting an intracellular target or an intracellular portion of a target in a subject comprising: (a) administering to the subject an exogenous molecule that covalently binds to the target or the intracellular portion of a target; and (b) administering to the subject a subject polypeptide, and/or the multivalent antigen binding unit disclosed herein, wherein an epitope to which the polypeptide or the multivalent antigen binding unit binds is accessible for said binding, thereby targeting the intracellular target or the intracellular portion of the target. In some embodiments, provided is a method of targeting an intracellular target or an intracellular portion of a target in a subject comprising: administering to the subject a polypeptide comprising an antigen binding unit, wherein the antigen binding unit: (a) exhibits specific binding to an intracellular target or an intracellular portion of a target, which target being covalently bound (bound target) by an exogenous molecule that is a covalent inhibitor of the target; and (b) lacks specific binding to the intracellular target or the intracellular portion of the target, which is not bound to the exogenous molecule (unbound target); wherein the subject has been exposed to the covalent inhibitor that covalently binds to the intracellular target or the intracellular portion of the target to induce formation of an epitope upon covalently binding to said target or the intracellular portion, and wherein the epitope becomes accessible to said antigen binding unit upon cell death. The target being targeted is a tumor associated polypeptide including but not limited to a cell surface protein. Where desired, the antigen binding unit being utilized comprises a functional unit that mediates apoptosis, cell proliferation, cell differentiation, cell migration, cytotoxicity, release or trafficking of intercellular molecules, growth factor, metabolite, chemical compound, or a combination thereof. Exemplary antigen binding unit comprising a functional unit can be one having a cytokine, a chemokine, a radioisotope, a fluorophore, a toxin, or a binding unit exhibits specific binding to an immune cell antigen. Where desired, the functional unit can binds to an immune cell antigen and modulates an activity of the immune cell selected from the group consisting of: cytokine release; cytotoxicity of the immune cell; proliferation of the immune cell; differentiation, dedifferentiation or transdifferentiation of the immune cell; clonal expansion of the immune cell; trafficking of the immune cell; exhaustion and/or reactivation of the immune cell; and a combination thereof. Of particular interest is an antigen binding unit comprising a functional unit that exhibits specific binding to a cluster of differentiation 3 (CD3) polypeptide expressed on an immune cell (including but not limited to an epsilon chain, a delta chain, and/or a gamma chain of CD3), PDL1, TNF beta, CD2, CD3, CD5, CD7, CD137, PD1, Siglec-15 (S15), CTLA-4, LAG3, TIM3, TIGIT, OX40, and/or CD93.

In another aspect, the present disclosure provides a method of labeling a tumor cell comprising: (a) contacting the tumor cell with a covalent inhibitor; and (b) contacting the tumor cell with a subject polypeptide, and/or subject multivalent antigen binding unit, wherein an epitope to which the polypeptide or the multivalent antigen binding unit binds is accessible for said binding, thereby labeling said tumor cell. In some embodiment, an epitope to which the polypeptide or the multivalent antigen binding unit binds is accessible as evidenced by or as a result of cell death. In some embodiments, the binding of the exogenous molecule to the cellular target is associated with, or otherwise causing cell death or apoptosis.

In yet another aspect, the present disclosure provides a method of treating cancer in a subject in need thereof comprising: administering to the subject a polypeptide comprising an antigen binding unit, wherein the antigen binding unit: (a) exhibits specific binding to an intracellular portion of a target, which target being covalently bound (bound target) by an exogenous molecule that is a covalent inhibitor of the target; and (b) lacks specific binding to the intracellular portion of the target, which is not bound to the exogenous molecule (unbound target); wherein the subject has been exposed to the covalent inhibitor that covalently binds to the intracellular portion of the target to induce formation of an epitope upon covalently binding to the intracellular portion thereof, and wherein the epitope becomes accessible to said antigen binding unit upon death of cancer cells comprising said target, and further wherein the covalent inhibitor is a compound selected from the group consisting of Osimertinib, Afatinib, Dacomitinib, and Neratinib. The structures of these molecules are shown as follows:

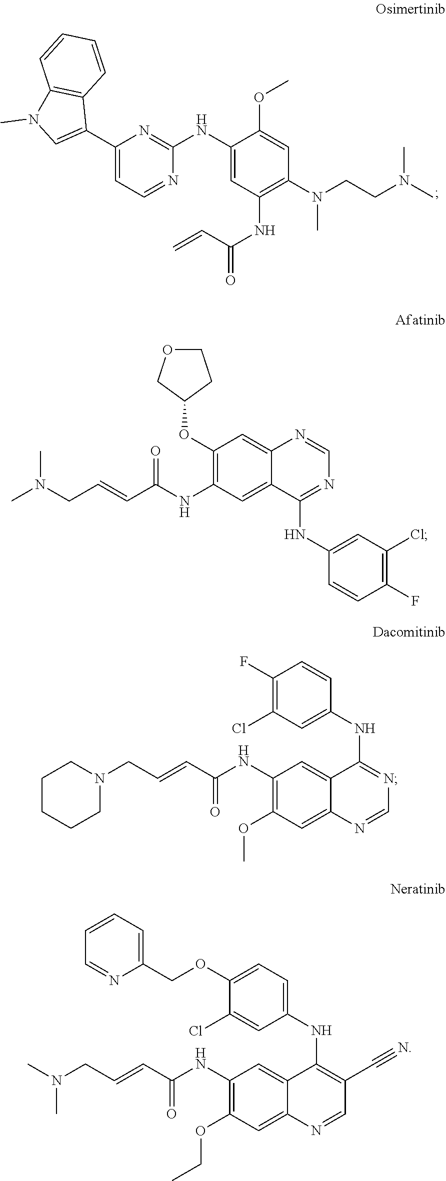

In some embodiments, a subject being treated is exposed to a therapy that causes death of the cancer cells and exposes the epitope to which the antigen binding unit specifically binds. In some instances, the epitope is accessible only upon cell death. For example, the subject is exposed to chemotherapy, radiation, cell therapy, or a combination thereof. In some embodiment, death of cancer cells occurs upon administering the covalent inhibitor to said subject. For instance, the exogenous molecule (including but not limited to a covalent inhibitor itself when administered to a subject induces death of cancer cells). In some embodiments, the subject is administered a therapy simultaneously, concurrently or sequentially with administering the polypeptide comprising the antigen binding unit, wherein the therapy causes death of cancer cells. In some embodiments, the subject is administered a therapy prior to administering the polypeptide comprising the antigen binding unit, wherein the therapy causes death of cancer cells. Where desired, the the intracellular portion of the target chosen comprises the intracellular portion of a receptor (e.g., a receptor kinase including but not limited to EGFR, PDGF, and FGF). Where desired, the polypeptide administered comprises a multivalent antigen binding unit disclosed herein. Where also desired, the polypeptide administered to the subject is incorporated into a CAR or chimeric TCR that is in turn administered into the subject.

In some embodiments, the treatment, targeting or labeling methods apply to a subject suffering from a hematological or a solid cancer. Various types of cancer can be treated including without limitation: chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL). In some embodiments, the lymphoma is mantle cell lymphoma (MCL), T cell lymphoma, Hodgkin's lymphoma, and/or non-Hodgkin's lymphoma, nephroblastoma, Ewing's sarcoma, neuroendocrine tumor, glioblastoma, neuroblastoma, melanoma, skin cancer, breast cancer, colon cancer, rectal cancer, prostate cancer, liver cancer, kidney cancer, pancreatic cancer, lung cancer, biliary tract cancer, cervical cancer, endometrial cancer, esophageal cancer, gastric cancer, head and neck cancer, medullary thyroid carcinoma, ovarian cancer, glioma, or bladder cancer. In some embodiments, the subject is exposed to chemotherapy, radiation, cell therapy, or a combination thereof.

In yet another aspect, the present disclosure provides a method of developing a subject polypeptide disclosed herein. The method typically comprises: (a) contacting a plurality of antigen binding units with an intracellular target or an intracellular portion of a target, which is covalently bound by an exogenous molecule capable of specific and covalent binding to said target (bound target); (b) selecting an antigen binding unit from said plurality, said selected antigen binding unit exhibits specific binding to the bound target, but not the same target without being bound to the exogenous molecule (unbound target), thereby developing the polypeptide. Any of the exogenous molecules disclosed herein can be utilized for development of a subject polypeptide. In some embodiments, the plurality of antigen binding units are presented on a cell, a phage, a surface, or in solution.

Also provided is a complex comprising: (a) a modified intracellular target or a modified intracellular portion of a target in a cell, (b) an exogenous molecule, and (c) a polypeptide comprising an antigen binding unit, wherein the exogenous molecule is a covalent inhibitor of the target, and wherein the polypeptide comprising the antigen binding unit specifically binds to an epitope (i) formed by binding of said covalent inhibitor to said intracellular target or a modified intracellular portion of a target and (ii) becomes accessible upon death of the cell. In some embodiments, the antigen binding unit in the complex (a) exhibits specific binding to the intracellular target or the intracellular portion of the target covalently bound by an exogenous molecule (bound target), but (b) lacks specific binding to the intracellular target or the intracellular portion of the target that is not bound to the exogenous molecule (unbound target). In some embodiments, the target in the complex is a tumor associated polypeptide or any other target disclosed herein. For instance, the target is an EGFR bound by a covalent inhibitor of EGFR, and a polypeptide comprising an antigen binding unit that exhibits specific binding to the EGFR bound by said covalent inhibitor. In some embodiments, the complex is present in a dead cell. In some embodiment, complex is detectable in a tumor undergoing necrosis.

The exogenous molecules as applied to any of the compositions or methods disclosed herein (including but not limited to methods for developing a subject polypeptide (comprising a subject antigen binding unit disclosed herein) or a cell comprising the same, or methods of using the polypeptides and cells), can have the structure: R-L-E; wherein: R is a target binding moiety; L is a bond or a divalent radical chemical linker; and E is an electrophilic chemical moiety capable of forming a covalent bond with a nucleophile. In some embodiment, R is an optionally substituted monocyclic heteroaryl ring, an optionally substituted bicyclic aryl ring, an optionally substituted monocyclic aryl ring, or an optionally substituted bicyclic aryl ring. In some embodiment, E is an electrophilic group capable of forming a covalent bond with a cysteine residue of a protein, or an electrophilic group capable of forming a covalent bond with an aspartate residue of a protein. In some embodiments, E is an electrophilic group capable of forming a covalent bond with a cysteine residue or an aspartate residue of a Ab1, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, KDR, Kit, Lck, Lyn, MAP2K1, MAP2K2, MAP4K4, MAPKAPK2, Met, Mnk1, MLK1, p38, PDGFRA, PDGFRB, PDPK1, PI3Kinase, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, or Zap70 protein. In some embodiments, E is an electrophilic group capable of forming a covalent bond with a cysteine residue or an aspartate residue of a RAS, EGFR, Her2, BTK2, FGFR, or PI3Kinase protein. In other embodiments, E is an electrophilic group capable of forming a covalent bond with a cysteine residue or an aspartate residue of RAS, KRAS, HRAS, NRAS, KRAS G12C, KRAS G12D, HRAS G12C, NRAS G12C, EGFR, EGFR del E746-A750, EGFR del E747-E749/A750P, EGFR del E747-S752/P753S, EGFR del E747-T751/Sins/A750P, EGFR del 5752-1759, EGFR G719S, EGFR G719C, EGFR L861Q, EGFR L858R, EGFR T790M, EGFR L858R/T790M, Her2, BTK2, FGFR, or PI3Kinase protein.

In some embodiment, the exogenous molecule has a structure represented by:

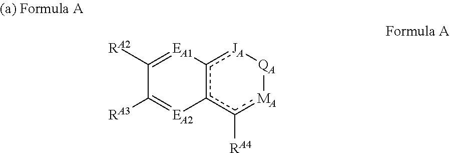

wherein:
E_{A1} and E_{A2} are each independently N or CR^{A1};
J_{A} is N, NR^{A10} or CR^{A10};
M_{A} is N, NR^{A13} or CR^{A13};
[symbol] is a single or double bond as necessary to give every atom its normal valence;
R^{A1} is independently H, hydroxy, C₁₋₄alkyl, C₁₋₄haloalkyl, C₁₋₄alkoxy, —NH—C₁₋₄alkyl, —N(C₁₋₄alkyl)₂, cyano, or halo;
R^{A1} is halo, C₁₋₆alkyl, C₁₋₆haloalkyl, —OR^{A′}, —N(R^{A′})₂, C₂₋₃alkenyl, C₂₋₃alkynyl, C₀₋₃alkylene-C₃₋₁₄cycloalkyl, C₀₋₃alkylene-C₂₋₁₄heterocycloalkyl, aryl, heteroaryl, C₀₋₃alkylene-C₆₋₁₄aryl, or C₀₋₃alkylene-C₂₋₁₄heteroaryl, and each R^{A′} is independently H, C₁₋₆alkyl, C₁₋₆haloalkyl, C₃₋₁₄cycloalkyl, C₂₋₁₄heterocycloalkyl, C₂₋₃alkenyl, C₂₋₃alkynyl, aryl, or heteroaryl, or two R^{A′} substituents, together with the nitrogen atom to which they are attached, form a 3-7-membered ring;
R^{A3} is halo, C₁₋₃alkyl, C₁₋₂haloalkyl, C₁₋₃alkoxy, C₃₋₄cycloalkyl, C₂₋₃alkenyl, C₂₋₃alkynyl, aryl, or heteroaryl;
R^{A4} is

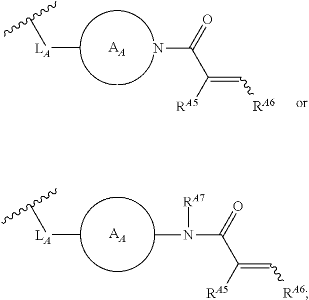

Ring A_{A} is a monocyclic 4-7 membered ring or a bicyclic, fused, or spiro 6-11 membered ring;
L_{A} is a bond, C₁₋₆alkylene, —O—C₀₋₅alkylene, —S—C₀₋₅alkylene, or —NH—C₀₋₅alkylene, and for C₂₋₆alkylene, —O—C₂₋₅alkylene, —S—C₂₋₅alkylene, and —NH—C₂₋₅alkylene, one carbon atom of the alkylene group can optionally be replaced with O, S, or NH;
R^{A5} and R^{A6} are each independently H, halo, C₁₋₆alkyl, C₂₋₆alkynyl, C₁₋₆alkylene-O—C₁₋₄alkyl, C₁₋₆alkylene-OH, C₁₋₆haloalkyl, C₁₋₆alkyleneamine, C₀₋₆alkylene-amide, C₀₋₃alkylene-C(O)OH, C₀₋₃alkylene-C(O)OC₁₋₄alkyl, C₁₋₆alkylene-O-aryl, C₀₋₃alkylene-C(O)C₁₋₄alkylene-OH, cycloalkyl, heterocycloalkyl, aryl, heteroaryl, C₀₋₃alkylene-C₃₋₁₄cycloalkyl, C₀₋₃alkylene-C₂₋₁₄heterocycloalkyl, C₀₋₃alkylene-C₆₋₁₄ aryl, C₀₋₃alkylene-C₂₋₁₄heteroaryl, or cyano, or R^{A5} and R^{A6}, together with the atoms to which they are attached, form a 4-6 membered ring;
R^{A7} is H or C₁₋₈alkyl, or R^{A7} and R^{A5}, together with the atoms to which they are attached, form a 4-6 membered ring;
Q_{A} is CR^{A8}R^{A9}, C═CR^{A8}R^{A9}, C═O, C═S, or C═NR^{A8};
R^{A8} and R^{A9} are each independently H, C₁₋₃alkyl, hydroxy, C₁₋₃alkoxy, cyano, nitro, or C₃₋₆cycloalkyl, or R^{A8} and R^{A9}, taken together with the carbon atom to which they are attached, can form a 3-6 membered ring; and
R^{A10} is C₁₋₈alkyl, C₀₋₃alkylene-C₆₋₁₄aryl, C₀₋₃alkylene-C₃₋₁₄heteroaryl, C₀₋₃alkylene-C₃₋₁₄cycloalkyl, C₀₋₃alkylene-C₂₋₁₄heterocycloalkyl, C₁₋₆alkoxy, —O—C₀₋₃alkylene-C₆₋₁₄aryl, —O—C₀₋₃alkylene-C₃₋₁₄heteroaryl, —O—C₀₋₃alkylene-C₃₋₁₄cycloalkyl, —O—C₀₋₃alkylene-C₂₋₁₄heterocycloalkyl, —NH—C₁₋₈alkyl, —N(C₁₋₈alkyl)₂, —NH—C₀₋₃alkylene-C₆₋₁₄aryl, —NH—C₀₋₃alkylene-C₃₋₁₄heteroaryl, —NH—C₀₋₃alkylene-C₃₋₁₄cycloalkyl, —NH—C₀₋₃alkylene-C₂₋₁₄heterocycloalkyl, halo, cyano, or C₁₋₆alkylene-amine;
or

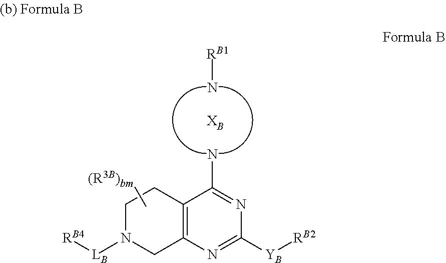

wherein:
X_{B} is a 4-12 membered saturated or partially saturated monocyclic, bridged or spirocyclic ring, wherein the saturated or partially saturated monocyclic ring is optionally substituted with one or more R^{B8};
Y_{B} is a bond, O, S, or NR^{B5};
R^{B1} is —C(O)C(R^{BA}) [symbol] C(R^{BB})_{bp} or —S(O)₂C(R^{BA}) [symbol] C(R^{BB})_{bp};
R^{B2} is hydrogen, alkyl, hydroxyalkyl, dihydroxyalkyl, alkylaminylalkyl, dialkylaminylalkyl, —Z_{B}—NR^{B5}R^{B10}, heterocyclyl, heterocyclylalkyl, aryl, heteroaryl, or heteroarylalkyl, wherein each of the Z_{B}, heterocyclyl, heterocyclylalkyl, aryl, heteroaryl, and heteroarylalkyl may be optionally substituted with one or more R^{B9};
Z_{B} is C₁-C₄ alkylene;
each R^{B3} is independently C₁-C₃ alkyl, oxo, or haloalkyl;
L_{B} is a bond, —C(O)—, or C₁-C₃ alkylene;
R^{B4} is hydrogen, cycloalkyl, heterocyclyl, aryl, aralkyl, or heteroaryl, wherein each of the cycloalkyl, heterocyclyl, aryl, aralkyl, and heteroaryl may be optionally substituted with one or more R^{B6} or R^{B7};
each R^{B5} is independently hydrogen or C₁-C₃ alkyl;
R^{B6} is cycloalkyl, heterocyclyl, heterocyclylalkyl, aryl, or heteroaryl, wherein each of the cycloalkyl, heterocyclyl, aryl, or heteroaryl may be optionally substituted with one or more R^{B7};
each R^{B7} is independently halogen, hydroxyl, C₁-C₆ alkyl, cycloalkyl, alkoxy, haloalkyl, amino, cyano, heteroalkyl, hydroxyalkyl, or -Q_{B}-haloalkyl, wherein Q_{B} is O or S;
R^{B8} is oxo, C₁-C₃ alkyl, C₂-C₄ alkynyl, heteroalkyl, cyano, —C(O)OR^{B5}, —C(O)N(R^{B5})₂, or —N(R^{B5})₂, wherein the C₁-C₃ alkyl may be optionally substituted with cyano, halogen, —OR^{B5}, —N(R^{B5})₂, or heteroaryl;
each R^{B9} is independently hydrogen, oxo, acyl, hydroxyl, hydroxyalkyl, cyano, halogen, C₁-C₆ alkyl, aralkyl, haloalkyl, heteroalkyl, cycloalkyl, heterocyclyl, heterocyclylalkyl, alkoxy, dialkylaminyl, dialkylamidoalkyl, or dialkylaminylalkyl, wherein the C₁-C₆ alkyl may be optionally substituted with cycloalkyl;
each R^{B10} is independently hydrogen, acyl, C₁-C₃ alkyl, heteroalkyl, or hydroxyalkyl;
R^{BA} is absent, hydrogen, or C₁-C₃ alkyl;
each R^{BB} is independently hydrogen, C1-C3 alkyl, alkylaminylalkyl, dialkylaminylalkyl, or heterocyclylalkyl;
bm is 0, 1, or 2; and
bp is 1 or 2;
wherein when [symbol] is a triple bond then R^{BA} is absent, R^{BB} is present, and bp is 1,
and wherein when [symbol] is a double bond then R^{BA} is present, R^{BB} is present, and bp is 2, or R^{BA}, R^{BB} and the carbon atoms to which they are attached form a 5-8 membered partially saturated cycloalkyl optionally substituted with one or more R^{B7};
or

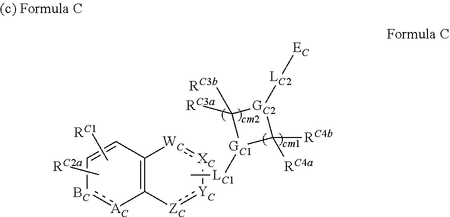

wherein:
A_{C} is CR1, CR^{C2b}, NR^{C7} or S;
B_{C} is a bond, CR^{C1} or CR^{C2c};
G_{C1} and G_{C2} are each independently N or CH;
W_{C}, X_{C} and Y_{C} are each independently N, NR^{C5} or CR^{C6};
Z_{C} is a bond, N or CR^{C6}, or Z_{C} is NH when Y is C═O;
L_{C1} is a bond or NR^{C7};
L_{C2} is a bond or alkylene;
R1 is H, cyano, halo, —CF₃, C₁-C₆alkyl, C₁-C₈alkylaminyl, C₃-C₈cycloalkyl, C₂-C₆alkenyl, or C₃-C₈cycloalkenyl, heterocyclyl, heteroaryl, aryloxy, heteroaryloxy, or aryl;
R^{C2a}, R^{C2b}, and R^{C2c} are each independently H, halo, hydroxyl, C₁-C₆alkyl, C₁-C₆haloalkyl, C₁-C₆alkoxy, C₃-C₈cycloalkyl, heteroaryl or aryl;
R^{C3a} and R^{C3b} are, at each occurrence, independently H. —OH, —NH₂, —CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl; or R^{C3a} and R^{C3b} join to form a carbocyclic or heterocyclic ring; or R^{C3a} is H, —OH, —NH₂, —CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl, and R^{C3b} joins with R^{C4b} to form a carbocyclic or heterocyclic ring;
R^{C4a} and R^{C4b} are, at each occurrence, independently H. —OH, —NH₂, CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl: or R^{C4a} and R^{C4b} join to form a carbocyclic or heterocyclic ring; or R^{C4a} is H, —OH, —NH₂, —CO₂H, halo, cyano, C₁-C₆alkyl, C₁-C₆alkynyl, hydroxylalkyl, aminylalkyl, alkylaminylalkyl, cyanoalkyl, carboxyalkyl, aminylcarbonylalkyl or aminylcarbonyl, and R^{C4b} joins with R^{C3b} to form a carbocyclic or heterocyclic ring;
R^{C5} is, at each occurrence, independently H, C₁-C₆alkyl or a bond to L_{C1};
R^{C6} is, at each occurrence, independently H, oxo, cyano, cyanoalkyl, amino, aminylalkyl, aminylalkylaminyl, aminylcarbonyl, aminylsulfonyl, —CO₂NR^{Ca}R^{Cb}, wherein R^{Ca} and R^{Cb}, are each independently H or C₁-C₆alkyl or R^{Ca} and R^{Cb} join to form a carbocyclic or heterocyclic ring, alkylaminyl, haloalkylaminyl, hydroxylalkyaminyl, amindinylalkyl, amidinylalkoxy, amindinylalkylaminyl, guanidinylalkyl, guanidinylalkoxy, guanidinylalkylaminyl, C₁-C₆alkoxy, aminylalkoxy, alkylcarbonylaminylalkoxy, C₁-C₆alkyl, heterocyclyl, heterocyclyloxy, heterocyclylalkyloxy, heterocyclylaminyl, heterocyclylalkylaminyl, heteroaryl, heteroaryloxy, heteroarylalkyloxy, heteroarylaminyl, heteroarylalkylaminyl, aryl, aryloxy, arylaminyl, arylalkylaminyl, arylalkyloxy or a bond to L_{C1};
R^{C7} is H or C₁-C₆alkyl;
cm1 and cm2 are each independently 1, 2, or 3;
[symbol] indicates a single or a double bond such that all valances are satisfied; and
E_{C} is an electrophilic moiety capable of forming a covalent bond with the cysteine residue at position 12 of a KRAS, HRAS, or NRAS G12C mutant protein;
wherein at least one of W_{C}, X_{C}, Y_{C}, and Z_{C}, is CR⁶ where R6 is a bond to L_{C1};
or

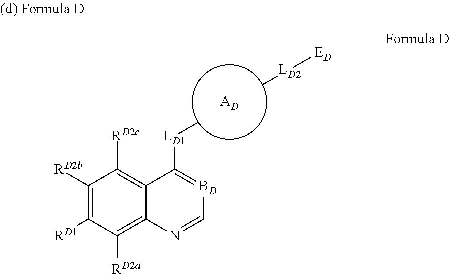

wherein:
A_{D} is a monocyclic or bicyclic moietyl;
B_{D} is N or CR^{D}′;
L_{D1} is a bond or NR^{D5};
L_{D2} is a bond or alkylene;
R^{D}′ is H, cyano, alkyl, cycloalkyl, amino, aminylakyl, alkoxy, alkoxualkyl, alkoxycarbonyl, aminylalkoxy, alkylaminylalkoxy, alkylaminyl, alkylaminylalkyl, aminylaklylaminyl, carboxyalkyl, alkylcarbonylaminyl, aminylcarbonyl, alkylaminylcarbonyl, or aminylcarbonylalkyl;
R^{D1} is aryl or heteroaryl;
R^{D2a}, R^{D2b} and R^{D2c} are each independently H, amino, halo, hydroxyl, cyano, C₁-C₆ alkyl, C₁-C₆ alkyl amino, C₁-C₆ haloalkyl (e.g., CF₃), C₁-C₆ alkoxy, C₁-C₆ haloalkoxy, C₃-C₈ cycloalkyl, heterocyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl; heteroaryl, or aryl;
R^{D5} is, at each occurrence, independently H, C₁-C₆ alkyl, C₃-C₈ cycloalkyl, or heterocyclcylalkyl; and
E_{D} is an electrophilic moiety capable of forming a covalent bond with the cysteine residue at position 12 of a KRAS, HRAS, or NRAS G12C mutant protein;
or

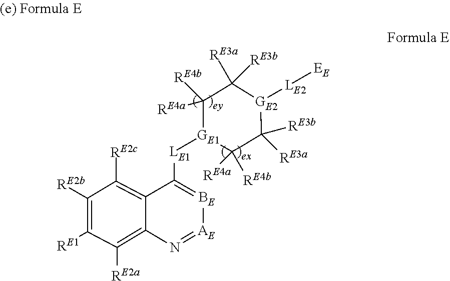

wherein:
A_{E} is N or CH;
B_{E} is N or CR^{E}′;
G^{E1} and G^{E2} are each independently N or CH;
L^{E2} is a bond or alkylene;
R^{E}′ is H, cyano, alkyl, cycloalkyl, amino, aminylalkyl, alkoxy, alkoxyalkyl, alkoxycarbonyl, aminylalkoxy, alkylaminylalkoxy, alkylaminyl, alkylaminylalkyl, aminylalkylaminyl, carboxyalkyl, alkylcarbonylaminyl, aminylcarbonyl, alkylaminylcarbonyl or aminylcarbonylalkyl;
R^{E1} is aryl or heteroaryl;
R^{E2a} and R^{E2b} are each independently amino, halo, hydroxyl, cyano, C₁-C₆ alkyl, C₁-C₆ alkyl amino, C₁-C₆ haloalkyl, C₁-C₆ alkoxy, C₁-C₆haloalkoxy, C₃-C₈ cycloalkyl, heterocycyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{E2c} is H, amino, halo, hydroxyl, cyano, C₁-C₆ alkyl, C₁-C₆ alkyl amino, C₁-C₆ haloalkyl, C₁-C₆ alkoxy, C₁-C₆ haloalkoxy, C₃-C₈ cycloalkyl, heterocycyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{E3a} and R^{E3b} are, at each occurrence, independently H, —OH, —NH₂, —CO₂H, halo, cyano, unsubstituted C₁-C₆ alkyl, C₁-C₆ haloalkyl, C₃-C₈ cycloalkyl, heterocyclylalkyl, C₁-C₆ haloalkyl, C₁-C₆ haloalkoxy, hydroxylalkly, alkoxyalkyl, aminylalkyl, carboxyalkyl, aminylcarbonylalkyl or aminylcarbonyl; or
R^{E3a} and R^{E3b} join to form oxo, a carbocyclic or heterocyclic ring; or R^{E3a} is H, —OH, —NH₂, —CO₂H, halo, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl, and R^{E3b} joins with R^{E4b} to form a carbocyclic or heterocyclic ring;
R^{E4a} and R^{E4b} are, at each occurrence, independently H, —OH, —NH₂, —CO₂H, halo, cyano, unsubstituted C₁-C₆ alkyl, C₁-C₆ haloalkyl, C₃-C₈ cycloalkyl, heterocyclylalkyl, C₁-C₆ haloalkyl, C₁-C₆ haloalkoxy, hydroxylalkly, alkoxyalkyl, aminylalkyl, carboxyalkyl, aminylcarbonylalkyl or aminylcarbonyl; or
R^{E4a} and R^{E4b} join to form oxo, a carbocyclic or heterocyclic ring; or R^{E4a} is H, —OH, —NH₂, —CO₂H, halo, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl, and R^{E4b} joins with R^{E3b} to form a carbocyclic or heterocyclic ring;
R^{E5} is, at each occurrence, independently H, C₁-C₆ alkyl, C₃-C₈cycloalkyl or heterocyclylalkyl;
ex and ey are independently integers ranging from 0 to 2; and
E_{E} is an electrophilic moiety capable of forming a covalent bond with the cysteine residue at position 12 of a KRAS, HRAS or NRAS G12C mutant protein;
or

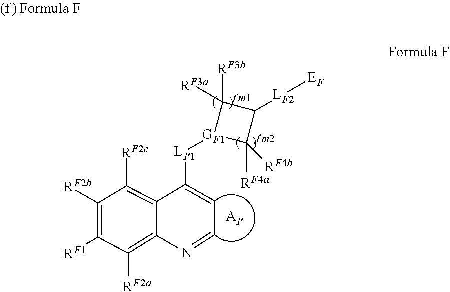

wherein:
A_{F} is a carbocyclic, heterocyclic or heteroaryl ring;
G_{F1} and G_{F2} are each independently N or CH;
L_{F1} is a bond or NR⁵;
L_{F2} is a bond or alkylene;
R^{F1} is aryl or heteroaryl;
R^{F2a}, R^{F2b} and R^{F2c} are each independently H, amino, halo, hydroxyl, cyano, C₁-C₆ alkyl, C₁-C₆ alkyl amino, C₁-C₆haloalkyl, C₁-C₆ alkoxy, C₁-C₆ haloalkoxy; C₃-C₈ cycloalkyl, heterocyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{F3a} and R^{F3b} are, at each occurrence, independently H, —OH, —NH₂, —CO₂H, halo, cyano, C₁-C₆ alkyl, C₁-C₆haloalkyl, C₁-C₆ haloalkoxy, C₃-C₈ cycloalkyl, heterocyclylalkyl, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl; or R^{F3a} and R^{F3b} join to form a carbocyclic or heterocyclic ring; or R^{F3a} is H, OH, NH₂, CO₂H, halo, cyano, C₁-C₆ alkyl, C₁-C₆ haloalkyl, C₁-C₆ haloalkoxy, C₃-C₈ cycloalkyl, heterocyclylalkyl, C₁-C₆alkynyl, hydroxylalkly, alkoxyalkyl, aminylalkyl, alkylaminylalkyl, cyanoalkyl, carboxyalkyl, aminylcarbonylalkyl or aminylcarbonyl, and R^{F3b} joins with R^{F4b} to form a carbocyclic or heterocyclic ring;
R^{F4a} and R^{F4b} are, at each occurrence, independently H, —OH, —NH₂, —CO₂H, halo, cyano, C₁-C₆ alkyl, C₁-C₆haloalkyl, C₁-C₆ haloalkoxy, C₃-C₈ cycloalkyl, heterocyclylalkyl, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl; or R^{F4a} and R^{F4b} join to form a carbocyclic or heterocyclic ring; or R^{F4a} is H, —OH, —NH₂, —CO₂H, halo, cyano, C₁-C₆ alkyl, C₁-C₆ haloalkyl, C₁-C₆ haloalkoxy, C₃-C₈ cycloalkyl, heterocyclylalkyl, C₁-C₆alkynyl, hydroxylalkly, alkoxyalkyl, aminylalkyl, alkylaminylalkyl, cyanoalkyl, carboxyalkyl, aminylcarbonylalkyl or aminylcarbonyl, and R^{F4b} joins with R^{F3b} to form a carbocyclic or heterocyclic ring;
R^{F5} is, at each occurrence, independently H, C₁-C₆ alkyl, C₃-C₈ cycloalkyl or heterocycloalkyl;
fm1 and fm2 are each independently 1, 2 or 3; and
E_{F} is an electrophilic moiety capable of forming a covalent bond with the cysteine residue at position 12 of a KRAS, HRAS or NRAS G12C mutant protein;
or

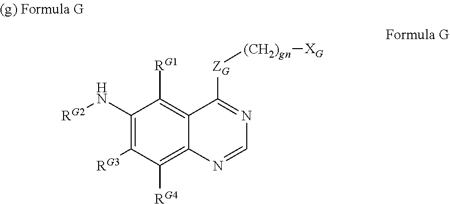

wherein:
X_{G} is cycloalkyl of 3 to 7 carbon atoms, which may be optionally substituted with one or more alkyl of 1 to 6 carbon atom groups, or is a pyridinyl, pyrimidinyl, or phenyl ring wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminomethyl, N-alkylaminomethyl of 2-7 carbon atoms, N,N-dialkylaminomethyl of 3-7 carbon atoms, mercapto, methylmercapto, and benzoylamino;
Z_{G} is —NH—, —O—, —S—, or —NR^{G}—;
R^{G} is alkyl of 1-6 carbon atoms, or carboalkyl of 2-7 carbon atoms;
R^{G1}, R^{G3}, and R^{G4} are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2-12 carbon atoms, N-alkylcarbamoyl, N,N-dialkylcarbamoyl, N-alkyl-N-alkenylamino of 4-12 carbon atoms, N,N-dialkenylamino of 6-12 carbon atoms, phenylamino, benzylamino, R^{G7}—(C(R^{G6})₂)_{gg}—Y_{G}—, R^{G7}—(C(R^{G6})₂)_{gp}-M_{G}-(C(R^{G6})₂)_{gk}—Y_{G}—, or Het_{G}-W_{G}—(C(R^{G6})₂)_{gk}—Y_{G}—;
Y_{G} is a divalent radical selected from the group consisting of —(CH₂)_{ga}—, —O—, and —NR^{G6}—;
R^{G7} is —NR^{G6}R^{G6} or —OR^{G6};
M_{G} is —N(R^{G6})—, —O—, —N[(C(R^{G6})₂)_{gp}—NR^{G6}R^{G6}]—, or —N[(C(R^{G6})₂)_{gp}—OR^{G6}]—;
W_{G} is —N(R^{G6})—, —O—, or a bond;
Het_{G} is a heterocycle, optionally mono- or di-substituted on carbon or nitrogen with R^{G6} and optionally mono-substituted on carbon with —CH₂OR^{G6}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran;
each R^{G6} is, independently, hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms;
R^{G2} is selected from the group consisting of

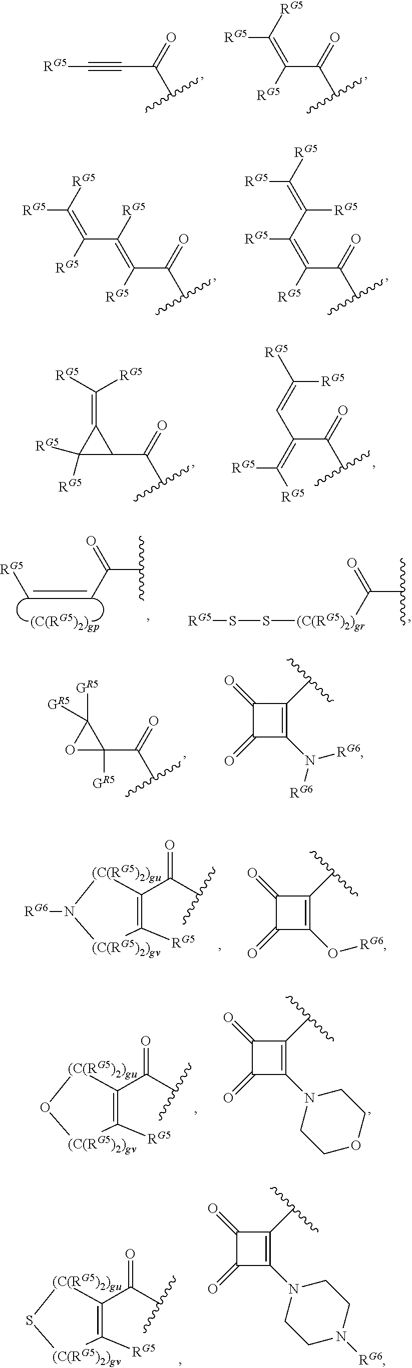

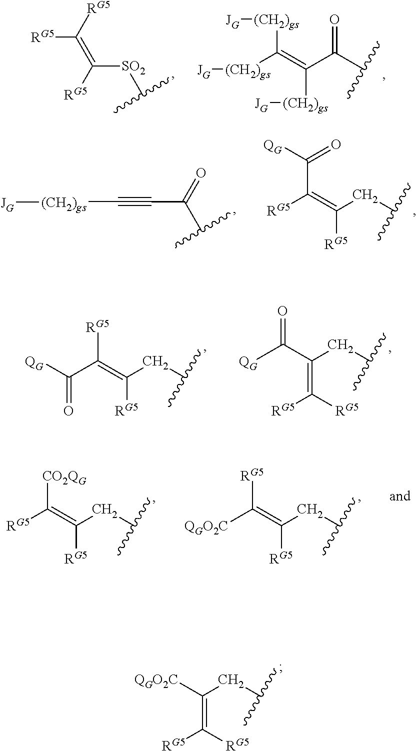

each R^{G} is independently hydrogen, alkyl of 1-6 carbon atoms, carboxy, carboalkoxy of 1-6 carbon atoms, phenyl, carboalkyl of 2-7 carbon atoms, R^{G7}—(C(R^{G6})₂)_{gs}—, R^{G7}—(C(R^{G6})₂)_{gp}-M_{G}-(C(R^{G6})₂)_{gr}—, (R^{G8})(R^{G9})CH-M_{G}-(C(R^{G6})₂)_{gr}—, or Het_{G}-W_{G}—(C(R^{G6})₂)_{gr}—;
R^{G5} and R^{G9} are each, independently, —(C(R^{G6})₂)_{gr}—NR^{G6}R^{G6}, or —(C(R^{G6})₂)_{gr}—OR^{G6};
J_{G} is independently hydrogen, chlorine, fluorine, or bromine;
Q_{G} is alkyl of 1-6 carbon atoms or hydrogen;
ga is 0 or 1;
gg is 1-6;
gk is 0-4;
gn is 0-1;
gp is 2-4;
gq is 0-4;
gr is 1-4;
gs is 1-6;
gu is 0-1; and
gv is 0-4, wherein the sum of gu+gv is 2-4;
or

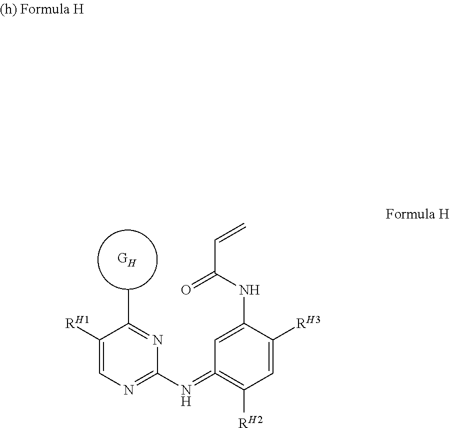

wherein:
G_{H} is selected from 4,5,6,7-tetrahydropyrazolo[1,5-a]pyridin-3-yl, 1H-indol-3-yl, 1-methyl-1H-indol-3-yl, and pyrazolo[1,5-a]pyridin-3-yl;
R^{H1} is selected from hydrogen, fluoro, chloro, methyl and cyano;
R^{H2} is selected from methoxy and methyl; and
R^{H3} is selected from (3R)-3-(dimethylamino)pyrrolidin-1-yl, (3S)-3-(dimethylamino)pyrrolidin-1-yl, 3-(dimethylamino)azetidin-1-yl, [2-(dimethylamino)ethyl]-(methyl)amino, [2-(methylamino)ethyl](methyl)amino, 5-methyl-2,5-diazaspiro[3.4]oct-2-yl, (3aR,6aR)-5-methylhexahydropyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl, and 4-[(2S)-2-aminopropanoyl]piperazin-1-yl;
or

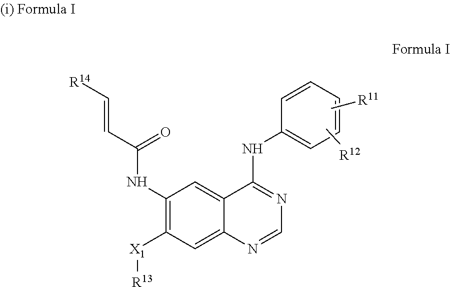

wherein:
R^{I1} is selected from F, Br, Cl, or I;
R^{I2} is selected from H, F, Br, Cl, or I;
R^{I3} is selected from:
   a) C₁-C₃ straight or branched alkyl, optionally substituted by halogen; or
   b) —(CH₂)ᵢₙ-morpholino, —(CH₂)ᵢₙ-piperidine, —(CH₂)ᵢₙ-piperazine, —(CH₂)ᵢₙ-piperazine-N(C₁-C₃ alkyl), —(CH)ᵢₙ-pyrrolidine, or —(CH₂)ᵢₙ-imidazole;
in is 1-4;
R^{I4} is —(CH₂)ᵢₘ-Het₁;
Het₁ is a heterocyclic moiety selected from the group of morpholine, piperidine, piperazine, piperazine-N(C₁-C₃ alkyl), imidazole, pyrrolidine, azepane, 3,4-dihydro-2H-pyridine, or 3,6-dihydro-2H-pyridine, wherein each heterocyclic moiety is optionally substituted by from 1 to 3 groups selected from C₁-C₃ alkyl, halogen, —OH, —NH₂, —NH(C₁-C₃ alkyl) or —N(C₁-C₃ alkyl)₂;
im is 1-3; and
X_{I} is O, S, or NH;
or

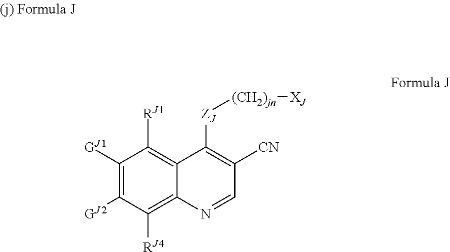

wherein:
X_{J} is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2-12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X_{J} is a radical having the formula:

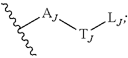

wherein
A_{J} is a pyridinyl, pyrimidinyl, or phenyl ring, wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2-12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T_{J} is bonded to a carbon of A_{J} and is: —NH(CH₂)ⱼₘ—, —O(CH₂)ⱼₘ—, —S(CH₂)ⱼₘ—, —NR(CH₂)ⱼₘ, —(CH₂)ⱼₘ—, —(CH₂)ⱼₘ—NH—, —(CH₂)ⱼₘ—O—, —(CH₂)ⱼₘ—S—, or —(CH₂)ⱼₘ—NR—;
L_{J} is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2-12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
or L_{J} is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2-12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
Z_{J} is —NH—, —O—, —S—, or —NR^{J}—;
R^{J} is alkyl of 1-6 carbon atoms, or carboalkyl of 2-7 carbon atoms;
G^{J1}, G^{J2}, R^{J1}, and R^{J4} are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2-12 carbon atoms, N-alkylcarbamoyl, N,N-dialkylcarbamoyl, N-alkyl-N-alkenylamino of 4-12 carbon atoms, N,N-dialkenylamino of 6-12 carbon atoms, phenylamino, benzylamino, (R^{J8})(R^{J9})CH-M_{J}-(C(R^{J6})₂)ⱼₖ—Y_{J}—, R^{J7}—(C(R^{J6})₂)_{jg}—Y_{J}—, R^{J7}—(C(R^{J6})₂)ⱼₚ-M_{J}- (C(R^{J6})₂)ⱼₖ—Y_{J}—, Het_{J}-(C(R^{J6})₂)_{jg}—W_{J}—(C(R^{J6})₂)ⱼₖ—Y_{J}—, or

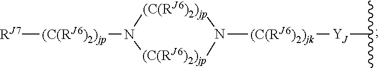

or R^{J1} and R^{J4} are as defined above and G^{J1} or G^{J2} or both are R^{J2}—NH—;
or if any of the substituents R^{J1}, G1, G^{J2}, or R^{J} are located on contiguous carbon atoms then they may be taken together as the divalent radical —O—C(R^{J6})₂—O—;
   Y_{J} is a divalent radical Selected from the group consisting of —(CH₂)ⱼₐ—, —O—, and —NR^{J6}—;
   R^{J7} is —NR^{J6}R^{J6}, —OR^{J6}, -J_{J}, —N(R^{J6})₃⁺, or —NR^{J6}(OR^{J6}),
   M_{J} is —N(R^{J6})—, —O—, —N[(C(R^{J6})₂)ⱼₚ—NR^{J6}R^{J6}]—, or —N[(C(R^{J6})₂)ⱼₚ—OR^{J6}]—,
   W_{J} is —N(R^{J6})—, —O—, or a bond;
   Het_{J} is is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and

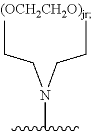 [SMILES: *N(CCC)CCC]

wherein Het_{J} is optionally mono- or di-substituted on carbon or nitrogen with R₆, optionally mono- or di-substituted on carbon with hydroxy, —N(R^{J6})₂, or —OR^{J6}, optionally mono or di-substituted on carbon with the mono-valent radicals —(C(R^{J6})₂)ⱼₛ—OR^{J6} or —(C(R^{J6})₂)ⱼₛ—N(R^{J6})₂, and optionally mono or di-substituted on a saturated carbon with divalent radicals —O— or —O—(C(R^{J6})₂)ⱼₛ—O—;
R^{J6} is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R^{J4} is selected from the group consisting of

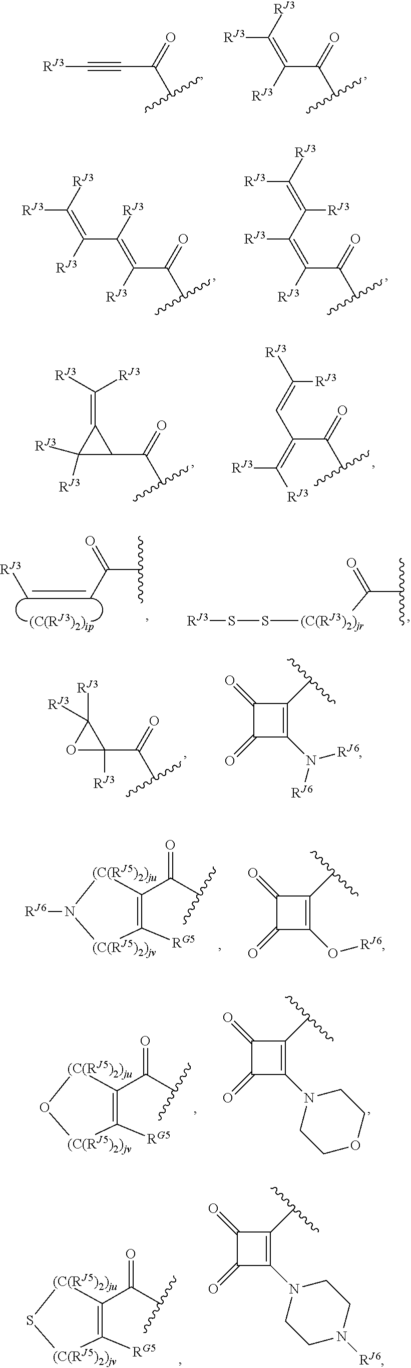

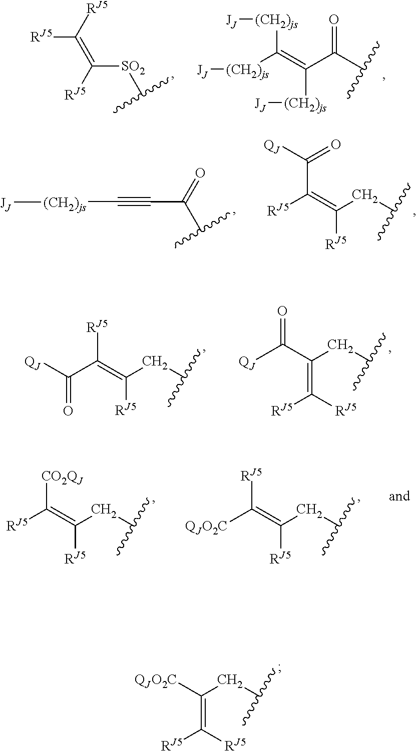

R^{J3} is independently hydrogen, alkyl of 1-6 carbon atoms, carboxy, carboalkoxy of 1-6 carbon atoms, phenyl, carboalkyl of 2-7 carbon atoms, R^{J7}—(C(R^{J6})₂)ⱼₛ—, R^{J7}—(C(R^{J6})₂)ⱼₚ-M_{J}-(C(R^{J6})₂)ⱼᵣ—, (R^{J8})(R^{J9})CH- M_{J}-(C(R^{J6})₂)ⱼᵣ—, Het_{J}-(C(R^{J6})₂)_{jq}—W_{J}—(C(R^{J6})₂)ⱼᵣ—, or

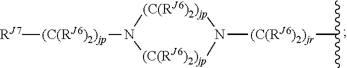

R^{J5} is independently hydrogen, alkyl of 1-6 carbon atoms, carboxy, carboalkoxy of 1-6 carbon atoms, phenyl, carboalkyl of 2-7 carbon atoms, R^{J7}—(C(R^{J6})₂)ⱼₛ—, R^{J7}—(C(R^{J6})₂)ⱼₚ-M_{J}-(C(R^{J6})₂)ⱼᵣ—, (R^{J8})(R^{J9})CH-M_{J}-(C(R^{J6})₂)ⱼᵣ—, Het-(C(R^{J6})₂)_{jq}—W_{J}—(C(R^{J6})₂)ⱼᵣ—, or

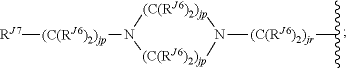

R^{J8} and R^{J9} are each, independently, —(C(R^{J6})₂)ⱼᵣ—NR^{J6}R^{J6} or —(C(R^{J6})₂)ⱼᵣ—OR^{J6},
J_{J} is independently hydrogen, chlorine, fluorine, or bromine;
Q_{J} is alkyl of 1-6 carbon atoms or hydrogen;
ja is 0 or 1;
jg is 1-6;
jk is 0-4;
jn is 0-1;
jm is 0-3
jp is 2-4;
jq is 0-4;
jr is 1-4;
js is 1-6;
ju is 0-4; and
jv is 0-4, wherein the sum of ju+jv is 2-4;
provided that when R¹⁶ is alkenyl of 2-7 carbon atoms or alkynyl of 2-7 carbon atoms, such alkenyl or alkynyl moiety is bound to a nitrogen or oxygen atom through a saturated carbon atom;
or

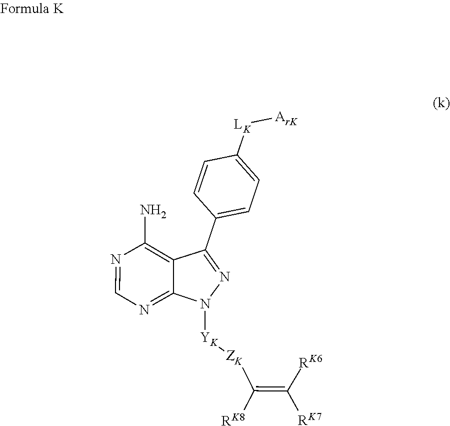

wherein:
L_{K} is CH₂, O, NH, or S;
Ar_{K} is an optionally substituted aromatic carbocycle or aromatic heterocycle;
Y_{K} is an optionally substituted alkyl, heteroalkyl, carbocycle, or heterocycle;
Z_{K} is C(O), OC(O), NHC(O), C(S), S(O)ₖₓ, OS(O)ₖₓ, or NHS(O)ₖₓ, where kx is 1 or 2; and
R^{K6}, R^{K7}, and R^{K8} are independently selected from H, alkyl, heteroalkyl, carbocycle, or heterocycle;
or

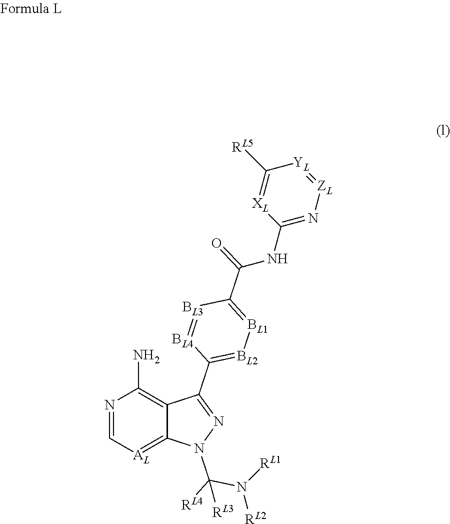

wherein:
X_{L} is CH, N, O or S;
Y_{L} is C(R^{L6}), N, O or S;
Z_{L} is CH, N or bond;
A_{L} is CH or N;
B_{L1} is N or C(R^{L7});
B_{L2} is N or C(R^{L8});
B_{L3} is N or C(R^{L9});
B_{L4} is N or C(R^{L10});
R^{L1} is R^{L11}C(O)—, R^{L12}S(O)—, R^{L13}SO₂— or (1-6C)alkyl optionally substituted with R^{L4};
R^{L2} is H, (1-3C)alkyl or (3-7C)cycloalkyl;
R^{L3} is H, (1-6C)alkyl or (3-7C)cycloalkyl); or
R^{L2} and R^{L3} form, together with the N and C atom they are attached to, a (3-7C)heterocycloalkyl optionally substituted with one or more fluorine, hydroxyl, (1-3C)alkyl, (1-3C)alkoxy or oxo;
R^{L4} is H or (1-3C)alkyl;
R^{L5} is H, halogen, cyano, (1-4C)alkyl, (1-3C)alkoxy, (3-6C)cycloalkyl; wherein all alkyl groups of R^{L5} are optionally substituted with one or more halogen;
or R^{L5} is (6-10C)aryl or (2-6C)heterocycloalkyl;
R^{L6} is H or (1-3C)alkyl; or
R^{L5} and R^{L6} together may form a (3-7C)cycloalkenyl, or (2-6C)heterocycloalkenyl; each optionally substituted with (1-3C)alkyl, or one or more halogen;
R^{L7} is H, halogen or (1-3C)alkoxy;
R^{L8} is H or (1-3C)alkyl; or
R^{L7} and R^{L8} form, together with the carbon atom they are attached to a (6-10C)aryl or (1-9C)heteroaryl;
R^{L9} is H, halogen or (1-3C)alkoxy;
R^{L10} is H, halogen, or (1-3C)alkoxy;
R^{L11} is independently selected from a group consisting of (1-6C)alkyl, (2-6C)alkenyl and (2-6C)alkynyl; wherein each alkyl, alkenyl or alkynyl optionally substituted with one or more groups selected from hydroxyl, (1-4C)alkyl, (3-7C)cycloalkyl, [(1-4C)alkyl]amino, di[(1-4C)alkyl]amino, (1-3C)alkoxy, (3-7C)cycloalkoxy, (6-10C)aryl or (3-7C)heterocycloalkyl;
or R^{L11} is (1-3C)alkyl-C(O)—S-(1-3C)alkyl;
or R^{L11} is (1-5C)heteroaryl optionally substituted with one or more groups selected from halogen or cyano;
R^{L12} and R^{L13} are independently selected from a group consisting of (2-6C)alkenyl or (2-6C)alkynyl, wherein the alkenyl and alkynyl is optionally substituted with one or more groups selected from hydroxyl, (1-4C)alkyl, (3-7C)cycloalkyl, [(1-4C)alkyl]amino, di[(1-4C)alkyl]amino, (1-3C)alkoxy, (3-7C)cycloalkoxy, (6-1° C.)aryl, or (3-7C)heterocycloalkyl; or (1-5C)heteroaryl optionally substituted with one or more groups selected from halogen or cyano; and
R^{L14} is independently selected from a group consisting of halogen, cyano or (2-6C)alkenyl or (2-6C)alkynyl, wherein the alkenyl and alkynyl is optionally substituted with one or more groups selected from hydroxyl, (1-4C)alkyl, (3-7C)cycloalkyl, [(1-4C)alkyl]amino, di[(1-4C)alkyl]amino, (1-3C)alkoxy, (3-7C)cycloalkoxy, (6-1° C.)aryl, (1-5C)heteroaryl or (3-7C)heterocycloalkyl;
with the provisos that:
0 to 2 atoms of X_{L}, Y_{L}, and Z_{L} can simultaneously be a heteroatom;
when one atom selected from X_{L} and Y_{L} is O or S, then Z_{L} is a bond and the other atom selected from X_{L} and Y_{L} can not be 0 or S;
when Z_{L} is C or N then Y_{L} is C(R^{L6}) or N and X_{L} is C or N; and
0 to 2 atoms of B_{L1}, B_{L2}, B_{L3} and B_{L4} are N;
or

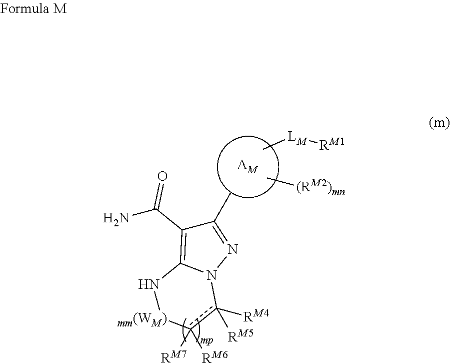

wherein:
A_{M} is a 5- or 6-membered aromatic ring comprising 0-3 heteroatoms of N, S or O;
each W_{M} is independently —(CH₂)— or —C(O)—;
L_{M} is a bond, CH₂, NR^{M12}, O, or S;
[symbol] is a single or double bond, and when a double bond, R^{M5} and R^{M7} are absent
mm is 0-4;
mn is 0-4, wherein when mn is more than 1, each R^{M2} may be different;
mp is 0-2, wherein when mp is 0, mm is 1-4, and when mp is 2, each R^{M6} and each R^{M7} may be different;
R^{M1}, R^{M4}, R^{M5}, R^{M6}, and R^{M7} are each independently H, halogen, heteroalkyl, alkyl, alkenyl, cycloalkyl, aryl, saturated or unsaturated heterocyclyl, heteroaryl, alkynyl, —CN, —NR^{M13}R^{M14}, —OR^{M13}, —COR^{M13}, —CO₂R^{M13}, —CONR^{M13}R^{M14}, —C(═NR^{M13})NR^{M14}R^{M15}, —NR^{M13}COR^{M14}, —NR^{M13}CONR^{M14}R^{M15}, —NR^{M13}CO₂R^{M14}, —SO₂R^{M13}, —NR^{M13}SO₂NR^{M14}R^{M15}, or —NR^{M13}SO₂R^{M14} wherein the alkyl, alkenyl, alkynyl, cycloalkyl, heteroaryl, aryl, and saturated or unsaturated heterocyclyl are optionally substituted with at least one substituent R^{M16}, wherein (R^{M4} and R^{M5}), or (R^{M4} and R^{M6}), or (R^{M6} and R^{M7}), or (R^{M6} and R^{M6} when mp is 2), together with the atoms to which they are attached, can form a ring selected from cycloalkyl, saturated or unsaturated heterocycle, aryl, and heteroaryl rings optionally substituted with at least one substituent R^{M16};
R^{M2} is halogen, alkyl, —S-alkyl, —CN, —NR^{M13}R^{M14}, —OR^{M13}, —COR^{M13}, —CO₂R^{M13}, —CONR^{M13}R^{M14}, —C(═NR^{M13})NR^{M14}R^{M15}, —NR^{M13}COR^{M14}, —NR^{M13}CONR^{M14}R^{M15}, —NR^{M13}CO₂R^{M14}, —SO₂R^{M13}, —NR^{M13}SO₂NR^{M14}R^{M15} or —NR^{M13}SO₂R^{M14}.
R^{M12} is H or lower alkyl;
R^{M13}, R^{M14} and R^{M15} are each independently H, heteroalkyl, alkyl, alkenyl, alkynyl, cycloalkyl, saturated or unsaturated heterocyclyl, aryl, or heteroaryl; wherein (R^{M13} and R^{M14}), and/or (R^{M14} and R^{M15}) together with the atom(s) to which they are attached, each can form a ring selected from cycloalkyl, saturated or unsaturated heterocycle, aryl, and heteroaryl rings optionally substituted with at least one substituent R^{M16}; and
R^{M16} is halogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted heterocyclyl, oxo, —CN, —OR^{M}′, —NR^{M}′R^{M}″, —COR^{M}′, —CO₂R^{M}′, —CONR^{M}′R^{M}″, —C(═NR^{M}′)NR^{M}″R^{M}′″, —NR^{M}′COR^{M}″—NR^{M}′CONR^{M}′R^{M}″, —NR^{M}′CO₂R^{M}″, —SO₂R^{M}′, —SO₂aryl, —NR^{M}′SO₂NR^{M}″R^{M}′″, or —NR^{M}′SO₂R^{M}″, wherein R^{M}′, R^{M}″, and R^{M}′″ are independently hydrogen, halogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted heterocyclyl, wherein (R^{M}′ and R^{M}″), and/or (R^{M}″ and R^{M}′″) together with the atoms to which they are attached, can form a ring selected from cycloalkyl, saturated or unsaturated heterocycle, aryl, and heteroaryl rings;
or

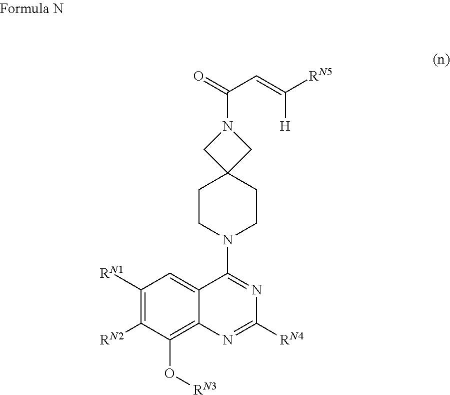

wherein:
R^{N1} is vinyl, (E)-1-propenyl or cyclopropyl;
R^{N2} is the following formula (II) or (III):

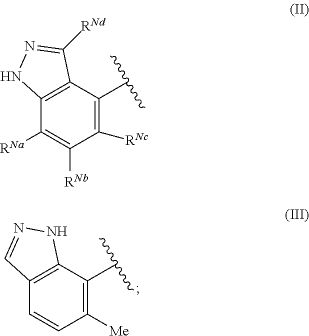

R^{N3} is C₃₋₄alkyl, methyl or n-propyl each of which may be substituted with two or more F's, ethyl or C₃-4cycloalkyl each of which may be substituted with F, benzyl which may be substituted with C₁₋₃alkyl, benzyl which may be substituted with —O—C₁₋₃alkyl alkyl, or benzyl which may be substituted with —O—(C₁₋₃alkyl which is substituted with F);
R^{N4} is, —O-optionally substituted C₃₋₅alkyl, —O-optionally substituted cycloalkyl, or the following formula

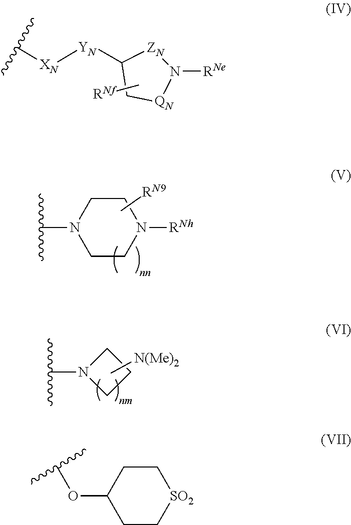

R^{N5} is H or CF₃;
R^{Na} is H or F;
R^{Nb} is H or F;
R^{Nc} is, H, methyl, vinyl or Cl;
R^{Nd} is H or Cl;
R^{Ne} is CO₂Me, COMe, CON(Me)₂, SO₂Me, C₃₋₄cycloalkyl, optionally substituted 4- to 6-membered non-aromatic heterocyclic ring, or C₁₋₃alkyl optionally substituted with a group selected from group G_{N};
Group G_{N}; —OC₁₋₃alkyl, —O—(C₁₋₃ alkyl substituted with F or C₃₋₄cycloalkyl), C₃₋₄cycloalkyl, —F, —CN, —SO₂Me, aromatic heterocyclic group, 4- to 6-membered non-aromatic heterocyclic ring, —N(C₁₋₃alkyl)₂, and —C(Me)₂OH;
R^{Nf} is, H, methyl or F;
R^{Ng} is, H, methyl or ethyl;
R^{Nh} is a good C1-3 alkyl optionally substituted with —OMe;
X_{N} is, O, NH, S or methylene;
Y_{N} is a bond or methylene;
Z_{N} is a bond, methylene or ethylene;
Q_{N} is methylene or ethylene;
nn is an integer of 1 or 2; and
nm is an integer from 1 to 3;
or

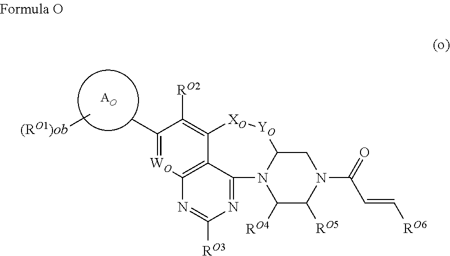

wherein:
Ring A_{O} is selected from aryl, monocyclic heteroaryl and bicyclic heteroaryl;
R^{O1} is independently selected from C₁₋₄alkyl, halo, hydroxy, C₁₋₄alkoxy, C₁₋₃fluoroalkyl, C₁₋₃fluoroalkoxy, cyano, acetylenyl, NR^{O7}R^{O1}, C(O)NR^{O9}R^{O10}, CH₂R^{O11}, N═S(O)Me₂, S(O)Me and SO₂R¹²;
ob is 0, 1, 2 or 3;
W^{O} is N or CR¹³;
X^{O} is O or NR¹⁴;
Y_{O} is CR^{O15}R^{O16}, CR^{O17}R^{O18}CR^{O19}R^{O20}, C═O, or C(O)CR^{O21}R^{O22}.
R^{O2} is H, cyano, halo, C₁₋₄alkyl, C₁₋₄alkoxy, C₁₋₃fluoroalkyl, NR^{O23}R^{O24}, acetylenyl or CH₂OR^{O21};
R^{O3} is H, C₁₋₃fluoroalkyl, OR^{O26}, NR^{O27}R^{O28}, CH₂R^{O29}, SR^{O30} or C(O)R^{O31}.
R^{O4} is H or Me;
R^{O5} is H or Me;
R^{O6} is H or CH₂NMe₂;
R^{O7} is H, C₁₋₄alkyl, C(O)C₁₋₃alkyl or CO₂C₁₋₃alkyl;
R^{O11} is hydroxy, cyano, heterocyclyl, NR^{O32}R^{O33}, C(O)NR^{O34}R^{O35} or SO₂C₁₋₃alkyl;
R^{O12} is C₁₋₃alkyl, C₁₋₃fluoroalkyl or NR^{O36}R^{O37};
R^{O13} is H, C₁₋₄alkyl, halo, C₁₋₃fluoroalkyl or C₁₋₄alkoxy;
R^{O15}, R^{O16}, R^{O17} and R^{O18} are independently selected from H and C₁₋₃alkyl;
R^{O19}, R^{O20}, R^{O21} and R^{O22} are independently selected from H, C₁₋₃alkyl, and fluoro;
R^{O26} is selected from the group consisting of:
   H;
   C₁₋₄alkyl optionally substituted with 1 or 2 substituents selected from hydroxy, C₁₋₃ alkoxy, halo, NR^{O38}R^{O39}, C(O)NR^{O40}R^{O41}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
   C₃₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃ fluoroalkyl, C₃₋₇cycloalkyl, heterocyclyl or heteroaryl; and
   heteroaryl optionally substituted with C₁₋₄alkyl, hydroxy, halo, cyano or C₁₋₄alkoxy;
R²⁷ is selected from the group consisting of:
   H;
   C(O)R^{O42}.
   C₁₋₄alkyl optionally substituted with 1 or 2 substituents selected from hydroxy, C₁₋₃alkoxy, halo, NR^{O43}R^{O44}, C(O)NR^{O45}R^{O46}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
   C₃₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃ fluoroalkyl, C₃₋₇cycloalkyl, CH₂cyclopropyl, heterocyclyl or heteroaryl; and
   heteroaryl optionally substituted with C₁₋₄alkyl, hydroxy, halo, cyano or C₁₋₄alkoxy;
R^{O28} is H or Me; or
R^{O27} and R^{O28} taken together with the nitrogen atom to which they are attached form a 4-, 5-, 6- or 7-membered heterocyclic ring, wherein said ring is optionally substituted with C₁₋₄alkyl, hydroxy, halo, C(O)Me, NR^{O47}R^{O48}, C₁₋₃alkoxy, C₁₋₃fluoroalkyl, C₃₋₇cycloalkyl, CH₂cyclopropyl, heterocyclyl or heteroaryl;
R^{O29} is selected from the group consisting of:
   H;
   NR^{O49}R^{O50};
   C₁₋₃alkyl optionally substituted with 1 or 2 substituents selected from hydroxy, C₁₋₃alkoxy, halo, NR^{O51}R^{O52}, C(O)NR^{O53}R^{O54}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl; C₃₋₇cycloalkyl optionally substituted with C₁₋₄alkyl, hydroxy or halo; heterocyclyl optionally substituted with C₁₋₄ alkyl, hydroxy, halo, C(O)Me, C₁₋₃alkoxy, C₁₋₃fluoroalkyl, C₃₋₇cycloalkyl, CH₂cyclopropyl, heterocyclyl or heteroaryl; and
   heteroaryl optionally substituted with C₁₋₄alkyl, hydroxy, halo, cyano or C₁₋₄alkoxy;
R^{O30} is selected from the group consisting of:
   C₁₋₄alkyl optionally substituted with 1 or 2 substituents selected from hydroxy, C₁₋₃alkoxy, halo, NR^{O55}R^{O56}, C(O)NR^{O57}R^{O58}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
   C₃₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl; and
   heteroaryl optionally substituted with C₁₋₄alkyl, hydroxy, halo, cyano or C₁₋₄alkoxy;
R^{O31} is NR^{O59}R^{O60};
R^{O42} is optionally substituted heteroaryl or optionally substituted C₁₋₄alkyl;
R^{O49} and R^{O51} are independently selected from H, C₁₋₄alkyl, heterocyclyl and heteroaryl;
R^{O59} and R^{O60} are independently selected from H and C₁₋₄alkyl; or
R^{O59} and R^{O60} taken together with the nitrogen atom to which they are attached form a 4-, 5- or 6-membered heterocyclic ring, wherein said ring is optionally substituted with C₁₋₄alkyl, hydroxy, halo or C(O)Me;
R^{O8}, R^{O9}, R^{O10}, R^{O14}, R^{O23}, R^{O24}, R^{O25}, R^{O32}, R^{O33}, R^{O34}, R^{O35}, R^{O36}, R^{O37}, R^{O38}, R^{O39}, R^{O40}, R^{O41}, R^{O43}, R^{O44}, R^{O45}, R^{O46}, R^{O47}, R^{O48}, R^{O50}, R^{O52}, R^{O53}, R^{O54}, R^{O55}, R^{O56}, R^{O57}, R^{O5}s, R^{O61}, and R^{O62} are independently selected from H and C₁₋₄alkyl;
or

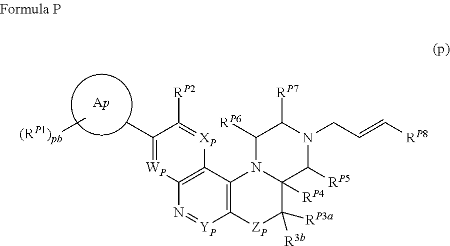

wherein:
A_{P} is selected from C₆-C₁₀ aryl, monocyclic heteroaryl and bicyclic heteroaryl;
R^{P1} is in each instance independently selected from F, Cl, Br, OH, CN, C₁-C₄ alkyl, C₁-C₄ alkoxy, C₁-C₃ fluoroalkyl, C₁-C₃ fluoroalkoxy, acetylenyl, NR^{P9}R^{P10}, C(O)NR^{P11}R^{P12}, CH₂R^{P13} and N═S(O)Me₂;
pb is 0, 1, 2 or 3;
W_{P} is CR¹⁴ or N;
X_{P} is CR¹⁵ or N;
Y_{P} is CH or N;
Z_{P} is O or NR^{P16};
R^{P2} is H, CN, F, Cl, Br, C₁-C₄ alkyl, C₁-C₄ alkoxy, C₁-C₃ fluoroalkyl, C₁-C₂ fluoroalkoxy or acetylenyl;
R^{P3a} and R^{P3b} are each independently selected from H or Me or, in the case where Z_{P} is NR^{P16}, can also together be ═O;
R^{P4}, R^{P5}, R^{P6} and R^{P7} are each independently selected from H or Me;
R^{P8} is H or CH₂NMe₂;
R^{P9} is H, C₁-C₄ alkyl, C(O)C₁-C₃ alkyl or CO₂C₁-C₃ alkyl;
R^{P10}, R¹¹ and R^{P12} are each independently selected from H and C₁-C₄ alkyl; or
R^{P9} and R^{P10} together, or R^{P1} and R^{P12} together, form a 4-, 5-, 6- or 7-membered saturated heterocycle optionally incorporating O, NH or N(C₁-C₄ alkyl) group;
R^{P13} is OH, CN, NR^{P17}R^{P18}, C(O)NR^{P19}R^{P20} or SO₂C₁-C₃alkyl;
R^{P14} and R^{P15} are each independently selected from H, F, Cl, MeO and Me;
R^{P16} is H, C₁-C₃ fluoroalkyl or CH₂R^{P21};
R^{P17}, R^{P18}, R^{P19} and R^{P20} are each independently selected from H and C₁-C₄ alkyl;
or R^{P17} and R^{P18} together, or R^{P19} and R^{P20} together, form a 4-, 5-, 6- or 7-membered saturated heterocycle optionally incorporating O, NH or N(C₁-C₄ alkyl) group;
R²¹ is selected from the group consisting of:
   C₁-C₃ alkyl optionally substituted with 1 or 2 substituents selected from hydroxy, C₁-C₃ alkoxy, halo, NR^{P22}R^{P23}, C(O)NR^{P24}R^{P25}, SO₂Me, heteroaryl, C₃₋₇cycloalkyl or heterocyclyl, wherein said heteroaryl or C₃-C₇cycloalkyl is optionally further substituted with C₁-C₄ alkyl, hydroxy, halo, cyano, or C₁-C₄ alkoxy and said heterocyclyl is optionally further substituted with C₁-C₄ alkyl, hydroxy, halo, C(O)Me, C₁-C₃ alkoxy, C₁-C₃fluoroalkyl, C₃-C₇cycloalkyl, heterocyclyl or heteroaryl and wherein R^{P22}, R^{P23}, R^{P24} and R^{P25} are in each instance independently selected from H and C₁-C₄ alkyl;
   C₃-C₇cycloalkyl optionally substituted with C₁-C₄ alkyl, hydroxy or halo;
   heterocyclyl optionally substituted with C₁-C₄ alkyl, hydroxy, halo, C(O)Me, C₁-C₃ alkoxy, C₁-C₃ fluoroalkyl, C₃-C₇ cycloalkyl, CH₂cyclopropyl, heterocyclyl or heteroaryl; and
   heteroaryl optionally substituted with C₁-C₄ alkyl, hydroxy, halo, cyano or C₁-C₄ alkoxy;
or

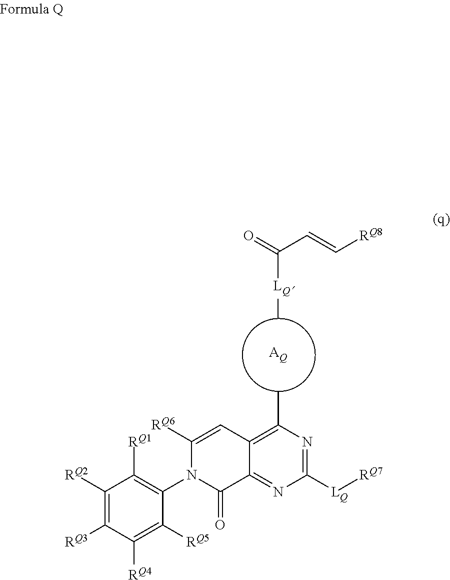

wherein:
Ring A_{Q} is 3-8 membered heterocycloalkyl, the 3-8 membered heterocycloalkyl is optionally substituted with 1, 2 or 3 of the R^{Q};
R^{O1}, R^{Q2}, R^{Q3}, R^{Q4} and R^{Q5} are independently selected from H, halogen, OH, NH₂, CN, C₁₋₆alkyl and C₁₋₆ heteroalkyl, wherein the C₁₋₆alkyl and C₁₋₆heteroalkyl is optionally substituted with 1, 2 or 3 of the R^{Q};
or, R^{Q1} and the R^{Q2} are joined together to form ring B_{Q};
or, R^{Q2} and the R^{Q3} are joined together to form ring B_{Q};
or, R^{Q3} and the R^{Q4} are joined together to form ring B_{Q};
or, R^{Q4} and the R^{Q5} are joined together to form ring B_{Q};
Ring B_{Q} is selected from the group consisting of phenyl ring, C₅₋₆Cycloalkenyl, 5-6 membered heterocycloalkenyl and the 5-6 membered aryl, phenyl, C₅₋₆Bicycloalkenyl and 5-6 membered heterocyclenyl, 5-6 membered heteroaryl ring is optionally substituted with 1, 2 or 3 R^{Qa};
R^{Qa} is selected from halogen, OH, NH₂, CN, C₁₋₆alkyl group and C₁₋₆heteroalkyl, wherein the C₁₋₆alkyl and C₁₋₆heteroalkyl is optionally substituted with 1, 2 or 3 R^{Q};
R^{Q6} is selected from H, halogen and C₁₋₆alkyl, wherein the C₁₋₆alkyl is optionally substituted with 1, 2 or 3 of the R^{Q};
R^{Q7} is selected from the group H, CN, NH₂, C₁₋₈alkyl, C₁₋₈heteroalkyl, 4-6 membered heterocylcoalkyl, 5-6 membered aryl and C₅₋₆Cycloalkyl, C₁₋₈Alkyl, C₁₋₈Heteroalkyl, 4-6 membered heterocylcoalkyl, 5-6 membered aryl and C₅₋₆Cycloalkyl is optionally substituted with 1, 2 or 3 of the R^{Q};
L_{Q} is selected from single bonds, —NH—, —S—, —O—, —C(═O)—, —C(═S)—, —CH₂—, —CH(R^{Qb})— and —C(R^{Qb})₂—;
L_{Q}′ is selected from a single bond and —NH—;
R^{Qb} is selected from C₁₋₃alkyl and C₁₋₃heteroalkyl, wherein the C₁₋₃alkyl and C₁₋₃heteroalkyl is optionally substituted with 1, 2 or 3 of the R^{Q};
R^{Q}′ is selected from H, C₁₋₆alkyl and C₁₋₆heteroalkyl, wherein the C₁₋₆alkyl and C₁₋₆heteroalkyl is optionally substituted with 1, 2 or 3 of the R^{Q};
R^{Q} is selected from halogen, OH, NH₂, CN, C₁₋₆alkyl, C₁₋₆heteroalkyl and C₃₋₆cycloalkyl, wherein the C₁₋₆ alkyl, C₁₋₆heteroalkyl, and C₃₋₆cycloalkyl is optionally substituted with 1, 2 or 3 R^{Q}′;
R^{Q}′ is selected from: F, Cl, Br, I, OH, NH₂, CN, CH₃, CH₃CH₂, CH₃O, CF₃, CHF₂, CH₂F, Cycloproyl, propyl, isopropyl, N(CH₃)₂, NH(CH₃);
each 3-8 membered heterocyclic alkyl, C₁₋₆Heteroalkyl, 5-6 membered heterocycloalkenyl, 5-6 membered heteroaryl, C₁₋₈Heteroalkyl, 4-6 membered heterocycloalkyl, C₁-3 Heteroalkyl contains 1, 2, or 3, “heteroatom” groups independently selected from the group of —C(═O)N(R)—, —N(R)—, —NH—, N, —O—, —S—, —C(═O)O—, —C(═O)—, —C(═S)—, —S(═O)—, —S(═O)₂— and —N(R)C(═O)N(R)—;
or

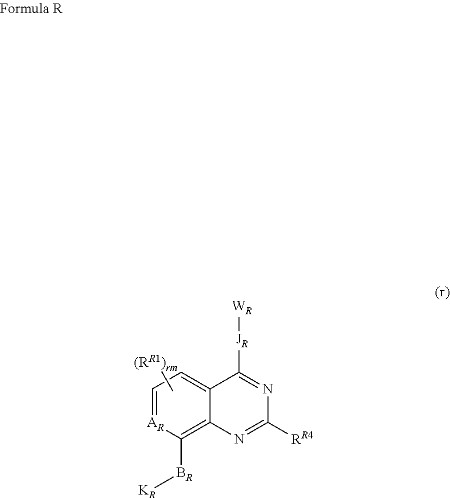

wherein:
A_{R} is —C(H)— or nitrogen;
B_{R} is oxygen, sulfur, NR^{R6} or C(R^{R6})₂;
J_{R} is a heterocycle having 3-12 ring atoms, where J_{R} is optionally substituted with 1, 2, 3, 4, 5 or 6 R²;
K_{R} is C₆-C₁₂aryl, or K_{R} is heteroaryl having 5-12 ring atoms, where K_{R} is optionally substituted with 1, 2, 3, 4, 5, 6 or 7 R^{R3};
W_{R} is selected from the group consisting of:

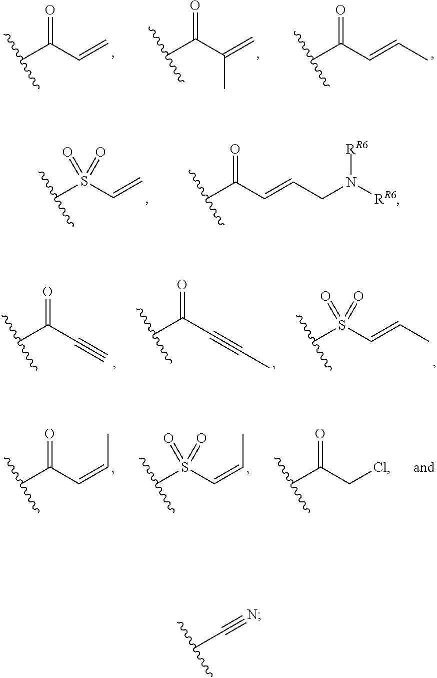

each R^{R1} is independently selected from the group consisting of C₁-C₆ alkyl, C₃-C₆ cycloalkyl, C₁-C₆ alkyl-hydroxy, C₁-C₆ alkoxy, C₁-C₆ alkyl-C₁-C₆ alkoxy, hydroxy, C₂-C₆ alkenyl, C₂-C₆ alkynyl, halogen, C₁-C₆ haloalkyl, cyano, and N(R^{R6})₂, or two R^{R1} optionally join to form a heterocycle having 3-12 ring atoms or a C₃-C₆ cycloalkyl;
each R^{R2} is independently selected from the group consisting of C₁-C₆ alkyl, C₃-C₆ cycloalkyl, hydroxy, C₁-C₆ alkyl-hydroxy, C₁-C₆ alkoxy, halogen, C₁-C₆ haloalkyl, cyano, C₁-C₆ alkylcyano, and oxo, or two R^{R2} optionally join to form a heterocycle having 3-12 ring atoms or a C₃-C₆ cycloalkyl;
each R^{R3} is independently selected from the group consisting of C₁-C₆ alkyl, C₃-C₆ cycloalkyl, hydroxy, C₁-C₆ alkoxy, halogen, C₁-C₆ halo-alkyl, —N(R^{R6})₂, oxo, and cyano, or two R^{R3} optionally join to forma heterocycle having 3-12 ring atoms or C₃-C₆ cycloalkyl;
R^{R4} is —X_{R}—Y_{R}—Z_{R} where:
   X_{R} is absent or is selected from the group consisting of oxygen, sulfur and —NR^{R6}—;
   Y_{R} is absent or C₁-C₆ alkylenyl; and
   Z_{R} is selected from H, —N(R^{R6})₂, —C(O)—N(R^{R6})₂, —OR^{R6}, heterocycle having 3-12 ring atoms, heteroaryl having 5-12 ring atoms, and C₃-C₆ cycloalkyl;
   where R^{R4} is optionally substituted with one or more R^{R7};
each R^{R5} is independently selected from the group consisting of: C₁-C₆ alkyl, hydroxy, C₁-C₆ alkoxy, halogen and —N(R^{R6})₂;
each R^{R6} is independently selected from the group consisting of hydrogen, hydroxyl, C₁-C₆ alkoxy and C₁-C₆ alkyl, or two R^{R6} optionally join to form heterocycle having 3-12 ring atoms or C₃-C₆ cycloalkyl;
each R^{R7} is independently R^{R7} or C₁-C₆ alkyl-R^{R7}, where each R^{R7} is independently selected from the group consisting of: C₁-C₆ alkyl, hydroxy, C₁-C₆ alkoxy, halogen, —N(R^{R6})₂, heterocycle having 3-12 ring atoms, and oxo; and
rm is 0, 1, 2 or 3;
or

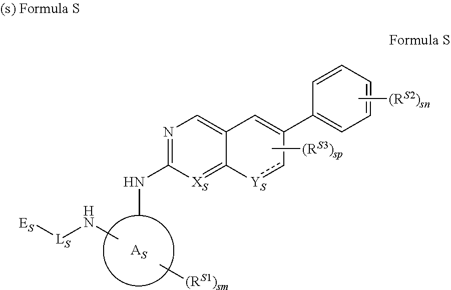

wherein:
E_{S} is a moiety that is capable of forming a covalent bond with a nucleophile;
Ring A_{S} is a 3-8 membered aryl, heteroaryl, heterocyclic or alicyclic group;
X_{S} is CH or N;
Y_{S} is CH or N—R^{S4}, where R^{S4} is H or C₁₋₆ alkyl;
L_{S} is —[C(R^{S5})(R^{S6})]_{sq}—, where each of R^{S5} and R^{S6} is, independently, H or C₁₋₆ alkyl; and sq is 0-4;
each R^{S1}, R^{S2}, and R^{S3} is, independently, halo, cyano, optionally substituted C₁₋₆ alkoxy, hydroxy, oxo, amino, amido, alkylurea, optionally substituted C₁₋₆ alkyl, or optionally substituted C₂₋₆ heterocyclyl;
sm is 0-3;
sn is 0-4; and
sp is 0-2;
or

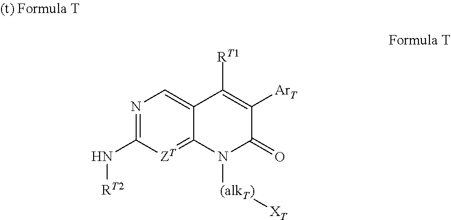

wherein:
Ar_{T} is phenyl or heteroaryl, each ring optionally substituted with one, two, three, or four substituents independently selected from alkyl, cycloalkyl, hydroxy, alkoxy, halo, haloalkyl, alkylsulfonyl, haloalkoxy, and cyano;
R^{T1} is hydrogen, halo, or alkyl;
R^{T2} is hydrogen, alkyl, cycloalkyl substituted with amino, alkylamino, or dialkylamino, hydroxyalkyl, alkoxyalkyl, aminoalkyl, heterocyclyl (wherein heterocyclyl is optionally substituted with one or two substituents independently selected from alkyl, hydroxyalkyl, alkoxyalkyl, aminoalkyl, optionally substituted aryl, optionally substituted heteroaryl, and optionally substituted heterocyclyl), heterocyclylalkyl (wherein the heterocyclyl ring in heterocyclylalkyl is optionally substituted with one or two substituents independently selected from alkyl, hydroxyalkyl, aminoalkyl, optionally substituted aryl, optionally substituted heteroaryl, and optionally substituted heterocyclyl), phenyl or heteroaryl (wherein phenyl or heteroaryl is optionally substituted with one, two, or three substituents where two of the phenyl or heteroaryl optional substituents are independently selected from alkyl, hydroxy, alkoxy, halo, haloalkyl, haloalkoxy, and cyano and one of the phenyl or heteroaryl optional substituents is alkyl, cycloalkyl, hydroxy, alkoxy, halo, haloalkyl, haloalkoxy, cyano, hydroxyalkyl, alkoxyalkyl, aminoalkyl, optionally substituted aryl, optionally substituted heteroaryl or optionally substituted heterocyclyl);
alk_{T} is alkylene;
X_{T} is a group of formula (a_{T}) or (b_{T}):

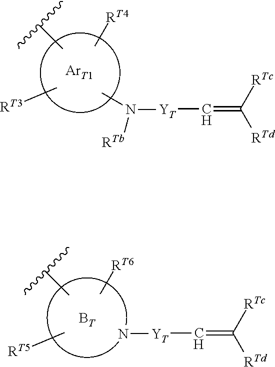

wherein:
Ar_{T1} is 5- or 6-membered cycloalkylene, phenylene, or 5- or 6-membered heteroarylene;
Ring B_{T} is azetidinyl, pyrrolidinyl, or piperidinyl where the nitrogen atom of the azetidinyl, pyrrolidinyl, or piperidinyl ring is attached to YT;
R^{T3} is hydrogen, alkyl, hydroxy, alkoxy, halo, haloalkyl, haloalkoxy, or cyano;
R^{T4} is hydrogen, alkyl, cycloalkyl, hydroxy, alkoxy, halo, haloalkyl, haloalkoxy, or cyano;
R^{T5} and R^{T6} are independently hydrogen, alkyl, or halo;
Y_{T} is —CO— or —SO₂—;
R^{Tb} is hydrogen or alkyl;
R^{Tc} is hydrogen, alkyl, or substituted alkyl; and
R^{Td} is hydrogen or alkyl;
provided that when (i) Ar_{T1} is phenylene or 6-membered heteroarylene then alk_{T} and —NR-YT-CH═CR^{Tc}R^{Td} are meta or para to each other; and when (ii) B_{T} is piperidinyl, then alk_{T} and —Y_{T}—CH═CR^{Tc}R^{Td} are meta or para to each other;
or

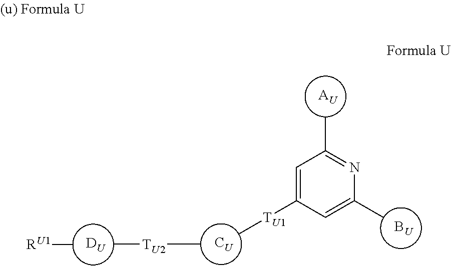

wherein:
R^{U1} is a moiety that is capable of forming a covalent bond with a nucleophile;
Ring A^{U} is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
Ring B_{U} is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T_{U1} is a covalent bond or a bivalent straight or branched, saturated or unsaturated C₁₋₆hydrocarbon chain wherein one or more methylene units of T_{U1} are optionally and independently replaced by —O—, —S—, —N(R^{U})—, —C(O)—, —OC(O)—, —C(O)O—, —C(O)N(R^{U})—, —N(R^{U})C(O)—, —N(R^{U})C(O)N(R^{U})—, —SO₂—, —SO₂N(R^{U})—, —N(R^{U})SO₂—, or —N(R^{U})SO₂N(R^{U})—;
Ring C_{U} is absent or an optionally substituted group selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged or spiro bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; wherein when Ring C_{U} is absent, T_{U2} is directly attached to T_{U1};
T_{U2} is a covalent bond or a bivalent straight or branched, saturated or unsaturated C₁₋₆hydrocarbon chain wherein one or more methylene units of T_{U2} are optionally and independently replaced by —O—, —S—, —N(R^{U})—, —C(O)—, —OC(O)—, —C(O)O—, —C(O)N(R^{U})—, —N(R^{U})C(O)—, —N(R^{U})C(O)N(R^{U})—, —SO₂—, —SO₂N(R^{U})—, —N(R^{U})SO₂—, or —N(R^{U})SO₂N(R^{U})—;
Ring D_{U} is absent or an optionally substituted group selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; wherein when Ring D_{U} is absent, R^{U1} is directly attached to T_{U2}; and
each R^{U} is independently hydrogen or an optionally substituted group selected from C₁₋₆aliphatic, phenyl, a 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
or two R^{U} groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur.

Further provided in the disclosure is a subject polypeptide or multivalent antigen binding unit specifically binding to a target bound by an exogenous molecule disclosed herein. Also provided are subject polypeptides coupled to (e.g., covalently conjugated to or non-covalently bound to) a particle, including but not limited to microparticles or nanoparticles. In some embodiments, a subject polypeptide or multivalent antigen binding unit specifically bind to a target bound by a compound selected from the following structures.

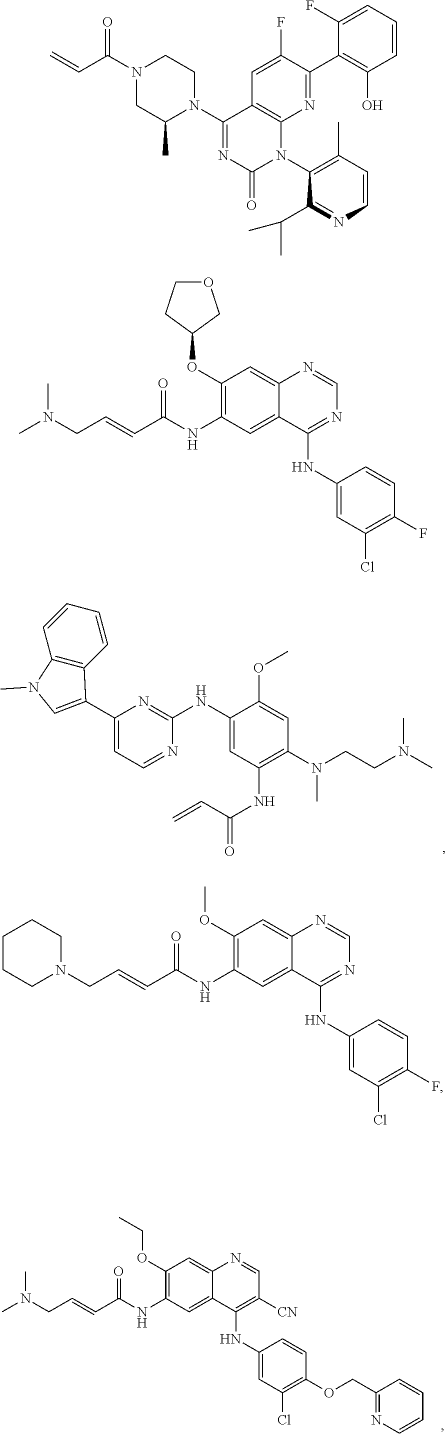

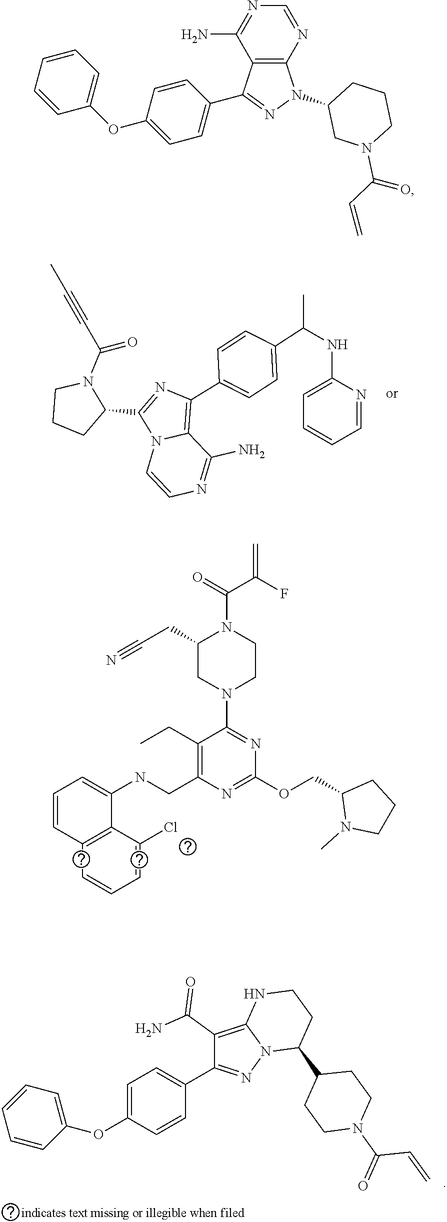 [SMILES: C=CC(=O)N1CCC[C@@H](n2nc(-c3ccc(Oc4ccccc4)cc3)c3c(N)ncnc32)C1 ; CC#CC(=O)N1CCC[C@H]1c1nc(-c2ccc(C(C)Nc3ccccn3)cc2)c2c(N)nccn12 ; "or" ; C=C(F)C(=O)N1CCN(c2nc(OC[C@@H]3CCCN3C)nc(CNc3cccc4cccc(Cl)c34)c2CC)C[C@@H]1CC#N ; C=CC(=O)N1CCC([C@@H]2CCNc3c(C(N)=O)c(-c4ccc(Oc5ccccc5)cc4)nn32)CC1]

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary scheme by which an antibody specifically binding to tumor-associated intracellular target is generated. The exemplary process proceeds with binding an exogenous molecule to the intracellular target associated with a tumor. Illustrated here is the binding of a small molecule that covalently and specifically binds to a tumor-associated intracellular target or an intracellular portion of a membrane bound target. Such covalent interaction creates a new and unique epitope, or makes an existing epitope more assessable for generating an antibody that can in turn specifically recognize the intracellular epitope. Upon exposing the intracellular epitope (e.g., due to cell death or apoptosis), the resulting antibody can specifically target the tissues or cells expressing the intracellular target. The binding of the resulting antibodies creates, e.g., a tumor “GPS” signal, representative of the in situ or in vivo location and identity of the target (conferred by the exogenous molecule specific for such target and the new epitope generated upon binding of such exogenous molecule), and optionally the expression level of such target. Illustrated also are antibody conjugates (e.g., radio-labeled, toxin conjugated, cytokine-linked) that confer additional functionalities including, e.g., cell cytotoxicity, imaging capability, and immune cell activation.

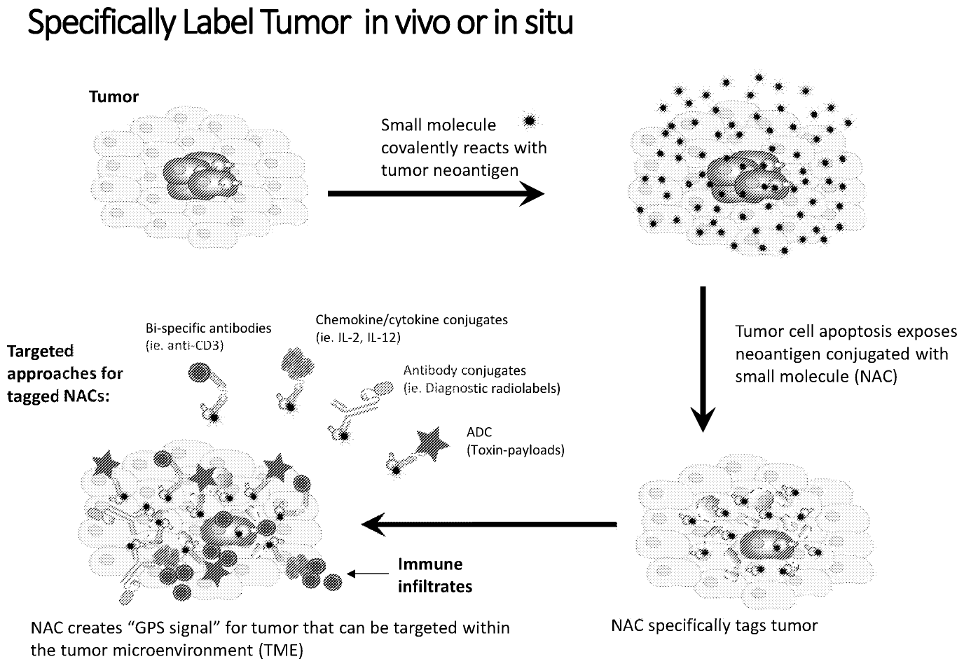

### DETAILED DESCRIPTION

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)).

Unless defined otherwise, all technical and scientific terms used herein have the same meaning as is commonly understood by one of skill in the art to which this invention belongs.

The term “a” and “an” refers to one or to more than one (i.e., to at least one) of the grammatical object of the article. By way of example, “an element” means one element or more than one element.

“About” as used herein when referring to a measurable value such as an amount, a duration, and the like, is meant to encompass variations of ±10% of a stated number or value.

“Amino” refers to the —NH2 radical.

“Cyano” refers to the —CN radical.

“Nitro” refers to the —NO2 radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” or “alkoxyl” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₈ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C5-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atom (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where Re is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Carbocyclylalkynyl” refers to a radical of the formula —R^{c}-carbocyclyl where R^{c} is an alkynylene chain as defined above. The alkynylene chain and the carbocyclyl radical are optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical are optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

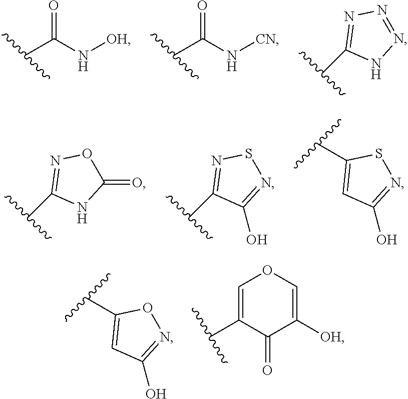

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

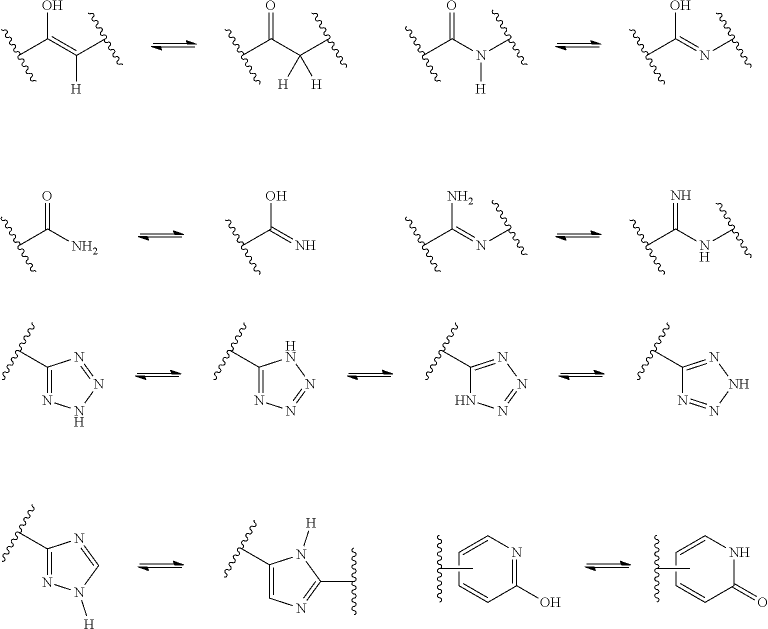

In some instances, the heterocyclic LpxC inhibitory compounds disclosed herein exist in tautomeric forms. The structures of said compounds are illustrated in the one tautomeric form for clarity. The alternative tautomeric forms are expressly included in this disclosure, such as, for example, the structures illustrated below.

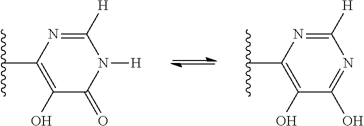 [SMILES: *c1nc[nH]c(=O)c1O ; *c1ncnc(O)c1O]

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (3H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Vanma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

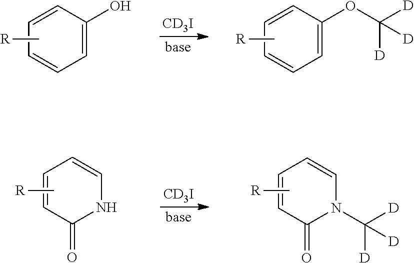

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

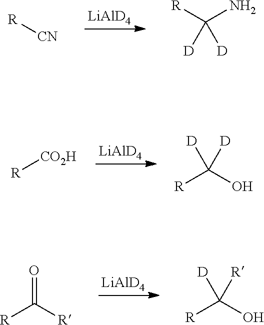

Deuterium gas and palladium catalyst are employed to reduce unsaturated carbon-carbon linkages and to perform a reductive substitution of aryl carbon-halogen bonds as illustrated, by way of example only, in the reaction schemes below.

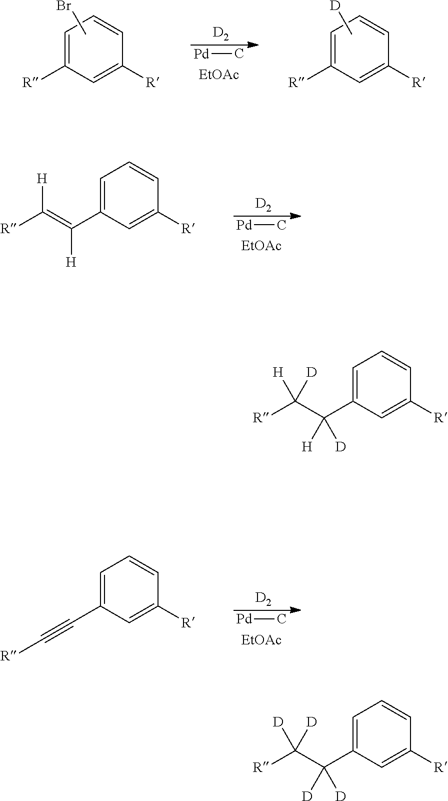

“Pharmaceutically acceptable salt” includes both acid and base addition salts. A pharmaceutically acceptable salt of any one of the heterocyclic LpxC inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), Morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

The term “epitope” as used herein generally refers to at least a portion of an antigen that is recognized by an antigen binding unit. An epitope may be referred to as an antigenic determinant. In an example, an epitope may interact with a specific antigen binding unit in a variable region of an antibody molecule, i.e. a paratope. An epitope may be a surface-accessible portion of an antigen, be buried in the interior portion of the antigen. An epitope may be a part of an active site of an antigen. Alternatively, the epitope may be close to the active site of the antigen, e.g., an active site of a target protein. In an example, an epitope may be at least 1, 2, 3, 4, 5, 6, 7, 8, 9, 10, or more amino acid sequences away from an active site of the target protein. The epitope may be at most 10, 9, 8, 7, 6, 5, 4, 3, 2, or 1 amino acid sequence away from the active site of the target protein. In another alternative, the epitope may not be a part of the active site of the antigen. An epitope may be a single portion of an antigen. Alternatively, an epitope may be a conformational combination of a plurality of portions of an antigen, e.g., at least 2, 3, 4, 5, or more portions of the antigen. In an example, a plurality of epitopes from a plurality of antigens may coalesce to form a new epitope. An epitope may be two-dimensional (i.e., linear) or three-dimensional (i.e., conformational). In an example, an epitope may be a linear chain of amino acid sequences (i.e., a linear polypeptide) of a target protein. In another example, an epitope may be a conformational epitope that is produced by spatially juxtaposed amino acids from different segments of a target protein. Interaction (e.g., binding or complexation) between an epitope and an antigen binding unit may induce a change in a function of an antigen comprising the epitope. In some examples, the antigen binding unit may bind the epitope and initiate or halt a biological activity of the antigen. Alternatively, such interaction between the epitope and the antigen binding unit may not induce any biological effect in the antigen. An antigen may be an extracellular portion of an antigen, a transmembrane portion of an antigen, an intracellular portion of an antigen, or a combination thereof. A single antigen may comprise at least 1, 2, 3, 4, 5, or more epitopes. An epitope may comprise 2, 3, 4, 5, 6, 7, 8, 9, 10, 11, 12, 13, 14, 15 or more consecutive amino acids of an antigen.

An “antigen” is a moiety or molecule that contains an epitope, and, as such, also specifically binds to an antibody.

An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or VHH), a heavy chain found in sharks (V-NAR domain), a single domain antibody (sdAb, i.e., “nanobody”) that comprises a single antigen-binding domain, Fv, Fd, Fab, Fab′, F(ab′)2, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH-VL-CH3)2, (scFv-CH3)2, ((scFv)2-CH3+CH3), ((scFv)2-CH3) or (scFv-CH3-scFv)2), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

The term “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat Acad. Sci, USA 91:3809-3813 (1994); Schier et al. Gene 169:147-155 (1995); Yelton et al. J. Immunol 155:1994-2004 (1995); Jackson et al., J. Immunol. 154(7):3310-9 (1995); and Hawkins et al., J. Mol. Biol. 226:889-896 (1992).

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FW). The variable domains of native heavy and light chains each comprise four FW regions, largely adopting a β-sheet configuration, connected by three CDRs, which form loops connecting, and in some cases forming part of, the β-sheet structure. The CDRs in each chain are held together in close proximity by the FW regions and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991)). The constant domains are generally not involved directly in antigen binding, but may influence antigen binding affinity and may exhibit various effector functions, such as participation of the antibody in ADCC, CDC, and/or apoptosis.

The term “human antibody” refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al., Nature Biotechnology 14:309-314 (1996): Sheets et al. PNAS (USA) 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

The term “hypervariable region” when used herein refers to the amino acid residues of an antibody which are associated with its binding to antigen. The hypervariable regions encompass the amino acid residues of the “complementarity determining regions” or “CDRs” (e.g., residues 24-34 (L1), 50-56 (L2) and 89-97 (L3) of the light chain variable domain and residues 31-35 (H1), 50-65 (H2) and 95-102 (H3) of the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol., 196:901-917 (1987)). “Framework” or “FW” residues are those variable domain residues flanking the CDRs. FW residues are present in chimeric, humanized, human, domain antibodies, single chain diabodies, vaccibodies, linear antibodies, and bispecific antibodies. Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1 (including non-A and A allotypes), IgG2, IgG3, IgG4, IgA, and IgA2. The heavy-chain constant domains that correspond to the different classes of antibodies are called α, δ, ε, γ and μ, respectively. The subunit structures and three-dimensional configurations of different classes of immunoglobulins are well known.

The modifier “monoclonal” indicates the character of the antibody as being obtained from a substantially homogeneous population of antibodies, and is not to be construed as requiring engineering of the antibody by any particular method. The term “monoclonal” is used herein to refer to an antibody that is derived from a clonal population of cells, including any eukaryotic, prokaryotic, or phage clone, and not the method by which the antibody was engineered. For example, the monoclonal antibodies to be used in accordance with the present invention may be made by the hybridoma method first described by Kohler et al., Nature, 256:495 (1975), or may be made by any recombinant DNA method (see, e.g., U.S. Pat. No. 4,816,567), including isolation from phage antibody libraries using the techniques described in Clackson et al., Nature, 352:624-628 (1991) and Marks et al., J. Mol. Biol., 222:581-597 (1991), for example. These methods can be used to produce monoclonal mammalian, chimeric, humanized, human, domain antibodies, single chain diabodies, vaccibodies, and linear antibodies.

The term “chimeric” antibodies includes antibodies in which at least one portion of the heavy and/or light chain is identical with or homologous to corresponding sequences in antibodies derived from a particular species or belonging to a particular antibody class or subclass, and at least one other portion of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

The term “humanized” can refer to forms of nonhuman (e.g., murine) antibodies are chimeric antibodies that contain minimal sequence derived from nonhuman immunoglobulin. For the most part, humanized antibodies are human immunoglobulins (recipient antibody) in which the native CDR residues are replaced by residues from the corresponding CDR of a nonhuman species (donor antibody) such as mouse, rat, rabbit or nonhuman primate having the desired specificity, affinity, and capacity. In some instances, FW region residues of the human immunoglobulin are replaced by corresponding nonhuman residues. Furthermore, humanized antibodies may comprise residues that are not found in the recipient antibody or in the donor antibody. These modifications are made to further refine antibody performance. In general, a humanized antibody heavy or light chain will comprise substantially all of at least one or more variable domains, in which all or substantially all of the CDRs correspond to those of a nonhuman immunoglobulin and all or substantially all of the FWs are those of a human immunoglobulin sequence. In certain embodiments, the humanized antibody will comprise at least a portion of an immunoglobulin constant region (Fc), typically that of a human immunoglobulin. For further details, see, Jones et al., Nature, 321:522-525 (1986); Riechmann et al., Nature, 332:323-329 (1988); and Presta, Curr. Op. Struct. Biol., 2:593-596 (1992).

The term “Fc region” can refer to the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The Fc region of an immunoglobulin generally comprises two constant domains, a CH2 domain and a CH3 domain, and optionally comprises a CH4 domain. By “Fc region chain” herein is meant one of the two polypeptide chains of an Fc region.

The term “CH2 domain” can refer to a human IgG Fc region (also referred to as “Cγ2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protroberance” in one chain thereof and a corresponding introduced “cavity” in the other chain thereof; see U.S. Pat. No. 5,821,333, expressly incorporated herein by reference). Such variant CH3 domains may be used to make multispecific (e.g. bispecific) antibodies as herein described.

The term “efficacy” of a treatment or method, as used herein, can be measured based on changes in the course of disease or condition in response to such treatment or method. For example, the efficacy of a treatment or method of the present disclosure may be measured by its impact on signs or symptoms of a disease or condition of a subject, e.g., a tumor or cancer of the subject. A response may be achieved when a subject having the disease or condition experiences partial or total alleviation of the disease or condition, or reduction of one or more symptoms of the disease or condition. In an example, a response is achieved when a subject suffering from a tumor exhibits a reduction in the tumor size after the treatment or method, as provided in the present disclosure. In some examples, the efficacy may be measured by assessing cancer cell death, reduction of tumor (e.g., as evidenced by tumor size reduction), and/or inhibition of tumor growth, progression, and dissemination.

The term “in vivo” refers to an event that takes place in a subject's body.

The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

### Compositions:

The polypeptides comprising antigen binding units disclosed herein have a wide range of applications in therapeutics, diagnostics, and other biomedical researches. The subject polypeptides, cells comprising the polypeptides are effective tools for targeting or labeling cellular targets of interest, especially cellular targets associated with a disease or disease condition. Of particular interest are the applications of the subject polypeptides and cells containing the same for targeting or labeling tumors, cancer tissues, or cancer cells, and optionally killing the cancer cells being targeted.

In one aspect, the disclosure provides an isolated polypeptide comprising an antigen binding unit, wherein the antigen binding unit (a) exhibits specific binding to a cellular target covalently bound by an exogenous molecule (bound target), but (b) lacks specific binding to the cellular target that is not bound to the exogenous molecule (unbound target).

In another aspect, the present disclosure provides an isolated polypeptide comprising an antigen binding unit, wherein the antigen binding unit (a) exhibits specific binding to an intracellular target or an intracellular portion of a target, which target being bound by an exogenous molecule (bound target), but (b) lacks specific binding to the intracellular target or the intracellular portion of the target, which is not bound to the exogenous molecule (unbound target).

In general, the antigen binding unit utilized in a subject polypeptide typically exhibits the ability to distinguish a bound target from an unbound target. Not wishing to be bound by a particular theory, the binding of an exogenous molecule to a cellular target of interest via a covalent bond creates a new epitope on the bound target, which is otherwise absent or inaccessible by the antigen binding unit when the target is not bound with the exogenous molecule. The formation of the epitopes on the bound target provides a unique identifier that permits the generation of antigen binding units specifically binding to such identifier on the bound target of interest, and not the unbound target. In some embodiments, the binding of the exogenous molecule to the target via a covalent bond renders an existing epitope on the target more accessible or recognizable by the antigen binding unit. In yet some embodiments, formation of the epitope on the bound target does not require a covalent interaction between the exogenous molecule and the target of interest, so long as the interaction (including, without limitation, hydrogen bonding, ironic bonding, van de walls or other non-covalent interactions) creates or induces a stable epitope that becomes recognizable by an antigen binding unit. By “stable” is meant that the epitope is sufficiently long-lasting to persist or accessible, thus permit binding and formation of antigen-epitope complex. For example, the complex can withstand whatever conditions exist or are introduced between the moment of formation and the moment of detection, these conditions being a function of the assay or reaction (whether in vivo or in vitro), which is being performed. In some instances, the formation of the complex is carried out under physiological buffer conditions and at physiological body temperatures ranging from approximately room temperature to approximately 37° C.

Also provided herein is a complex described herein comprising: (1) a modified intracellular target or a modified intracellular portion of a target in a cell, (2) an exogenous molecule, and (3) a polypeptide comprising an antigen binding unit, wherein the exogenous molecule is a covalent inhibitor of the target, and wherein the polypeptide comprising the antigen binding unit specifically binds to an epitope that is (i) formed by binding of said covalent inhibitor to said intracellular target or a modified intracellular portion of a target, and (ii) becomes accessible upon death of the cell. In some embodiments, the antigen binding unit in the complex (x) exhibits specific binding to the intracellular target or the intracellular portion of the target covalently bound by an exogenous molecule (bound target), but (y) lacks specific binding to the intracellular target or the intracellular portion of the target that is not bound to the exogenous molecule (unbound target). In some embodiments, the target in the complex is a tumor associated polypeptide or any other target disclosed herein. For instance, the target is an EGFR bound by a covalent inhibitor of EGFR, and a polypeptide comprising an antigen binding unit that exhibits specific binding to the EGFR bound by said covalent inhibitor. In some embodiments, the complex is present in a dead cell. In some embodiment, complex is detectable in a tumor undergoing necrosis.

An epitope to which a subject antigen binding unit bind may comprise 2, 3, 4, 5, 6, 7, 8, 9, 10, 11, 12, 13, 14, 15 or more consecutive amino acids of the target. An epitope may be two-dimensional (i.e., linear) or three-dimensional (i.e., conformational). In an example, an epitope may be a linear chain of amino acid sequences (i.e., a linear polypeptide) of a target protein. In another example, an epitope may be a conformational epitope that is produced by spatially juxtaposed amino acids from different segments of a target protein. Of particular interest are epitopes defined by both the amino acids of the target and the chemical structure of the exogenous molecule to which the target is bound.

Interaction (e.g., binding or complexing) between an epitope and an antigen binding unit may induce a change in a function of the target comprising the epitope. In some examples, the antigen binding unit may bind the epitope and initiate or halt a biological activity of the antigen. Alternatively, such interaction between the epitope and the antigen binding unit may not induce any biological effect in the target, but merely providing a signal indicative of the in vivo or in situ location of the target being expressed. In some examples, such interaction between the epitope and the antigen binding unit indicates the in vivo or in situ expression level of the target. In some other examples, a single antigen binding unit may bind to a plurality of epitopes induced or formed upon binding of the exogenous molecules to the target.

Specific binding by an antigen binding unit to the bound target can be established by a wide variety of methods and techniques known in the art, including but not limited to direct binding assays, in-direct sandwich assays, ligand binding assays, immunoprecipitation, real-time cell-binding assays, imaging analysis, and competition assays. In an aspect, a binding assay can comprise the use of surface plasmon resonance (SPR), bio-layer interference (BLI), scanning probe microscopy, attenuated total reflective infrared spectroscopy, spectral ellipsometry, mass spectrometry, and any combinations thereof. Conversely, the lack of specific binding to the cellular target that is not bound to the exogenous molecule (unbound target) can be established by similar methods. In an aspect, SPR is used to determine affinity of an antigen binding unit to a target or a portion of a target. Additionally, SPR can be used to determine a physical property or biological property of a subject antigen binding unit provided herein. Physical properties include but are not limited to dielectric properties, adsorption processes, surface degradation, hydration, X-ray crystallography, NMR, interferometry, computer modeling and any combination thereof. Biological properties that can be determined with SPR include but are not limited to adsorption kinetics, desorption kinetics, antigen binding, affinity, epitope mapping, biomolecular structure, protein interaction, biocompatibility, tissue engineering, lipid biolayers, and any combination thereof.

An antigen binding unit embodied herein typically exhibits a higher binding affinity to the bound target relative to the unbound target. In some embodiments, a subject antigen binding unit exhibits about 2, 3, 4, 5, 6, 7, 8, 9, 10, 15, 20, 25, 50, 75, 100, 150, 200, 300, 400, 500, 1000, 1×10⁴, 1×10⁵, 1×10⁶, 1×10⁷, 1×10⁸, or more fold greater affinity towards a bound target than an unbound target.

The terms “dissociation constant,” “equilibrium dissociation constant,” and “K_{D},” as used interchangeably herein, generally refer to an equilibrium constant that measures the propensity of a larger object to dissociate reversibly into smaller components, as when a complex falls apart into a plurality of component molecules. In the context of an interaction between an antibody (Ab) and an antigen of interest (Ag), the dissociation constant is expressed in molar units [M] and corresponds to the concentration of [Ab] at which the binding sites of [Ag] are half occupied, i.e., the concentration of unbound [Ab] equals the concentration of the [AbAg] complex. The dissociation constant can be calculated according to the following formula:

$${K}_{D} = \frac{[ A b ] * [ A g ]}{[ A b A g ]} \qquad ( Equation 1 )$$

In the present disclosure, the dissociation constant can also be expressed in the context of a rate constant that measures the dissociation (K_{off}; [1/sec]) and association (Kₒₙ; [1/sec*M]) of an antibody with an antigen of interest. The dissociation constant can be calculated according to the following formula:

$${K}_{D} = \frac{[ {K}_{off} ]}{[ {K}_{on} ]} \qquad ( Equation 2 )$$

A smaller K_{D}, may indicate a stronger affinity of binding between the antibody and the antigen of interest (e.g., a bound target disclosed herein). In an example, a K_{D} of 1 mM indicates weak binding affinity compared to a K_{D} of 1 nM. Such dissociation constant values for antibodies can be determined by techniques such as, for example, enzyme-linked immunosorbent assay (ELISA), surface plasmon resonance (SPR) (e.g., the Biacore® or the ProteOn® system), isothermal titration calorimetry (ITC), fluorescence depolarization, one or more computer simulations, etc.

In some embodiments, a subject antigen binding unit specific for the bound target lacks specific binding to the unbound target, as evidenced by a dissociation constant toward the unbound target (K_{D, unbound}) that is greater than a dissociation constant toward the bound target (K_{D, bound}) by a factor of at least about 5 fold, 10 fold, 20 fold, 30 fold, 40 fold, 50 fold, 60 fold, 70 fold, 80 fold, 90 fold, 100 fold, 200 fold, 250 fold, 500 fold, 1000 fold, 5000 fold, or more. In some embodiments, the antigen binding unit's dissociation constant toward the bound target (K_{D, bound}) may be smaller than the antigen binding unit's dissociation constant toward the unbound target (K_{D, unbound}) by a factor of at least about 5 fold, 10 fold, 20 fold, 30 fold, 40 fold, 50 fold, 60 fold, 70 fold, 80 fold, 90 fold, 100 fold, 200 fold, 250 fold, 500 fold, 1000 fold, 5000 fold, or more.

In some embodiments, a subject antigen binding unit exhibits specific binding as evidenced by having K_{D} for bound target in the range of about 100 nM to about 0.001 pM but with a K_{D} for unbound target that is at least 1 uM or higher. In some embodiments, a subject antigen binding unit exhibits specific binding as evidenced by having K_{D} for bound target in the range of about 1 nM to about 0.001 pM but with a K_{D} for unbound target that is at least 5 uM or higher. In some embodiments, a subject antigen binding unit exhibits specific binding as evidenced by having K_{D} for bound target in the range of about 1 pM to about 0.001 pM but with a K_{D} for unbound target that is at least 10 uM or higher.

The lack of specific binding to the unbound target is observed when there is no or little detectable complex of the unbound target and antigen binding unit when, for example, the unbound target is presented at a saturation concentration. In some embodiments, the antigen binding unit's dissociation constant toward the unbound target (K_{D, unbound}) may be at least about 500 nM, 1 μM, 5 μM, 10 μM, 50 μM, 100 μM, 500 μM, 1 mM, or more.

In some embodiments, the antigen binding unit's dissociation constant toward the bound target (K_{D, bound}) may be lower than about 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, 1 pM, or less.

Encompassed in the present disclosure are antigen binding units capable of specific binding to an epitope defined by both the amino acids of the bound target (proteinaceous portion of the epitope) and the chemical structure of the exogenous molecule (chemical portion of the epitope). In some embodiments, a subject antigen binding unit specifically binds to both the proteinaceous portion and the chemical portion of the epitope. Also contemplated are antigen binding units capable of specific binding to the proteinaceous portion of an epitope that is induced upon binding of the exogenous molecule to the target via, e.g., covalent bonding. In some embodiments, a subject antigen binding unit lacks specific binding to the exogenous as evidenced by a binding affinity (K_{D}) for the unbound target that is at least 500 nM, 1 uM, 5 uM, 10 uM or higher.

In some embodiments, a subject antigen binding unit exhibits preferential binding to the bound target as compared to that to the exogenous molecule alone. In some cases, the antigen binding unit's dissociation constant toward the bound target (K_{D, bound}) may be smaller than the antigen binding unit's dissociation constant toward the exogenous molecule (K_{D, exogenous molecule}) by a factor of at least about 5 fold, 10 fold, 20 fold, 30 fold, 40 fold, 50 fold, 60 fold, 70 fold, 80 fold, 90 fold, 100 fold, 200 fold, 250 fold, 500 fold, 1000 fold, 5000 fold, or more. In some examples, the ratio of K_{D, bound} over K_{D, exogenous molecule} is at most about 0.5, 0.2, 0.1, 0.05, 0.02, 0.01, 0.005, 0.002, 0.001, 0.0005, or less. Where desired, these antigen binding units are obtained by counter-screening against the exogenous molecules.

Affinity of an antigen binding unit to a target or portion thereof can be measured by any suitable method known in the art, including for example SPR. In brief, SPR allows for real-time analysis of interactions between targets and antigen binding units. For example, a target or portion of a target can be immobilized on a sensor surface of an SPR equipment while the antigen binding unit is injected in an aqueous solution and run through a flow cell of the SPR equipment. Target and antigen binding unit interactions increase refractive index which is in turn measured in real-time and results plotted as response or resonance units (RUs) vs. time. Target and antigen binding unit interactions can be determined using SPR at various settings. SPR may be performed at any temperature. A surface plasmon resonance may be performed at a temperature from about 200 C, 21° C., 22° C., 23° C., 24° C., 25° C., 26° C., 30° C. or up to about 37° C. or 40° C.). A surface plasmon resonance may be performed at a temperature of 25° C. In an aspect, multiple SPR assays may be performed and an average affinity taken for an antigen binding unit provided herein. In an aspect, SPR can be performed on a Biacore instrument. In addition to affinity measurements of antigen binding units and targets or portion thereof, SPR can also be employed to determine binding kinetics, analysis of mutant targets, enthalpy measurements, analyze macromolecular binding.

The subject antigen binding units can comprise sequences of different species origins and can adopt various formats known in the art. Non-limiting examples of subject antigen binding unites include, but are not limited to, a monoclonal antibody, a polyclonal antibody, a recombinant antibody, a human antibody, a humanized antibody, a murine antibody, or a functional derivative, variant or fragment thereof, including, but not limited to, a Fab, a Fab′, a F(ab′)₂, an Fv, a single-chain Fv (scFv), minibody, a diabody, and a single-domain antibody such as a heavy chain variable domain (VH), a light chain variable domain (VL) and a variable domain (VHH) of camelid derived nanobody. In another embodiment, the antigen binding unit includes Camelid single domain antibody, or portions thereof. In one embodiment, Camelid single-domain antibodies include heavy-chain antibodies found in camelids, or VHH antibody. A VHH antibody of camelid (for example camel, dromedary, llama, and alpaca) refers to a variable fragment of a camelid single-chain antibody (See Nguyen et al, 2001; Muyldermans, 2001), and also includes an isolated VHH antibody of camelid, a recombinant VHH antibody of camelid, or a synthetic VHH antibody of camelid. Additional formats of antigen binding units are known in the art, including without limitation those described in US20090155275A1, US20160289343A1, and US20160289341A1 each of which is incorporated herein by reference in its entirety. In some embodiments, the antigen binding unit comprises at least one of a Fab, a Fab′, a F(ab′)₂, an Fv, and a scFv. In some embodiments, the antigen binding unit comprises an antibody mimetic. Antibody mimetics refer to molecules which can bind a target molecule with an affinity comparable to an antibody, and include single-chain binding molecules, cytochrome b562-based binding molecules, fibronectin or fibronectin-like protein scaffolds (e.g., adnectins), lipocalin scaffolds, calixarene scaffolds, A-domains and other scaffolds. In some embodiments, an antigen binding unit comprises a transmembrane receptor, or any derivative, variant, or fragment thereof. For example, an antigen binding domain can comprise at least a ligand binding domain of a transmembrane receptor that recognizes specifically the bound target.

The various units disclosed herein (e.g., antigen binding units, functional units, immune cell signaling units (e.g., primary signaling units and co-stimulatory units), can be linked by means of chemical bond, e.g., an amide bond or a disulfide bond; a small, organic molecule (e.g., a hydrocarbon chain); an amino acid sequence such as a peptide linker (e.g., an amino acid sequence about 3-200 amino acids in length), or a combination of a small, organic molecule and peptide linker. Peptide linkers can provide desirable flexibility to permit the desired expression, activity and/or conformational positioning of the chimeric polypeptide. The peptide linker can be of any appropriate length to connect at least two domains of interest and is preferably designed to be sufficiently flexible so as to allow the proper folding and/or function and/or activity of one or both of the domains it connects. The peptide linker can have a length of at least 3, 5, 10, 15, 20, 25, 30, 35, 40, 45, 50, 55, 60, 65, 70, 75, 80, 85, 90, 95, or 100 amino acids. In some embodiments, a peptide linker has a length between about 0 and 200 amino acids, between about 10 and 190 amino acids, between about 20 and 180 amino acids, between about 30 and 170 amino acids, between about 40 and 160 amino acids, between about 50 and 150 amino acids, between about 60 and 140 amino acids, between about 70 and 130 amino acids, between about 80 and 120 amino acids, between about 90 and 110 amino acids. In some embodiments, the linker sequence can comprise an endogenous protein sequence. In some embodiments, the linker sequence comprises glycine, alanine and/or serine amino acid residues. In some embodiments, a linker can contain motifs, e.g., multiple or repeating motifs, of GS, GGS, GGGGS, GGSG, or SGGG. The linker sequence can include any naturally occurring amino acids, non-naturally occurring amino acids, or combinations thereof.

In some embodiments, the antigen binding unit can comprise a scFV. A scFv is about 30 kDa and comprises variable regions of heavy (VH) and light (VL) chains that are joined together by a flexible peptide linker. In the scFv, the order of the domains can be either VH-linker-VL or VL-linker-VH and in both orientations. In an aspect, a linker can be of any length as previously described. A scFv linker can be rich in glycine for flexibility, as well as serine or threonine for solubility, and can either connect the N-terminus of the VH with the C-terminus of the VL, or vice versa. The peptide linker can be a 15-aa linker with the sequence (Gly₄Ser)₃. Amino acids to be used in linkers can be natural amino acids, amino acid derivatives, D-amino acids, modified amino acids, β-amino acid derivatives, α,α-substituted amino acid derivatives, N-substituted a-amino acid derivatives, aliphatic or cyclic amines, amino- and carboxyl-substituted cycloalkyl derivatives, amino- and carboxyl-substituted aromatic derivatives, γ-amino acid derivatives, aliphatic a-amino acid derivatives, diamines and polyamines. Further modified amino acids are known to the skilled artisan.

A larger Fab is a heterodimer comprising variable and first constant domains of heavy (VH-CH) and light chain (VL-CL) segments linked by disulfide bonds. These fragments show similar binding specificities as the original antibodies and a low degree of immunogenicity and are more easily manipulated than the bivalent parent antibody. In an aspect, a scFv or portion thereof such as Fab, VH, or VL can be directly used as fragments or reconverted into different antibody formats such as full-length antibodies, scFv-CH3 (minibody), scFv-Fc, or diabodies, among others. In some cases, divalent (or bivalent) single-chain variable fragments (di-scFvs, bi-scFvs) can be engineered by linking two scFvs. This can be done by producing a single peptide chain with two VH and two VL regions, yielding tandem scFvs. Another possibility is the creation of scFvs with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target or a portion of a target. The orientation of the variable domains within the scFv, depending on the structure of the scFv, may contribute to whether a scFv will be expressed on a cell surface or whether cells expressing a scFv bind a target and also signal. In addition, the length and/or composition of the variable domain linker may be an important contribution to the stability of the scFv; in studies generating scFvs from a TAG72 antibody (clone B72.3), linkers up to 6×GGGGS demonstrated higher molecular weight dimers and multimers, with clustering decreasing with increasing linker length. In some embodiments, a scFv can be altered. For instance, a scFv may be modified in a variety of ways. In some cases, an scFv can be mutated, so that the scFv may have higher affinity to its target as compared to the parent antibody. In an aspect, a scFv can be modified to reduce clustering on a cell surface to reduce target-independent signaling, or “tonic signaling.” In another aspect, a scFv can be modified to increase proteolytic stability, for example a linker may be used to enhance affinity. For example, the linker: GSTGSGSKPGSGEGSTKG can enhance affinity to a target. A scFv can be derived from an antibody for which the sequences of the variable regions are known. In some embodiments, a scFv can be derived from an antibody sequence obtained from an available mouse hybridoma generated against the bound target.

The procedures for creating scFv libraries are known in the art. Generally, the procedures involve amplification of the variable regions of nucleic acids encoding an antibody, commonly from a hybridoma producing an antibody of interest. Generic primers associated with the constant regions of such antibodies are available commercially. The amplified fragments are then further amplified with primers selected to introduce appropriate restriction sites for introduction of the scFv into an expression vector, phage, or fusion protein. Cells producing the scFv are screened and a scFv with the desired selectivity is identified.

A polypeptide comprising an antigen binding unit may be coupled to (e.g., covalently conjugated to or non-covalently bound to) a particle. The particle may be a carrier for at least the polypeptide. The polypeptide comprising the antigen binding unit may be part of an inner portion (e.g., core) of the particle and/or part of a surface of the particle. In some cases, the polypeptide may be an antibody (or a functional variant thereof), and one or more binding specificities of the antibody may be substantially maintained when the antibody is incorporated into an antibody-particle (e.g., antibody-nanoparticle) package. In some cases, the antibody may be released from the package to achieve its intended biological activities. In some cases, the antibody may be biologically functional while being coupled to the package. A particle may have various shapes and sizes. For example, a particle may be in the shape of a sphere, spheroid, cone, cuboid, or disc, or any partial shape or combination of shapes thereof. The particle may have a cross-section that is circular, triangular, square, rectangular, pentagonal, hexagonal, or any partial shape or combination of shapes thereof. A particle may be solid, at least partially hollow (e.g., solid outer core with a hollow inner core), or be multilayered. For example, a particle may include a solid core region and at least one solid outer region (i.e., an encapsulating layer). Two or more regions of the particle may be cross-linked. Alternatively, the two or more regions of the particle may not be cross-linked (e.g., bound by ionic bond, hydrogen bond, van der Waals interaction, etc.).

A particle may be composed of one substance or any combination of a variety of substances, including lipids, polymers, ceramic materials, magnetic materials, or metallic materials, such as silica, gold, silver, platinum, aluminum, iron oxide, and the like. Lipids may include fats, waxes, sterols, cholesterol, fat-soluble vitamins, monoglycerides, diglycerides, phospholipids, sphingolipids, glycolipids, cationic or anionic lipids, derivatized lipids, cardiolipin, and the like. Polymers may include block copolymers generally, poly(lactic acid), poly(lactic-co-glycolic acid), polyethylene (e.g., polyethylene glycol), acrylic polymers, cationic polymers, polypeptides, polypeptoids, polynucleotides, and the like. Ceramics may include alumina, zirconia, and titania. In some cases, ceramics may be metal oxides capable of forming hydroxyl groups. Metals may include gold, silver, platinum, titanium, chromium, etc. Metals may include alloys, such as Cr alloys and titanium alloys. Examples of Cr alloys may include Co—Cr alloys or Co—Cr—Mo alloys. Examples of titanium alloys may include Ti-6A1-4V alloy, Ti-15Mo-5Zr-3A1 alloy, Ti-6A1-7Nb alloy, Ti-6A1-2Nb-1Ta alloy, Ti-15Zr-4Nb-4Ta alloy, Ti-15Mo-5Zr-3A1 alloy, Ti-13Nb-13Zr alloy, Ti-12Mo-6Zr-2Fe alloy, Ti-15Mo alloy, and Ti-6A1-2Nb-1Ta-0.8Mo alloy. In some embodiments, a particle may be composed of at least a pharmaceutically acceptable material. The term “pharmaceutically acceptable” material generally refers a material suitable for administration to a subject (e.g., humans, animals, insects, plants, etc.) without giving rise to unduly deleterious side effects (e.g., inflammation, blood coagulation, fibrous tissue formation, etc.). In some cases, a particle may be biodegradable and/or biocompatible.

Examples of a particle may include, but are not limited to, a liposome, a micelle, a lipoprotein, a lipid-coated bubble, a block copolymer micelle, a polymersome, a niosome, a quantum dot, an iron oxide particle, a gold particle, a dendrimer, a silica particle, and a circular nucleic acid. In certain embodiments, a lipid monolayer or bilayer can fully or partially coat a nanoparticle composed of a material capable of being coated by lipids, e.g., polymer nanoparticles. In some cases, liposomes may be multilamellar vesicles (MVLV), large unilamellar vesicles (LUV), small unilamellar vesicles (SUV), or variations thereof.

In some embodiments, a polypeptide comprising an antigen binding unit to a substrate may be chemically conjugated to one or more components of a particle via a cross-linker. The term “cross-linker” as used herein generally refers to a bifunctional or multi-functional chemical or biological moiety that is capable of linking two separate moieties together (e.g., a first antibody and a second antibody, an antibody and a polymer, antibody and a substrate surface, an antibody and a label, etc.). A cross-linker may promote self-conjugation, intramolecular cross-linking, and/or polymerization of one or more moieties. A reactive group of the one or moieties (e.g., a polypeptide comprising an antigen binding domain) that may be targeted for cross-linking may include, but are not limited to, primary amines, sulfhydryls, carbonyls, carbohydrates, and carboxylic acids. Cross-linkers may comprise varying lengths of spacer arms or bridges. Bridges may connect two reactive ends, e.g., a first reactive end of a polypeptide comprising the antigen binding unit and a second reactive end of a composition of a particle. Examples of homobifunctional cross-linkers include, but are not limited to, imidoesters, N-hydroxysuccinimidyl (NHS) esters, maleimides, alkyl and aryl halides, α-haloacyls, pyridyl disulfides, carbodiimides, arylazides, glyoxals, and carbonyls.

In some embodiments, formation of the particle may at least partially involve self-assembly. The term “self-assembly” as used herein generally refers to a process in which particles spontaneously gather (or coalesce) to form a mass to minimize the surface energy in the total system. Self-assembly may lead to the formation of an aggregate without any covalent bonds, but rather using non-covalent bonds, e.g., hydrophobic interactions, hydrogen bonding, ionic bonding, etc. A self-assembly may be a spontaneous process occurring without any energy input when environmental conditions such as composition, pH, temperature, and concentration of solvent are appropriate. Alternatively, self-assembly may benefit from or require some energy input, such as temperature. In some examples, a polypeptide comprising an antigen binding unit may be conjugated to a hydrophobic moiety or an amphiphilic moiety, in which binding interactions between a plurality of the hydrophobic moiety or the amphiphilic moiety may drive formation of a self-assembled particle comprising the polypeptide (e.g., presenting the polypeptide on an outer surface of the self-assembled particle). Examples of self-assembled aggregate may include micelles, liposomes, peptide amphiphiles, drug amphiphiles, DNA origami particles, etc.

A particle may be a microparticle. The term “microparticle” generally refers to a particle that is about 1 micrometer (μm) to about 1 millimeter (mm) in diameter. In some cases, the microparticle may be at least about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 975, 999 μm, or more in diameter. In some cases, the microparticle may be at most about 999, 975, 950, 900, 850, 800, 750, 700, 650, 600, 550, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 20, 15, 10, 9, 8, 7, 6, 5, 4, 3, 2 μm, or less in diameter. Alternatively, a particle may be a nanoparticle. The term “nanoparticle” generally refers to a particle that is about 0.5 nanometer (nm) to about 1 μm in diameter. In some cases, the nanoparticle may be at least about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 975, 999 nm, or more in diameter. In some cases, the nanoparticle may be at most about 999, 975, 950, 900, 850, 800, 750, 700, 650, 600, 550, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6 μm, or less in diameter.

The present disclosure also provides multivalent antigen binding units. A multivalent antigen binding unit typically comprises more than one antigen binding domain, arranged in a single contiguous polypeptide or multiple polypeptides that are linked together. For example, a multivalent antigen binding unit is typically multispecific, possessing the ability to bind to two or more distinct epitopes via two or more of its antigen-binding domains. Examples of multivalent antigen binding units include, but are not limited to, a diabody (db), a single-chain diabody (scDb), a tandem scDb (Tandab), a linear dimeric scDb (LD-scDb), a circular dimeric scDb (CD-scDb), a di-diabody, a tandem scFv, a tandem di-scFv (e.g., a bispecific T cell engager or “BiTE”), a tandem tri-scFv, a tri(a)body, a bispecific Fab₂, a di-miniantibody, a tetrabody, an scFv-Fc-scFv fusion, a dual-affinity retargeting (DART) antibody, a dual variable domain (DVD) antibody, an IgG-scFab, an scFab-ds-scFv, an Fv2-Fc, an IgG-scFv fusion, a dock and lock (DNL) antibody, a knob-into-hole (KiH) antibody (bispecific IgG prepared by the KiH technology), a DuoBody (bispecific IgG prepared by the Duobody technology), a heteromultimeric antibody, a heteroconjugate antibody, functional variants thereof, and combinations thereof.

The term “diabody” generally refers to polypeptide chains that complex with one another (e.g., non-covalently) to form two antigen binding units. Each polypeptide chain may comprise two domains: VH and VL. By using a linker that may be too short (or too rigid) to allow pairing between the two domains of the same polypeptide chain, the two domains may be forced to pair with complementary domains of another polypeptide chain and form two antigen binding units. A diabody may be bispecific.

In some embodiments, a bivalent antigen binding unit comprises two antigen binding domains that exhibit specific binding affinities to different target antigens, different target epitopes of the same antigen, or different epitopes of different antigens. In some examples, a first antigen binding domain may specifically recognize a bound target (e.g., a tumor antigen bound an exogenous molecule), and a second antigen binding unit may specifically recognize a cell surface molecule (e.g., CD3 on T lymphocytes) on an immune cell including an effector cell, or vice versa.

In some embodiments, a first antigen binding domain may specifically recognize a bound target, and the second antigen binding domain may specifically recognize another antigen distinct from the bound target, or vice versa. Such distinct antigen can be a tumor associated polypeptide (including without limitation PDL1 and TNF beta), a cellular protein associated with other diseases or conditions, and/or a cellular target that is intracellular, secreted, membrane bound, differentially expressed in a specific organelle within a cell (e.g., nucleus, ER or Golgi). For example, the second binding domain incorporated into a subject antigen binding unit can comprise an anti-PDL1 or anti-TNF beta binding domain, linked in frame with the antigen binding domain specific for the bound target.

The distinct antigen can also be an immune cell antigen, a cytokine, a chemokine, a radioisotope, a fluorophore, or a toxin. Exemplary immune cell antigen includes but are not limited to check point antigens such PD1, CTLA-4, Siglec-15 (S15), LAG3, TIM3, TIGIT, OX40, cluster of differentiation 93 (CD93), ADORA2A, cluster of differentiation 276 (CD276), VTCN1, BTLA, IDO1, KIR3DL1, VISTA, cluster of differentiation 244 (CD244), CISH, HPRT1, AAVS1, CCR5, CD160, cluster of differentiation 96 (CD96), cluster of differentiation 355 (CD355), SIGLEC7, SIGLEC9, TNFRSF10A, TNFRSF10B, CASP3, CASP6, CASP7, CASP8, CASP10, FADD, FAS, TGFBRII, TGFBRI, SMAD2, SMAD3, SMAD4, SKI, SKIL, TGIF1, IL10RA, IL10RB, CSK, PAG1, EGLN3, or combinations thereof.

In some cases, a bivalent antigen binding unit may comprise two scFvs (i.e., a bispecific scFv), and each scFv may comprise one VH and one VL region. In such cases, the bivalent scFv may be a tandem bi-scFv or a diabody. A bivalent scFv may comprise four domains: VH1 and VL1 of a first antigen binding unit; and VH2 and VL2 of a second antigen binding unit. Such bivalent scFv may be arranged in different formats selected from the group consisting of: VH1-Lx-VL1-Ly-VH2-Lz-VL2; VL1-Lx-VH1-Ly-VH2-Lz-VL2; VL1-Lx-VH1-Ly-VL2-Lz-VH2; VH1-Lx-VL1-Ly-VL2-Lz-VH2; VH1-Lx-VL2-Ly-VH2-Lz-VL1; VL1-Lx-VL2-Ly-VH2-Lz-VH1; VH1-Lx-VH2-Ly-VL2-Lz-VL1; VL1-Lx-VH2-Ly-VL2-Lz-VH1; VH2-Lx-VL1-Ly-VH1-Lz-VL2; VL2-Lx-VL1-Ly-VH1-Lz-VH2; VH2-Lx-VH1-Ly-VL1-Lz-VL2; and VL2-Lx-VH1-Ly-VL1-Lz-VH2. Linkers Lx, Ly, and Lz may be the same or different. A functional form of a tandem bi-scFv may comprise the four domains in a single linear polypeptide, as illustrated in the abovementioned formats. On the contrary, a functional form of a diabody may not comprise the linker Ly, thus splitting the bispecific scFv into two polypeptide chains that are non-covalently coupled to one another. In an example, Ly may be a self-cleaving peptide sequence (e.g., T2A, P2A, E2A, F2A, etc.) that is cleaved after translation of a single polypeptide comprising the four domains. Alternatively, a diabody may be expressed as two separate polypeptide chains, wherein each polypeptide is preceded by a promoter e.g., (i) a first polypeptide chain comprising what is left of Ly and (ii) a second polypeptide chain comprising what is right of Ly in the abovementioned formats. Exemplary promoters for use in the latter approach may have skipping activity such as self-cleavage promoters, T2A, P2A, E2A, F2A, and IRES. In the case where promoters have skipping activity, the first polypeptide and second polypeptide may be expressed at different levels. For example, the first polypeptide may be expressed at higher amounts than the second polypeptide. In some cases, the first polypeptide and the second polypeptide are expressed at equal amounts.

In some embodiments, a subject multivatlent antigen binding unit comprises one or more functional units, particularly those functional units exhibiting specific binding or affinity to an antigen distinct from the bound target.

Linkers of a multivalent antigen binding unit (e.g., Lx, Ly, and Lz, as abovementioned) may be peptide linkers of any length. In some cases, a peptide linker between VH and VL of an antigen binding unit (e.g., scFv) may be from 1 amino acids to 20 amino acids long, from 2 amino acids to 19 amino acids long, from 3 amino acids to 18 amino acids long, from 4 amino acids to 17 amino acids long, from 5 amino acids to 17 amino acids long, from 6 amino acids to 17 amino acids long, from 7 amino acids to 18 amino acids long, from 8 amino acids to 17 amino acids long, from 9 amino acids to 17 amino acids long, from 10 amino acids to 17 amino acids long, from 11 amino acids to 16 amino acids long, from 12 amino acids to 17 amino acids long, from 13 amino acids to 16 amino acids long, from 14 amino acids to 16 amino acids long, or from 14 amino acids to 15 amino acids long. In some cases, a peptide linker between VH and VL of an antigen binding unit may be at least 1, 2, 3, 4, 5, 6, 7, 8, 9, 10, 11, 12, 13, 14, 15, 16, 17, 18, 19, 20, or more amino acids long. In some cases, a peptide linker between VH and VL of an antigen binding unit may be at most 20, 19, 18, 17, 16, 15, 14, 13, 12, 11, 10, 9, 8, 7, 6, 5, 4, 3, 2, or 1 amino acid long. In some cases, a peptide linker between a first antigen binding unit and a second antigen binding unit of a multispecific antigen binding unit may be at least 1, 2, 3, 4, 5, 6, 7, 8, 9, 10, 11, 12, 13, 14, 15, 16, 17, 18, 19, 20, or more amino acids long. In some cases, a peptide linker between a first antigen binding unit and a second antigen binding unit of a multispecific antigen binding unit may be at most 20, 19, 18, 17, 16, 15, 14, 13, 12, 11, 10, 9, 8, 7, 6, 5, 4, 3, 2, or 1 amino acid long. In some cases, such peptide linker may not comprise any polymerization activity to prevent undesired aggregation of a plurality of the multispecific antigen binding units. Linkers may be a stable linker. Linkers may not be cleavable by protease, e.g., matrix metalloproteinases (MMPs). Linkers may be rigid linkers. Alternatively, linkers may be flexible linkers. Examples of flexible linkers may include, but are not limited to, glycine polymers (G)ₙ, glycine-serine polymers (including, for example, (GS)ₙ, (GSGGS)ₙ and (GGGS)ₙ, where n is an integer of at least one), glycine-alanine polymers, alanine-serine polymers, functional variants thereof, and combinations thereof. Other examples of flexible linkers may include GGGGSGGGGSGGGGS, GGGGSGGGGSGGSA, and GGGGSGGGGSGGGGS.

In some embodiments, a subject polypeptide further comprises a functional unit that confers an additional function besides the specific binding by the antigen binding unit to the bound target of interest. For example, a functional unit can be incorporated into a subject polypeptide can be a label to effect detection of the antigen binding unit and/or the bound target in vivo or in vitro. A wide variety of labels are known in the art, including without limitation, a radioisotope, a fluorophore, magnetic or paramagnetic particle, biotin, tags, conjugates, and an enzyme that mediates a reaction upon exposure to substrate to provide a detectable readout.

In some cases, the label may be expressed as a part of the subject polypeptide during or subsequent to biosynthesis of the polypeptide. In some examples, the label (e.g., an amino acid sequence) may be incorporated to the polypeptide during post-translation modification (PTM), e.g., via an enzymatic modification of the polypeptide. In other examples, the polypeptide may be conjugated or tagged with the label. In some cases, the label may be non-covalently bound to or adjacent to the antigen binding unit.

Non-limiting exemplary radioisotope labels include ⁹⁰Y, ¹¹¹In, ¹⁷⁷Lu, ^{99m}Tc, ¹³¹I, ¹²³I, ¹²⁵I, ¹²¹I, ¹³¹Im, Na¹²⁵I, Na¹³¹I, carbon (¹⁴C), sulfur (³⁵S), tritium (3H), indium (¹¹⁵In, ¹¹³In ¹¹²In, ¹¹¹In,), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Y, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ^{U7}Tin, and any combination thereof. Non-limiting exemplary fluorophores include Alexa fluor 488, alexa fluor 555, alexa fluor 568, alexa fluor 594, alexa fluor 647, alexa fluor 700, AMCA, Cy3B, Cy3, Cy3.5, Cy5, Cy 5.5, Cy7, Cy7.5, ATTO 700, ATTO 680, ATTO 655, PerCP, APC/Cy7, APC, BPE, R-PE, RPC, Dylight 633, ATTO 633, ATTO 594, PE/ATTO 594, rhodamine, Texas red, Dylight 594, ATTO 565, R-PE, Dylight 488, TMR, Eosin, Marina blue, Oregon Green, rhodol green, ATTO 488, FITC, ATTO390, Dylight 405, and Dylight 350. In an aspect, a fluorophore may have an excitation Max of about 353, 400, 390, 494, 501, 493, 565, 563, 593, 535, 601, 629, 638, 650, 652, 482, 663, 680, or 700. In an aspect, a fluorophore may have an emission max of 432, 420, 479, 520, 523, 518, 575, 592, 618, 627, 657, 658, 660, 790, 677, 684, 700, or 719.

Non-limiting exemplary enzymes that can be incorporated into a subject antigen binding unit are horseradish peroxidase, alkaline phosphotase (APase), beta-galactosidase, urease, glucose oxidase, and combinations thereof.

The various types of labels as the functional units can be directly conjugated or indirectly linked to a subject antigen binding unit via a linker. A wide variety of chemical linkers are available in the art. For example, reagents including maleimide, disulfide and the process of acylation can be used to form a direct covalent bond with a cysteine on an antigen binding unit. Amide coupling can be used at an aspartamate and glutamate to form an amide bond. Diazonium coupling, acylation, and alkylation can be used at a tyrosine on antigen binding unit to form an amide bond linkage. It is possible that any of the amino acids (20 amino acids or any unnatural amino acids) can be used to form the direct covalent bond that is the attachment of a functional unit to the antigen binding unit. In some embodiments, the linker may be conjugated to a subject antigen binding unit using a coupling group. For example, the coupling group can be an activated ester (e.g. NHS ester, 1-ethyl-3-(3-dimethylaminopropyl) carbodiimide (EDC) ester, dicyclohexylcarbodiimide (DCC) ester, etc.), or an alkyl or acyl halide (e.g. —Cl, —Br, —I).

In some embodiments, a functional unit can be incorporated into an antigen binding unit using a bifunctional crosslinker. The bifunctional crosslinker can comprise two different reactive groups capable of coupling to two different functional targets such as peptides, proteins, macromolecules, semiconductor nanocrystals, or substrate. The two reactive groups can be the same or different and include but are not limited to such reactive groups as thiol, carboxylate, carbonyl, amine, hydroxyl, aldehyde, ketone, active hydrogen, ester, sulfhydryl or photoreactive moieties. In some embodiments, a cross-linker can have one amine-reactive group and a thiol-reactive group on the functional ends. In other embodiments, the bifuncitonal crosslinker can be an NHS-PEO-Maleimide, which comprise an N-hydroxysuccinimide (NHS) ester and a maleimide group that allow covalent conjugation of amine- and sulfhydryl-containing molecules. Further examples of heterobifunctional cross-linkers that may be used to conjugate the linker to the targeting unit or therapeutic unit include but are not limited to: amine-reactive+sulfhydryl-reactive crosslinkers, carbonyl-reactive+sulfhydryl-reactive crosslinkers, amine-reactive+photoreactive crosslinkers, sulfhydryl-reactive+photoreactive crosslinkers, carbonyl-reactive+photoreactive crosslinkers, carboxylate-reactive+photoreactive crosslinkers, and arginine-reactive+photoreactive crosslinkers.

Typical crosslinkers can be classified in the following categories (with exemplary functional groups): (a) Amine-reactive: the cross-linker couples to an amine (NH2) containing molecule, e.g. isothiocyanates, isocyanates, acyl azides, NHS esters, sulfonyl chlorides, aldehydes and glyoxals, epoxides and oxiranes, carbonates, arylating agents, imidoesters, carbodiimides, anhydrides, alkynes; (b) Hydroxyl-reactive: the cross-linker couple to a hydroxyl (—OH) containing molecule, e.g. epoxides and oxiranes, carbonyldiimidazole, oxidation with periodate, N,N′-disuccinimidyl carbonate or N-hydroxylsuccimidyl chloroformate, enzymatic oxidation, alkyl halogens, isocyanates; (c) Thiol-reactive: the cross-linker couple to a sulfhydryl (SH) containing molecule, e.g. haloacetyl and alkyl halide derivates, maleimides, aziridines, acryloyl derivatives, arylating agents, thiol-disulfides exchange reagents; (d) Carboxylate-reactive: the cross-linker couple to a carboxylic acid (COOH) containing molecule, e.g. diazoalkanes and diazoacetyl compounds, such as carbonyldiimidazoles and carbodiimides; (e) Aldehyde- and ketone-reactive: the cross-linker couple to an aldehyde (—CHO) or ketone (R2CO) containing molecule, e.g. hydrazine derivatives for schiff base formation or reduction amination; (f) Active hydrogen-reactive, e.g. diazonium derivatives for mannich condensation and iodination reactions; and (f) Photo-reactive, e.g. aryl azides and halogenated aryl azides, benzophenones, diazo compounds, diazirine derivatives.

Where desired, a subject antigen binding unit may incorporate a toxin by any methods known in the art. For instance, an antigen binding unit may be conjugated to a toxin or if the toxin comprises amino acids, the toxin can be recombinantly produced as part of the antigen binding unit. A subject antigen binding unit can comprise one or more of the following exemplary toxins: CPX-351, cytarabine, daunorubicin, vosaroxin, sapacitabine, idarubicin, or mitoxantrone. Other examples of toxins and fragments thereof may include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and tricothecenes.

In some embodiments, a subject polypeptide comprises a functional unit to improve the biological and/or physiological properties of the resulting antigen binding units. For instance, a functional unit may increase solubility, thermal stability, conformational flexibility or rigidity (whichever is more desirable), and/or half-life of the antigen binding unit.

In an aspect, an antigen binding unit or portion thereof can be modified. Modifications can improve properties of subject antigen binding units. In some embodiments, a modification can improve a physical property of a subject antigen binding unit. Physical properties can include but are not limited to immunogenicity, water solubility, bioavailability, serum half-life, therapeutic half-life, or combinations thereof. In some cases, a modification may assist in isolating antigen binding units, for example purification and/or detection. A property can also be biological such that the modification improves a function of the antigen binding unit. In an aspect, a modification of an antigen binding unit can comprise use of a nonproteinaceous polymer. A nonproteinaceous polymer can comprise polyethylene glycol (PEG), polypropylene glycol, or polyoxyalkylene, such as poly (2-alkyl-2-oxazoline). Modifications can include PEGylation. PEGylation may improve plasma half-life and reduce susceptibility to protease degradation of subject antigen binding units. Such PEG-conjugated biomolecules can possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

Polyethylene glycol molecules can be covalently attached to at least one amino acid residue of a subject antigen binding unit. PEGylation of an antigen binding unit can generally occur via a linker. PEGs suitable for conjugation to a polypeptide sequence of an antigen binding unit as provided herein are generally soluble in water at room temperature, and have the general formula R(0-CH2-CH2)nO-R, where R is hydrogen or a protective group such as an alkyl or an alkanol group, and where n is an integer from 1 to 1000. When R is a protective group, it generally has from 1 to 8 carbons. PEGylation most frequently occurs at the alpha amino group at the N-terminus of a polypeptide, for example an antigen binding unit polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein such as those provided in WO2017123557A1 incorporated herein by reference. Two widely used first generation activated monomethoxy PEGs (mPEGs) are succinimdyl carbonate PEG (SC-PEG; see, e.g., Zalipsky, et al. (1992) Biotehnol. Appl. Biochem 15: 100-114; and Miron and Wilcheck (1993) Bio-conjug. Chem. 4:568-569) and benzotriazole carbonate PEG (BTC-PEG; see, e.g., Dolence, et al. U.S. Pat. No. 5,650,234), which react preferentially with lysine residues to form a carbamate linkage, but are also known to react with histidine and tyrosine residues. The linkage to histidine residues on certain molecules (e.g., IFNα) has been shown to be a hydrolytically unstable imidazolecarbamate linkage (see, e.g., Lee and McNemar, U.S. Pat. No. 5,985,263). Second generation PEGylation technology has been designed to avoid these unstable linkages as well as the lack of selectivity in residue reactivity. Use of a PEG-aldehyde linker targets a single site on the N-terminus of a polypeptide through reductive amination. PEG can be bound to an antigen binding unit of the present disclosure via a terminal reactive group (a “spacer” or “linker”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide. Another activated polyethylene glycol which can be bound to a free amino group is 2,4-bis(0-methoxypolyethyleneglycol)-6-chloro-s-triazine, which can be prepared by reacting polyethylene glycol monomethyl ether with cyanuric chloride. The activated polyethylene glycol which is bound to the free carboxyl group includes polyoxyethylenediamine. Conjugation of one or more of the polypeptide sequences, comprising antigen binding unit sequences, of the present disclosure to PEG having a linker can be carried out by various conventional methods described in, e.g., U.S. Pat. Nos. 5,252,714; 5,643,575; 5,919,455; 5,932,462; and 5,985,263.

The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range; by way of example, certain embodiments have molecular weights between 5 kDa and 20 kDa, while other embodiments have molecular weights between 4 kDa and 10 kDa. Representative PEG molecular weights can include 300 Da, 600 Da, 1 kDa, 2 kDa, 3 kDa, 4 kDa, 6 kDa, 8 kDa, 10 kDa, 15 kDa, 20 kDa, 30 kDa, 50 kDa, 100 kDa, 200 kDa, 500 kDa, and 1 MDa and all values within the range of 300 Daltons to 1 MDa. PEG of any given molecular weight may vary in other characteristics such as length, density, and branching.

In some embodiments, a polyethylene glycol (PEG) is incorporated into an antigen binding unit in accordance to any known methods in the art. Of particular interest are crosslinkers comprising polyethylene glycol (PEG), or PEG-containing hydrocarbon spacers that can improve water solubility of antigen binding units, reduce the potential for aggregation, and increases flexibility of the crosslink, resulting in reduced immunogenic response to the spacer itself.

Where desired, an antigen binding unit can be conjugated to an XTEN polypeptide or recombinantly produced in-frame with one or more XTEN polypeptides. A large number of XTEN sequences are known and shown to improve half-life, stability and/or solubility of a polypeptide to which it is attached. See, for example, U.S. Pat. Nos. 8,673,860, 9,371,369, 9,976,166, each of which is incorporated herein in its entirety. Not wishing to be bound by any particularly theory, an XTEN-linked antigen binding unit exhibits longer half-life in vivo and in vitro than the one without the XTEN. One or more XTEN sequences can be inserted at the N-terminus, C-terminus, or within the antigen binding units, so long as such insertion does not abolish the specific binding ability of the antigen binding unit to the intended bound target. An XTEN can comprise a cleavage sequence that permits cleavage of XTEN from the antigen binding unit via the action of a cleavage enzyme such as a proteinase. A wide range of cleavage sequences that can be incorporated into an XTEN are described in WO 2017040344 and WO 2019126567 (all of which are incorporated herein by reference in their entirety), as well as a range of antibody-XTEN formats. In some embodiments, a subject antigen binding unit is linked in frame with a one or more XTEN via a cleavage sequence, such that binding of the bound target is activated upon cleaving the XTEN by a proteinase that is preferentially expressed in a tissue, a cell type of interest. In some instances, an XTEN-linked antigen binding unit specifically targets a tumor associated polypeptide that is bound by an exogenous molecule. Cleaving the XTEN by a proteinase present at a tumor site exposes the binding domain of the antigen binding unit and hence activating target binding at the tumor site. The approach may can (a) yield a long lasting antigen binding unit of small size, typically a single chain antibody with the aid of an XTEN; (b) reduce non-specific or off-target binding of the long lasting antigen binding unit while it is circulating in vivo; and/or (c) increase penetration of the antigen binding unit at the tumor site, particularly for solid tumor, because of the reduced size of the antigen binding unit upon cleavage by the proteinase at the tumor site.

In some embodiments, a functional unit contained in a subject polypeptide confers a biological function, including but not limited to apoptosis, cell proliferation, cell differentiation, cell migration, cytotoxicity, release or trafficking of intercellular molecules, growth factor, metabolite, anti-angiogenic, anti-hypoxic, chemical compound, or a combination thereof.

In some instances, the functional unit contained in a subject polypeptide comprises an apoptosis-inducing agent including without limitation: caspase-1 ICE, caspase-3 YAMA, inducible Caspase 9 (iCasp9), AP1903, HSV-TK, CD19, RQR8, tBID, CD20, truncated EGFR, Fas, FKBP12, CID-binding domain (CBD), and any combination thereof. Examples of further suicide systems include those described by Jones et al. (Jones B S, Lamb L S, Goldman F and Di Stasi A (2014) Improving the safety of cell therapy products by suicide gene transfer. Front. Pharmacol. 5:254. doi: 10.3389/fphar.2014.00254), which is incorporated herein by reference in its entirety.

In some instances, the functional unit comprises a cell differentiation agent including without limitation: ANGPT1, ANGPT2, ANGPTL2, ANGPTL3, ANGPTL5, ANGPTL7, BDNF, BMP2, BMP3, BMP4, BMP7, CCL2, CCL3, CNTF, CSF2, CSF3, CXCL12, CXCL8, DKK1, DLL1, DLL4, EGF, EPO, FGF1, FGF10, FGF18, FGF19, FGF2, FGF4, FGF5, FGF6, FGF7, FGF8B, FGF9, FLT3LG, GDF3, GDF5, HGF, IFNA1, IFNG, IGF1, IGF2, IL10, IL11, IL12B, IL13, IL15, IL16, IL17A, IL18, IL1A, IL1B, IL2, IL27, IL3, IL32, IL33, IL34, IL4, IL6, IL7, IL9, INHBA, JAG1, KITLG, LGALS1, LIF, MFAP4, MSTN, NGF, NOG, OSM, PDGFB, PTN, RSPO1, RSPO2, RSPO3, RSPO4, SHH, SOX2, TGFA, TGFB1, TGFB2, TGFB3, TNF, TPO, VEGFA, VEGFC, VTN, WNT1, WNT5A, WNT7A, or combination thereof.

In some instances, the functional unit comprises a cell migration agent including without limitation: ARMCX2, BCA-1/CXCL13, CCL11, CCL12/MCP-5, CCL13/MCP-4, CCL15/MIP-5/MIIP-1 delta, CCL16/HCC-4/NCC4, CCL17/TARC, CCL18/PARC/MIP-4, CCL19/MIP-3b, CCL2/MCP-1, CCL20/MIP-3 alpha/MIP3A, CCL21/6Ckine, CCL22/MDC, CCL23/MIP 3, CCL24/Eotaxin-2/MPIF-2, CCL25/TECK, CCL26/Eotaxin-3, CCL27/CTACK, CCL28, CCL3/Mip1a, CCL4/MIP1B, CCL4L1/LAG-1, CCL5/RANTES, CCL6/C10, CCL8/MCP-2, CCL9, CML5, CXCL1, CXCL10/Crg-2, CXCL12/SDF-1 beta, CXCL14/BRAK, CXCL15/Lungkine, CXCL16/SR-PSOX, CXCL17, CXCL2/MIP-2, CXCL3/GRO gamma, CXCL4/PF4, CXCL5, CXCL6/GCP-2, CXCL9/MIG, FAM19A1, FAM19A2, FAM19A3, FAM19A4/TAFA4, FAM19A5, Fractalkine/CX3CL1, I-309/CCL1/TCA-3, IL-8/CXCL8, MCP-3/CCL7, NAP-2/PPBP/CXCL7, XCL2, or combinations thereof.

In some instances, the functional unit comprises a toxin. A toxin can be a cytotoxic agent including without limitation: CPX-351, cytarabine, daunorubicin, vosaroxin, sapacitabine, idarubicin, or mitoxantrone. Other examples of toxins and fragments thereof may include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and tricothecenes.

In some instances, the functional unit confers the ability to stimulate cell proliferation. Such functional units include without limitation: cytokines, interleukins, interferons, tumor necrosis factors, colony stimulating factors. In an aspect a growth factor can be: Rantes/CCL5, VEGF, HER2, EGFR, c-met/HGFR, ANGP1 or 2, CCL2, CCR1, CCR2, CCR3, CCR4, CD27, CD40, CD40LG, CD70, CSF1R, CSF2, CX3CL1, CXCL10, CXCL12, CXCL13, CXCL8, CXCR2, CXCR3, CXCR4, DDR2, DLL3, DLL4, ENG, EPHA3, EPHA4, ERBB2, ERBB3, ERBB4, FGF2, FGFR1, FGFR2, FGFR3, FGFR4, FLT1, FLT3, FLT4, GPC3, HGF, IFNB1, IFNG, IGF1R, KDR, KIT, LGALS9, MAPK, MET, NFKB1, NTRK, PDGFRA, PDGFRB, RET, STAT3, TEK, TGFB1, TNF. Growth factors can also include hormones. Examples of such hormones include, e.g., erythropoietin (EPO), insulin, secretins, glucagon-like polypeptide 1 (GLP-1), and the like. Further examples of such hormones include, but are not limited to, activin, inhibin, adiponectin, adipose-derived hormones, adrenocorticotropic hormone, afamelanotide, agouti signaling peptide, allatostatin, amylin, angiotensin, atrial natriuretic peptide, gastrin, somatotropin, bradykinin, brain-derived neurotrophic factor, calcitonin, cholecystokinin, ciliary neurotrophic factor, corticotropin-releasing hormone, cosyntropin, endothelian, enteroglucagon, fibroblast growth factor 15 (FGF15), GFG15/19, follicle-stimulating hormone, gastrin, gastroinhibitory peptide, ghrelin, glucagon, glucagon-like peptide-1, gonadotropin, gonadotropin-releasing hormone, granulocyte-colony-stimulating factor, growth hormone, growth-hormone-releasing hormone, hepcidin, human chorionic gonadotropin, human placental lactogen, incretin, insulin, insulin analog, insulin aspart, insulin degludec, insulin glargine, insulin lispro, insulin-like growth factor, insulin-like growth factor-1, insulin-like growth factor-2, leptin, liraglutide, luteinizing hormone, melanocortin, melanocyte-stimulating hormone, alpha-melanocyte-stimulating hormone, melanotin II, minigastrin, N-terminal prohormone of brain natriuretic peptide, nerve growth factor, neurotrophin-3, neurotrophin-4, NPH insulin, obestatin, orexin, osteocalcin, pancreatic hormone, parathyroid hormone, peptide hormone, peptide YY, plasma renin activity, pramlintide, preprohormone, prolactin, relaxin, relaxin family peptide hormone, renin, salcatonin, secretin, secretin family peptide hormone, sincalide, teleost leptins, temporin, tesamorelin, thyroid-stimulating hormone, thyrotropin-releasing hormone, urocortin, urocortin II, urocortin III, vasoactive intestinal peptide, and vitellogenin.

In other instances, a cell proliferation function unit comprises an interleukin. Non limiting examples of interleukins are IL-1, IL-1a, IL-2, IL-3, IL-4, IL-5, IL-6, IL-7, IL-8, IL-9, IL-10 IL-11, IL-12; IL-13, IL-14, IL-15, IL-16, IL-17, IL-17A, IL-18, IL-19, IL-20, IL-24, and combinations thereof. In an aspect, a growth factor can comprise an interferon such as: B4GALT7, IFN gamma, IFN omega, IFN-alpha, IFNA10, IFNA4, IFNA5/IFNaG, IFNA7, IFNB1/IFN-beta, IFNE, IFNZ, IL-28B/IFN-lambda-3, IL-29, IFNA8, LOC100425319, MEMO1, and combinations thereof. In an aspect, a growth factor can be a tumor necrosis factor (TNF) such as: BLyS/TNFSF138, CD70, LTB, TL1A, TRAIL, CD40L, Fas Ligand, RANKL, TNFSF1, LIGHT, CD30L, EDA-A1, OX-40L, TNFA, TNFSF13, and any combination thereof. In some embodiments, a growth factor comprises a colony-stimulating factor such as: granulocyte-macrophage colony-stimulating factor (GM-CSF), granulocyte colony-stimulating factor (G-CSF), macrophage colony-stimulating factor (M-CSF) and multipotential colony-stimulating factor (most commonly termed interleukin-3), and any combination thereof.

In some instances, the functional unit comprises a metabolite including without limitation: tetrahydrobiopterin (BH4), carbonic anhydrase IX (CA-IX), lactate transporters (MCT), glucose, ACAT-1 inhibitor, anti-cholesterol, L-arginine, Indoleamine 2,3 dioxygenase-1 (IDO-1), Epacadostat, glutamine, arginine, fatty acids, and combinations thereof.

In some instances, the functional unit comprises an anti-angiogenic agent including without limitation: Bevacizumab, thromobospondin-1 (TSP1), anti-PlGF, anti-VEGF, anti-FGF, ANG-1, ANG-2, ANG-3, ANG-4, TIE-1, TIE-2, c-MET, Notch-1, Notch-2, Notch-3 and Notch-4, Jagged-1, Jagged-2, Dll-1, Dll-3, Dll-4, ephrinA1/EphA2, ephrinB2/EphB4, α5β1, αvβ3, αvβ5, MCAM, TGFβ-1, TGFβ-2, TGFβ-3, Sema, Rho-J, CLEC14A, ramucirumab, cetuximab (anti-EGFR antibody), volociximab (anti-integrin-αvβ1 monoclonal antibody), etaricizumab or vitaxin (anti-integrin-αvβ3 antibody), MEDI3617 or REGN910 (anti-Ang-2 antibody), GAL-F2 (anti-FGF-2 antibody), and combinations thereof.

In some instances, the functional unit comprises an anti-hypoxic agent that can be metformin or anti-HIF1α.

In some instances, the functional unit comprises a chemical compound including without limitation: small molecule drugs, peptides, proteins, antibodies, DNA (minicircle DNA for example), double stranded DNA, single stranded DNA, double stranded RNA, single stranded RNA, RNAs (including shRNA and siRNA (which may also be expressed by the plasmid DNA incorporated as cargo within a liposome), antiviral agents such as acyclovir, zidovudine and the interferons; antibacterial agents such as aminoglycosides, cephalosporins and tetracyclines; antifungal agents such as polyene antibiotics, imidazoles and triazoles; antimetabolic agents such as folic acid, and purine and pyrimidine analogs; sterols such as cholesterol; carbohydrates, e.g., sugars and starches; amino acids, peptides, proteins such as cell receptor proteins, immunoglobulins, enzymes, hormones, neurotransmitters and glycoproteins; radiolabels such as radioisotopes and radioisotope-labeled compounds; radiopaque compounds; fluorescent compounds; mydriatic compounds; bronchodilators; local anesthetics; dyes, fluorescent dyes, including fluorescent dye peptides, or any combination thereof.

In some embodiments, a functional unit comprises a cytokine or a chemokine.

In some embodiments, a functional unit comprises another binding agent capable of specific binding to an antigen distinct from the cellular target. The antigen can be a tumor associated polypeptide (including without limitation PDL1 and TNF beta), a cellular protein associated with other diseases or conditions, and/or a cellular target that is intracellular, secreted, membrane bound, differentially expressed in a specific organelle within a cell (e.g., nucleus, ER or Golgi). For example, the functional unit incorporated into a subject antigen binding unit can comprise an anti-PDL1 or anti-TNF beta binding domain, linked in frame with the antigen binding domain specific for the bound target.

In some embodiments, a functional unit comprises a binding unit that exhibits specific binding to an immune cell antigen, a cytokine, a chemokine, a radioisotope, a fluorophore, or a toxin. In an aspect, an immune cell antigen is expressed by an immune cell. An immune cell antigen can also be an epitope of the antigen or a part of the antigen. An immune cell antigen can also be secreted by an immune cell. In some embodiments, an immune cell antigen is differentially expressed on an immune cell (over expressed or under expressed). An immune cell antigen can comprise any endogenous antigen and can be expressed on a surface of a cell or be internally expressed. An immune cell antigen can be expressed on the surface of an immune cell in the context of major histocompatibility antigen (MHC). In some cases, an immune cell antigen is an endogenously expressed cell surface protein or portion thereof. In some embodiments, an endogenous expressed cell surface protein can be selected from the group consisting of: cluster of differentiation 2 (CD2), cluster of differentiation 3 (CD3), cluster of differentiation 4 (CD4), cluster of differentiation 5 (CD5), cluster of differentiation 7 (CD7), cluster of differentiation 8 (CD8), cluster of differentiation 52 (CD52), cluster of differentiation 137 (CD137), and any portions thereof. An endogenous cell surface protein can also comprise an endogenous cellular receptor selected from but is not limited to: T cell receptor (TCR), B cell receptor (BCR) and portions thereof such as TCRα chain or TCRβ chain, human leukocyte antigen (HLA) or portions thereof. In some cases, a functional unit provided herein comprises a binding unit that exhibits specific binding to a CD3 polypeptide expressed on an immune cell. In an aspect, a CD3 polypeptide that is bound comprises an epsilon chain, a delta chain, and/or a gamma chain of CD3.

In some embodiments, an immune cell antigen is a check point antigen selected from the group consisting of: PD1, CTLA-4, Siglec-15 (S15), LAG3, TIM3, TIGIT, OX40, cluster of differentiation 93 (CD93), ADORA2A, cluster of differentiation 276 (CD276), VTCN1, BTLA, IDO1, KIR3DL1, VISTA, cluster of differentiation 244 (CD244), CISH, HPRT1, AAVS1, CCR5, CD160, cluster of differentiation 96 (CD96), cluster of differentiation 355 (CD355), SIGLEC7, SIGLEC9, TNFRSF10A, TNFRSF10B, CASP3, CASP6, CASP7, CASP8, CASP10, FADD, FAS, TGFBRII, TGFBRI, SMAD2, SMAD3, SMAD4, SKI, SKIL, TGIF1, IL10RA, IL10RB, CSK, PAG1, EGLN3, or combinations thereof.

In some cases, binding of the functional unit to a cellular antigen modulates an activity of an immune cell. An activity of an immune cell can be selected from the group consisting of: cytokine release; cytotoxicity of the immune cell; proliferation of the immune cell; differentiation, dedifferentiation or transdifferentiation of the immune cell; clonal expansion of the immune cell; trafficking of the immune cell; exhaustion and/or reactivation of the immune cell; and a combination thereof. In some cases, immune cells can release a cytokine in response to binding to an immune cell antigen or portion thereof. Cytokines that can be released or detected by immune cells that comprise a bound functional unit can be: IL-2, sIL-2R, IL-4, IL-6, IL-8, IL-10, IL-12, IL-17, gamma interferon, granulocyte-macrophage colony-stimulating factor, CCL2, CCL22, basic fibroblast growth factor (FGF-basic), hepatocyte growth factor (HGF), and migration inhibition factor (MIF), TNF-alpha, and combinations thereof. Cytokine release or detection can be determined and quantified by ELISA. In another aspect, an activity of an immune cell can be cytotoxicity of the immune cell. Cytotoxicity can be evaluated using co-culture assays, ⁵¹Cr-release assay, 125I- or 3H-labeling of target DNA to test ‘bulk’ DNA degradation, Europium- and samarium-release assays, CFDA- and BCECF-based assays, Measurement of alkaline phosphatase activity, LDH: enzyme-release assay, Fluorometric method based on hydrolysis of MUH, Calcein-AM-based assay, MTT assay, Release of firefly luciferase or bacterial β-gal (colorimetric or luminometric methods), Lysispot assay, Biophotonic cytotoxicity assay, Bicistronic vector-based assay, BLT assay, ELISA, calcium flux assay, LDA, IFN-γ ELISpot assay, and various other killing assays known to the skilled artisan. In some cases, binding of the functional unit to the immune cell antigen modulates proliferation of an immune cell that is measured by 5- and 6-carboxyfluorescein diacetate succinimidyl ester [CFSE], hemocytometry, flow cytometry, spectrophotometry, impedance microbiology, stereologic cell counting, image analysis, electrical resistance, colony forming unit (CFU) count, and any combinations thereof. In some cases, binding of the functional unit to the immune cell antigen modulates differentiation of an immune cell that can be determined by gene expression analysis, flow cytometry analysis, functionality testing, ELISA, imaging, and any combinations thereof. In an aspect, binding of the functional unit to an immune cell antigen modulates dedifferentiation. Dedifferentiation refers to cells that can lose properties they originally had, such as protein expression, or change shape. Dedifferentiation can be determined via imaging, flow cytometry, immunodetection, ELISA, microscopy, epigenome analysis, transcriptome analysis, proteome profile, and combinations thereof. In an aspect, binding of a functional unit to an immune cell antigen modulates transdifferentiation. Transdifferentiation occurs when a mature somatic cell transforms into another mature somatic cell without undergoing an intermediate pluripotent state or progenitor cell type. Transdifferentiation can be determined by gene expression analysis, immunodetection, epigenome analysis, transcriptome analysis, proteome profile, flow cytometry, microscopy, ELISA, and any combinations thereof. In an aspect, binding of a functional unit to an immune cell antigen modulates clonal expansion of the immune cell. Clonal expansion can refer to the production of daughter cells all arising originally from a single cell, for example an immune cell. For example, in a clonal expansion of lymphocytes, all progeny share the same antigen specificity. Clonal expansion can be determined via flow cytometry analysis. In some embodiments, binding of a functional unit to an immune cell antigen modulates trafficking of an immune cell. Trafficking can be determined by immunohistochemistry of tissue sections, flow cytometry, imaging analysis, bioluminescence imaging, and microscopy. Immune cell trafficking can refer to tethering/rolling, adhesion, arrest, crawling, transmigration of immune cells. In some aspects, binding of a functional unit to an immune cell antigen modulates exhaustion of the immune cell. Immune cell exhaustion can be characterized by poor effector function, sustained expression of inhibitory receptors and a transcriptional state distinct from that of functional cells, such as effector T cells or memory T cells. In an aspect, an immune cell is a T cell. Exhausted T cells can have sequential phenotypic and functional changes as compared to effector T cells. For example, exhausted T cells express inhibitory molecules and distinctive patterns of cytokine receptors, transcription factors and effector molecules, which distinguish these cells from conventional effector, memory and anergic T cells. In an exhausted T cell, IL-2 production is one of the first effector activities to be extinguished, followed by tumor necrosis factor-α (TNF-α) production and IFNγ secretion. This expression profile can be the result of several factors including shifts in the expression of pro- and anti-apoptotic factors as well as an inability to respond to IL-7 and IL-15. Immune cell exhaustion can be determined using flow cytometry, cytotoxicity testing, ELISA, proliferation analysis, cytometry, and any combinations thereof.

Antigen binding units disclosed herein can be incorporated into a chimeric polypeptide receptor comprising an engineered T cell receptor or a chimeric antigen receptors (CARs). A subject TCR comprises an extracellular domain capable of specific binding to an antigen, and an intracellular signaling domain, and is capable of forming a T cell receptor (TCR) complex. A subject antigen binding unit is typically incorporated into the extracellular domain of the TCR. In some embodiments, the TCR extracellular domain comprises element (1) a subject antigen binding unit, and element (2) an extracellular domain or portion thereof of a protein selected from the group consisting of a TCR alpha chain, a TCR beta chain, a CD3 epsilon TCR subunit, a CD3 gamma TCR subunit, a CD3 delta TCR, wherein elements (1) and (2) are operatively linked together.

In some instances, the TCR extracellular domain comprises in addition to a subject antigen binding unit, sequences of either or both of the a and 3 chains of a TCR. In other instances, the TCR extracellular domain comprises sequences the alpha chain and/or the p chain (VP). In yet other instances, the TCR extracellular domain comprises sequences the gamma chain and/or delta chain.

An intracellular signaling domain can be responsible for activation of at least one of the normal effector functions of the immune cell in which the TCR has been introduced. The term “effector function” refers to a specialized function of a cell. Effector function of a T cell, for example, may be cytolytic activity or helper activity including the secretion of cytokines. While usually the entire intracellular signaling domain can be employed, in some cases it is not necessary to use the entire chain. To the extent that a truncated portion of the intracellular signaling domain is used, such truncated portion may be used in place of the intact chain as long as it transduces a desired function signal, such as the effector functional signal. Examples of intracellular signaling domains for use in the engineered TCR include the cytoplasmic sequences of the T cell receptor and co-receptors that act in concert to initiate signal transduction following antigen receptor engagement, as well as any derivative or variant of these sequences and any recombinant sequence that has the same functional capability. In some embodiments, the engineered TCR intracellular domain comprising a stimulatory domain from an intracellular signaling domain of epsilon chain, delta chain, and/or a gamma chain of cluster of differentiation 3 (CD3). In other embodiments, the TCR intracellular domain comprising a stimulatory domain from an intracellular signaling domain of TCR alpha, or from an intracellular signaling domain of TCR beta.

In some embodiments, the TCR comprises a costimulatory domain, including without limitation, a functional signaling domains of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2Rbeta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

A subject TCR typically comprises a transmembrane domain linking the extracellular domain of the TCR comprising an antigen binding unit to the intracellular signaling domain. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region). In some embodiments, a transmembrane domain of the present disclosure may include at least the transmembrane sequences of e.g., the alpha, beta or zeta chain of a T-cell receptor.

A TCR can be functional and can maintain at least substantial biological activity in the case where it is not a full TCR, including but not limited to binding to the specific peptide-MHC complex, and/or maintaining functional signal transduction upon peptide activation or binding to an antigen.

Provided herein is a CAR comprising a subject polypeptide that comprises: an antigen binding unit, wherein the antigen binding unit (a) exhibits specific binding to a cellular target covalently bound by an exogenous molecule (bound target), but (b) lacks specific binding to the cellular target that is not bound to the exogenous molecule (unbound target).

Also provided is a CAR comprising a subject polypeptide that comprises: an antigen binding unit, wherein the antigen binding unit (a) exhibits specific binding to an intracellular target or an intracellular portion of a target, which target being bound by an exogenous molecule (bound target), but (b) lacks specific binding to the intracellular target or the intracellular portion of the target, which is not bound to the exogenous molecule (unbound target).

Further provided is a CAR comprising a subject multivalent antigen binding unit. In one embodiment, the multivalent antigen binding unit comprises a first binding domain and a second binding domain, wherein the first binding domain exhibits (a) specific binding to a cellular target covalently bound by an exogenous molecule (bound target), but (b) lacks specific binding to the cellular target that is not bound to the exogenous molecule (unbound target); and the second antigen binding domain comprises a functional unit capable of modulating one or more cellular functions including apoptosis, cell proliferation, cell differentiation, cell migration, cytotoxicity, release or trafficking of intercellular molecules, growth factor, metabolite, chemical compound, or a combination thereof. In another embodiment, the multivalent antigen binding unit comprises a first and a second binding domain, wherein the first binding domain exhibits (a) specific binding to an intracellular target or an intracellular portion of a target, which target being bound by an exogenous molecule (bound target), but (b) lacks specific binding to the intracellular target or the intracellular portion of the target, which is not bound to the exogenous molecule (unbound target); and the second antigen binding domain comprises a functional unit capable of modulating one or more cellular functions including apoptosis, cell proliferation, cell differentiation, cell migration, cytotoxicity, release or trafficking of intercellular molecules, growth factor, metabolite, chemical compound, or a combination thereof.

A subject CAR can comprise an intracellular region having an immune cell signaling unit. An intracellular signaling unit typically refers to the portion of a CAR which transduces the effector function signal and directs the immune cell to which CAR is introduced to perform a specialized function. A CAR can induce the effector function of an immune cell, for example, which may be cytolytic activity or helper activity including the secretion of cytokines. While usually the entire intracellular signaling region can be employed, in many cases it is not necessary to use the entire chain of a signaling unit. In some cases, a truncated portion of the intracellular signaling region is used. In some cases, the term intracellular signaling unit is thus meant to include any truncated portion of the intracellular signaling unit sufficient to transduce the effector function signal. Exemplary signaling unit for use in a CAR can include a cytoplasmic sequence of the T cell receptor (TCR) and co-receptors that act in concert to initiate signal transduction following target-receptor engagement, as well as any derivative or variant of these sequences and any synthetic sequence that has the same functional capability. In some cases, an intracellular signaling unit may contain signaling motifs which are known as immunoreceptor tyrosine-based activation motifs (ITAMs). Examples of ITAM containing cytoplasmic signaling sequences include those derived from TCR zeta, FcR gamma, FcR beta, CD3 gamma, CD3 delta, CD3 epsilon, CD5, CD22, CD79a, CD79b, and CD66d. However, in preferred embodiments, the intracellular signaling unit is derived from CD3 zeta chain. An example of a T cell signaling domain containing one or more ITAM motifs is the CD3 zeta domain, also known as T-cell receptor T3 zeta chain or CD247. This domain is part of the T-cell receptor-CD3 complex and plays an important role in coupling antigen recognition to several intracellular signal-transduction pathways with primary effector activation of the T cell. As used herein, CD3 zeta is primarily directed to human CD3 zeta and its isoforms as known by GRCh38.p13 (GCF_000001405.39), including proteins having a substantially identical sequence. As part of the chimeric antigen receptor, again the full T cell receptor T3 zeta chain is not required and any derivatives thereof comprising the signaling domain of T-cell receptor T3 zeta chain are suitable, including any functional equivalents thereof. In an aspect, an immune cell signaling unit comprises a primary signaling unit of a protein selected from the group consisting of: an Fcγ receptor (FcγR), an Fcε receptor (FcεR), an Fcα receptor (FcαR), neonatal Fc receptor (FcRn), CD2, CD3, CD3 ζ, CD3 γ, CD3 δ, CD3 ε, CD4, CD5, CD7, CD8, CD21, CD22, CD27, CD28, CD30, CD32, CD40L (CD154), CD45, CD66d, CD79a, CD79b, CD80, CD86, CD278 (also known as ICOS), CD247 ζ, CD247 η, DAP10, DAP12, FYN, LAT, Lck, MAPK, MHC complex, NFAT, NF-κB, PLC-γ, iC3b, C3dg, C3d, CD83, LGALS9, HAVCR1, TNFRSF9, TNFRSF4, TNFRSF14, TNFRSF18, KLRC2, ITGB2, ICOS, and Zap70. In an aspect, a primary signaling unit comprises a CD3 ζ signaling unit. In another aspect, the primary signaling unit comprises an immunoreceptor tyrosine-based activation motif (ITAM), for example from CD3 ζ. In some embodiments, the primary signaling unit comprises a CD3 ζ signaling unit. In some embodiments, the primary signaling unit comprises an immunoreceptor tyrosine-based activation motif (ITAM) of CD3 ζ. In some embodiments, the primary signaling unit comprises a signaling unit of an FcγR. In some embodiments, the primary signaling unit comprises a signaling unit of an FcγR selected from FcγRI (CD64), FcγRIIA (CD32), FcγRIIB (CD32), FcγRIIIA (CD16a), and FcγRIIIB (CD16b). In some embodiments, the primary signaling unit comprises a signaling unit of an FcεR. In some embodiments, the primary signaling unit comprises a signaling unit of an FcεR selected from FcεRI and FcεRII (CD23). In some embodiments, the primary signaling unit comprises a signaling unit of an FcαR. In some embodiments, the primary signaling unit comprises a signaling unit of an FcαR selected from FcαRI (CD89) and Fcα/μR.

In some cases, an immune cell signaling unit further comprises a co-stimulatory unit. In an aspect, one or more costimulatory units are included in an immune cell signaling unit. An intracellular signaling region can comprise a single co-stimulatory unit, for example a zeta-chain (1st generation CAR), or CD28 or 4-1BB (2nd generation CAR). In other examples, an intracellular signaling region can comprise two co-stimulatory units, such as CD28/OX40 or CD28/4-1BB (3rd generation). Together with intracellular signaling domains such as CD8, these co-stimulatory units can produce downstream activation of kinase pathways, which support gene transcription and functional cellular responses. In an aspect, a co-stimulatory unit comprises a signaling unit of a MHC class I molecule, a TNF receptor protein, an immunoglobulin-like protein, a cytokine receptor, an integrin, a signaling lymphocytic activation molecule (SLAM protein), an activating NK cell receptor, or a Toll ligand receptor. Co-stimulatory units of CARs can activate proximal signaling proteins related to either CD28 (Phosphatidylinositol-4, 5-bisphosphate 3-kinase) or 4-1BB/OX40 (TNF-receptor-associated-factor adapter proteins) pathways, and MAPK and Akt activation. In some cases, intracellular signaling units can be complexed with co-stimulatory units. With respect to the co-stimulatory units, the chimeric antigen receptor like complex can be designed to comprise several possible co-stimulatory signaling units. As is known in the art, in naïve T-cells the mere engagement of the T-cell receptor is not sufficient to induce full activation of T-cells into cytotoxic T-cells. Full, productive T cell activation benefits from a co-stimulatory signal provided by a co-stimulatory unit. In an aspect, a co-stimulatory unit comprises a signaling unit of a MHC class I molecule, a TNF receptor protein, an immunoglobulin-like protein, a cytokine receptor, an integrin, a signaling lymphocytic activation molecule (SLAM protein), an activating NK cell receptor, or a Toll ligand receptor. Any number of co-stimulatory units can be utilized in a CAR, for example from 1, 2, 3, 4, or up to 5 co-stimulatory units can be utilized. In an aspect, a CAR provided herein can have at least two co-stimulatory units. In an aspect, a CAR provided herein can have at least three co-stimulatory units.

Several receptors or units that have been reported to provide co-stimulation for T-cell activation, including signaling units of a MHC class I molecule, a TNF receptor protein, an immunoglobulin-like protein, a cytokine receptor, an integrin, a signaling lymphocytic activation molecule (SLAM protein), an activating NK cell receptor, or a Toll ligand receptor, such as those including but not limited to 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8 α, CD8 β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R 3, IL2R γ, IL7R C, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, VLA-6, CD28, OX40, CD27, CD2, CD5, ICAM-1, LFA-1 (CD11a/CD18), 4-1BBL, MyD88 and 4-1BB. The signaling pathways utilized by these co-stimulatory molecules share the common property of acting in synergy with the primary T cell receptor activation signal. These co-stimulatory signaling units provide a signal that can be synergistic with the primary effector activation signal originating from one or more ITAM motifs, for example a CD3 zeta signaling unit, and can complete the requirements for activation of the T cell. In some cases, addition of co-stimulatory units to a chimeric antigen receptor can enhance efficacy and durability of engineered cells. In another embodiment the intracellular signaling unit and the co-stimulatory domain are fused to one another thereby composing an intracellular signaling region.

In some embodiments, a subject CAR or TCR comprises a subject multivalent antigen binding unit having two or more antigen binding domains. The multivalent antigen binding unit can be bivalent or trivalent antigen.

In some embodiments, (i) a first binding domain exhibits (a) specific binding to an intracellular target or an intracellular portion of a target, which target being bound by an exogenous molecule (bound target), but lacks specific binding to the intracellular target or the intracellular portion of the target, which is not bound to the exogenous molecule (unbound target), and (ii) the second antigen binding domain comprises a functional unit capable of modulating one or more cellular functions including apoptosis, cell proliferation, cell differentiation, cell migration, cytotoxicity, release or trafficking of intercellular molecules, growth factor, metabolite, chemical compound, or a combination thereof. In some embodiments, the first antigen binding domain exhibits specific binding to a tumor associated polypeptide, and the second antigen binding domain exhibits binding to immune cell antigen as the functional unit that mediates one or more of the aforementioned biological functions.

In some embodiments, the second antigen binding domain of a subject CAR or TCR exhibits specific binding to a cell antigen (including but not limited to immune cell antigen) or a portion thereof that is expressed extracellularly, intracellularly or transmembranely, wherein the cell antigen is distinct from the bound target. In an aspect, the cell distinct antigen is an endogenously expressed protein, can also be an exogenous protein or portion of a protein, or a secreted protein. In some embodiment, such distinct antigen can be a tumor associated polypeptide (including without limitation PDL1 and TNF beta), a cellular protein associated with other diseases or conditions, and/or a cellular target that is intracellular, secreted, membrane bound, differentially expressed in a specific organelle within a cell (e.g., nucleus, ER or Golgi). For example, a subject CAR or TCR exhibits comprises a binding domain specifically binding to PDL1 or TNF beta.

In some embodiments, the ability of a subject CAR or TCR to target the bound target as well as one or more other distinct cell antigen is conferred by utilizing multivalent antigen binding unit disclosed herein.

In some embodiments, a subject CAR or TCR exhibits specific binding to an immune cell antigen. Where desired, the immune cell antigen is differentially expressed on an immune cell (over expressed or under expressed). An immune cell antigen can be expressed on the surface of an immune cell in the context of major histocompatibility antigen (MHC). In some cases, an immune cell antigen is an endogenously expressed cell surface protein or portion thereof. In some embodiments, an endogenous expressed cell surface protein can be selected from the group consisting of: cluster of differentiation 2 (CD2), cluster of differentiation 3 (CD3), cluster of differentiation 4 (CD4), cluster of differentiation 5 (CD5), cluster of differentiation 7 (CD7), cluster of differentiation 8 (CD8), cluster of differentiation 52 (CD52), cluster of differentiation 137 (CD137), and any portions thereof. An endogenous cell surface protein can also comprise an endogenous cellular receptor selected from, but is not limited to: T cell receptor (TCR), B cell receptor (BCR) and portions thereof such as TCRα chain or TCRβ chain, human leukocyte antigen (HLA) or portions thereof. In some cases, a functional unit provided herein comprises a binding unit that exhibits specific binding to a CD3 polypeptide expressed on an immune cell. In an aspect, a CD3 polypeptide that is bound comprises an epsilon chain, a delta chain, and/or a gamma chain of CD3.

In some embodiments, an immune cell antigen is a check point antigen or portion thereof. Non-limiting examples of check point antigen include Siglec-15 (S15), PD1, CTLA-4, LAG3, TIM3, TIGIT, OX40, cluster of differentiation 93 (CD93), ADORA2A, cluster of differentiation 276 (CD276), VTCN1, BTLA, IDO1, KIR3DL1, VISTA, cluster of differentiation 244 (CD244), CISH, HPRT1, AAVS1, CCR5, CD160, cluster of differentiation 96 (CD96), cluster of differentiation 355 (CD355), SIGLEC7, SIGLEC9, TNFRSF10A, TNFRSF10B, CASP3, CASP6, CASP7, CASP8, CASP10, FADD, FAS, TGFBRII, TGFBRI, SMAD2, SMAD3, SMAD4, SKI, SKIL, TGIF1, IL10RA, IL10RB, CSK, PAG1, EGLN3, or combinations thereof.

In some embodiments, the CAR or TCR further comprises a linker. A linker can be considered a portion of a CAR used to provide flexibility to an antigen binding unit. In some cases, a linker can be used to detect a CAR or TCR on the cell surface of a cell, particularly when antibodies to detect the antigen binding unit are not functional or available. In an aspect, the length of the linker derived from an immunoglobulin may require optimization depending on the location of the epitope on the target that the extracellular antigen binding region is targeting. In some embodiments, the linker is from CD28, IgG1 and/or CD8a. In some cases, a linker may not belong to an immunoglobulin but instead to another molecule such the native linker of a CD8 alpha molecule. A CD8 alpha linker can contain cysteine and proline residues known to play a role in the interaction of a CD8 co-receptor and MHC molecule. In an aspect, cysteine and proline residues can influence the performance of a CAR. A CAR or TCR linker can be size tunable and can compensate to some extent in normalizing the orthogonal synapse distance between CAR immunoresponsive cell and a target antigen or portion thereof. This topography of the immunological synapse between an immunoresponsive cell and a target cell also defines a distance that cannot be functionally bridged by a CAR due to a membrane-distal epitope on a cell-surface target molecule that, even with a short linker CAR, cannot bring the synapse distance in to an approximation for signaling. Likewise, membrane-proximal CAR targets, such as an antigen or portion thereof, have been described for which signaling outputs are only observed in the context of a long linker CAR. A linker can be tuned according to the extracellular antigen binding unit that is used. A linker can be of any length. A linker from a subject CAR can be from about 5 to about 30 amino acids in length. A linker can be 5, 6, 7, 8, 9, 10, 11, 12, 13, 14, 15, 16, 17, 18, 19, 20, 21, 22, 23, 24, 25, 26, 27, 28, 29, or up to 30 amino acids in length. A linker can be rich in glycine for flexibility, as well as serine or threonine for solubility, and can either connect the N-terminus of the VH with the C-terminus of the VL, or vice versa. The linker can be a 15-aa linker with the sequence (Gly₄Ser)₃. Amino acids to be used in linkers can be natural amino acids, amino acid derivatives, D-amino acids, modified amino acids, β-amino acid derivatives, α,α-substituted amino acid derivatives, N-substituted a-amino acid derivatives, aliphatic or cyclic amines, amino- and carboxyl-substituted cycloalkyl derivatives, amino- and carboxyl-substituted aromatic derivatives, γ-amino acid derivatives, aliphatic α-amino acid derivatives, diamines and polyamines. Further modified amino acids are known to the skilled artisan.

A subject CAR can further comprise a transmembrane unit. A transmembrane unit can anchor a CAR to the plasma membrane of an immune cell. A native transmembrane portion of CD28 can be used in a CAR. In other cases, a native transmembrane portion of CD8 alpha can also be used in the CAR. By “CD8” it can be meant a protein having at least 85, 90, 95, 96, 97, 98, 99 or 100% identity to NCBI Reference No: GRCh38.p13 (GCF_000001405.39) or a fragment thereof that has stimulatory activity. By “CD8 nucleic acid molecule” it can be meant a polynucleotide encoding a CD8 polypeptide. In some cases, a transmembrane region can be a native transmembrane portion of CD28. By “CD28” it can be meant a protein having at least 85, 90, 95, 96, 97, 98, 99 or 100% identity to NCBI Reference No: GRCh38.p13 (GCF_000001405.39) or a fragment thereof that has stimulatory activity. By “CD28 nucleic acid molecule” can be meant a polynucleotide encoding a CD28 polypeptide. In some cases, the transmembrane portion can comprise CD8a region. A transmembrane unit can be derived from an immune cell or synthetically generated. In some embodiments, a transmembrane unit is from CD8a, CD4, CD28, CD45, PD-1, and/or CD152.

The present disclosure further provides cells comprising the subject polypeptides comprising the antigen binding units disclosed herein. Exemplary subject polypeptides include multivalent antigen binding units, CARs and TCRs. Encompassed without limitation are prokaryotic (e.g. bacterial cells) and eukaryotic cells (including mammalian and human cells) comprising the subject polypeptides having the antigen binding units. In some embodiments, provided are modified immune cells comprising one or more TCR or CAR, or a combination of TCR and CAR disclosed herein. Immune cells can be lymphocytes including but not limited to T cells, B cells, NK cells, KHYG cells, tumor infiltration T cell (TIL), T helper cells, regulatory T cells, and memory T cells. In some embodiments, the lymphocyte is an immune effector cell including without limitation CD4+ and CD8+ and a natural killer cell (NK cell).

Where desired, an immune cell comprising an antigen binding unit provided herein can further modulating moiety including without limitation and enhancer and/or an inducible death moiety. In some embodiments, an enhancer suitable for incorporating into a subject immune cells can be cytokines and growth factors capable of stimulating the growth, clonal expansion, and/or enhancing persistence of the immune cell in vivo. Non-limiting examples of enhancers are IL-2, IL-3, IL-4, IL-6, IL-7, IL-10, IL-11, IL-12, IL-15, IL-17, IL-18, IL-21, IL-23, PD-1, PD-L1, CD122, CSF1R, CTAL-4, TIM-3, TGFR beta, receptors for the same, functional fragments thereof, functional variants thereof, and combinations thereof.

In some embodiments, a modified immune cell provided herein exhibits reduced expression or activity of an endogenous TCR. For example, an endogenous TCR may have reduced functionality. In certain cases, expression of an endogenous TCR may be silenced or knocked out, or substantially reduced as compared to a comparable unmodified immune cell. In certain cases, expression of an endogenous TCR may be reduced 1 fold, 2 fold, 3 fold, 5 fold, 10 fold, 30 fold, 60 fold, 80 fold, 100 fold, or 300 fold as compared to expression of an endogenous TCR in an unmodified immune cell.

In some embodiment, a subject immune cell comprises an inducible death moiety that allows for elimination of antigen binding unit expressing immune cells. In some instances, the inducible death moiety protein expression is conditionally controlled to address safety concerns for transplanted engineered immune cells that have permanently incorporated the gene encoding the safety switch protein into its genome. This conditional regulation could be variable and might include control through a small molecule-mediated post-translational activation and tissue-specific and/or temporal transcriptional regulation. The inducible death moiety could mediate induction of apoptosis, inhibition of protein synthesis, DNA replication, growth arrest, transcriptional and post-transcriptional genetic regulation and/or antibody-mediated depletion. In some instance, the inducible death moiety protein is activated by an exogenous molecule, e.g. a prodrug, that when activated, triggers apoptosis and/or cell death of a therapeutic cell. Examples of inducible death moiety proteins, include, but are not limited to suicide genes such as caspase 9 (or caspase 3 or 7), thymidine kinase, cytosine deaminase, B-cell CD20, modified EGFR, and any combination thereof. In this strategy, a prodrug that is administered in the event of an adverse event is activated by the suicide-gene product and kills the engineered cell. In some cases, an inducible death moiety can be selected from the group consisting of: rapaCasp9, iCasp9, HSV-TK, ΔCD20, mTMPK, ΔCD19, RQR8, and EGFRt. In an aspect, an inducible cell death moiety is HSV-TK, and the cell death activator is GCV. In an aspect, an inducible cell death moiety is iCasp9, and the cell death activator is AP1903.

A subject immune cell can be autologous or allogeneic immune cell. Preparation of allogenic or autologous cells can be carried out utilizing methods known in the art as well as those disclosed herein.

In some embodiments, a subject immune cell comprises one or more subject CARs, TCRs or both. In some embodiments, the immune cell comprises a single CAR or TCR complex directed to the bound target. In some embodiments, the immune cell comprises a single CAR or TCR complex having two or more binding domains capable of specifically binding collectively to different target antigens, different target epitopes of the same antigen, or different epitopes of different antigens. In some embodiments, the immune cell comprises multiple CARs or TCR complexes having two or more distinct binding domains capable of specifically binding collectively to different target antigens, different target epitopes of the same antigen, or different epitopes of different antigens. As such, encompassed herein are multispecific CARs or TCRs expressed as a single polypeptide or expressed as multiple polypeptides each conferring a distinct binding specificity.

Numerous target-specific molecules have been developed and shown to be able to target a wide range of intracellular targets or the intracellular portions of membrane-bound targets. Of particular interest are the exogenous molecules (e.g., small molecules) capable of specifically and covalently binding to the intended intracellular targets or the intracellular portions of a membrane bound target. Of also interest are the non-covalent exogenous molecules capable of forming a stable complex with their intracellular targets. Not wishing to be bound by any particular theory, the binding of an exogenous molecule to the cellular target of interest via covalent or no-covalent bond creates a new epitope on the bound target, thus permitting the generation of a subject antigen binding unit.

Non-limiting examples of exogenous molecules include those being capable of specifically binding to an antigen associated with a disease or condition, including without limitation tumor or cancer, viral, bacterial and parasitic infections, autoimmune disease, cardiovascular diseases, muscular diseases, degenerative diseases, inflammation, and metabolic disease. Of particular interest are exogenous molecules targeting tumor associated polypeptides.

A large number of cellular targets implicated in various diseases and conditions are known and characterized. Listed below are non-limiting examples: (a) Neoplasia: PTEN; ATM; ATR; EGFR; ERBB2; ERBB3; ERBB4; Notch1; Notch2; Notch3; Notch4; AKT; AKT2; AKT3; HIF; HIF1a; HIF3a; Met; HRG; Bcl2; PPAR alpha; PPAR gamma; WT1 (Wilms Tumor); FGF Receptor Family members (5 members: 1, 2, 3, 4, 5); CDKN2a; APC; RB (retinoblastoma); MEN1; VHL; BRCA1; BRCA2; AR (Androgen Receptor); TSG101; IGF; IGF Receptor; Igf1 (4 variants); Igf2 (3 variants); Igf 1 Receptor; Igf 2 Receptor; Bax; Bcl2; caspases family (9 members: 1, 2, 3, 4, 6, 7, 8, 9, 12); Kras; Apc; (b) Age-related Macular Degeneration: Aber; Ccl2; Cc2; cp (ceruloplasmin); Timp3; cathepsinD; Vldlr; Ccr2; (c) Schizophrenia: Neuregulin1 (Nrg1); Erb4 (receptor for Neuregulin); Complexin1 (Cplx1); Tph1 Tryptophan hydroxylase; Tph2 Tryptophan hydroxylase 2; Neurexin 1; GSK3; GSK3a; GSK3b; (d) Trinucleotide Repeat Disorders: HTT (Huntington's Dx); SBMA/SMAX1/AR (Kennedy's Dx); FXN/X25 (Friedrich's Ataxia); ATX3 (Machado-Joseph's Dx); ATXN1 and ATXN2 (spinocerebellar ataxias); DMPK (myotonic dystrophy); Atrophin-1 and Atn1 (DRPLA Dx); CBP (Creb-BP—global instability); VLDLR (Alzheimer's); Atxn7; Atxn10; (e) Fragile X Syndrome: FMR2; FXR1; FXR2; mGLUR5; (f) Secretase Related Disorders: APH-1 (alpha and beta); Presenilin (Psen1); nicastrin (Ncstn); PEN-2; (g) ALS: SOD1; ALS2; STEX; FUS; TARDBP; VEGF (VEGF-a; VEGF-b; VEGF-c); (h) Drug addiction: Prkce (alcohol); Drd2; Drd4; ABAT (alcohol); GRIA2; Gnn5; Grin1; Htr1b; Grin2a; Drd3; Pdyn; Gria1 (alcohol); (i) Autism: Mecp2; BZRAP1; MDGA2; Sema5A; Neurexin 1; Fragile X (FMR2 (AFF2); FXR1; FXR2; Mglur5); j) Alzheimer's Disease: E1; CHIP; UCH; UBB; Tau; LRP; PICALM; Clusterin; PSI; SORL1; CR1; Vldlr; Uba1; Uba3; CHIP28 (Aqp1, Aquaporin 1); Uchl1; Uchl3; APP, (k) Inflammation: IL-10; IL-1 (IL-1a; IL-1b); IL-13; IL-17 (IL-17a (CTLA8); IL-17b; IL-17c; IL-17d; IL-17f); 11-23; Cx3crl; ptpn22; TNFa; NOD2/CARD15 for IBD; IL-6; IL-12 (IL-12a; IL-12b); CTLA4; Cx3cl1; (1) Parkinson's Disease: x-Synuclein; DJ-1; LRRK2; Parkin; PINK1; (m) Blood and coagulation diseases and disorders: Anemia (CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, ASAT); Bare lymphocyte syndrome (TAPBP, TPSN, TAP2, ABCB3, PSF2, RING11, MHC2TA, C2TA, RFX5, RFXAP, RFX5), Bleeding disorders (TBXA2R, P2RX1, P2X1); Factor H and factor H-like 1 (HF1, CFH, HUS); Factor V and factor VIII (MCFD2); Factor VII deficiency (F7); Factor X deficiency (F10); Factor XI deficiency (F11); Factor XII deficiency (F12, HAF); Factor XIIIA deficiency (F13A1, F13A); Factor XIIIB deficiency (F13B); Fanconi anemia (FANCA, FACA, FA1, FA, FAA, FAAP95, FAAP90, FLJ34064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, KIAA1596); Hemophagocytic lymphohistiocytosis disorders (PRF1, HPLH2, UNC13D, MUNC13-4, HPLH3, HLH3, FHL3); Hemophilia A (F8, F8C, HEMA); Hemophilia B (F9, HEMB), Hemorrhagic disorders (PI, ATT, F5); Leukocyde deficiencies and disorders (ITGB2, CD18, LCAMB, LAD, EIF2B1, EIF2BA, EIF2B2, EIF2B3, EIF2B5, LVWM, CACH, CLE, EIF2B4); Sickle cell anemia (HBB); Thalassemia (HBA2, HBB, HBD, LCRB, HBA1); (n) Cell dysregulation and oncology diseases and disorders: B-cell non-Hodgkin lymphoma (BCL7A, BCL7); Leukemia (TAL1 TCL5, SCL, TAL2, FLT3, NBS1, NBS, ZNFN1A1, IK1, LYF1, HOXD4, HOX4B, BCR, CML, PHL, ALL, ARNT, KRAS2, RASK2, GMPS, AF10, ARHGEF12, LARG, KIAA0382, CALM, CLTH, CEBPA, CEBP, CHIC2, BTL, FLT3, KIT, PBT, LPP, NPM1, NUP214, D9S46E, CAN, CAIN, RUNX1, CBFA2, AML1, WHSC1L1, NSD3, FLT3, AF1Q, NPM1, NUMA1, ZNF145, PLZF, PML, MYL, STAT5B, AF10, CALM, CLTH, ARL11, ARLTS1, P2RX7, P2X7, BCR, CML, PHL, ALL, GRAF, NF1, VRNF, WSS, NFNS, PTPN11, PTP2C, SHP2, NS1, BCL2, CCND1, PRAD1, BCL1, TCRA, GATA1, GF1, ERYF1, NFE1, ABL1, NQO1, DIA4, NMOR1, NUP214, D9S46E, CAN, CAIN); (o) Inflammation and immune related diseases and disorders: AIDS (KIR3DL1, NKAT3, NKB1, AMB11, KIR3DS1, IFNG, CXCL12, SDF1); Autoimmune lymphoproliferative syndrome: TNFRSF6, APT1, FAS, CD95, ALPS1A; Combined immunodeficiency: IL2RG, SCIDX1, SCIDX, IMD4); HIV-1 (CCL5, SCYA5, D17S136E, TCP228), HIV susceptibility or infection (IL10, CSIF, CMKBR2, CCR2, CMKBR5, CCCKR5 (CCR5); Immunodeficiencies: CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, TACI); Inflammation: IL-10, IL-1 (IL-1a, IL-1b), IL-13, IL-17 (IL-17a (CTLA8), IL-17b, IL-17c, IL-17d, IL-17f), II-23, Cx3crl, ptpn22, TNFa, NOD2/CARD15 for IBD, IL-6, IL-12 (IL-12a, IL-12b), CTLA4, Cx3cl1; Severe combined immunodeficiencies: SCIDs, JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, IMD4; (p) Metabolic, liver, kidney and protein diseases and disorders: Amyloid neuropathy (TTR, PALB); Amyloidosis (APOA1, APP, AAA, CVAP, AD1, GSN, FGA, LYZ, TTR, PALB); Cirrhosis (KRT18, KRT8, CIRH1A, NAIC, TEX292, KIAA1988); Cystic fibrosis (CFTR, ABCC7, CF, MRP7); Glycogen storage diseases (SLC2A2, GLUT2, G6PC, G6PT, G6PT1, GAA, LAMP2, LAMPB, AGL, GDE, GBE1, GYS2, PYGL, PFKM); Hepatic adenoma, 142330 (TCF1, HNF1A, MODY3), Hepatic failure, early onset, and neurologic disorder (SCOD1, SCO1), Hepatic lipase deficiency (LIPC), Hepatoblastoma, cancer and carcinomas (CTNNB1, PDGFRL, PDGRL, PRLTS, AXINI, AXIN, CTNNB1, TP53, P53, LFS1, IGF2R, MPRI, MET, CASP8, MCH5; Medullary cystic kidney disease (UMOD, HNFJ, FJHN, MCKD2, ADMCKD2); Phenylketonuria (PAH, PKU1, QDPR, DHPR, PTS); Polycystic kidney and hepatic disease (FCYT, PKHD1, ARPKD, PKD1, PKD2, PKD4, PKDTS, PRKCSH, G19P1, PCLD, SEC63); (q) Muscular/Skeletal diseases and disorders: Becker muscular dystrophy (DMD, BMD, MYF6), Duchenne Muscular Dystrophy (DMD, BMD); Emery-Dreifuss muscular dystrophy (LMNA, LMN1, EMD2, FPLD, CMD1A, HGPS, LGMD1B, LMNA, LMN1, EMD2, FPLD, CMD1A); Facioscapulohumeral muscular dystrophy (FSHMD1A, FSHD1A); Muscular dystrophy (FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, EBS1); Osteopetrosis (LRP5, BMND1, LRP7, LR3, OPPG, VBCH2, CLCN7, CLC7, OPTA2, OSTM1, GL, TCIRG1, TIRC7, OC116, OPTB1); Muscular atrophy (VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, SMARD1); (r) Neurological and neuronal diseases and disorder: ALS (SOD1, ALS2, STEX, FUS, TARDBP, VEGF (VEGF-a, VEGF-b, VEGF-c); Alzheimer disease (APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, AD3); Autism (Mecp2, BZRAP1, MDGA2, Sema5A, Neurexin 1, GLO1, MECP2, RTT, PPMX, MRX16, MRX79, NLGN3, NLGN4, KIAA1260, AUTSX2); Fragile X Syndrome (FMR2, FXR1, FXR2, mGLUR5); Huntington's disease and disease like disorders (HD, IT15, PRNP, PRIP, JPH3, JP3, HDL2, TBP, SCA17); Parkinson disease (NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, NDUFV2); Rett syndrome (MECP2, RTT, PPMX, MRX16, MRX79, CDKL5, STK9, MECP2, RTT, PPMX, MRX16, MRX79, x-Synuclein, DJ-1); Schizophrenia (Neuregulin1 (Nrg1), Erb4 (receptor for Neuregulin), Complexin1 (Cplx1), Tph1 Tryptophan hydroxylase, Tph2, Tryptophan hydroxylase 2, Neurexin 1, GSK3, GSK3a, GSK3b, 5-HTT (Slc6a4), COMT, DRD (Drd1a), SLC6A3, DAOA, DTNBP1, Dao (Daol)); Secretase Related Disorders (APH-1 (alpha and beta), Presenilin (Psen1), nicastrin, (Ncstn), PEN-2, Nos1, Parp1, Nat1, Nat2); Trinucleotide Repeat Disorders (HTT (Huntington's Dx), SBMA/SMAX1/AR (Kennedy's Dx), FXN/X25 (Friedrich's Ataxia), ATX3 (Machado-Joseph's Dx), ATXN1 and ATXN2 (spinocerebellar ataxias), DMPK (myotonic dystrophy), Atrophin-1 and Atn1 (DRPLA Dx), CBP (Creb-BP—global instability), VLDLR (Alzheimer's), Atxn7, Atxn10); (s) Ocular diseases and disorders: Age-related macular degeneration (Aber, Cc12, Cc2, cp (ceruloplasmin), Timp3, cathepsinD, Vldlr, Ccr2); Cataract (CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, KRIT1); Corneal clouding and dystrophy (APOA1, TGFBI, CSD2, CDGG1, CSD, BIGH3, CDG2, TACSTD2, TROP2, M1S1, VSX1, RINX, PPCD, PPD, KTCN, COL8A2, FECD, PPCD2, PIP5K3, CFD); Cornea plana congenital (KERA, CNA2); Glaucoma (MYOC, TIGR, GLC1A, JOAG, GPOA, OPTN, GLC1E, FIP2, HYPL, NRP, CYP1B1, GLC3A, OPA1, NTG, NPG, CYP1B1, GLC3A); Leber congenital amaurosis (CRB1, RP12, CRX, CORD2, CRD, RPGRIP1, LCA6, CORD9, RPE65, RP20, AIPL1, LCA4, GUCY2D, GUC2D, LCA1, CORD6, RDH12, LCA3); Macular dystrophy (ELOVL4, ADMD, STGD2, STGD3, RDS, RP7, PRPH2, PRPH, AVMD, AOFMD, VMD2).

A vast diversity of tumor associated polypeptides are known in the art. As used herein, tumor associated polypeptides include the full-length gene products as well as fragments (functional or non functional) thereof. In some instances, the tumor associated polypeptides are differentially expressed (either underexpressed or overexpressed) in tumor tissues as compared to normal tissues or cells. In some instances, the tumor associated polypeptides are wild type, and in other instances, they contain mutation(s) at the amino acid sequence level and/or at the nucleotide sequence, including without limitation missense, nonsense, insertion, deletion, duplication, frameshift, and repeat expansion mutations.

In an aspect, a tumor associated polypeptide confers microsatellite instability, Cpg island methylator phenotype, chromosomal instability, or combinations thereof.

In some cases, a target to which the exogenous molecule binds is implicated in one or more cell signalling pathways associated with cell proliferation, cell differentiation, apoptosis, and/or cell migration. Provided below are non-limiting examples of targets involved in various signalling pathways: (a) PI3K/AKT Signaling: PRKCE; ITGAM; ITGA5; IRAK1; PRKAA2; EIF2AK2; PTEN; EIF4E; PRKCZ; GRK6; MAPK1; TSC1; PLK1; AKT2; IKBKB; PIK3CA; CDK8; CDKN1B; NFKB2; BCL2; PIK3CB; PPP2R1A; MAPK8; BCL2L1; MAPK3; TSC2; ITGA1; KRAS; EIF4EBP1; RELA; PRKCD; NOS3; PRKAA1; MAPK9; CDK2; PPP2CA; PIM1; ITGB7; YWHAZ; ILK; TP53; RAF1; IKBKG; RELB; DYRK1A; CDKN1A; ITGB1; MAP2K2; JAK1; AKT1; JAK2; PIK3R1; CHUK; PDPK1; PPP2R5C; CTNNB1; MAP2K1; NFKB1; PAK3; ITGB3; CCND1; GSK3A; FRAP1; SFN; ITGA2; TTK; CSNK1A1; BRAF; GSK3B; AKT3; FOXO1; SGK; HSP90AA1; RPS6KB1; (b) ERK/MAPK Signaling: PRKCE; ITGAM; ITGA5; HSPB1; IRAK1; PRKAA2; EIF2AK2; RAC1; RAP1A; TLN1; EIF4E; ELK1; GRK6; MAPK1; RAC2; PLK1; AKT2; PIK3CA; CDK8; CREB1; PRKCI; PTK2; FOS; RPS6KA4; PIK3CB; PPP2R1A; PIK3C3; MAPK8; MAPK3; ITGA1; ETS1; KRAS; MYCN; EIF4EBP1; PPARG; PRKCD; PRKAA1; MAPK9; SRC; CDK2; PPP2CA; PIM1; PIK3C2A; ITGB7; YWHAZ; PPP1CC; KSR1; PXN; RAF1; FYN; DYRK1A; ITGB1; MAP2K2; PAK4; PIK3R1; STAT3; PPP2R5C; MAP2K1; PAK3; ITGB3; ESR1; ITGA2; MYC; TTK; CSNK1A1; CRKL; BRAF; ATF4; PRKCA; SRF; STAT1; SGK; (c) Glucocorticoid Receptor Signaling: RAC1; TAF4B; EP300; SMAD2; TRAF6; PCAF; ELK1; MAPK1; SMAD3; AKT2; IKBKB; NCOR2; UBE2I; PIK3CA; CREB1; FOS; HSPA5; NFKB2; BCL2; MAP3K14; STAT5B; PIK3CB; PIK3C3; MAPK8; BCL2L1; MAPK3; TSC22D3; MAPK10; NRIP1; KRAS; MAPK13; RELA; STAT5A; MAPK9; NOS2A; PBX1; NR3C1; PIK3C2A; CDKN1C; TRAF2; SERPINE1; NCOA3; MAPK14; TNF; RAF1; IKBKG; MAP3K7; CREBBP; CDKN1A; MAP2K2; JAK1; IL8; NCOA2; AKT1; JAK2; PIK3R1; CHUK; STAT3; MAP2K1; NFKB1; TGFBR1; ESR1; SMAD4; CEBPB; JUN; AR; AKT3; CCL2; MMP1; STAT1; IL6; HSP90AA1; (d) Axonal Guidance Signaling: PRKCE; ITGAM; ROCK1; ITGA5; CXCR4; ADAM12; IGF1; RAC1; RAP1A; E1F4E; PRKCZ; NRP1; NTRK2; ARHGEF7; SMO; ROCK2; MAPK1; PGF; RAC2; PTPN11; GNAS; AKT2; PIK3CA; ERBB2; PRKCI; PTK2; CFL1; GNAQ; PIK3CB; CXCL12; PIK3C3; WNT11; PRKD1; GNB2L1; ABL1; MAPK3; ITGA1; KRAS; RHOA; PRKCD; PIK3C2A; ITGB7; GLI2; PXN; VASP; RAF1; FYN; ITGB1; MAP2K2; PAK4; ADAM17; AKT1; PIK3R1; GLI1; WNT5A; ADAM10; MAP2K1; PAK3; ITGB3; CDC42; VEGFA; ITGA2; EPHA8; CRKL; RND1; GSK3B; AKT3; PRKCA; (e) Ephrin Receptor Signaling: PRKCE; ITGAM; ROCK1; ITGA5; CXCR4; IRAK1; PRKAA2; EIF2AK2; RAC1; RAP1A; GRK6; ROCK2; MAPK1; PGF; RAC2; PTPN11; GNAS; PLK1; AKT2; DOK1; CDK8; CREB1; PTK2; CFL1; GNAQ; MAP3K14; CXCL12; MAPK8; GNB2L1; ABL1; MAPK3; ITGA1; KRAS; RHOA; PRKCD; PRKAA1; MAPK9; SRC; CDK2; PIM1; ITGB7; PXN; RAF1; FYN; DYRK1A; ITGB1; MAP2K2; PAK4, AKT1; JAK2; STAT3; ADAM10; MAP2K1; PAK3; ITGB3; CDC42; VEGFA; ITGA2; EPHA8; TTK; CSNK1A1; CRKL; BRAF; PTPN13; ATF4; AKT3; SGK; (f) Apoptosis Signaling: PRKCE; ROCK1; BID; IRAK1; PRKAA2; EIF2AK2; BAK1; BIRC4; GRK6; MAPK1; CAPNS1; PLK1; AKT2; IKBKB; CAPN2; CDK8; FAS; NFKB2; BCL2; MAP3K14; MAPK8; BCL2L1; CAPN1; MAPK3; CASP8; KRAS; RELA; PRKCD; PRKAA1; MAPK9; CDK2; PIM1; TP53; TNF; RAF1; IKBKG; RELB; CASP9; DYRK1A; MAP2K2; CHUK; APAF1; MAP2K1; NFKB1; PAK3; LMNA; CASP2; BIRC2; TTK; CSNK1A1; BRAF; BAX; PRKCA; SGK; CASP3; BIRC3; PARP1; (g) B Cell Receptor Signaling: RAC1; PTEN; LYN; ELK1; MAPK1; RAC2; PTPN11; AKT2; IKBKB; PIK3CA; CREB1; SYK; NFKB2; CAMK2A; MAP3K14; PIK3CB; PIK3C3; MAPK8; BCL2L1; ABL1; MAPK3; ETS1; KRAS; MAPK13; RELA; PTPN6; MAPK9; EGR1; PIK3C2A; BTK; MAPK14; RAF1; IKBKG; RELB; MAP3K7; MAP2K2; AKT1; PIK3R1; CHUK; MAP2K1; NFKB1; CDC42; GSK3A; FRAP1; BCL6; BCL10; JUN; GSK3B; ATF4; AKT3; VAV3; RPS6KB1; (h) Leukocyte Extravasation Signaling: ACTN4; CD44; PRKCE; ITGAM; ROCK1; CXCR4; CYBA; RAC1; RAP1A; PRKCZ; ROCK2; RAC2; PTPN11; MMP14; PIK3CA; PRKCI; PTK2; PIK3CB; CXCL12; PIK3C3; MAPK8; PRKD1; ABL1; MAPK10; CYBB; MAPK13; RHOA; PRKCD; MAPK9; SRC; PIK3C2A; BTK; MAPK14; NOX1; PXN; VIL2; VASP; ITGB1; MAP2K2; CTNND1; PIK3R1; CTNNB1; CLDN1; CDC42; F11R; ITK; CRKL; VAV3; CTTN; PRKCA; MMP1; MMP9; (i) Integrin Signaling: ACTN4; ITGAM; ROCK1; ITGA5; RAC1; PTEN; RAP1A; TLN1; ARHGEF7; MAPK1; RAC2; CAPNS1; AKT2; CAPN2; PIK3CA; PTK2; PIK3CB; PIK3C3; MAPK8; CAV1; CAPN1; ABL1; MAPK3; ITGA1; KRAS; RHOA; SRC; PIK3C2A; ITGB7; PPP1CC; ILK; PXN; VASP; RAF1; FYN; ITGB1; MAP2K2; PAK4; AKT1; PIK3R1; TNK2; MAP2K1; PAK3; ITGB3; CDC42; RND3; ITGA2; CRKL; BRAF; GSK3B; AKT3; (j) PTEN Signaling: ITGAM; ITGA5; RAC1; PTEN; PRKCZ; BCL2L11; MAPK1; RAC2; AKT2; EGFR; IKBKB; CBL; PIK3CA; CDKN1B; PTK2; NFKB2; BCL2; PIK3CB; BCL2L1; MAPK3; ITGA1; KRAS; ITGB7; ILK; PDGFRB; INSR; RAF1; IKBKG; CASP9; CDKN1A; ITGB1; MAP2K2; AKT1; PIK3R1; CHUK; PDGFRA; PDPK1; MAP2K1; NFKB1; ITGB3; CDC42; CCND1; GSK3A; ITGA2; GSK3B; AKT3; FOXO1; CASP3; RPS6KB1; (k) p53 Signaling: PTEN; EP300; BBC3; PCAF; FASN; BRCA1; GADD45A; BIRC5; AKT2; PIK3CA; CHEK1; TP53INP1; BCL2; PIK3CB; PIK3C3; MAPK8; THBS1; ATR; BCL2L1; E2F1; PMAIP1; CHEK2; TNFRSF10B; TP73; RB1; HDAC9; CDK2; PIK3C2A; MAPK14; TP53; LRDD; CDKN1A; HIPK2; AKT1; PIK3R1; RRM2B; APAF1; CTNNB1; SIRT1; CCND1; PRKDC; ATM; SFN; CDKN2A; JUN; SNAI2; GSK3B; BAX; AKT3; (1) SAPK/JNK Signaling: PRKCE; IRAK1; PRKAA2; EIF2AK2; RAC1; ELK1; GRK6; MAPK1; GADD45A; RAC2; PLK1; AKT2; PIK3CA; FADD; CDK8; PIK3CB; PIK3C3; MAPK8; RIPK1; GNB2L1; IRS1; MAPK3; MAPK10; DAXX; KRAS; PRKCD; PRKAA1; MAPK9; CDK2; PIM1; PIK3C2A; TRAF2; TP53; LCK; MAP3K7; DYRK1A; MAP2K2; PIK3R1; MAP2K1; PAK3; CDC42; JUN; TTK; CSNK1A1; CRKL; BRAF; SGK; (m) PPAr/RXR Signaling: PRKAA2; EP300; INS; SMAD2; TRAF6; PPARA; FASN; RXRA; MAPK1; SMAD3; GNAS; IKBKB; NCOR2; ABCA1; GNAQ; NFKB2; MAP3K14; STAT5B; MAPK8; IRS1; MAPK3; KRAS; RELA; PRKAA1; PPARGC1A; NCOA3; MAPK14; INSR; RAF1; IKBKG; RELB; MAP3K7; CREBBP; MAP2K2; JAK2; CHUK; MAP2K1; NFKB1; TGFBR1; SMAD4; JUN; ILIR1; PRKCA; IL6; HSP90AA1; ADIPOQ; (n) NF-κB Signaling: IRAK1; EIF2AK2; EP300; INS; MYD88; PRKCZ: TRAF6; TBK1; AKT2; EGFR; IKBKB; PIK3CA; BTRC; NFKB2; MAP3K14; PIK3CB; PIK3C3; MAPK8; RIPK1; HDAC2; KRAS; RELA; PIK3C2A; TRAF2; TLR4: PDGFRB; TNF; INSR; LCK; IKBKG; RELB; MAP3K7; CREBBP; AKT1; PIK3R1; CHUK; PDGFRA; NFKB1; TLR2; BCL10; GSK3B; AKT3; TNFAIP3; ILIR1; (o) Neuregulin Signaling: ERBB4; PRKCE; ITGAM; ITGA5: PTEN; PRKCZ; ELK1; MAPK1; PTPN1: 1; AKT2; EGFR; ERBB2; PRKCI; CDKN1B; STAT5B; PRKD1; MAPK3; ITGA1; KRAS; PRKCD; STAT5A; SRC; ITGB7; RAF1; ITGB1; MAP2K2; ADAM17; AKT1; PIK3R1; PDPK1; MAP2K1; ITGB3; EREG; FRAP1; PSEN1; ITGA2; MYC; NRG1; CRKL; AKT3; PRKCA; HSP90AA1; RPS6KB1; (p) Wnt & Beta catenin Signaling: CD44; EP300; LRP6; DVL3; CSNK1E; GJA1; SMO; AKT2; PIN1; CDH1; BTRC; GNAQ; MARK2; PPP2R1A; WNT11; SRC; DKK1; PPP2CA; SOX6; SFRP2: ILK; LEF1; SOX9; TP53; MAP3K7; CREBBP; TCF7L2; AKT1; PPP2R5C; WNT5A; LRP5; CTNNB1; TGFBR1; CCND1; GSK3A; DVL1; APC; CDKN2A; MYC; CSNK1A1; GSK3B; AKT3; SOX2; (q) Insulin Receptor: PTEN; INS; EIF4E; PTPN1; PRKCZ; MAPK1; TSC1; PTPN11; AKT2; CBL; PIK3CA; PRKCI; PIK3CB; PIK3C3; MAPK8; IRS1; MAPK3; TSC2; KRAS; EIF4EBP1; SLC2A4; PIK3C2A; PPP1CC; INSR; RAF1; FYN; MAP2K2; JAKI; AKT1; JAK2; PIK3R1; PDPK1; MAP2K1; GSK3A; FRAP1; CRKL; GSK3B; AKT3; FOXO1; SGK; RPS6KB1; (r) IL-6 Signaling: HSPB1; TRAF6; MAPKAPK2; ELK1; MAPK1; PTPN11; IKBKB; FOS; NFKB2: MAP3K14; MAPK8; MAPK3; MAPK10; IL6ST; KRAS; MAPK13; IL6R; RELA; SOCS1; MAPK9; ABCB1; TRAF2; MAPK14; TNF; RAF1; IKBKG; RELB; MAP3K7; MAP2K2; IL8; JAK2; CHUK; STAT3; MAP2K1; NFKB1; CEBPB; JUN; IL1R1; SRF; IL6; (s) PPAR Signaling: EP300; INS; TRAF6; PPARA; RXRA; MAPK1; IKBKB; NCOR2; FOS; NFKB2; MAP3K14; STAT5B; MAPK3; NRIP1; KRAS; PPARG; RELA; STAT5A; TRAF2; PPARGC1A; PDGFRB; TNF; INSR; RAF1; IKBKG; RELB; MAP3K7; CREBBP; MAP2K2; CHUK; PDGFRA; MAP2K1; NFKB1; JUN; IL1R1; HSP90AA; (t) G-Protein Coupled Receptor Signaling: PRKCE; RAP1A; RGS16; MAPK1; GNAS; AKT2; IKBKB; PIK3CA; CREB1; GNAQ; NFKB2; CAMK2A; PIK3CB; PIK3C3; MAPK3; KRAS; RELA; SRC; PIK3C2A; RAF1; IKBKG; RELB; FYN; MAP2K2; AKT1; PIK3R1; CHUK; PDPK1; STAT3; MAP2K1; NFKB1; BRAF; ATF4; AKT3; PRKCA; (u) Cell Cycle: G1/S Checkpoint Regulation: HDAC4; SMAD3; SUV39H1; HDAC5; CDKN1B; BTRC; ATR; ABL1; E2F1; HDAC2; HDAC7A; RB1; HDAC11; HDAC9; CDK2; E2F2; HDAC3; TP53; CDKN1A; CCND1; E2F4; ATM; RBL2; SMAD4; CDKN2A; MYC; NRG1; GSK3B; RBL1; HDAC6; (v) IL-2 Signaling: ELK1; MAPK1; PTPN11; AKT2; PIK3CA; SYK; FOS; STAT5B; PIK3CB; PIK3C3; MAPK8; MAPK3; KRAS; SOCS1; STAT5A; PIK3C2A; LCK; RAF1; MAP2K2; JAKI; AKT1; PIK3R1; MAP2K1; JUN; AKT3.

In some embodiments, the targets to which the exogenous molecule binds are: EGFR, FGFR, PDGF receptor, WNT, MAPK/PI3K, TGF-β, TP53 and mutations in different genes including c-MYC, BRAF, PI-3 kinase, MAP kincase, BTK, Her2, Erk, LCK, AKT, mTOR, PTEN, SMAD2, SMAD4, and RAS (including without limitation H-RAS, K-RAS, and N-RAS).

The three human RAS genes (KRAS, NRAS and HRAS) are the most frequently mutated oncogenes in human cancer appearing in 90% of pancreatic, 35% of lung and in 45% of colon cancers. In particular, KRAS is the isoform prevalently mutated in pancreas, lung and colon cancer, while NRAS is the predominant isoform mutated in cutaneous melanomas and acute myelogenous leukemia and HRAS is the predominant isoform mutated in the bladder. The three human RAS genes that encode four small guanosine triphosphatase (GTPases) are KRAS4A, KRAS4B, HRAS and NRAS. RAS is the component of the mitogen activated protein kinase (MAPK) signaling pathway, which is activated by a ligand binding to a receptor tyrosine kinase (RTK) such as the epidermal growth factor receptor (EGFR). RAS exists in the non-active (GDP, guanosine diphosphatase) or active-state (GTP) and the transition between these two states is responsible for signal transduction events occurring from the cell surface receptor to the inside of the cell which is utilized for cell growth and differentiation. In cancer patients, oncogenic K-Ras mutations are recurrently observed at positions 12, 13 and 61. G12 is the most frequently mutated residue (89%), which most prevalently mutates to aspartate (G12D, 36%) followed by valine (G12V, 23%) and cysteine (G12C, 14%). This residue is located at the protein active site, which consists of a phosphate binding loop (P-loop, residues 10-17) and switch I (SI, residues 25-40) and II (SII, residues 60-74) regions. In some cases, an antigen binding unit provided herein binds to a switch unit of K-ras that comprises two or more residues selected from the group consisting of cysteine 12, K16, D69, M72, Y96, and Q99. The active site residues are bound to the phosphate groups of GTP and are responsible for the GTPase function of K-Ras. In its side-chain, G12 has only a single hydrogen. However, the mutation to aspartate (G12D) leads to the projection of a bulkier side group into the active site, which causes a steric hindrance in GTP hydrolysis16, impairs the GTPase function and locks K-Ras in its active GTP-bound state.

In an aspect, a mutated KRAS is a major driver for malignant transformation in, as G12C mutations are detected in early lesions and generally retained in metastases. In an aspect, a subject tumor associated polypeptide can be or can be a portion of: Ras, EGFR, FGFR, PI3Kinase, BTK, Her2, or combinations thereof. In an aspect, the tumor associated polypeptide is a K-ras polypeptide having a G to C mutation at residue 12, G12C. Additional K-ras polypeptides can have mutations at: G12C, G12D, G12V, G13C, G13D, A18D, Q61H, K117N, and combinations thereof.

In some cases, a tumor associated polypeptide is generated from a mutation that is a hotspot driver mutation. In an aspect, a tumor associated polypeptide is generated from a mutant PIK3 CA gene. In some cases, the mutation is selected from the group comprising E542K, E545K, or H1047R.

In another aspect, a tumor associated polypeptide is a BRAF polypeptide having a V600E mutation at residue 600. In another aspect, a tumor associated polypeptide is a MEK1 polypeptide having a K57T mutation at residue 57. In some embodiments, additional subject tumor associated polypeptides are proteins coded by genes selected from the group consisting of: 1-40-β-amyloid, 4-1BB, 5AC, 5T4, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta-amyloid, B-lymphoma cell, C242 antigen, C5, CA-125, Canis lupus familiaris IL31, carbonic anhydrase 9 (CA-IX), cardiac myosin, CCL11 (eotaxin-1), CCR4, CCR5, CD11, CD18, CD125, CD140a, CD147 (basigin), CD15, CD152, CD154 (CD40L), CD19, CD2, CD20, CD200, CD22, CD221, CD23 (IgE receptor), CD25 (a chain of IL-2receptor), CD27, CD274, CD28, CD3, CD3 epsilon, CD30, CD33, CD37, CD38, CD4, CD40, CD40 ligand, CD41, CD44 v6, CD5, CD51, CD52, CD56, CD6, CD70, CD74, CD79B, CD80, CEA, CEA-related antigen, CFD, ch4D5, CLDN18.2, Clostridium difficile, clumping factor A, CSF1R, CSF2, CTLA-4, C—X—C chemokine receptor type 4, cytomegalovirus, cytomegalovirus glycoprotein B, dabigatran, DLL4, DPP4, DR5, E. coli shiga toxin type-1, E. coli shiga toxin type-2, EGFL7, EGFR, endotoxin, EpCAM, episialin, ERBB3, Escherichia coli, F protein of respiratory syncytial virus, FAP, fibrin II beta chain, fibronectin extra domain-B, folate hydrolase, folate receptor 1, folate receptor alpha, Frizzled receptor, ganglioside GD2, GD2, GD3 ganglioside, glypican 3, GMCSF receptor α-chain, GPNMB, growth differentiation factor 8, GUCY2C, hemagglutinin, hepatitis B surface antigen, hepatitis B virus, HER1, HER2/neu, HER3, HGF, HHGFR, histone complex, HIV-1, HLA-DR, HNGF, Hsp90, human scatter factor receptor kinase, human TNF, human beta-amyloid, ICAM-1 (CD54), IFN-α, IFN-7, IgE, IgE Fc region, IGF-1 receptor, IGF-1, IGHE, IL 17A, IL 17F, IL 20, IL-12, IL-13, IL-17, IL-1β, IL-22, IL-23, IL-31RA, IL-4, IL-5, IL-6, IL-6 receptor, IL-9, ILGF2, influenza A hemagglutinin, influenza A virus hemagglutinin, insulin-like growth factor I receptor, integrin α4β7, integrin α4, integrin α5β1, integrin α7β7, integrin αIIbβ3, integrin αvβ3, interferon α/β receptor, interferon gamma-induced protein, ITGA2, ITGB2 (CD18), KIR2D, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch receptor, NRP1, Oryctolagus cuniculus, OX-40, oxLDL, PCSK9, PD-1, PDCD1, PDGF-R α, phosphate-sodium co-transporter, phosphatidylserine, platelet-derived growth factor receptor beta, prostatic carcinoma cells, Pseudomonas aeruginosa, rabies virus glycoprotein, RANKL, respiratory syncytial virus, RHD, Rhesus factor, RON, RTN4, sclerostin, SDC1, selectin P, SLAMF7, SOST, sphingosine-1-phosphate, Staphylococcus aureus, STEAP1, TAG-72, T-cell receptor, TEM1, tenascin C, TFPI, TGF-β1, TGF-β2, TGF-β, TNF-α, TRAIL-R1, TRAIL-R2, tumor antigen CTAA16.88, tumor specific glycosylation of MUC1, tumor-associated calcium signal transducer 2, TWEAK receptor, TYRP1 (glycoprotein 75), VEGFA, VEGFR1, VEGFR2, vimentin, VWF, 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain.

In an aspect, a tumor-associated polypeptide generated from a mutated gene has greater immunogenicity as compared to a WT polypeptide generated from an unmutated gene.

In an aspect, binding of a small molecule to a cellular target modulates an activity of the target. For example, binding of an exogenous small molecule inhibits or activates the activity and/or expression of the target. In some embodiments, the exogenous molecules utilized for generating the antigen binding units disclosed herein include but are not limited to a Ras inhibitor, an EGFR inhibitor, an FGFR inhibitor, a PI3Kinase inhibitor, a BTK inhibitor, or a Her2 inhibitor. A subject exogenous molecule can bind any residue in in RAS, EGFR, FGFR, PI3Kinase, BTK, and/or HER2. A subject exogenous molecule can bind a residue present in any one of an R-spine, C-spine, shell residue, or combinations thereof. For example in EGFR, an R-spine residue can be: L777, M766, F856, H835, or D896; a C-spine residue can be: A743, V726, L844, V845, V843, L798, L907, or T903; a shell residue can be: L788, T790, or V774. In some aspects, an exogenous molecule is a small molecule covalent inhibitor. In an aspect, a covalent inhibitor has a structure represented by: R-L-E; wherein: R is a kinase binding moiety; L is a bond or a divalent radical chemical linker; and E is an electrophilic chemical moiety capable of forming a covalent bond with a nucleophile. In an aspect, R is an optionally substituted monocyclic heteroaryl ring, an optionally substituted bicyclic aryl ring, an optionally substituted monocyclic aryl ring, or an optionally substituted bicyclic aryl ring. In an aspect, E is an electrophilic group capable of forming a covalent bond with a cysteine residue of a protein, or an electrophilic group capable of forming a covalent bond with an aspartate residue of a protein. In an aspect, E is an electrophilic group capable of forming a covalent bond with a cysteine residue or an aspartate residue of a Ab1, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, KDR, Kit, Lck, Lyn, MAP2K1, MAP2K2, MAP4K4, MAPKAPK2, Met, Mnk1, MLK1, p38, PDGFRA, PDGFRB, PDPK1, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, or Zap70 protein. In some embodiments, E is an electrophilic group capable of forming a covalent bond with a cysteine residue or an aspartate residue of a RAS, EGFR, Her2, or BTK2 protein. In some instances, E is an electrophilic group capable of forming a covalent bond with a cysteine residue or an aspartate residue of RAS, KRAS, HRAS, NRAS, KRAS G12C, HRAS G12C, NRAS G12C, EGFR, EGFR del E746-A750, EGFR del E747-E749/A750P, EGFR del E747-S752/P753S, EGFR del E747-T751/Sins/A750P, EGFR del 5752-1759, EGFR G719S, EGFR G719C, EGFR L861Q, EGFR L858R, EGFR T790M, EGFR L858R/T790M, Her2, or BTK2 protein.

In some embodiments, the exogenous molecules inhibit an enzymatic activity of the cellular target. For example, Ras inhibitor as the exogenous molecule may bind to a Ras target and inhibit its GTPase activity. An EGFR inhibitor can bind to EGFR and inhibit the kinase activity of the receptor and reduce its signalling output. A PI3Kinase inhibitor as the exogenous molecule may bind to a PI3Kinase and inhibit its lipid and/or kinase activity. A BTK inhibitor as the exogenous molecule may bind to BTK and inhibits its kinase activity. A Her2 inhibitor as the exogenous molecule may bind to an intracelluar portion of Her2 and inhibits its signalling. In some embodiments, a subject exogenous molecule inhibits its cellular target with an IC50 value less than 1 uM, 500 nM, 100 nM, 10 nM, 1 nM or even less when assayed in an in vitro inhibition assay. In some embodiments, a subject exogenous molecule inhibits its cellular target with an IC50 value less than 1 uM, 500 nM, 100 nM, 10 nM, 1 nM or even less when assayed in an in vivo inhibition assay. In some embodiments, a subject exogenous molecule inhibits cell proliferation with an EC50 value less than 10 uM, 1 uM, 500 nM, 100 nM, 10 nM, or even less.

In some embodiments, disclosed herein are covalent inhibitors of cellular targets. In some embodiments, the cellular targets are kinases. In some embodiments, the kinase is a Ab1, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, KDR, Kit, Lck, Lyn, MAP2K1, MAP2K2, MAP4K4, MAPKAPK2, Met, Mnk1, MLK1, p38, PDGFRA, PDGFRB, PDPK1, PI3Kinase, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, or Zap70 protein. In some embodiments, the kinase is a RAS, EGFR, Her2, or BTK2, FGFR, or PI3Kinase protein. In some embodiments, the kinase is a mutant form.

In some embodiments, as disclosed herein, the covalent inhibitor has a structure represented by: R-L-E; wherein: R is a kinase binding moiety; L is a bond or a divalent radical chemical linker; and E is an electrophilic chemical moiety capable of forming a covalent bond with a nucleophile. In some embodiments, R is an optionally substituted monocyclic heteroaryl ring, an optionally substituted bicyclic aryl ring, an optionally substituted monocyclic aryl ring, or an optionally substituted bicyclic aryl ring. In some embodiments, E is an electrophilic group capable of forming a covalent bond with a cysteine residue of a protein, or an electrophilic group capable of forming a covalent bond with an aspartate residue of a protein. In some embodiments, E is an electrophilic group capable of forming a covalent bond with a cysteine residue or an aspartate residue of a Ab1, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, KDR, Kit, Lck, Lyn, MAP2K1, MAP2K2, MAP4K4, MAPKAPK2, Met, Mnk1, MLK1, p38, PDGFRA, PDGFRB, PDPK1, PI3Kinase, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, or Zap70 protein. In some embodiments, E is an electrophilic group capable of forming a covalent bond with a cysteine residue or an aspartate residue of a RAS, EGFR, Her2, BTK2, FGFR, or PI3Kinase protein. In some embodiments, E is an electrophilic group capable of forming a covalent bond with a cysteine residue or an aspartate residue of RAS, KRAS, HRAS, NRAS, KRAS G12C, KRAS, G12D, HRAS G12C, NRAS G12C, EGFR, EGFR del E746-A750, EGFR del E747-E749/A750P, EGFR del E747-S752/P753S, EGFR del E747-T751/Sins/A750P, EGFR del S752-1759, EGFR G719S, EGFR G719C, EGFR L861Q, EGFR L858R, EGFR T790M, EGFR L858R/T790M, Her2, BTK2 FGFR or PI3Kinase protein. In some embodiments E is selected from the group consisting of

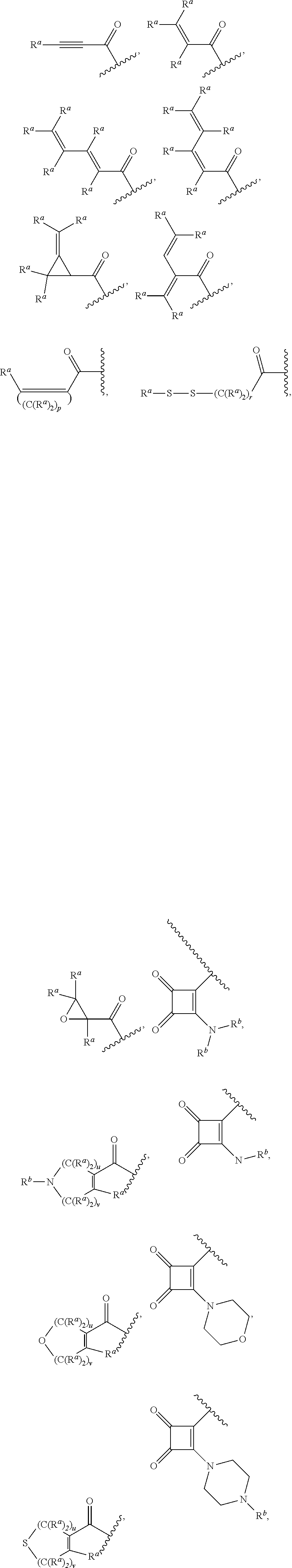

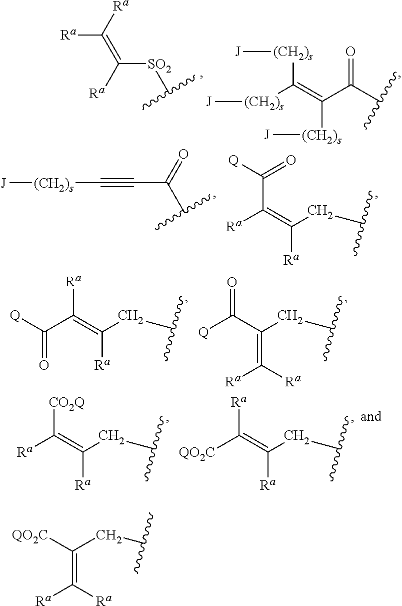

where each R^{a} is independently hydrogen, C₁₋₆alkyl, carboxy, C₁₋₆carboalkoxy, phenyl, C₂₋₇carboalkyl, R^{c}—(C(R^{b})₂)ₛ—, R^{c}—(C(R^{b})₂)ₚ-M-(C(R^{b})₂)ᵣ—, (Rd)(R^{e})CH-M-(C(R^{b})₂)ᵣ—, or Het-W—(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, cyano, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆ alkyl; R^{c} is —NR^{b}R^{b} or —OR^{b}; R^{d} and Re are each, independently, —(C(R^{b})₂)ᵣ—NR^{b}R^{b}, or —(C(R^{b})₂)ᵣ—OR^{b}; J is independently hydrogen, chlorine, fluorine, or bromine; Q is C₁₋₆alkyl or hydrogen; M is —N(R^{b})—, —O—, —N[(C(R^{b})₂)ₚ—NR^{b}R^{b}]—, or —N[(C(R^{b})₂)ₚ—OR^{b}]—; W is —N(R^{b})—, —O—, or a bond; Het is a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran; p is 2-4; r is 1-4; s is 1-6; u is 0-1; and v is 0-4, wherein the sum of u+v is 2-4. In some embodiments, E is selected from the group consisting of: R

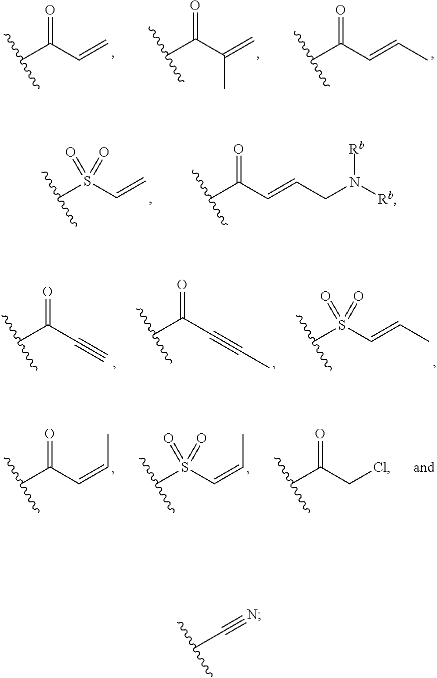

where each R^{b} is independently selected from the group consisting of hydrogen, hydroxyl, C₁-C₆ alkoxy and C₁-C₆ alkyl, or two R^{b} optionally join to form heterocycle having 3-12 ring atoms or C₃-C₆ cycloalkyl.

### RAS-Binding Exogenous Molecules

In some embodiments, the covalent inhibitor is a covalent inhibitor of a RAS protein. In some embodiments, the the covalent inhibitor is a covalent inhibitor of a KRAS, HRAS, or NRAS protein. In some embodiments, the covalent inhibitor is a covalent inhibitor of RAS, KRAS, HRAS, NRAS, KRAS G12C, KRAS, G12D, HRAS G12C, or NRAS G12C. In some embodiments, the covalent inhibitor is as described in US20180334454, US20190144444, US20150239900, U.S. Ser. No. 10/246,424, US20180086753, WO2018143315, WO2018206539, WO20191107519, WO2019141250, WO2019150305, U.S. Pat. No. 9,862,701, US20170197945, US20180086753, U.S. Ser. No. 10/144,724, US20190055211, US20190092767, US20180127396, US20180273523, U.S. Ser. No. 10/280,172, US20180319775, US20180273515, US20180282307, US20180282308, or related parents and applications, each of which is incorporated by reference in their entirety.

In some embodiments, the covalent inhibitor has the structure of Formula A:

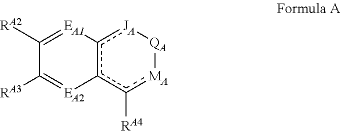

wherein:
E_{A1} and E_{A2} are each independently N or CR^{A1};
J_{A} is N, NR^{A10} or CR^{A10};
M_{A} is N, NR^{A13} or CR^{A13};
[symbol] is a single or double bond as necessary to give every atom its normal valence;
R^{A1} is independently H, hydroxy, C₁₋₄alkyl, C₁₋₄haloalkyl, C₁₋₄alkoxy, —NH—C₁₋₄alkyl, —N(C₁₋₄alkyl)₂, cyano, or halo;
R^{A2} is halo, C₁₋₆alkyl, C₁₋₆haloalkyl, —OR^{A′}, —N(R^{A′})₂, C₂₋₃alkenyl, C₂₋₃alkynyl, C₀₋₃alkylene-C₃₋₁₄cycloalkyl, C₀₋₃alkylene-C₂₋₁₄heterocycloalkyl, aryl, heteroaryl, C₀₋₃alkylene-C₆₋₁₄aryl, or C₀₋₃alkylene-C₂₋₁₄heteroaryl, and each R^{A′} is independently H, C₁₋₆alkyl, C₁₋₆haloalkyl, C₃₋₁₄cycloalkyl, C₂₋₁₄heterocycloalkyl, C₂₋₃alkenyl, C₂₋₃alkynyl, aryl, or heteroaryl, or two R^{A′} substituents, together with the nitrogen atom to which they are attached, form a 3-7-membered ring;
R³ is halo, C₁₋₃alkyl, C₁₋₂haloalkyl, C₁₋₃alkoxy, C₃₋₄cycloalkyl, C₂₋₃alkenyl, C₂₋₃alkynyl, aryl, or heteroaryl;
R^{A4} is

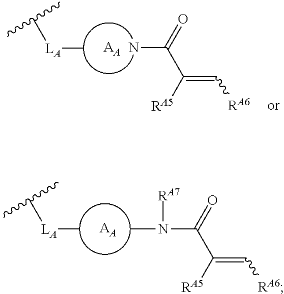

Ring A_{A} is a monocyclic 4-7 membered ring or a bicyclic, fused, or spiro 6-11 membered ring;
L_{A} is a bond, C₁₋₆alkylene, —O—C₀₋₅alkylene, —S—C₀₋₅alkylene, or —NH—C₀₋₅alkylene, and for C₂₋₆alkylene, —O—C₂₋₅alkylene, —S—C₂₋₅alkylene, and —NH—C₂₋₅alkylene, one carbon atom of the alkylene group can optionally be replaced with 0, S, or NH;
R^{A5} and R^{A6} are each independently H, halo, C₁₋₆alkyl, C₂₋₆alkynyl, C₁₋₆alkylene-O—C₁₋₄alkyl, C₁₋₆alkylene-OH, C₁₋₆haloalkyl, C₁₋₆alkyleneamine, C₀₋₆alkylene-amide, C₀₋₃alkylene-C(O)OH, C₀₋₃alkylene-C(O)OC₁₋₄alkyl, C₁₋₆alkylene-O-aryl, C₀₋₃alkylene-C(O)C₁₋₄alkylene-OH, cycloalkyl, heterocycloalkyl, aryl, heteroaryl, C₀₋₃alkylene-C₃₋₁₄cycloalkyl, C₀₋₃alkylene-C₂₋₁₄heterocycloalkyl, C₀₋₃alkylene-C₆₋₁₄ aryl, C₀₋₃alkylene-C₂₋₁₄heteroaryl, or cyano, or R^{A5} and R^{A6}, together with the atoms to which they are attached, form a 4-6 membered ring;
R^{A7} is H or C₁₋₈alkyl, or R^{A7} and R^{A5}, together with the atoms to which they are attached, form a 4-6 membered ring;
Q_{A} is CR^{A8}R^{A9}, C═CR^{A8}R^{A9}, C═O, C═S, or C=NRAS;
R^{A8} and R^{A9} are each independently H, C₁₋₃alkyl, hydroxy, C₁₋₃alkoxy, cyano, nitro, or C₃₋₆cycloalkyl, or R^{A8} and R^{A9}, taken together with the carbon atom to which they are attached, can form a 3-6 membered ring; and
R^{A10} is C₁₋₈alkyl, C₀₋₃alkylene-C₆₋₁₄aryl, C₀₋₃alkylene-C₃₋₁₄heteroaryl, C₀₋₃alkylene-C₃₋₁₄cycloalkyl, C₀₋₃alkylene-C₂₋₁₄heterocycloalkyl, C₁₋₆alkoxy, —O—C₀₋₃alkylene-C₆₋₁₄aryl, —O—C₀₋₃alkylene-C₃₋₁₄heteroaryl, —O—C₀₋₃alkylene-C₃₋₁₄cycloalkyl, —O—C₀₋₃alkylene-C₂₋₁₄heterocycloalkyl, —NH—C₁₋₈alkyl, —N(C₁₋₈alkyl)₂, —NH—C₀₋₃alkylene-C₆₋₁₄aryl, —NH—C₀₋₃alkylene-C₃₋₁₄heteroaryl, —NH—C₀₋₃alkylene-C₃₋₁₄cycloalkyl, —NH—C₀₋₃alkylene-C₂₋₁₄heterocycloalkyl, halo, cyano, or C₁₋₆alkylene-amine.

In some embodiments, the covalent inhibitor is selected from:

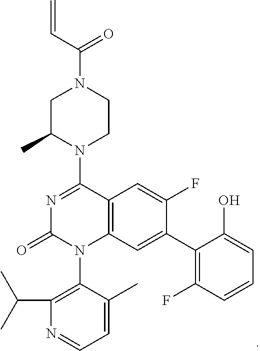 [SMILES: C=CC(=O)N1CCN(c2nc(=O)n(-c3c(C)ccnc3C(C)C)c3cc(-c4c(O)cccc4F)c(F)cc23)[C@@H](C)C1]

In some embodiments, the covalent inhibitor is selected from:

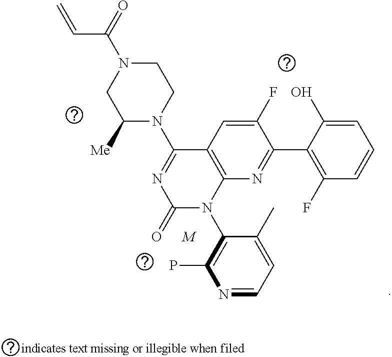

In some embodiments, the covalent inhibitor has the structure of Formula B:

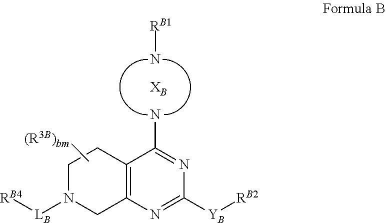

wherein:
X_{B} is a 4-12 membered saturated or partially saturated monocyclic, bridged or spirocyclic ring, wherein the saturated or partially saturated monocyclic ring is optionally substituted with one or more R^{B8};
Y_{B} is a bond, O, S, or NR^{B5};
R^{B1} is —C(O)C(R^{BA}) [symbol] C(R^{BB})_{bp} or —S(O)₂C(R^{BA}) [symbol] C(R^{BB})_{bp}; R^{B2} is hydrogen, alkyl, hydroxyalkyl, dihydroxyalkyl, alkylaminylalkyl, dialkylaminylalkyl, —Z_{B}—NR^{B5}R^{B10}, heterocyclyl, heterocyclylalkyl, aryl, heteroaryl, or heteroarylalkyl, wherein each of the Z_{B}, heterocyclyl, heterocyclylalkyl, aryl, heteroaryl, and heteroarylalkyl may be optionally substituted with one or more R^{B9};
Z_{B} is C₁-C₄ alkylene;
each R^{B3} is independently C₁-C₃ alkyl, oxo, or haloalkyl;
L_{B} is a bond, —C(O)—, or C₁-C₃ alkylene;
R^{B4} is hydrogen, cycloalkyl, heterocyclyl, aryl, aralkyl, or heteroaryl, wherein each of the cycloalkyl, heterocyclyl, aryl, aralkyl, and heteroaryl may be optionally substituted with one or more R^{B6} or R^{B7};
each R^{B5} is independently hydrogen or C₁-C₃ alkyl;
R^{B6} is cycloalkyl, heterocyclyl, heterocyclylalkyl, aryl, or heteroaryl, wherein each of the cycloalkyl, heterocyclyl, aryl, or heteroaryl may be optionally substituted with one or more R^{B7};
each R^{B7} is independently halogen, hydroxyl, C₁-C₆ alkyl, cycloalkyl, alkoxy, haloalkyl, amino, cyano, heteroalkyl, hydroxyalkyl, or -Q_{B}-haloalkyl, wherein Q_{B} is O or S;
R^{B8} is oxo, C₁-C₃ alkyl, C₂-C₄ alkynyl, heteroalkyl, cyano, —C(O)OR^{B5}, —C(O)N(R^{B5})₂, or —N(R^{B5})₂, wherein the C₁-C₃ alkyl may be optionally substituted with cyano, halogen, —OR^{B5}, —N(R^{B5})₂, or heteroaryl;
each R^{B9} is independently hydrogen, oxo, acyl, hydroxyl, hydroxyalkyl, cyano, halogen, C₁-C₆ alkyl, aralkyl, haloalkyl, heteroalkyl, cycloalkyl, heterocyclyl, heterocyclylalkyl, alkoxy, dialkylaminyl, dialkylamidoalkyl, or dialkylaminylalkyl, wherein the C₁-C₆ alkyl may be optionally substituted with cycloalkyl;
each R^{B10} is independently hydrogen, acyl, C₁-C₃ alkyl, heteroalkyl, or hydroxyalkyl;
R^{BA} is absent, hydrogen, or C₁-C₃ alkyl;
each R^{BB} is independently hydrogen, C1-C3 alkyl, alkylaminylalkyl, dialkylaminylalkyl, or heterocyclylalkyl;
bm is 0, 1, or 2; and
bp is 1 or 2;
wherein when [symbol] is a triple bond then R^{BA} is absent, R^{BB} is present, and bp is 1,
and wherein when [symbol] is a double bond then R^{BA} is present, R^{BB} is present, and bp is 2, or R^{BA}, R^{BB} and the carbon atoms to which they are attached form a 5-8 membered partially saturated cycloalkyl optionally substituted with one or more R^{B7}.

In some embodiments, the covalent inhibitor is any one of the following:

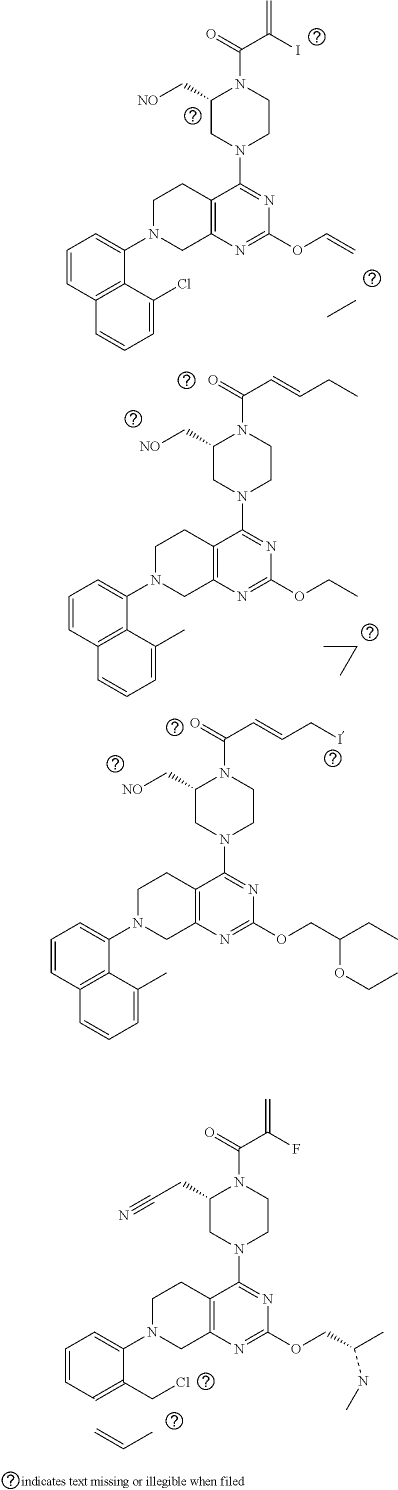

In some embodiments, the covalent inhibitor has the structure of Formula C:

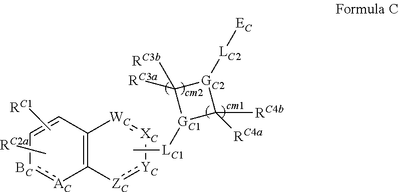

wherein:
A_{C} is CR1, CR^{C2b}, NR^{C7} or S;
B_{C} is a bond, CR^{C1} or CR^{C2c};
G_{C1} and G_{C2} are each independently N or CH;
W_{C}, X_{C} and Y_{C} are each independently N, NR^{C5} or CR^{C6};
Z_{C} is a bond, N or CR^{C6}, or Z_{C} is NH when Y is C═O;
L_{C1} is a bond or NR^{C7};
L_{C2} is a bond or alkylene;
R1 is H, cyano, halo, —CF₃, C₁-C₆alkyl, C₁-C₆alkylaminyl, C₃-C₈cycloalkyl, C₂-C₆alkenyl, or C₃-C₈cycloalkenyl, heterocyclyl, heteroaryl, aryloxy, heteroaryloxy, or aryl;
R^{C2a}, R^{C2b}, and R^{C2c} are each independently H, halo, hydroxyl, C₁-C₆alkyl, C₁-C₆haloalkyl, C₁-C₆alkoxy, C₃-C₈cycloalkyl, heteroaryl or aryl;
R^{C3a} and R^{C3b} are, at each occurrence, independently H. —OH, —NH₂, —CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl; or R^{C3a} and R^{C3b} join to form a carbocyclic or heterocyclic ring; or R^{C3a} is H, —OH, —NH₂, —CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl, and R^{C3b} joins with R^{C4b} to form a carbocyclic or heterocyclic ring;
R^{C4a} and R^{C4b} are, at each occurrence, independently H. —OH, —NH₂, CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl: or R^{C4a} and R^{C4b} join to form a carbocyclic or heterocyclic ring; or R^{C4a} is H, —OH, —NH₂, —CO₂H, halo, cyano, C₁-C₆alkyl, C₁-C₆alkynyl, hydroxylalkyl, aminylalkyl, alkylaminylalkyl, cyanoalkyl, carboxyalkyl, aminylcarbonylalkyl or aminylcarbonyl, and R^{C4b} joins with R^{C3b} to form a carbocyclic or heterocyclic ring;
R^{C5} is, at each occurrence, independently H, C₁-C₆alkyl or a bond to L_{C1};
R^{C6} is, at each occurrence, independently H, oxo, cyano, cyanoalkyl, amino, aminylalkyl, aminylalkylaminyl, aminylcarbonyl, aminylsulfonyl, —CO₂NR^{Ca}R^{Cb}, wherein R^{Ca} and R^{Cb}, are each independently H or C₁-C₆alkyl or R^{Ca} and R^{Cb} join to form a carbocyclic or heterocyclic ring, alkylaminyl, haloalkylaminyl, hydroxylalkyaminyl, amindinylalkyl, amidinylalkoxy, amindinylalkylaminyl, guanidinylalkyl, guanidinylalkoxy, guanidinylalkylaminyl, C₁-C₈alkoxy, aminylalkoxy, alkylcarbonylaminylalkoxy, C₁-C₆alkyl, heterocyclyl, heterocyclyloxy, heterocyclylalkyloxy, heterocyclylaminyl, heterocyclylalkylaminyl, heteroaryl, heteroaryloxy, heteroarylalkyloxy, heteroarylaminyl, heteroarylalkylaminyl, aryl, aryloxy, arylaminyl, arylalkylaminyl, arylalkyloxy or a bond to L_{C1};
R^{C7} is H or C₁-C₆alkyl;
cm1 and cm2 are each independently 1, 2, or 3;
[symbol] indicates a single or a double bond such that all valances are satisfied; and
E_{C} is an electrophilic moiety capable of forming a covalent bond with the cysteine residue at position 12 of a KRAS, HRAS, or NRAS G12C mutant protein;
wherein at least one of W_{C}, X_{C}, Y_{C}, and Z_{C}, is CR⁶ where R6 is a bond to L_{C1}.

In some embodiments, the covalent inhibitor has the structure of Formula D:

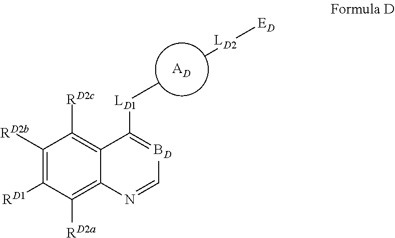

wherein:
A_{D} is a monocyclic or bicyclic moietyl;
B_{D} is N or CR^{D}′;
L_{D1} is a bond or NR^{D5};
L_{D2} is a bond or alkylene;
R^{D}′ is H, cyano, alkyl, cycloalkyl, amino, aminylakyl, alkoxy, alkoxualkyl, alkoxycarbonyl, aminylalkoxy, alkylaminylalkoxy, alkylaminyl, alkylaminylalkyl, aminylaklylaminyl, carboxyalkyl, alkylcarbonylaminyl, aminylcarbonyl, alkylaminylcarbonyl, or aminylcarbonylalkyl;
R^{D1} is aryl or heteroaryl;
R^{D2a}, R^{D2b} and R^{D2c} are each independently H, amino, halo, hydroxyl, cyano, C₁-C₆ alkyl, C₁-C₆ alkyl amino, C₁-C₆ haloalkyl (e.g., CF₃), C₁-C₆ alkoxy, C₁-C₆ haloalkoxy, C₃-C₈ cycloalkyl, heterocyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl; heteroaryl, or aryl;
R^{D5} is, at each occurrence, independently H, C₁-C₆ alkyl, C₃-C₈ cycloalkyl, or heterocyclcylalkyl; and
E_{D} is an electrophilic moiety capable of forming a covalent bond with the cysteine residue at position 12 of a KRAS, HRAS, or NRAS G12C mutant protein.

In some embodiments, the covalent inhibitor has the structure of Formula E:

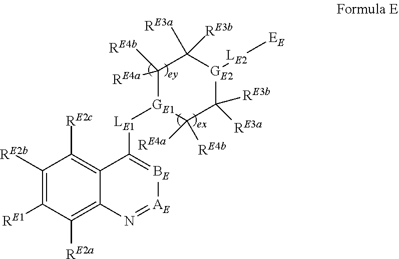

wherein:
A_{E} is N or CH;
B_{E} is N or CR^{E}′.
G^{E1} and G^{E2} are each independently N or CH;
L^{E2} is a bond or alkylene;
R^{E}′ is H, cyano, alkyl, cycloalkyl, amino, aminylalkyl, alkoxy, alkoxyalkyl, alkoxycarbonyl, aminylalkoxy, alkylaminylalkoxy, alkylaminyl, alkylaminylalkyl, aminylalkylaminyl, carboxyalkyl, alkylcarbonylaminyl, aminylcarbonyl, alkylaminylcarbonyl or aminylcarbonylalkyl;
R^{E1} is aryl or heteroaryl;
R^{E2a} and R^{E2b} are each independently amino, halo, hydroxyl, cyano, C₁-C₆ alkyl, C₁-C₆ alkyl amino, C₁-C₆ haloalkyl, C₁-C₆ alkoxy, C₁-C₆haloalkoxy, C₃-C₈ cycloalkyl, heterocycyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{E2c} is H, amino, halo, hydroxyl, cyano, C₁-C₆ alkyl, C₁-C₆ alkyl amino, C₁-C₆ haloalkyl, C₁-C₆ alkoxy, C₁-C₆ haloalkoxy, C₃-C₈ cycloalkyl, heterocycyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{E3a} and R^{E3b} are, at each occurrence, independently H, —OH, —NH₂, —CO₂H, halo, cyano, unsubstituted C₁-C₆ alkyl, C₁-C₆ haloalkyl, C₃-C₈ cycloalkyl, heterocyclylalkyl, C₁-C₆ haloalkyl, C₁-C₆ haloalkoxy, hydroxylalkly, alkoxyalkyl, aminylalkyl, carboxyalkyl, aminylcarbonylalkyl or aminylcarbonyl; or
R^{E3a} and R^{E3b}join to form oxo, a carbocyclic or heterocyclic ring; or R^{E3a} is H, —OH, —NH₂, —CO₂H, halo, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl, and R^{E3b}joins with R^{E4b} to form a carbocyclic or heterocyclic ring;
R^{E4a} and R^{E4b} are, at each occurrence, independently H, —OH, —NH₂, —CO₂H, halo, cyano, unsubstituted C₁-C₆ alkyl, C₁-C₆ haloalkyl, C₃-C₈ cycloalkyl, heterocyclylalkyl, C₁-C₆ haloalkyl, C₁-C₆ haloalkoxy, hydroxylalkly, alkoxyalkyl, aminylalkyl, carboxyalkyl, aminylcarbonylalkyl or aminylcarbonyl; or
R^{E4a} and R^{E4b}join to form oxo, a carbocyclic or heterocyclic ring; or R^{E4a} is H, —OH, —NH₂, —CO₂H, halo, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl, and R^{E4b} joins with R^{E3b} to form a carbocyclic or heterocyclic ring;
R^{E5} is, at each occurrence, independently H, C₁-C₆ alkyl, C₃-C₈cycloalkyl or heterocyclylalkyl;
ex and ey are independently integers ranging from 0 to 2; and
E_{E} is an electrophilic moiety capable of forming a covalent bond with the cysteine residue at position 12 of a KRAS, HRAS or NRAS G12C mutant protein.

In some embodiments, the covalent inhibitor has the structure of Formula F:

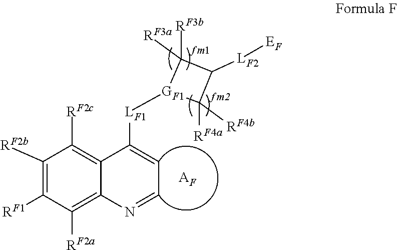

wherein:
A_{F} is a carbocyclic, heterocyclic or heteroaryl ring;
G_{F1} and G_{F2} are each independently N or CH;
L_{F1} is a bond or NR⁵;
L_{F2} is a bond or alkylene;
R^{F1} is aryl or heteroaryl;
R^{F2a}, R^{F2b} and R^{F2c} are each independently H, amino, halo, hydroxyl, cyano, C₁-C₆ alkyl, C₁-C₆ alkyl amino, C₁-C₆haloalkyl, C₁-C₆ alkoxy, C₁-C₆ haloalkoxy; C₃-C₈ cycloalkyl, heterocyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{F3a} and RF^{3b} are, at each occurrence, independently H, —OH, —NH₂, —CO₂H, halo, cyano, C₁-C₆ alkyl, C₁-C₆haloalkyl, C₁-C₆ haloalkoxy, C₃-C₈ cycloalkyl, heterocyclylalkyl, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl; or RF^{3a} and RF^{3b}join to form a carbocyclic or heterocyclic ring; or RF^{3a} is H, OH, NH₂, CO₂H, halo, cyano, C₁-C₆ alkyl, C₁-C₆ haloalkyl, C₁-C₆ haloalkoxy, C₃-C₈ cycloalkyl, heterocyclylalkyl, C₁-C₆alkynyl, hydroxylalkly, alkoxyalkyl, aminylalkyl, alkylaminylalkyl, cyanoalkyl, carboxyalkyl, aminylcarbonylalkyl or aminylcarbonyl, and R^{F3b}joins with R^{F4b} to form a carbocyclic or heterocyclic ring;
R^{F4a} and R^{F4b} are, at each occurrence, independently H, —OH, —NH₂, —CO₂H, halo, cyano, C₁-C₆ alkyl, C₁-C₆haloalkyl, C₁-C₆ haloalkoxy, C₃-C₈ cycloalkyl, heterocyclylalkyl, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl; or R^{F4a} and R^{F4b} join to form a carbocyclic or heterocyclic ring; or R^{F4a} is H, —OH, —NH₂, —CO₂H, halo, cyano, C₁-C₆ alkyl, C₁-C₆ haloalkyl, C₁-C₆ haloalkoxy, C₃-C₈ cycloalkyl, heterocyclylalkyl, C₁-C₆alkynyl, hydroxylalkly, alkoxyalkyl, aminylalkyl, alkylaminylalkyl, cyanoalkyl, carboxyalkyl, aminylcarbonylalkyl or aminylcarbonyl, and R^{F4b} joins with R^{F3b} to form a carbocyclic or heterocyclic ring;
R^{F5} is, at each occurrence, independently H, C₁-C₆ alkyl, C₃-C₈ cycloalkyl or heterocycloalkyl;
fm1 and fm2 are each independently 1, 2 or 3; and
E_{F} is an electrophilic moiety capable of forming a covalent bond with the cysteine residue at position 12 of a KRAS, HRAS or NRAS G12C mutant protein.

In some embodiments, the covalent inhibitor is selected from:

In some embodiments, the covalent inhibitor has the structure of Formula N:

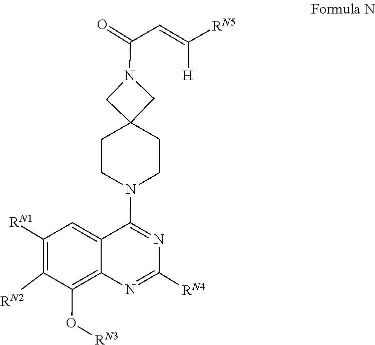

wherein:
R^{N1} is vinyl, (E)-1-propenyl or cyclopropyl;
R^{N2} is the following formula (II) or (III):

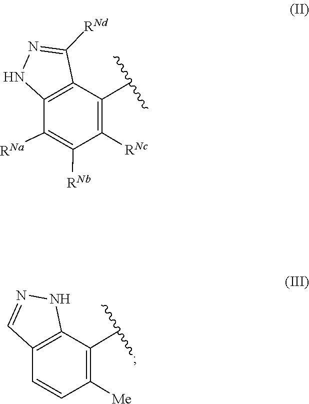

R^{N3} is C₃₋₄alkyl, methyl or n-propyl each of which may be substituted with two or more F's, ethyl or C₃₋₄cycloalkyl each of which may be substituted with F, benzyl which may be substituted with C₁₋₃alkyl, benzyl which may be substituted with —O—C₁₋₃alkyl alkyl, or benzyl which may be substituted with —O—(C₁₋₃alkyl which is substituted with F);
R^{N4} is, —O-optionally substituted C₃₋₅alkyl, —O-optionally substituted cycloalkyl, or the following formula (IV), (V), (VI) or (VII):

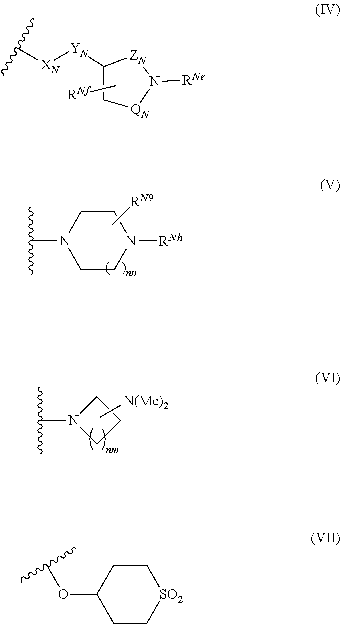

R^{N5} is H or CF₃;
R^{Na} is H or F;
R^{Nb} is H or F;
R^{Nc} is, H, methyl, vinyl or Cl;
R^{Nd} is H or Cl;
R^{Ne} is CO₂Me, COMe, CON(Me)₂, SO₂Me, C₃₋₄cycloalkyl, optionally substituted 4- to 6-membered non-aromatic heterocyclic ring, or C₁₋₃alkyl optionally substituted with a group selected from group G_{N};
Group G_{N}; —OC₁₋₃alkyl, —O—(C₁₋₃ alkyl substituted with F or C₃₋₄cycloalkyl), C₃₋₄cycloalkyl, —F, —CN, —SO₂Me, aromatic heterocyclic group, 4- to 6-membered non-aromatic heterocyclic ring, —N(C₁₋₃alkyl)₂, and —C(Me)₂OH;
R^{Nf} is, H, methyl or F;
R^{Ng} is, H, methyl or ethyl;
R^{Nh} is a good C1-3 alkyl optionally substituted with —OMe;
X_{N} is, O, NH, S or methylene;
Y_{N} is a bond or methylene;
Z_{N} is a bond, methylene or ethylene;
Q_{N} is methylene or ethylene;
nn is an integer of 1 or 2; and
nm is an integer from 1 to 3.

In some embodiments, the covalent inhibitor is selected from:
(+)-1-(7-{8-ethoxy-7-(5-methyl-1H-indazol-4-yl)-2-[(1-methylpiperidin-4-yl) oxy]-6-Binirukinazorin 4-yl}-2,7-diazaspiro [3.5] non-2-yl) prop-2-en-1-one,
(+)-1-{7-[6-cyclopropyl-2-{[1-(2-methoxyethyl) piperidin-4-yl] oxy}-7-(5-methyl-1H-indazol-4-yl)-8-(4-2,2,2-trifluoroethoxy) quinazoline yl]-2,7-diazaspiro [3.5] non-2-yl} prop-2-en-1-one,
(+)-1-{7-[2-{[1-(2-methoxyethyl) piperidin-4-yl] oxy}-7-(5-methyl-1H-indazol-4-yl)-8-(2,2,2-trifluoroethoxy)-6-Binirukinazorin-4-yl]-2,7-diazaspiro [3.5] non-2-yl} prop-2-en-1-one,
(+)-1-{7-[2-{[1-(2-ethoxyethyl) piperidin-4-yl] oxy}-7-(5-methyl-1H-indazol-4-yl)-8-(2,2,2-trifluoroethoxy)-6-Binirukinazorin-4-yl]-2,7-diazaspiro [3.5] non-2-yl} prop-2-en-1-one,
(+)-1-{7-[6-cyclopropyl-2-{[1-(3-methoxypropyl) piperidin-4-yl] oxy}-7-(5-methyl-1H-indazol-4-yl)-8-(4-2,2,2-trifluoroethoxy) quinazoline yl]-2,7-diazaspiro [3.5] non-2-yl} prop-2-en-1-one,
(+)-1-{7-[7-(5-methyl-1H-indazol-4-yl)-2-{[1-(tetrahydro-2H-pyran-4-yl) piperidin-4-yl] oxy}-8-(2,2,2-trifluoroethoxy)-6-Binirukinazorin-4-yl]-2,7-diazaspiro [3.5] non-2-yl} prop-2-en-1-one, and
(+)-1-{7-[2-{[1-(2-hydroxy-2-methylpropyl) piperidin-4-yl] oxy}-7-(5-methyl-1H-indazol-4-yl) consisting-8-(2,2,2-trifluoroethoxy)-6-Binirukinazorin-4-yl]-2,7-diazaspiro [3.5] non-2-yl} prop-2-en-1-one.

In some embodiments, the covalent inhibitor has the structure of Formula 0:

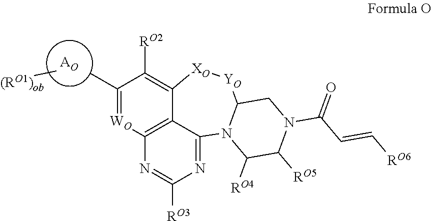

wherein:
Ring A_{O} is selected from aryl, monocyclic heteroaryl and bicyclic heteroaryl;
R^{O1} is independently selected from C₁₋₄alkyl, halo, hydroxy, C₁₋₄alkoxy, C₁₋₃fluoroalkyl, C₁₋₃fluoroalkoxy, cyano, acetylenyl, NR^{O7}R^{O8}, C(O)NR^{O9}R¹⁰, CH₂R^{O11}, N═S(O)Me₂, S(O)Me and SO₂R¹²;
ob is 0, 1, 2 or 3;
W_{O} is N or CR¹³;
X_{O} is O or NR¹⁴;
Y_{O} is CR^{O15}R^{O16}, CR^{O17}R^{O18}CR^{O19}R^{O20}, C═O, or C(O)CR^{O21}R^{O22};
R^{O2} is H, cyano, halo, C₁₋₄alkyl, C₁₋₄alkoxy, C₁₋₃fluoroalkyl, NR^{O23}R^{O24}, acetylenyl or CH₂OR^{O25};
R^{O3} is H, C₁₋₃fluoroalkyl, OR^{O26}, NR^{O27}R^{O28}, CH₂R^{O29}, SR^{O30} or C(O)R^{O31};
R^{O4} is H or Me;
R^{O5} is H or Me;
R^{O6} is H or CH₂NMe₂;
R^{O7} is H, C₁₋₄alkyl, C(O)C₁₋₃alkyl or CO₂C₁₋₃alkyl;
R^{O11} is hydroxy, cyano, heterocyclyl, NR^{O32}R^{O33}, C(O)NR^{O34}R^{O35} or SO₂C₁₋₃alkyl;
R^{O12} is C₁₋₃alkyl, C₁₋₃fluoroalkyl or NR^{O36}R^{O37};
R^{O13} is H, C₁₋₄alkyl, halo, C₁₋₃fluoroalkyl or C₁₋₄alkoxy;
R^{O15}, R^{O16}, R^{O17} and R^{O18} are independently selected from H and C₁₋₃alkyl;
R^{O19}, R^{O20}, R^{O21} and R^{O22} are independently selected from H, C₁₋₃alkyl, and fluoro;
R^{O26} is selected from the group consisting of:
   H;
   C₁₋₄alkyl optionally substituted with 1 or 2 substituents selected from hydroxy, C1-3 alkoxy, halo, NR^{O38}R^{O39}, C(O)NR^{O40}R^{O41}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
   C₃₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃ fluoroalkyl, C₃₋₇cycloalkyl, heterocyclyl or heteroaryl; and
   heteroaryl optionally substituted with C₁₋₄alkyl, hydroxy, halo, cyano or C₁₋₄alkoxy;
R²⁷ is selected from the group consisting of:
   H;
   C(O)R^{O42};
   C₁₋₄alkyl optionally substituted with 1 or 2 substituents selected from hydroxy, C₁₋₃alkoxy, halo, NR^{O43}R^{O44}, C(O)NR^{O45}R^{O46}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
   C₃₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃ fluoroalkyl, C₃₋₇cycloalkyl, CH₂cyclopropyl, heterocyclyl or heteroaryl; and
   heteroaryl optionally substituted with C₁₋₄alkyl, hydroxy, halo, cyano or C₁₋₄alkoxy;
R^{O28} is H or Me; or
R^{O27} and R^{O28} taken together with the nitrogen atom to which they are attached form a 4-, 5-, 6- or 7-membered heterocyclic ring, wherein said ring is optionally substituted with C₁₋₄alkyl, hydroxy, halo, C(O)Me, NR^{O47}R^{O48}, C₁₋₃alkoxy, C₁₋₃fluoroalkyl, C₃₋₇cycloalkyl, CH₂cyclopropyl, heterocyclyl or heteroaryl;
R^{O29} is selected from the group consisting of:
   H;
   NR^{O49}R^{O50};
   C₁₋₃alkyl optionally substituted with 1 or 2 substituents selected from hydroxy, C₁₋₃alkoxy, halo, NR^{O51}R^{O52}, C(O)NR^{O53}R^{O54}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl; C₃₋₇cycloalkyl optionally substituted with C₁₋₄alkyl, hydroxy or halo; heterocyclyl optionally substituted with C₁₋₄ alkyl, hydroxy, halo, C(O)Me, C₁₋₃alkoxy, C₁₋₃fluoroalkyl, C₃₋₇cycloalkyl, CH₂cyclopropyl, heterocyclyl or heteroaryl; and
   heteroaryl optionally substituted with C₁₋₄alkyl, hydroxy, halo, cyano or C₁₋₄alkoxy;
R^{O30} is selected from the group consisting of:
   C₁₋₄alkyl optionally substituted with 1 or 2 substituents selected from hydroxy, C₁₋₃alkoxy, halo, NR^{O55}R^{O56}, C(O)NR^{O57}R^{O58}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
   C₃₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃ fluoroalkyl, C₃₋₇cycloalkyl, heterocyclyl or heteroaryl; and
   heteroaryl optionally substituted with C₁₋₄alkyl, hydroxy, halo, cyano or C₁₋₄alkoxy;
R^{O31} is NR^{O59}R^{O60};
R^{O42} is optionally substituted heteroaryl or optionally substituted C₁₋₄alkyl;
R^{O49} and R^{O51} are independently selected from H, C₁₋₄alkyl, heterocyclyl and heteroaryl;
R^{O59} and R^{O60} are independently selected from H and C₁₋₄alkyl; or
R^{O59} and R^{O60} taken together with the nitrogen atom to which they are attached form a 4-, 5- or 6-membered heterocyclic ring, wherein said ring is optionally substituted with C₁₋₄alkyl, hydroxy, halo or C(O)Me;
R^{O8}, R^{O9}, R^{O1}, R^{O14}, R^{O23}, R^{O24}, R^{O25}, R^{O32}, R^{O33}, R^{O34}, R^{O35}, R^{O36}, R^{O37}, R^{O38}, R^{O39}, R^{O40}, R^{O41}, R^{O43}, R^{O44}, R^{O45}, R^{O46}, R^{O47}, R^{O48}, R^{O50}, R^{O52}, R^{O53}, R^{O54}, R^{O55}, R^{O56}, R^{O57}, R^{O58}, R^{O61}, and R^{O62} are independently selected from H and C₁₋₄alkyl.

In some embodiments, the covalent inhibitor has the structure of Formula Q:

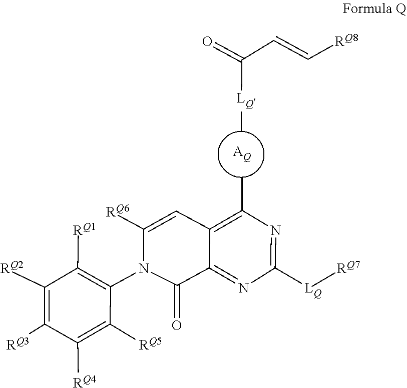

wherein:
Ring A_{Q} is 3-8 membered heterocycloalkyl, the 3-8 membered heterocycloalkyl is optionally substituted with 1, 2 or 3 of the R^{Q};
R^{O1}, R^{Q2}, R^{Q3}, R^{Q4} and R^{Q5} are independently selected from H, halogen, OH, NH₂, CN, C₁₋₆alkyl and C₁₋₆ heteroalkyl, wherein the C₁₋₆alkyl and C₁₋₆heteroalkyl is optionally substituted with 1, 2 or 3 of the R^{Q};
or, R^{Q1} and the R^{Q2} are joined together to form ring B_{Q};
or, R^{Q2} and the R^{Q3} are joined together to form ring B_{Q};
or, R^{Q3} and the R^{Q4} are joined together to form ring B_{Q};
or, R^{Q4} and the R^{Q5} are joined together to form ring B_{Q};
Ring B_{Q} is selected from the group consisting of phenyl ring, C₅₋₆Cycloalkenyl, 5-6 membered heterocycloalkenyl and the 5-6 membered aryl, phenyl, C₅₋₆Bicycloalkenyl and 5-6 membered heterocyclenyl, 5-6 membered heteroaryl ring is optionally substituted with 1, 2 or 3 R^{Qa};
R^{Qa} is selected from halogen, OH, NH₂, CN, C₁₋₆alkyl group and C₁₋₆heteroalkyl, wherein the C₁₋₆alkyl and C₁₋₆heteroalkyl is optionally substituted with 1, 2 or 3 R^{Q};
R^{Q6} is selected from H, halogen and C₁₋₆alkyl, wherein the C₁₋₆alkyl is optionally substituted with 1, 2 or 3 of the R^{Q};
R^{Q7} is selected from the group H, CN, NH₂, C₁₋₈alkyl, C₁₋₈heteroalkyl, 4-6 membered heterocylcoalkyl, 5-6 membered aryl and C₅₋₆Cycloalkyl, C₁₋₈Alkyl, C₁₋₈Heteroalkyl, 4-6 membered heterocylcoalkyl, 5-6 membered aryl and C₅₋₆Cycloalkyl is optionally substituted with 1, 2 or 3 of the R^{Q};
L_{Q} is selected from single bonds, —NH—, —S—, —O—, —C(═O)—, —C(═S)—, —CH₂—, —CH(R^{Qb})— and —C(R^{Qb})₂—;
L_{Q}′ is selected from a single bond and —NH—;
R^{Qb} is selected from C₁₋₃alkyl and C₁₋₃heteroalkyl, wherein the C₁₋₃alkyl and C₁₋₃heteroalkyl is optionally substituted with 1, 2 or 3 of the R^{Q};
R^{Q8} is selected from H, C₁₋₆alkyl and C₁₋₆heteroalkyl, wherein the C₁₋₆alkyl and C₁₋₆heteroalkyl is optionally substituted with 1, 2 or 3 of the R^{Q};
R^{Q} is selected from halogen, OH, NH₂, CN, C₁₋₆alkyl, C₁₋₆heteroalkyl and C₃₋₆cycloalkyl, wherein the C₁₋₆alkyl, C₁₋₆heteroalkyl, and C₃₋₆cycloalkyl is optionally substituted with 1, 2 or 3 R^{Q}′;
R^{Q}′ is selected from: F, Cl, Br, I, OH, NH₂, CN, CH₃, CH₃CH₂, CH₃O, CF₃, CHF₂, CH₂F, Cycloproyl, propyl, isopropyl, N(CH₃)₂, NH(CH₃);
each 3-8 membered heterocyclic alkyl, C₁₋₆Heteroalkyl, 5-6 membered heterocycloalkenyl, 5-6 membered heteroaryl, C₁₋₈Heteroalkyl, 4-6 membered heterocycloalkyl, C₁-3 Heteroalkyl contains 1, 2, or 3, “heteroatom” groups independently selected from the group of —C(═O)N(R)—, —N(R)—, —NH—, N, —O—, —S—, —C(═O)O—, —C(═O)—, —C(═S)—, —S(═O)—, —S(═O)₂— and —N(R)C(═O)N(R)—.

In some embodiments, the covalent inhibitor has the structure of Formula R:

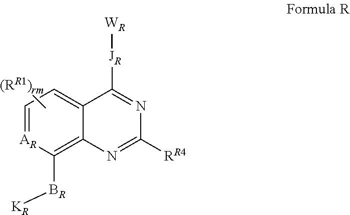

wherein:
A_{R} is —C(H)— or nitrogen;
B_{R} is oxygen, sulfur, NR^{R6} or C(R^{R6})₂;
J_{R} is a heterocycle having 3-12 ring atoms, where J_{R} is optionally substituted with 1, 2, 3, 4, 5 or 6 R^{R2};
K_{R} is C₆-C₁₂aryl, or K_{R} is heteroaryl having 5-12 ring atoms, where K_{R} is optionally substituted with 1, 2, 3, 4, 5, 6 or 7 R^{R3};
W_{R} is selected from the group consisting of:

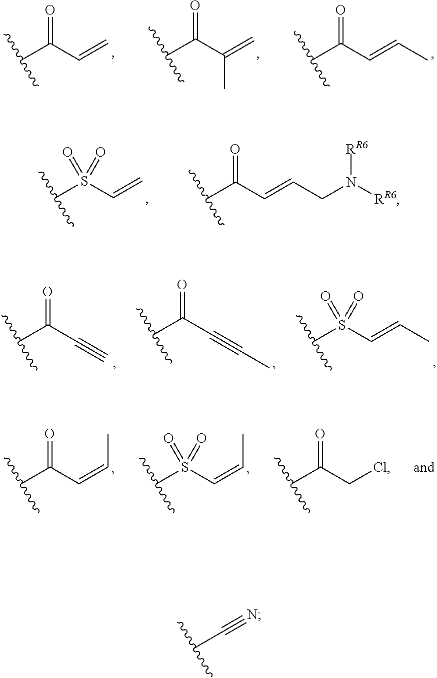

each R^{R1} is independently selected from the group consisting of C₁-C₆ alkyl, C₃-C₆ cycloalkyl, C₁-C₆ alkyl-hydroxy, C₁-C₆ alkoxy, C₁-C₆ alkyl-C₁-C₆ alkoxy, hydroxy, C2-C₆ alkenyl, C2-C₆ alkynyl, halogen, C₁-C₆ haloalkyl, cyano, and N(R^{R6})₂, or two R^{R1} optionally join to form a heterocycle having 3-12 ring atoms or a C₃-C₆ cycloalkyl;
each R^{R2} is independently selected from the group consisting of C₁-C₆ alkyl, C₃-C₆ cycloalkyl, hydroxy, C₁-C₆ alkyl-hydroxy, C₁-C₆ alkoxy, halogen, C₁-C₆ haloalkyl, cyano, C₁-C₆ alkylcyano, and oxo, or two R^{R2} optionally join to form a heterocycle having 3-12 ring atoms or a C₃-C₆ cycloalkyl;
each R^{R3} is independently selected from the group consisting of C₁-C₆ alkyl, C₃-C₆ cycloalkyl, hydroxy, C₁-C₆ alkoxy, halogen, C₁-C₆ halo-alkyl, —N(R^{R6})₂, oxo, and cyano, or two R^{R3} optionally join to form a heterocycle having 3-12 ring atoms or C₃-C₆ cycloalkyl;
R^{R4} is —X_{R}—Y_{R}—Z_{R} where:
   X_{R} is absent or is selected from the group consisting of oxygen, sulfur and —NR^{R6}—;
   Y_{R} is absent or C₁-C₆ alkylenyl; and
   Z_{R} is selected from H, —N(R^{R6})₂, —C(O)—N(R^{R6})₂, —OR^{R6}, heterocycle having 3-12 ring atoms, heteroaryl having 5-12 ring atoms, and C₃-C₆ cycloalkyl;
   where R^{R4} is optionally substituted with one or more R^{R7};
each R^{R5} is independently selected from the group consisting of: C₁-C₆ alkyl, hydroxy, C₁-C₆ alkoxy, halogen and —N(R^{R6})₂;
each R^{R6} is independently selected from the group consisting of hydrogen, hydroxyl, C₁-C₆ alkoxy and C₁-C₆ alkyl, or two R^{R6} optionally join to form heterocycle having 3-12 ring atoms or C₃-C₆ cycloalkyl;
each R^{R7} is independently R^{R7} or C₁-C₆ alkyl-R^{R7}, where each R^{R7} is independently selected from the group consisting of: C₁-C₆ alkyl, hydroxy, C₁-C₆ alkoxy, halogen, —N(R^{R6})₂, heterocycle having 3-12 ring atoms, and oxo; and
rm is 0, 1, 2 or 3.

### EGFR-Binding Exogenous Molecules

In some embodiments, an EGFR-binding exogenous molecule is an EGFR inhibitor. In some embodiments, the inhibitor covalently binds an EGFR protein, such as EGFR del E746-A750, EGFR del E747-E749/A750P, EGFR del E747-S752/P753S, EGFR del E747-T751/Sins/A750P, EGFR del S752-1759, EGFR G719S, EGFR G719C, EGFR L861Q, EGFR L858R, EGFR T790M, or EGFR L858R/T790M. In some embodiments, the covalent inhibitor is as described in U.S. Pat. No. 6,251,912, WO2013/014448, US2005/0250761, or related parents and applications, each of which is incorporated by reference in their entirety. In some embodiment, the covalent EGFR inhibitor binds to C773in mutant EGFR or C797 in wildtype EGFR.

In some embodiments, the covalent inhibitor has the structure of Formula G:

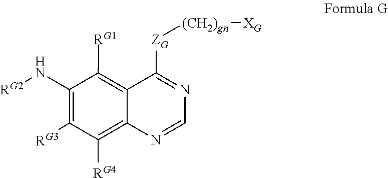

wherein:
X_{G} is cycloalkyl of 3 to 7 carbon atoms, which may be optionally substituted with one or more alkyl of 1 to 6 carbon atom groups, or is a pyridinyl, pyrimidinyl, or phenyl ring wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminomethyl, N-alkylaminomethyl of 2-7 carbon atoms, N,N-dialkylaminomethyl of 3-7 carbon atoms, mercapto, methylmercapto, and benzoylamino;
Z_{G} is —NH—, —O—, —S—, or —NR^{G}—;
R^{G} is alkyl of 1-6 carbon atoms, or carboalkyl of 2-7 carbon atoms;
R^{G1}, R^{G3}, and R^{G}a are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2-12 carbon atoms, N-alkylcarbamoyl, N,N-dialkylcarbamoyl, N-alkyl-N-alkenylamino of 4-12 carbon atoms, N,N-dialkenylamino of 6-12 carbon atoms, phenylamino, benzylamino, R^{G7}—(C(R^{G6})₂)_{gg}—Y_{G}—, R^{G7}—(C(R^{G6})₂)_{gp}-M_{G}-(C(R^{G6})₂)_{gk}—Y_{G}—, or Het_{G}-W_{G}—(C(R^{G6})₂)_{gk}—Y_{G}—;
Y_{G} is a divalent radical selected from the group consisting of —(CH₂)_{ga}—, —O—, and —NR^{G6}—;
R^{G7} is —NR^{G6}R^{G6} or —OR^{G6};
M_{G} is —N(R^{G6})—, —O—, —N[(C(R^{G6})₂)_{gp}—NR^{G6}R^{G6}]—, or —N[(C(R^{G6})₂)_{gp}—OR^{G6}]—;
W_{G} is —N(R^{G6})—, —O—, or a bond;
Het_{G} is a heterocycle, optionally mono- or di-substituted on carbon or nitrogen with R^{G6} and optionally mono-substituted on carbon with —CH₂OR^{G6}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran;
each R^{G6} is, independently, hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms;
R^{G2} is selected from the group consisting of

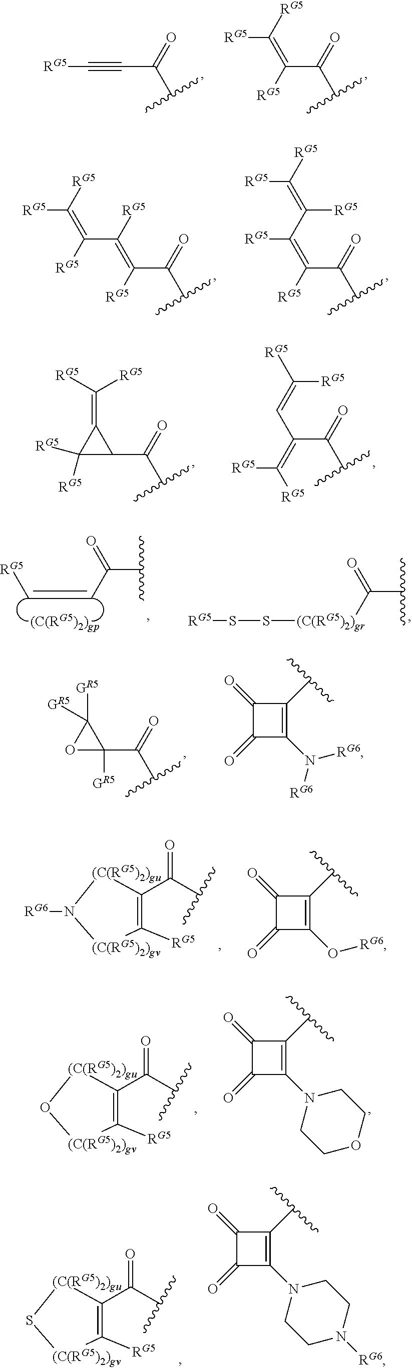

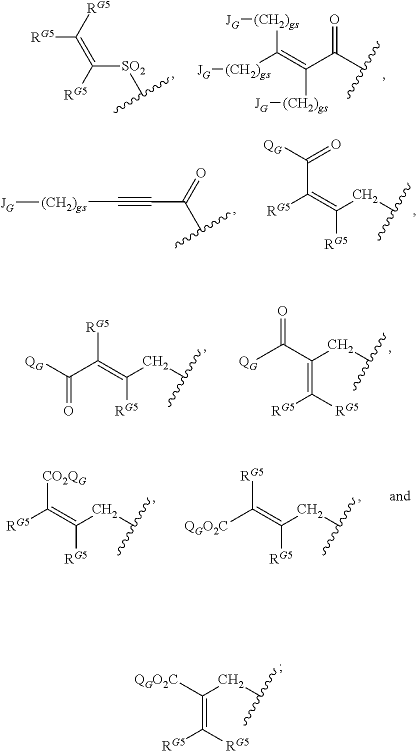

each R^{G5} is independently hydrogen, alkyl of 1-6 carbon atoms, carboxy, carboalkoxy of 1-6 carbon atoms, phenyl, carboalkyl of 2-7 carbon atoms, R^{G7}—(C(R^{G6})₂)_{gs}—, R^{G7}—(C(R^{G6})₂)_{gp}-M_{G}-(C(R^{G6})₂)_{gr}—, (R^{G8})(R^{G9})CH-M_{G}-(C(R^{G6})₂)_{gr}—, or Het_{G}-W_{G}—(C(R^{G6})₂)_{gr}—;
R^{G8} and R^{G9} are each, independently, —(C(R^{G6})₂)_{gr}—NR^{G6}R^{G6}, or —(C(R^{G6})₂)_{gr}—OR^{G6};
J_{G} is independently hydrogen, chlorine, fluorine, or bromine;
Q_{G} is alkyl of 1-6 carbon atoms or hydrogen;
ga is 0 or 1;
gg is 1-6;
gk is 0-4;
gn is 0-1;
gp is 2-4;
gq is 0-4;
gr is 1-4;
gs is 1-6;
gu is 0-1; and
gv is 0-4, wherein the sum of gu+gv is 2-4.

In some embodiments, the covalent inhibitor is Afatinib or a compound of the following structure:

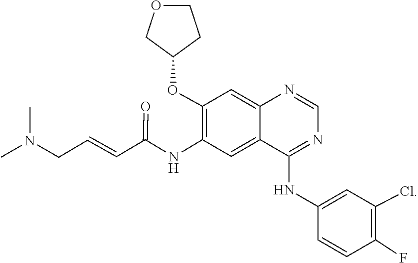 [SMILES: CN(C)C/C=C/C(=O)Nc1cc2c(Nc3ccc(F)c(Cl)c3)ncnc2cc1O[C@H]1CCOC1]

In some embodiments, the covalent inhibitor is selected from:
4-Chloro-but-2-ynoic acid [4-(3-bromo-phenylamino)-quinazolin-6yl]-amide;
4-(tert-Butyl-dimethyl-silanyloxy)-but-2-ynoic acid [4-(3-bromo-phenylamino)-quinazolin-6-yl]-amide;
4-Hydroxy-but-2-ynoic acid [4-(3-bromo-phenylamino)-quinazolin-6-yl]-amide;
4-Morpholin-4-yl-but-2-ynoic acid [4-(3-bromo-phenylamino)-quinazolin-6-yl]-amide;
4-Dimethylamino-but-2-ynoic acid [4-(3-bromo-phenylamino)-quinazolin-6-yl]-amide;
4-Methoxy-but-2-ynoic acid [4-(3-bromo-phenylamino)-quinazolin-6-yl]-amide;
4-Diethylamino-but-2-ynoic acid[4-(3-bromo-phenylamino)-quinazolin-6-yl]-amide;
4-(4-Ethyl-piperazin-1-yl)-but-2-ynoic acid [4-(3-bromo-phenylamino)-quinazolin-6-yl]-amide;
4-[3Bis-(2-methoxy-ethyl)-amino]-but-2-ynoic acid [4-(3-bromo-phenylamino)-quinazolin-6-yl]-amide;
4-(4-Methyl-piperazin-1-yl)-but-2-ynoic acid [4-(3-bromo-phenylamino)-quinazolin-6-yl]-amide;
(2-Methoxy-ethyl)-methyl-amino-but-2-ynoic acid [4-(3-bromo-phenylamino)-quinazolin-6-yl]-amide;
Isopropyl-methyl-amino-but-2-ynoic acid [4-(3-bromo-phenylamino)-quinazolin-6-yl]-amide;
Diisopropyl-amino-but-2-ynoic acid [4-(3-bromo-phenylamino)-quinazolin-6-yl]-amide;
N-[4-[(3-Bromophenyl)amino]-6-quinazolinyl]-3(E)-chloro-2-propenamide;
3-[4-(3-Bromo-phenylamino)-quinazolin-6-ylamino]-4-ethoxy-cyclobut-3-ene-1,2-dione;
3-[4-(3-Bromo-phenylamino)-quinazolin-6-ylamino]-4-dimethylamino-cyclobut-3-ene-1,2-dione;
3-[4-(3-Bromo-phenylamino)-quinazolin-6-ylamino]-4-methylamino-cyclobut-3-ene-1,2-dione;
3-Amino-4-[4-(3-bromo-phenylamino)-quinazolin-6-ylamino]-cyclobut-3-ene-1,2-dione;
3-[4-(3-Bromo-phenylamino)-quinazolin-6-ylamino]-4-morpholin-4-yl-cyclobut-3-ene-1,2-dione;
1-Methyl-1,2,5,6-tetrahydro-pyridine-3-carboxylic acid 4-(3-bromo-phenylamino)-quinazolin-6-yl]-amide;
4-(2-Methoxy-ethoxy)-but-2-ynoic acid [4-(3-bromo-phenylamino)-quinazolin-6-yl]-amide;
4-Methoxymethoxy-but-2-ynoic acid [4-(3-bromo-phenylamino)-quinazolin-6-yl]-amide;
4-Methoxy-but-2-enoic acid [4-(3-bromo-phenylamino)-quinazolin-6-yl]-amide;
2-{[4-(3-Bromo-phenylamino)-quinazolin-6-ylamino]-methyl}-acrylic acid methyl ester;
(E)-4-[4-(3-Bromo-phenylamino)-quinazolin-6-ylamino]-but-2-enoicacid methyl ester;
But-2-ynoic acid [4-(3-dimethylamino-phenylamino)-quinazolin-6-yl]-amide;
N-[4-(3-Bromo-phenylamino)-quinazolin-6-yl]-2-morpholin-4-ylmethyl-acrylamide;
4-Bromo-but-2-enoic acid [4-(3-bromo-phenylamino)-quinazolin-6-yl]-amide;
4-Dimethylamino-but-2-enoic acid [4-(3-bromo-phenylamino)-quinazolin-6-yl]-amide;
4-Diethylamino-but-2-enoic acid [4-(3-bromo-phenylamino)-quinazolin-6-yl]-amide;
N-[4-(3-Bromo-phenylamino)-quinazolin-6-yl]-2-methyldisulfanyl-acetamide;
N-[4-(3-Bromo-phenylamino)-quinazolin-6-yl]-3-methyldisulfanyl-propionamide;
N-[4-(3-Bromo-phenylamino)-quinazolin-6-yl]-2-methyldisulfanyl-propionamide;
N-[4-(3-Bromo-phenylamino)-quinazolin-6-yl]-2-tert-butyldisulfanyl-acetamide;
N-[4-(3-Bromo-phenylamino)-quinazolin-6-yl]-2-isobutyldisulfanyl-acetamide;
N-[4-(3-Bromo-phenylamino)-quinazolin-6-yl]-2-isopropyldisulfanyl-acetamide;
Oxirane-2-carboxylic acid [4-(3-bromo-phenylamino)-quinazolin-6-yl]-amide; and
Ethenesulfonic acid [4-(3-bromo-phenylamino)-quinazolin-6-yl]-amide.

In some embodiments, the covalent inhibitor has the structure of Formula H:

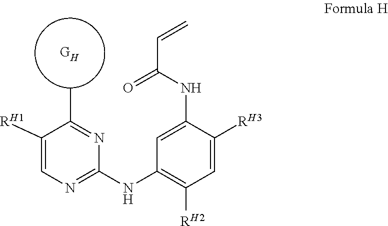

wherein:
G_{H} is selected from 4,5,6,7-tetrahydropyrazolo[1,5-a]pyridin-3-yl, 1H-indol-3-yl, 1-methyl-1H-indol-3-yl, and pyrazolo[1,5-a]pyridin-3-yl;
R^{H1} is selected from hydrogen, fluoro, chloro, methyl and cyano;
R^{H2} is selected from methoxy and methyl; and
R^{H3} is selected from (3R)-3-(dimethylamino)pyrrolidin-1-yl, (3S)-3-(dimethylamino)pyrrolidin-1-yl, 3-(dimethylamino)azetidin-1-yl, [2-(dimethylamino)ethyl]-(methyl)amino, [2-(methylamino)ethyl](methyl)amino, 5-methyl-2,5-diazaspiro[3.4]oct-2-yl, (3aR,6aR)-5-methylhexahydropyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl, and 4-[(2S)-2-aminopropanoyl]piperazin-1-yl.

In some embodiments, the covalent inhibitor is Osimertinib or a compound of the following structure:

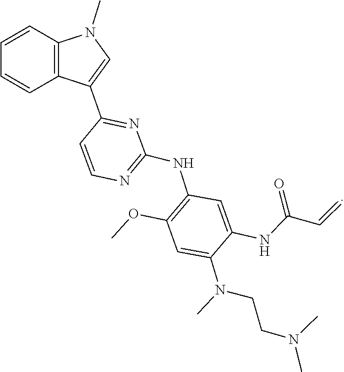

In some embodiments, the covalent inhibitor is selected from:
N-(2-{2-dimethylaminoethyl-methylamino}-4-methoxy-5-{[4-(1-methylindol-3-yl)pyrimidin-2-yl]amino}phenyl)prop-2-enamide;
N-(4-methoxy-5-{[4-(1-methylindol-3-yl)pyrimidin-2-yl]amino}-2-[methyl-(2-methylaminoethyl)amino]phenyl)prop-2-enamide;
N-(2-[2-dimethylaminoethyl-methylamino]-5-{[4-(1H-indol-3-yl)pyrimidin-2-yl]amino}-4-methoxyphenyl)-prop-2-enamide.

In some embodiments, the covalent inhibitor has the structure of Formula I:

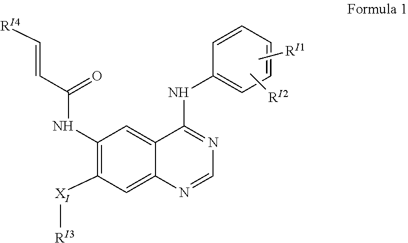

wherein:
R^{I1} is selected from F, Br, Cl, or I;
R^{I2} is selected from H, F, Br, Cl, or I;
R^{I3} is selected from:
   a) C₁-C₃ straight or branched alkyl, optionally substituted by halogen; or
   b) —(CH₂)ᵢₙ-morpholino, —(CH₂)ᵢₙ-piperidine, —(CH₂)ᵢₙ-piperazine, —(CH₂)ᵢₙ-piperazine-N(C₁-C₃ alkyl), —(CH₂)ᵢₙ-pyrrolidine, or —(CH₂)ᵢₙ-imidazole;
in is 1-4;
R^{I4} is —(CH₂)ᵢₘ-Het_{I};
Het_{I} is a heterocyclic moiety selected from the group of morpholine, piperidine, piperazine, piperazine-N(C₁-C₃ alkyl), imidazole, pyrrolidine, azepane, 3,4-dihydro-2H-pyridine, or 3,6-dihydro-2H-pyridine, wherein each heterocyclic moiety is optionally substituted by from 1 to 3 groups selected from C₁-C₃ alkyl, halogen, —OH, —NH₂, —NH(C₁-C₃ alkyl) or —N(C₁-C₃ alkyl)₂;
im is 1-3; and
X_{I} is O, S, or NH.

In some embodiments, the covalent inhibitor is Dacomitinib or a compound of the following structure:

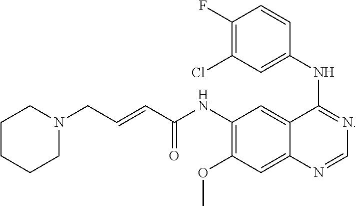 [SMILES: COc1cc2ncnc(Nc3ccc(F)c(Cl)c3)c2cc1NC(=O)/C=C/CN1CCCCC1]

In some embodiments, the covalent inhibitor is selected from the following:
4-Piperidin-1-yl-but-2-enoic acid [4(3-chloro-4-fluoro-phenylamino)-7-methoxy-quinazolin-6-yl]-amide;
4-Piperidin-1-yl-but-2-enoic acid [4(3-chloro-4-fluoro-phenylamino)-7-methylsulfanyl-quinazolin-6-yl]-amide;
4-Piperidin-1-yl-but-2-enoic acid [4(3-chloro-4-fluoro-phenylamino)-7-methylamino-quinazolin-6-yl]-amide;
4-Piperidin-1-yl-but-2-enoic acid [4(3-chloro-4-fluoro-phenylamino)-7-isopropoxy-quinazolin-6-yl]-amide;
4-Piperidin-1-yl-but-2-enoic acid [4(3-bromo-phenylamino)-7-methoxy-quinazolin-6-yl]-amide;
4-Piperidin-1-yl-but-2-enoic acid [4(3-chloro-4-fluoro-phenylamino)-7-ethoxy-quinazolin-6-yl]-amide;
4-Piperidin-1-yl-but-2-enoic acid [4(3-chloro-4-fluoro-phenylamino)-7-propoxy-quinazolin-6-yl]-amide;
4-(4-Fluoro-piperidin-1-yl)-but-2-enoic acid [4-(3-chloro-4-fluoro-phenylamino)-7-methoxy-quinazolin-6-yl]-amide;
4-(3-Fluoro-piperidin-1-yl)-but-2-enoic acid [4-(3-chloro-4-fluoro-phenylamino)-7-methoxy-quinazolin-6-yl]-amide;
4-(2-Fluoro-piperidin-1-yl)-but-2-enoic acid [4-(3-chloro4-fluoro-phenylamino)-7-methoxy-quinazolin-6-yl]-amide;
4-Morpholin-4-yl-but-2-enoic acid [4(3-chloro-4-fluoro-phenylamino)-7-methoxy-quinazolin-6-yl]-amide;
4-Azepan-1-yl-but-2-enoic acid [4-(3-chloro-4-fluoro-phenylamino)-7-methoxy-quinazolin-6-yl]-amide;
4-Piperidin-1-yl-but-2-enoic acid [4(3-chloro-4-fluoro-phenylamino)-7-trifluoromethoxy-quinazolin-6-yl]-amide;
4-Piperidin-1-yl-but-2-enoic acid [4(3-chloro-4-fluoro-phenylamino)-7-fluoromethoxy-quinazolin-6-yl]-amide;
4-Piperidin-1-yl-but-2-enoic acid [4(3-chloro-4-fluoro-phenylamino)-7-fluoroethoxy-quinazolin-6-yl]-amide;
4-Piperidin-1-yl-but-2-enoic acid [4(3-chloro-4-fluoro-phenylamino)-7-(2-fluoro-ethylsulfanyl)-quinazolin-6-yl]-amide;
4-Piperidin-1-yl-but-2-enoic acid [4(3-chloro-4-fluoro-phenylamino)-7-trifluoroethoxy-quinazolin-6-yl]-amide;
4-Piperidin-1-yl-but-2-enoic acid [4(3-chloro-4-fluoro-phenylamino)-7-difluoroethoxy-quinazolin-6-yl]-amide;
4-Piperidin-1-yl-but-2-enoic acid [4(3-chloro-4-fluoro-phenylamino)-7-(3-morpholin-4-yl-propoxy)-quinazolin-6-yl]-amide;
4-Piperidin-1-yl-but-2-enoic acid [4(3-chloro-4-fluoro-phenylamino)-7-(2-piperidin-1-yl-ethoxy)-quinazolin-6-yl]-amide;
4-(3,4-Dihydro-2H-pyridin-1-yl)-but-2-enoic acid [4(3-chloro-4-fluoro-phenylamino)-7-methoxy-quinazolin-6-yl]amide;
4-(3,6-Dihydro-2H-pyridin-1-yl)-but-2-enoic acid [4(3-chloro-4-fluoro-phenylamino)-7-methoxy-quinazolin-6-yl]amide;
4-Piperazin-1-yl-but-2-enoic acid [4(3-chloro-4-fluoro-phenylamino)-7-methoxy-quinazolin-6-yl]amide;
4-(4-Methyl-piperazin-1-yl)-but-2-enoic acid [4(3-chloro-4-fluoro-phenylamino)-7-methoxy-quinazolin-6-yl]amide;
4-Imidazol-1-yl-but-2-enoic acid [4(3-chloro-4-fluoro-phenylamino)-7-methoxy-quinazolin-6-yl]amide;
4-Pyrrolidin-1-yl-but-2-enoic acid [4(3-chloro-4-fluoro-phenylamino)-7-methoxy-quinazolin-6-yl]amide;
5-Piperidin-1-yl-pent-2-enoic acid [4(3-chloro-4-fluoro-phenylamino)-7-methoxy-quinazolin-6-yl]-amide;
5-Piperidin-1-yl-pent-2-enoic acid [4(3-chloro-4-fluoro-phenylamino)-7-methylsulfanyl-quinazolin-6-yl]-amide;
5-Piperidin-1-yl-pent-2-enoic acid [4(3-chloro-4-fluoro-phenylamino)-7-methylamino-quinazolin-6-yl]-amide;
5-Piperidin-1-yl-pent-2-enoic acid [4(3-chloro-4-fluoro-phenylamino)-7-isopropoxy-quinazolin-6-yl]-amide;
5-Piperidin-1-yl-pent-2-enoic acid [4(3-bromo-phenylamino)-7-methoxy-quinazolin-6-yl]-amide;
5-Piperidin-1-yl-pent-2-enoic acid [4(3-chloro-4-fluoro-phenylamino)-7-ethoxy-quinazolin-6-yl]-amide;
5-Piperidin-1-yl-pent-2-enoic acid [4(3-chloro-4-fluoro-phenylamino)-7-propoxy-quinazolin-6-yl]-amide;
5-(4-Fluoro-piperidin-1-yl)-pent-2-enoic acid [4-(3-chloro-4-fluoro-phenylamino)-7-methoxy-quinazolin-6-yl]-amide;
5-(3-Fluoro-piperidin-1-yl)-pent-2-enoic acid [4-(3-chloro-4-fluoro-phenylamino)-7-methoxy-quinazolin-6-yl]-amide;
5-(2-Fluoro-piperidin-1-yl)-pent-2-enoic acid [4-(3-chloro4-fluoro-phenylamino)-7-methoxy-quinazolin-6-yl]-amide;
5-Morpholin-4-yl-pent-2-enoic acid [4(3-chloro-4-fluoro-phenylamino)-7-methoxy-quinazolin-6-yl]-amide;
5-Azepan-1-yl-pent-2-enoic acid [4-(3-chloro-4-fluoro-phenylamino)-7-methoxy-quinazolin-6-yl]-amide;
5-Piperidin-1-yl-pent-2-enoic acid [4(3-chloro-4-fluoro-phenylamino)-7-trifluoromethoxy-quinazolin-6-yl]-amide;
5-Piperidin-1-yl-pent-2-enoic acid [4(3-chloro-4-fluoro-phenylamino)-7-fluoromethoxy-quinazolin-6-yl]-amide;
5-Piperidin-1-yl-pent-2-enoic acid [4(3-chloro-4-fluoro-phenylamino)-7-fluoroethoxy-quinazolin-6-yl]-amide;
5-Piperidin-1-yl-pent-2-enoic acid [4(3-chloro-4-fluoro-phenylamino)-7-(2-fluoro-ethylsulfanyl)-quinazolin-6-yl]-amide;
5-Piperidin-1-yl-pent-2-enoic acid [4(3-chloro-4-fluoro-phenylamino)-7-trifluoroethoxy-quinazolin-6-yl]-amide;
5-Piperidin-1-yl-pent-2-enoic acid [4(3-chloro-4-fluoro-phenylamino)-7-difluoroethoxy-quinazolin-6-yl]-amide;
5-Piperidin-1-yl-pent-2-enoic acid [4(3-chloro-4-fluoro-phenylamino)-7-(3-morpholin-4-yl-propoxy)-quinazolin-6-yl]-amide;
5-Piperidin-1-yl-pent-2-enoic acid [4(3-chloro-4-fluoro-phenylamino)-7-(2-piperidin-1-yl-ethoxy)-quinazolin-6-yl]-amide;
6-Piperidin-1-yl-hex-2-enoic acid [4-(3-chloro-4-fluoro-phenylamino)-7-methoxy-quinazolin-6-yl]-amide;
6-Piperidin-1-yl-hex-2-enoic acid [4-(3-chloro4-fluoro-phenylamino)-7-methylsulfanyl-quinazolin-6-yl]-amide;
6-Piperidin-1-yl-hex-2-enoic acid [4-(3-chloro-4-fluoro-phenylamino)-7-methylamino-quinazolin-6-yl]-amide;
6-Piperidin-1-yl-hex-2-enoic acid [4-(3-chloro-4-fluoro-phenylamino)-7-ethoxy-quinazolin-6-yl]-amide; and
6-Piperidin-1-yl-hex-2-enoic acid [4-(3-chloro-4-fluoro-phenylamino)-7-fluoroethoxy-quinazolin-6-yl]-amide.

### Her2-Binding Exogenous Molecules

In some embodiments, a Her-2 binding exogenous molecule is an inhibitor. In some embodiment, the Her-2 binding exogenous molecule is an inhibitor capable of covalently binding to a Her2 protein. In some embodiment, the covalent inhibitor binds to mutant Her 2 (S310F/Y mutation). In some embodiments, the inhibitor binds to C773 of Her2. In some embodiments, the covalent inhibitor is as described in U.S. Pat. No. 6,288,082, or related parents and applications, each of which is incorporated by reference in their entirety.

In some embodiments, the covalent inhibitor has the structure of Formula J:

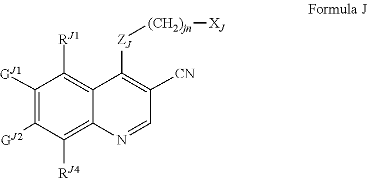

wherein:
X_{J} is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2-12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or

X_{J} is a radical having the formula:

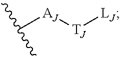

wherein
A_{J} is a pyridinyl, pyrimidinyl, or phenyl ring, wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2-12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T_{J} is bonded to a carbon of A_{J} and is: —NH(CH₂)ⱼₘ—, —O(CH₂)ⱼₘ—, —S(CH₂)ⱼₘ—, —NR(CH₂)ⱼₘ, —(CH₂)ⱼₘ—, —(CH₂)ⱼₘ—NH—, —(CH₂)ⱼₘ—O—, —(CH₂)ⱼₘ—S—, or —(CH₂)ⱼₘ—NR—;
L_{J} is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2-12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
or L_{J} is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2-12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
Z_{J} is —NH—, —O—, —S—, or —NR^{J}—;
R^{J} is alkyl of 1-6 carbon atoms, or carboalkyl of 2-7 carbon atoms;
G^{J1}, G^{J2}, R^{J1}, and R^{J4} are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2-12 carbon atoms, N-alkylcarbamoyl, N,N-dialkylcarbamoyl, N-alkyl-N-alkenylamino of 4-12 carbon atoms, N,N-dialkenylamino of 6-12 carbon atoms, phenylamino, benzylamino, (R^{J8})(R^{J9})CH-M_{J}-(C(R^{J6})₂)ⱼₖ—Y_{J}—, R^{J7}—(C(R^{J6})₂)_{jg}—Y_{J}—, R^{J7}—(C(R^{J6})₂)ⱼₚ-M_{J}- (C(R^{J6})₂)ⱼₖ—Y_{J}—, Het_{J}-(C(R^{J6})₂)_{jg}—W_{J}—(C(R^{J6})₂)ⱼₖ—Y_{J}—, or

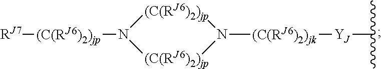

or R^{J1} and R^{J4} are as defined above and G^{J1} or G^{J2} or both are R^{J2}—NH—;
or if any of the substituents R^{J1}, G^{J1}, G^{J2}, or R^{J4} are located on contiguous carbon atoms then they may be taken together as the divalent radical —O—C(R^{J6})₂—O—;
   Y_{J} is a divalent radical Selected from the group consisting of —(CH₂)ⱼₐ—, —O—, and —NR^{J6}—;
   R^{J7} is —NR^{J6}R^{J6}, —OR^{J6}, -J_{J}, —N(R^{J6})₃*, or —NR^{J6}(OR^{J6}),
   M_{J} is —N(R^{J6})—, —O—, —N[(C(R^{J6})₂)ⱼₚ—NR^{J6}R^{J6}]—, or —N[(C(R^{J6})₂)ⱼₚ—OR^{J6}]—,
   W_{J} is —N(R^{J6})—, —O—, or a bond;
   Het_{J} is is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and

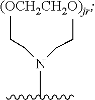

wherein Het_{J} is optionally mono- or di-substituted on carbon or nitrogen with R₆, optionally mono- or di-substituted on carbon with hydroxy, —N(R^{J6})₂, or —OR^{J6}, optionally mono or di-substituted on carbon with the mono-valent radicals —(C(R^{J6})₂)ⱼₛ—OR^{J6} or —(C(R^{J6})₂)ⱼₛ—N(R^{J6})₂, and optionally mono or di-substituted on a saturated carbon with divalent radicals —O— or —O—(C(R^{J6})₂)ⱼₛ—O—;
R^{J6} is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R^{J4} is selected from the group consisting of

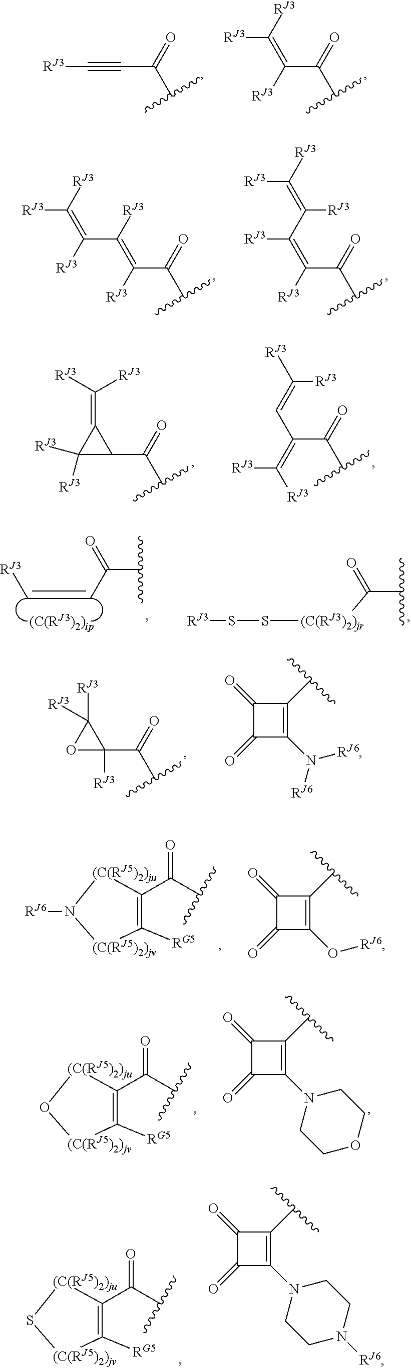

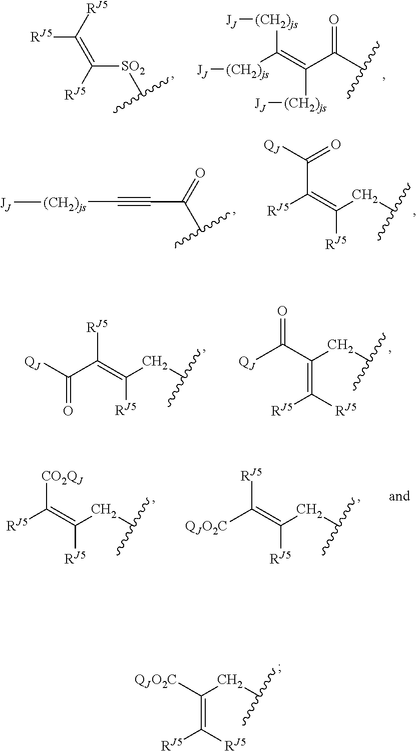

R^{J3} is independently hydrogen, alkyl of 1-6 carbon atoms, carboxy, carboalkoxy of 1-6 carbon atoms, phenyl, carboalkyl of 2-7 carbon atoms, R^{J7}—(C(R^{J6})₂)ⱼₛ—, R^{J7}—(C(R^{J6})₂)ⱼₚ-M_{J}-(C(R^{J6})₂)ⱼᵣ—, (R^{J8})(R^{J9})CH- M_{J}-(C(R^{J6})₂)ⱼᵣ—, Het_{J}-(C(R^{J6})₂)_{jg}—W_{J}—(C(R^{J6})₂)ⱼᵣ—, or

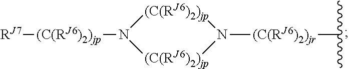

R^{J5} is independently hydrogen, alkyl of 1-6 carbon atoms, carboxy, carboalkoxy of 1-6 carbon atoms, phenyl, carboalkyl of 2-7 carbon atoms, R^{J7}—(C(R^{J6})₂)ⱼₛ—, R^{J7}—(C(R^{J6})₂)ⱼₚ-M_{J}-(C(R^{J6})₂)ⱼᵣ—, (R^{J8})(R^{J9})CH-M_{J}-(C(R^{J6})₂)ⱼᵣ—, Het_{J}-(C(R^{J6})₂)_{jg}—W_{J}—(C(R^{J6})₂)ⱼᵣ—, or

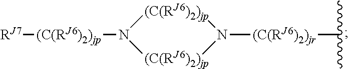

R^{J8} and R^{J9} are each, independently, —(C(R^{J6})₂)ⱼᵣ—NR^{J6}R^{J6} or —(C(R^{J6})₂)ⱼᵣ—OR^{J6};
J_{J} is independently hydrogen, chlorine, fluorine, or bromine;
Q_{J} is alkyl of 1-6 carbon atoms or hydrogen;
ja is 0 or 1;
jg is 1-6;
jk is 0-4;
jn is 0-1;
jm is 0-3
jp is 2-4;
jg is 0-4;
jr is 1-4;
js is 1-6;
ju is 0-4; and
jv is 0-4, wherein the sum of ju+jv is 2-4;
provided that when R¹⁶ is alkenyl of 2-7 carbon atoms or alkynyl of 2-7 carbon atoms, such alkenyl or alkynyl moiety is bound to a nitrogen or oxygen atom through a saturated carbon atom.

In some embodiments, the covalent inhibitor is Neratinib or a compound of the following structure:

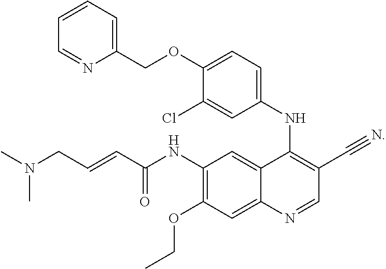 [SMILES: CCOc1cc2ncc(C#N)c(Nc3ccc(OCc4ccccn4)c(Cl)c3)c2cc1NC(=O)/C=C/CN(C)C]

### BTK Inhibitors

In some embodiments, the covalent inhibitor is a covalent inhibitor of a BTK protein. In some embodiments, the covalent inhibitor is a covalent inhibitor of BTK. In some embodiments, the covalent inhibitor is as described in US2008/0076921, WO2013/010868, WO2014/173289, or related parents and applications, each of which is incorporated by reference in their entirety.

In some embodiments, the covalent inhibitor has the structure of Formula M:

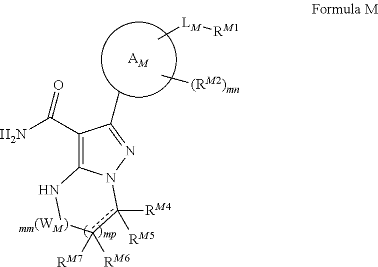

wherein:
A_{M} is a 5- or 6-membered aromatic ring comprising 0-3 heteroatoms of N, S or O;
each W_{M} is independently —(CH₂)— or —C(O)—;
L_{M} is a bond, CH₂, NR^{M12}, O, S;
[symbol] is a single or double bond, and when a double bond, R^{M5} and R^{M7} are absent;
mm is 0-4;
mn is 0-4, wherein when mn is more than 1, each R^{M2}mat be different;
mp is 0-2, wherein when mp is 0, mm is 1-4, and when mp is 2, each R^{M6} and each R^{M7}may be different;
R^{M1}, R^{M4}, R^{M5}, R^{M6}, and R^{M7} are each independently H, halogen, heteroalkyl, alkyl, alkenyl, cycloalkyl, aryl, saturated or unsaturated heterocyclyl, heteroaryl, alkynyl, —CN, —NR^{M13}R^{M14}, —OR^{M13}, —COR^{M13}, —CO₂R^{M13}, —CONR^{M13}R^{M14}, —C(═NR^{M13})NR^{M14}R^{M15}, —NR^{M13}COR^{M14}, —NR^{M13}CONR^{M14}R^{M15}, —NR^{M13}CO₂R^{M14}, —SO₂R^{M13}, —NR^{M13}SO₂NR^{M14}R^{M15}, or —NR^{M13}SO₂R^{M14}, wherein the alkyl, alkenyl, alkynyl, cycloalkyl, heteroaryl, aryl, and saturated or unsaturated heterocyclyl are optionally substituted with at least one substituent R^{M16}, wherein (R^{M4} and R^{M5}), or (R^{M4} and R^{M6}), or (R^{M6} and R^{M7}), or (R^{M6} and R^{M6} when mp is 2), together with the atoms to which they are attached, can form a ring selected from cycloalkyl, saturated or unsaturated heterocycle, aryl, and heteroaryl rings optionally substituted with at least one substituent R^{M16};
R^{M2} is halogen, alkyl, —S-alkyl, —CN, —NR^{M13}R^{M14}, —OR^{M13}, —COR^{M13}, —CO₂R^{M13}, —CONR^{M13}R^{M14}, —C(═NR^{M13})NR^{M14}R^{M15}, —NR^{M13}COR^{M14}, —NR^{M13}CONR^{M14}R^{M15}, —NR^{M13}CO₂R^{M14}, —SO₂R^{M13}, —NR^{M13}SO₂NR^{M14}R^{M15} or —NR^{M13}SO₂R^{M14}.
R^{M12} is H or lower alkyl;
R^{M13}, R^{M14} and R^{M15} are each independently H, heteroalkyl, alkyl, alkenyl, alkynyl, cycloalkyl, saturated or unsaturated heterocyclyl, aryl, or heteroaryl; wherein (R^{M13} and R^{M14}), and/or (R^{M14} and R^{M15}) together with the atom(s) to which they are attached, each can form a ring selected from cycloalkyl, saturated or unsaturated heterocycle, aryl, and heteroaryl rings optionally substituted with at least one substituent R^{M16}; and
R^{M16} is halogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted heterocyclyl, oxo, —CN, —OR^{M}′, —NR^{M}′R^{M}″, —COR^{M}′, —CO₂R^{M}′, —CONR^{M}′R^{M}″, —C(═NR^{M}′)NR^{M}″R^{M}′″, —NR^{M}′COR^{M}″—NR^{M}′CONR^{M}′R^{M}″, —NR^{M}′CO₂R^{M}″, —SO₂R^{M}′, —SO₂aryl, —NR^{M}′SO₂NR^{M}″R^{M}′″, or —NR^{M}′SO₂R^{M}″, wherein R^{M}′, R^{M}″, and R^{M}′″ are independently hydrogen, halogen, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, substituted or unsubstituted heterocyclyl, wherein (R^{M}′ and R^{M}″), and/or (R^{M}″ and R^{M}′″) together with the atoms to which they are attached, can form a ring selected from cycloalkyl, saturated or unsaturated heterocycle, aryl, and heteroaryl rings.

In some embodiments, the covalent inhibitor is Zanubrutinib or a compound having the structure of:

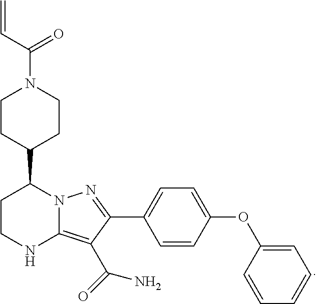 [SMILES: C=CC(=O)N1CCC([C@@H]2CCNc3c(C(N)=O)c(-c4ccc(Oc5ccccc5)cc4)nn32)CC1]

### FGFR-Binding Exogenous Molecules

In some embodiments, the FGFR-binding exogenous molecules are inhibitors. In some embodiments, the inhibitors are capable of covalently binding to a FGFR protein. In some embodiments, the covalent inhibitor binds to FGFR-1, FGFR-2, or FGFR-3. In some embodiments, the covalent inhibitor binds to a mutant FGFR, such as N546K & N546D mutant of FGFR-1, or N549K of FGFR2, or S249C of FGFR3. In some embodiments, the covalent inhibitor is as described in WO2014011900, WO2014182829, or related parents and applications, each of which is incorporated by reference in their entirety.

In some embodiments, the covalent inhibitor has the structure of Formula S:

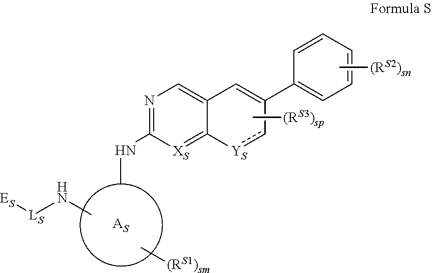

wherein:
E_{S} is a moiety that is capable of forming a covalent bond with a nucleophile;
Ring A_{S} is a 3-8 membered aryl, heteroaryl, heterocyclic or alicyclic group;
X_{S} is CH or N;
Y_{S} is CH or N—R^{S4}, where R^{S4} is H or C₁₋₆ alkyl;
L_{S} is —[C(R^{S5})(R^{S6})]_{sq}—, where each of R^{S5} and R^{S6} is, independently, H or C₁₋₆ alkyl; and sq is 0-4;
each R^{S1}, R^{S2}, and R^{S3} is, independently, halo, cyano, optionally substituted C₁₋₆ alkoxy, hydroxy, oxo, amino, amido, alkylurea, optionally substituted C₁₋₆ alkyl, or optionally substituted C₂₋₆ heterocyclyl;
sm is 0-3;
sn is 0-4; and
sp is 0-2.

In some embodiments, Ring A_{S} is phenyl, e.g., a 1,2-disubstituted phenyl; R^{S2} is halo or methoxy; sn is 2 or 4; X_{S} is N; R^{S1} is methyl; and/or sm is 1.

In some embodiments, the covalent inhibitor is selected from:

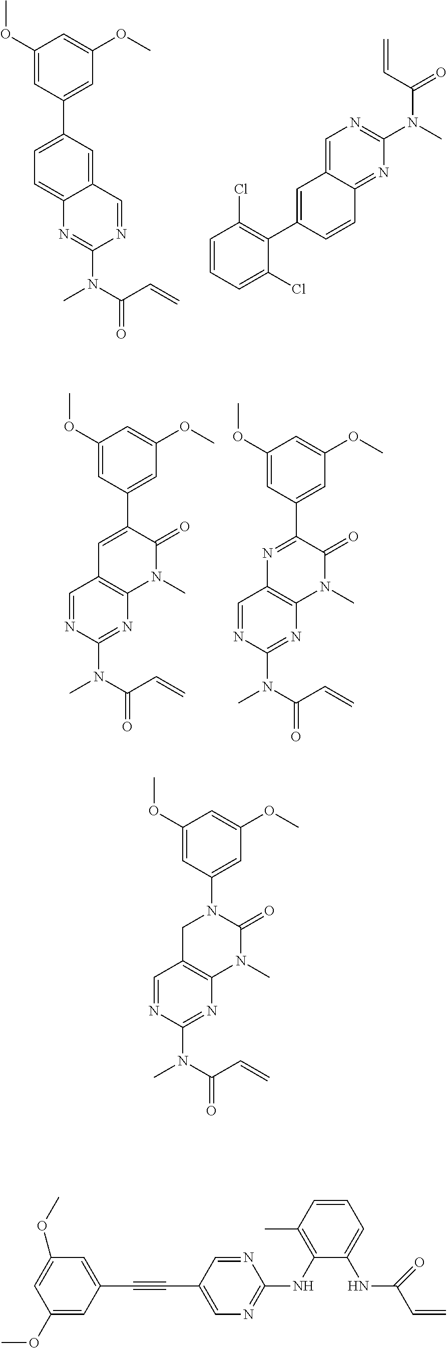

In some embodiments, the covalent inhibitor has the structure of Formula T:

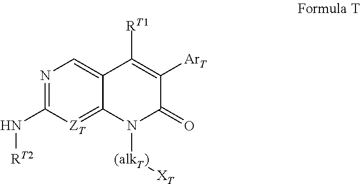

wherein:
Ar_{T} is phenyl or heteroaryl, each ring optionally substituted with one, two, three, or four substituents independently selected from alkyl, cycloalkyl, hydroxy, alkoxy, halo, haloalkyl, alkylsulfonyl, haloalkoxy, and cyano;
R^{T1} is hydrogen, halo, or alkyl;
R^{T2} is hydrogen, alkyl, cycloalkyl substituted with amino, alkylamino, or dialkylamino, hydroxyalkyl, alkoxyalkyl, aminoalkyl, heterocyclyl (wherein heterocyclyl is optionally substituted with one or two substituents independently selected from alkyl, hydroxyalkyl, alkoxyalkyl, aminoalkyl, optionally substituted aryl, optionally substituted heteroaryl, and optionally substituted heterocyclyl), heterocyclylalkyl (wherein the heterocyclyl ring in heterocyclylalky is optionally substituted with one or two substituents independently selected from alkyl, hydroxyalkyl, aminoalkyl, optionally substituted aryl, optionally substituted heteroaryl, and optionally substituted heterocyclyl), phenyl or heteroaryl (wherein phenyl or heteroaryl is optionally substituted with one, two, or three substituents where two of the phenyl or heteroaryl optional substituents are independently selected from alkyl, hydroxy, alkoxy, halo, haloalkyl, haloalkoxy, and cyano and one of the phenyl or heteroaryl optional substituents is alkyl, cycloalkyl, hydroxy, alkoxy, halo, haloalkyl, haloalkoxy, cyano, hydroxyalkyl, alkoxyalkyl, aminoalkyl, optionally substituted aryl, optionally substituted heteroaryl or optionally substituted heterocyclyl);
alk_{T} is alkylene;
X_{T} is a group of formula (a_{T}) or (b_{T}):

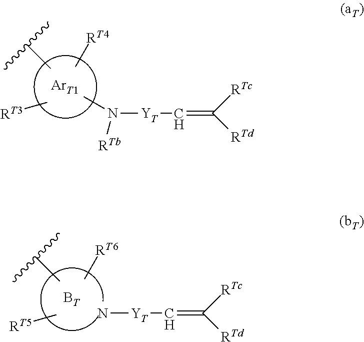

wherein:
Ar_{T1} is 5- or 6-membered cycloalkylene, phenylene, or 5- or 6-membered heteroarylene;
Ring B_{T} is azetidinyl, pyrrolidinyl, or piperidinyl where the nitrogen atom of the azetidinyl, pyrrolidinyl, or piperidinyl ring is attached to Y_{T};
R^{T3} is hydrogen, alkyl, hydroxy, alkoxy, halo, haloalkyl, haloalkoxy, or cyano;
R^{T4} is hydrogen, alkyl, cycloalkyl, hydroxy, alkoxy, halo, haloalkyl, haloalkoxy, or cyano;
R^{T5} and R^{T6} are independently hydrogen, alkyl, or halo;
Y_{T} is —CO— or —SO₂—;
R^{Tb} is hydrogen or alkyl;
R^{Tc} is hydrogen, alkyl, or substituted alkyl; and
R^{Td} is hydrogen or alkyl;
provided that when (i) Ar_{T1} is phenylene or 6-membered heteroarylene then alk_{T} and —NR—Y_{T}—CH═CR^{Tc}R^{Td} are meta or para to each other; and when (ii) B_{T} is piperidinyl, then alk_{T} and —Y_{T}—CH═CR^{Tc}R^{Td} are meta or para to each other.

### PI3Kinase-Binding Exogenous Molecules

In some embodiments, a PI3Kinase-binding exogenous molecule can be an inhibitor. In some embodiments, the inhibitor can bind covalently a PI3Kinase protein. In some embodiments, the covalent inhibitor is a covalent inhibitor of PIK3 CA. In some embodiments, the covalent inhibitor binds to mutant PI3Kinase such as PI3Kinase (H1047R/Y), PI3Kinase (E545K/D) and PI3Kinase (E542K). In some embodiments, the covalent inhibitor wildtype reside like K802 (in wildtype PI3Kinase), C862 of p110alpha subunit of PI3Kinase (CNX-1351), K779 in p110delta subsunit. In some embodiments, the covalent inhibitor is as described in WO2012122383, or related parents and applications, each of which is incorporated by reference in their entirety.

In some embodiments, the covalent inhibitor has the structure of Formula U:

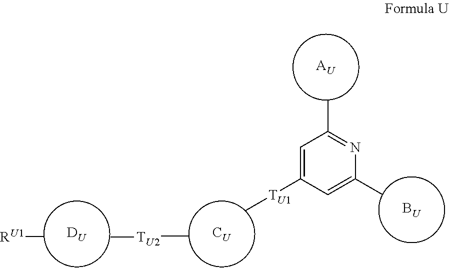

wherein:
R^{U1} is a moiety that is capable of forming a covalent bond with a nucleophile;
Ring A^{U} is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
Ring B^{U} is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T^{U1} is a covalent bond or a bivalent straight or branched, saturated or unsaturated C₁₋₆hydrocarbon chain wherein one or more methylene units of T^{U1} are optionally and independently replaced by —O—, —S—, —N(R^{U})—, —C(O)—, —OC(O)—, —C(O)O—, —C(O)N(R^{U})—, —N(R^{U})C(O)—, —N(R^{U})C(O)N(R^{U})—, —SO₂—, —SO₂N(R^{U})—, —N(R^{U})SO₂—, or —N(R^{U})SO₂N(R^{U})—;
Ring C_{U} is absent or an optionally substituted group selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged or spiro bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; wherein when Ring C_{U} is absent, T_{U2} is directly attached to T_{U1};
T_{U2} is a covalent bond or a bivalent straight or branched, saturated or unsaturated C₁₋₆hydrocarbon chain wherein one or more methylene units of T_{U2} are optionally and independently replaced by —O—, —S—, —N(R^{U})—, —C(O)—, —OC(O)—, —C(O)O—, —C(O)N(R^{U})—, —N(R^{U})C(O)—, —N(R^{U})C(O)N(R^{U})—, —SO₂—, —SO₂N(R^{U})—, —N(R^{U})SO₂—, or —N(R^{U})SO₂N(R^{U})—;
Ring D_{U} is absent or an optionally substituted group selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; wherein when Ring D_{U} is absent, R^{U}i is directly attached to T_{U2}; and
each R^{U} is independently hydrogen or an optionally substituted group selected from C₁₋₆aliphatic, phenyl, a 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
or two R^{U} groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur.

In some embodiments, the covalent inhibitor is selected from:

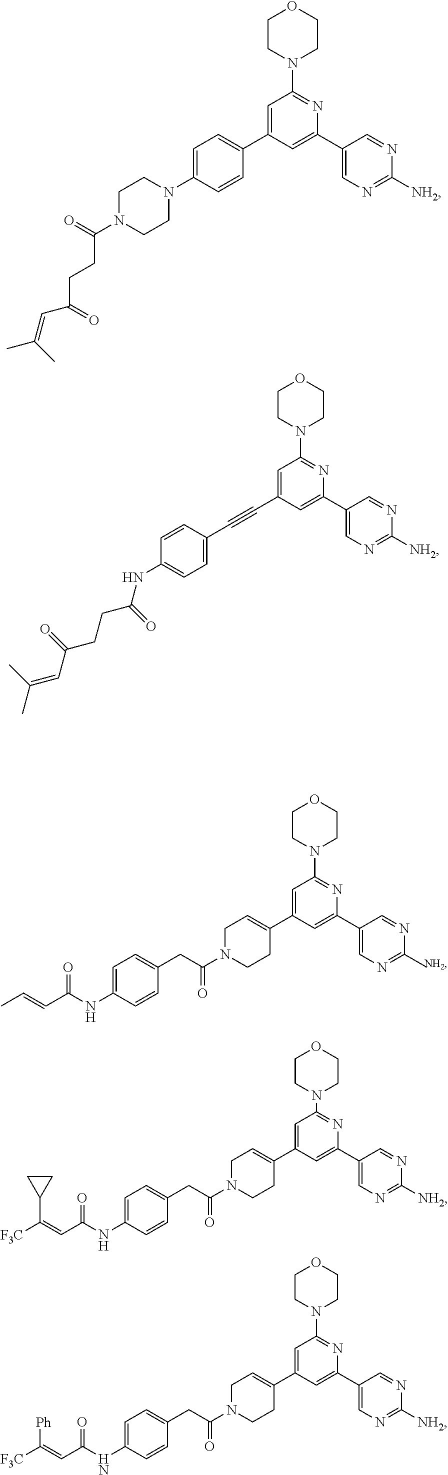 [SMILES: CC(C)=CC(=O)CCC(=O)N1CCN(c2ccc(-c3cc(-c4cnc(N)nc4)nc(N4CCOCC4)c3)cc2)CC1 ; CC(C)=CC(=O)CCC(=O)Nc1ccc(C#Cc2cc(-c3cnc(N)nc3)nc(N3CCOCC3)c2)cc1 ; C/C=C/C(=O)Nc1ccc(CC(=O)N2CC=C(c3cc(-c4cnc(C)nc4)nc(N4CCOCC4)c3)CC2)cc1 ; Nc1ncc(-c2cc(C3=CCN(C(=O)Cc4ccc(NC(=O)/C=C(\C5CC5)C(F)(F)F)cc4)CC3)cc(N3CCOCC3)n2)cn1 ; Nc1ncc(-c2cc(C3=CCN(C(=O)Cc4ccc(NC(=O)/C=C(\c5ccccc5)C(F)(F)F)cc4)CC3)cc(N3CCOCC3)n2)cn1]

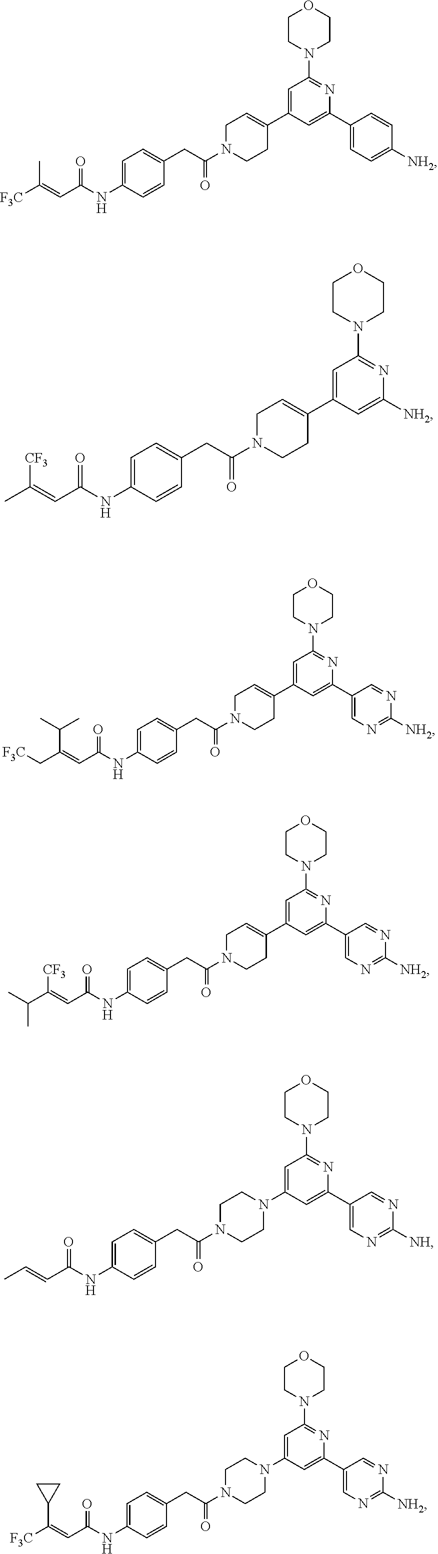

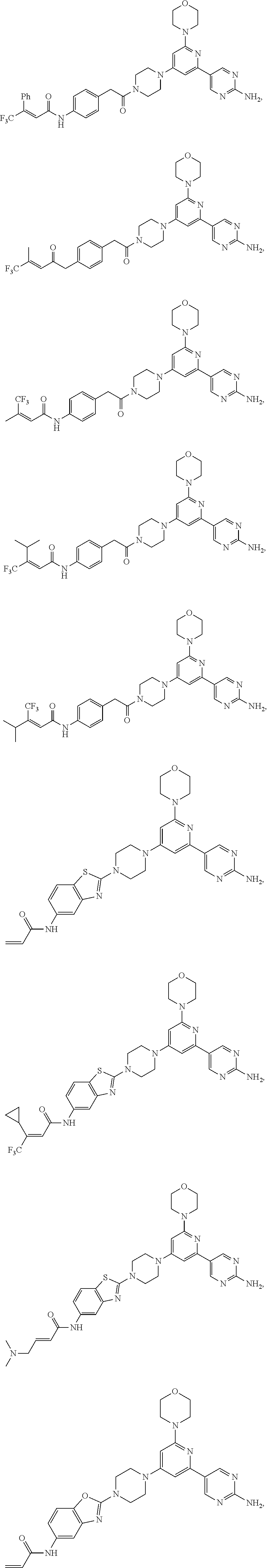

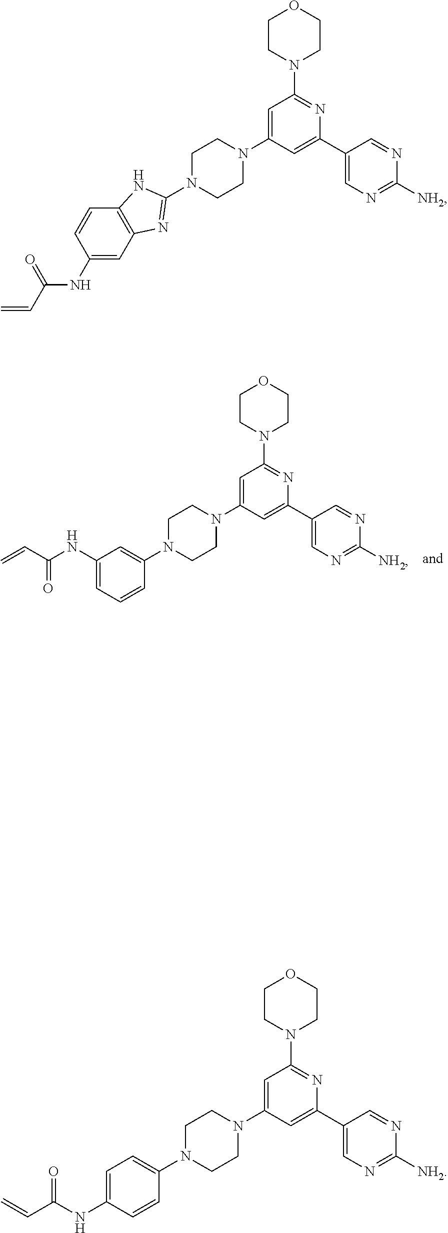 [SMILES: C=CC(=O)Nc1ccc2[nH]c(N3CCN(c4cc(-c5cnc(N)nc5)nc(N5CCOCC5)c4)CC3)nc2c1 ; C=CC(=O)Nc1cccc(N2CCN(c3cc(-c4cnc(N)nc4)nc(N4CCOCC4)c3)CC2)c1 ; C=CC(=O)Nc1ccc(N2CCN(c3cc(-c4cnc(N)nc4)nc(N4CCOCC4)c3)CC2)cc1]

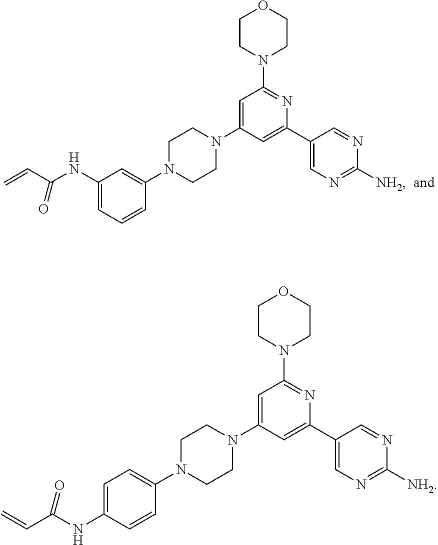 [SMILES: C=CC(=O)Nc1cccc(N2CCN(c3cc(-c4cnc(N)nc4)nc(N4CCOCC4)c3)CC2)c1 ; C=CC(=O)Nc1ccc(N2CCN(c3cc(-c4cnc(N)nc4)nc(N4CCOCC4)c3)CC2)cc1]

Provided in some aspect is an antigen binding unit capable of specifically binding to a cellular target that is covalently bound by an exogenous molecule provided herein. In some embodiments, the antigen binding unit is capable of specifically binding to a Ras protein covalently bound by a Ras inhibitor known in the art or disclosed herein. For example, antigen binding unit is capable of specifically binding to K-Ras G12C mutant that is bound by an inhibitor designated MRTX849, or an inhibitor having a structure

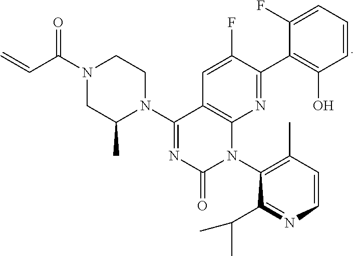

In some embodiments, a subject antigen binding unit is capable of specifically binding to EGFR (and preferably an intracellular portion of EGFR), which is covalently bound by an EGFR inhibitor known in the art or disclosed herein. In some embodiments, a subject antigen binding unit is capable of specifically binding to FGFR (and preferably an intracellular portion of FGFR), which is covalently bound by an FGFR inhibitor known in the art or disclosed herein. In some embodiments, a subject antigen binding unit is capable of specifically binding to Her2 (and preferably an intracellular portion of Her2), which is covalently bound by a Her2 inhibitor known in the art or disclosed herein. In some embodiments, a subject antigen binding unit is capable of specifically binding to PI3Kinase covalently bound by a PI3Kinase inhibitor known in the art or disclosed herein. In some embodiments, a subject antigen binding unit is capable of specifically binding to BTK covalently bound by a BTK inhibitor known in the art or disclosed herein.

### Methods:

In one aspect, the present invention provides a method of developing a subject polypeptide comprising: (a) contacting a plurality of antigen binding units with an intracellular target or an intracellular portion of a target, which is covalently bound by an exogenous molecule capable of specific and covalent binding to said target (bound target); and (b) selecting an antigen binding unit from said plurality, said selected antigen binding unit exhibits specific binding to the bound target, but not the same target without being bound to the exogenous molecule (unbound target), thereby developing the polypeptide. In some other embodiment, the plurality of antigen binding units are presented on a cell, a phage, a surface, or in solution. Methods for preparing antigen binding libraries and methods of screening such are available in the art.

The subject antigen binding units, multivalent antigen binding units, polypeptides (including but not limited to CAR and TCRs) as well as cells comprising any of the foregoing find a wide range of applications in therapeutics, diagnostics and biomedical research.

In one aspect, the present disclosure provides a method of treating cancer in a subject in need thereof comprising: administering to the subject an isolated polypeptide comprising an antigen binding unit, wherein the antigen binding unit (a) exhibits specific binding to a cellular target covalently bound by an exogenous molecule (bound target), but (b) lacks specific binding to the cellular target that is not bound to the exogenous molecule (unbound target), wherein the subject has been exposed to a covalent inhibitor. In another aspect, provided herein is a method of treating cancer in a subject in need thereof comprising: administering to the subject an isolated polypeptide comprising an antigen binding unit, wherein the antigen binding unit (a) exhibits specific binding to an intracellular target or an intracellular portion of a target, which target being bound by an exogenous molecule (bound target), but (b) lacks specific binding to the intracellular target or the intracellular portion of the target, which is not bound to the exogenous molecule (unbound target), wherein the subject has been exposed to a covalent inhibitor.

In yet another aspect, provided herein is a method of treating cancer in a subject in need thereof comprising: administering to the subject a multivalent antigen binding unit comprising a first binding domain and a second binding domain, wherein the first binding domain exhibits (a) specific binding to a cellular target covalently bound by an exogenous molecule (bound target), but (b) lacks specific binding to the cellular target that is not bound to the exogenous molecule (unbound target); and the second antigen binding domain comprises a functional unit capable of modulating one or more cellular functions including apoptosis, cell proliferation, cell differentiation, cell migration, cytotoxicity, release or trafficking of intercellular molecules, growth factor, metabolite, chemical compound, or a combination thereof.

In yet another aspect, provided herein is a method of treating cancer in a subject in need thereof comprising: administering to the subject a multivalent antigen binding unit comprising a first and a second binding domain, wherein the first binding domain exhibits (a) specific binding to an intracellular target or an intracellular portion of a target, which target being bound by an exogenous molecule (bound target), but (b) lacks specific binding to the intracellular target or the intracellular portion of the target, which is not bound to the exogenous molecule (unbound target); and the second antigen binding domain comprises a functional unit capable of modulating one or more cellular functions including apoptosis, cell proliferation, cell differentiation, cell migration, cytotoxicity, release or trafficking of intercellular molecules, growth factor, metabolite, chemical compound, or a combination thereof. In some embodiments, the multivalent antigen binding unit is bivalent or trivalent.

In still yet another aspect, provided herein is a method of treating cancer in a subject in need thereof comprising: administering a modified immune cell. The immune cell comprises one or more chimeric antigen receptors (CARs) comprising an antigen binding unit, wherein said antigen binding unit comprises: (a) a first antigen binding domain (i) exhibiting specific binding to a cellular target covalently bound by an exogenous molecule (bound target), but lacks specific binding to the cellular target that is not bound to the exogenous molecule (unbound target), or (ii) exhibiting specific binding to an intracellular target or an intracellular portion of a target, which target being bound by an exogenous molecule (bound target), but lacks specific binding to the intracellular target or the intracellular portion of the target, which is not bound to the exogenous molecule (unbound target); and (b) a second antigen binding domain exhibiting specific binding to an immune cell antigen, and wherein each CAR of said one or more CARs further comprises a transmembrane unit and an intracellular region comprising an immune cell signaling unit.

In still yet another aspect, provided herein is a method of treating cancer in a subject in need thereof comprising: administering a modified immune cell, comprising one or more T cell receptors (TCR) comprising an antigen binding unit, wherein said antigen binding unit comprises: (a) a first antigen binding domain (i) exhibiting specific binding to a cellular target covalently bound by an exogenous molecule (bound target), but lacks specific binding to the cellular target that is not bound to the exogenous molecule (unbound target), or (ii) exhibiting specific binding to an intracellular target or an intracellular portion of a target, which target being bound by an exogenous molecule (bound target), but lacks specific binding to the intracellular target or the intracellular portion of the target, which is not bound to the exogenous molecule (unbound target); and (b) a second antigen binding domain exhibiting specific binding to an immune cell antigen, and wherein each TCR of said one or more TCRs further comprises a transmembrane unit and an intracellular region comprising an immune cell signaling unit.

A subject in need of a treatment may suffer from a hematological cancer, a solid cancer, or a combination thereof. The cancer can be a hematologic cancer, e.g., a cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. The cancer can also be chosen from colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, a subject suffers from one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL). In some embodiments, the lymphoma is mantle cell lymphoma (MCL), T cell lymphoma, Hodgkin's lymphoma, and/or non-Hodgkin's lymphoma, nephroblastoma, Ewing's sarcoma, neuroendocrine tumor, glioblastoma, neuroblastoma, melanoma, skin cancer, breast cancer, colon cancer, rectal cancer, prostate cancer, liver cancer, kidney cancer, pancreatic cancer, lung cancer, biliary tract cancer, cervical cancer, endometrial cancer, esophageal cancer, gastric cancer, head and neck cancer, medullary thyroid carcinoma, ovarian cancer, glioma, and bladder cancer.

In some embodiments, the subject has been exposed to another cancer treatment including chemotherapy, radiation, gene therapy, cell therapy or a combination thereof. In some embodiments, the subject has been exposed to any known therapy that causes death of the cancer cells. It is known that chemotherapy and radiation often cause death of both normal and cancer cells. Not wishing to be bound by any particular theory, the death of the cancer cells can expose the epitope formed by the bound exogenous molecule specific for the tumor associated polypeptide, thereby allowing a subject antigen binding unit to interact with the epitope to mediate its therapeutic effect. This approach takes advantage of direct targeting epitopes exposed via cell death without resorting to other cellular mechanisms to express the epitopes on a surface of a live cell. Accordingly, a subject cancer treatment can comprise the steps of: administering to the subject a polypeptide comprising an antigen binding unit, wherein the antigen binding unit: (a) exhibits specific binding to an intracellular portion of a target, which target being covalently bound (bound target) by an exogenous molecule that is a covalent inhibitor of the target; and (b) lacks specific binding to the intracellular portion of the target, which is not bound to the exogenous molecule (unbound target); wherein the subject has been exposed to the covalent inhibitor that covalently binds to the intracellular portion of the target to induce formation of an epitope upon covalently binding to the intracellular portion thereof, and wherein the epitope becomes accessible to said antigen binding unit upon death of cancer cells comprising said target. Any target disclosed herein including intracellular target or cell surface proteins can be targeted so long as the epitope formed by a covalent binding to a respective covalent inhibitor is accessible upon death of the cell. Where desired, the covalent inhibitor utilized is an inhibitor directed to cell surface proteins including tyrosine kinases such as EGFR, PDGF, FGF and etc. Of particular interests are covalent inhibitors against EGFR, including without limitation Osimertinib, Afatinib, Dacomitinib, and Neratinib. The structures of these molecules are shown as follows:

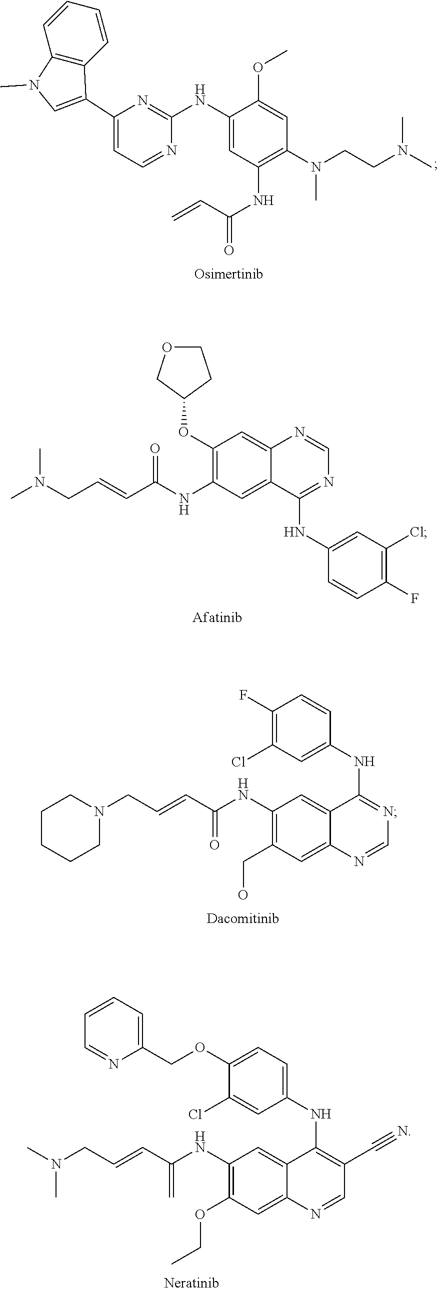

EGFR is a cellular target that helps cells grow and divide. When the EGFR gene is mutated it can cause the protein to be overactive resulting in cancer cells to form. EGFR mutations may occur in 10 to 35 percent of NSCLC tumors globally, and the most common activating mutations are deletions in exon 19 and exon 21 L858R substitution, which together account for more than 80 percent of known activating EGFR mutations. Approximately 10-15% of patients in the US and Europe, and 30-40% of patients in Asia have EGFRm NSCLC. These patients are particularly sensitive to treatment with EGFR-TKIs, which block the cell-signalling pathways that drive the growth of tumour cells. Tumours almost always develop resistance to EGFR-TKI treatment, however, leading to disease progression. Approximately half of patients develop resistance to approved EGFR-TKIs such as gefitinib, erlotinib and afatinib due to the EGFR T790M resistance mutation. There is also a need for medicines with improved CNS efficacy, since approximately 25% of patients with EGFRm NSCLC have brain metastases at diagnosis, increasing to approximately 40% within two years of diagnosis. A number of EGFR inhibitors have been developed and used to treat cancer patients, and they suffer from a number of profound drawback and side effects. Amongst them are decrease in white blood cells (total number; cells needed to fight infection), low platelets, anemia, diarrhea, skin rash, neutropenia (decrease in neutrophils—a type of white blood cell), and dry skin. While the third generation EGFR inhibitor such as Osimertinib is used to treat both EGFR-sensitising and EGFR T790M-resistance mutations, it still can cause resistance. In addition, the combination treatment with PDL1 inhibitor has been reported to be too toxic for some patients.

The subject antigen binding unit, multivalent antigen binding unit, CAR-T or chimeric TCR or immune cells containing the same, can be particularly useful in increasing efficacy, reducing side effect of EGFR inhibitor therapy, or addressing the resistance to EGFR inhibitor treatment. An increase in efficacy can be evidenced by reducing the effective dose of EGFR inhibitor therapy that is otherwise required in the absence of a treatment with a subject antigen binding unit, multivalent antigen binding unit, CAR-T or chimeric TCR or immune cells containing the same. An increased efficacy is achieved when there exists reduction of one or more symptoms of the disease or condition. In an example, a response is achieved when a subject suffering from a tumor exhibits a reduction in the tumor size after the treatment or method, to a greater degree or a longer period of time as compared to a control treatment. In some examples, the efficacy may be measured by assessing cancer cell death, reduction of tumor (e.g., as evidenced by tumor size reduction), and/or inhibition of tumor growth, progression, and dissemination, relative to a control treatment in the absence of a subject composition or without practicing a subject method. A reduction in a side effects is achieved when there is a decrease in any of the side effect associated with EGFR inhibitor disclosed herein or known in the art.

In some embodiments, a subject being treated is exposed to a therapy that causes death of the cancer cells and exposes the epitope to which the antigen binding unit specifically binds. In some instances, the epitope is accessible only upon cell death. For example, this is effectuated when the subject is exposed to chemotherapy, radiation, cell therapy, or a combination thereof. In some embodiment, death of cancer cells occurs upon administering the covalent inhibitor to said subject. For instance, the exogenous molecule (including but not limited to a covalent inhibitor itself when administered to a subject induces death of cancer cells). In some embodiments, the subject is administered a therapy simultaneously, concurrently or sequentially with administering the polypeptide comprising the antigen binding unit, wherein the therapy causes death of cancer cells. In some embodiments, the subject is administered a therapy prior to administering the polypeptide comprising the antigen binding unit, wherein the therapy causes death of cancer cells.

Where the treatment method involves immune cells, the immune cells can be obtained from humans, dogs, cats, mice, rats, and transgenic species thereof. Examples of samples from a subject from which cells, such as immune cells, can be derived include, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues.

In various embodiments of the aspects herein, an immune cell is a lymphocyte. Non-limiting examples of lymphocytes encompassed herein are T cells, B cells, NK cells, KHYG cells, tumor infiltration T cell (TIL), T helper cells, regulatory T cells, and memory T cells. In some embodiments, the lymphoid cell is an immune effector cell. In some embodiments, the lymphocyte is a natural killer cell (NK cell). In some embodiments, the lymphocyte is a T cell.

In some cases, an immune cell provided herein can be positive or negative for a given factor. In some embodiments, an immune cell may be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin− cell, a CD25+, a CD25− cell, a CD27+, a CD27− cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor G1 positive cell, a lectin-like receptor G1 negative cell, or an differentiated or de-differentiated immune cell thereof. The examples of factors expressed by immune cells is not intended to be limiting, and a person having skill in the art will appreciate that an immune cell may be positive or negative for any factor known in the art. In some embodiments, an immune cell may be positive for two or more factors. For example, an immune cell may be CD4+ and CD8+. In some embodiments, an immune cell may be negative for two or more factors. For example, an immune cell may be CD25−, CD44−, and CD69−. In some embodiments, an immune cell may be positive for one or more factors, and negative for one or more factors. For example, an immune cell may be CD4+ and CD8−. In some embodiments, the immune cells may be selected for having or not having one or more given factors (e.g., immune cells may be separated based on the presence or absence of one or more factors). In some embodiments, the selected immune cells can also be expanded in vitro. The selected immune cells can be expanded in vitro prior to infusion into a subject. It should be understood that immune cells used in any of the methods disclosed herein may be a mixture (e.g., two or more different immune cells) of any of the immune cells disclosed herein. For example, a method of the present disclosure may comprise immune cells, and the immune cells are a mixture of CD4+ immune cells and CD8+ immune cells. In another example, a method of the present disclosure may comprise immune cells, and the immune cells are a mixture of CD4+ cells and naïve cells. Subject immune cells can be stem memory T_{SCM} immune cells that can express: CD45RO (−), CCR7(+), CD45RA (+), CD62L+(L-selectin), CD27+, CD28+ and/or IL-7Rα+, said stem memory immune cells can also express CD95, IL-2R3, CXCR3, and/or LFA-1, and show numerous functional attributes distinctive of stem memory immune cells. Alternatively, immune cells can also be central memory T_{CM} immune cells comprising L-selectin and CCR7, where the central memory immune cells can secrete, for example, IL-2, but not IFNγ or IL-4. The immune cells can also be effector memory T_{EM} immune cells comprising L-selectin or CCR7 and produce, for example, effector cytokines such as IFNγ and IL-4. As a person of ordinary skill in the art would understand, both autologous and allogeneic immune cells can be used. For allogeneic transplantation, the isolated population of derived cells are either complete or partial HLA-match with a subject. In another embodiment, the cells are not HLA-matched to the subject, wherein the cells are NK cells or T cell with HLA I and HLA II null.

Subject immune cells can be obtained from a number of other sources, including peripheral blood mononuclear cells, bone marrow, lymph node tissue, spleen tissue, umbilical cord, and tumors. In some embodiments, any number of T cell lines available can be used. Immune cells such as lymphocytes (e.g., cytotoxic lymphocytes) can be autologous cells. Immune cells can also be allogeneic or xenogeneic. T cells can be obtained from a unit of blood collected from a subject using any number of techniques including Ficoll separation. Cells from the circulating blood of an individual can be obtained by apheresis or leukapheresis. The apheresis product comprises lymphocytes, including T cells, monocytes, granulocytes, B cells, other nucleated white blood cells, red blood cells, and platelets. In an aspect, cells collected by apheresis can be washed to remove the plasma fraction and to place the cells in an appropriate buffer or media, such as phosphate buffered saline (PBS), for subsequent processing steps. After washing, the cells can be resuspended in a variety of biocompatible buffers, such as Ca-free, Mg-free PBS. Alternatively, the undesirable components of the apheresis sample can be removed and the cells directly resuspended in culture media. Samples can be provided directly by the subject, or indirectly through one or more intermediaries, such as a sample collection service provider or a medical provider (e.g. a physician or nurse). In some embodiments, isolating T cells from peripheral blood leukocytes can include lysing the red blood cells and separating peripheral blood leukocytes from monocytes by, for example, centrifugation through, e.g., a PERCOL gradient. A specific subpopulation of T cells, such as CD4+ or CD8+ T cells can be further isolated by positive or negative selection techniques. Negative selection of a T cell population can be accomplished, for example, with a combination of antibodies directed to surface markers unique to the cells negatively selected. One suitable technique includes cell sorting via negative magnetic immunoadherence, which utilizes a cocktail of monoclonal antibodies directed to cell surface markers present on the cells negatively selected. For example, to isolate CD4+ cells, a monoclonal antibody cocktail can include antibodies to CD14, CD20, CD11b, CD16, HLA-DR, and CD8. The process of negative selection can be used to produce a desired T cell population that is primarily homogeneous. In some embodiments, a composition comprises a mixture of two or more (e.g. 2, 3, 4, 5, or more) different kind of T-cells.

In an aspect, an immune cell is a member of an enriched population of cells. One or more desired cell types can be enriched by any suitable method, non-limiting examples of which include treating a population of cells to trigger expansion and/or differentiation to a desired cell type, treatment to stop the growth of undesired cell type(s), treatment to kill or lyse undesired cell type(s), purification of a desired cell type (e.g. purification on an affinity column to retain desired or undesired cell types on the basis of one or more cell surface markers). In some embodiments, the enriched population of cells is a population of cells enriched in cytotoxic lymphocytes selected from cytotoxic T cells (also variously known as cytotoxic T lymphocytes, CTLs, T killer cells, cytolytic T cells, CD8+ T cells, and killer T cells), natural killer (NK) cells, and lymphokine-activated killer (LAK) cells.

For isolation of a desired population of cells by positive or negative selection, the concentration of cells and surface (e.g., particles such as beads) can be varied. In certain embodiments, it can be desirable to significantly decrease the volume in which beads and cells are mixed together (i.e., increase the concentration of cells), to ensure maximum contact of cells and beads. For example, a concentration of 2 billion cells/mL can be used. In some embodiments, a concentration of 1 billion cells/mL is used. In some embodiments, greater than 100 million cells/mL are used. A concentration of cells of 10, 15, 20, 25, 30, 35, 40, 45, or 50 million cells/mL can be used. In yet another embodiment, a concentration of cells from 75, 80, 85, 90, 95, or 100 million cells/mL can be used. In further embodiments, concentrations of 125 or 150 million cells/mL can be used. Using high concentrations can result in increased cell yield, cell activation, and cell expansion.

In an aspect, an immune cell provided herein can be activated prior to contact with isolated polypeptides provided herein. In an aspect, activation can refer to a process whereby a cell, such as an immune cell, transitions from a resting state to an active state. This process can comprise a response to an antigen, migration, and/or a phenotypic or genetic change to a functionally active state. For example, the term activation can refer to the stepwise process of T cell activation. For example, a T cell can require at least two signals to become fully activated. The first signal can occur after engagement of a TCR by the antigen-MHC complex, and the second signal can occur by engagement of co-stimulatory molecules or units. Anti-CD3 can mimic the first signal and anti-CD28 can mimic the second signal in vitro. T cell activation can refer to the state of a T cell that has been sufficiently stimulated to induce detectable cellular proliferation, cytokine production, and/or detectable effector function. In particular, immune cell populations, comprising T cells, can be stimulated in vitro such as by contact with an anti-CD3 antibody or antigen-binding fragment thereof, or an anti-CD2 antibody immobilized on a surface, or by contact with a protein kinase C activator (e.g., bryostatin) sometimes in conjunction with a calcium ionophore. For co-stimulation of an accessory molecule on the surface of the T cells, a ligand that binds the accessory molecule can be used. For example, a population of immune cells, such as T cells, can be contacted with an anti-CD3 antibody and an anti-CD28 antibody, under conditions that can stimulate proliferation of the T cells. In some cases, 4-1BB can be used to stimulate cells. For example, immune cells can be stimulated with 4-1BB and IL-21 or another cytokine. To stimulate proliferation of either CD4 T cells or CD8 T cells, an anti-CD3 antibody and an anti-CD28 antibody can be used. For example, the agents providing a signal may be in solution or coupled to a surface. The ratio of particles to cells may depend on particle size relative to the target cell. In further embodiments, the cells, such as T cells, can be combined with agent-coated beads, where the beads and the cells can be subsequently separated, and optionally cultured. Each bead can be coated with either anti-CD3 antibody or an anti-CD28 antibody, or in some cases, a combination of the two. In an alternative embodiment, prior to culture, the agent-coated beads and cells are not separated but are cultured together. Cell surface proteins may be ligated by allowing paramagnetic beads to which anti-CD3 and anti-CD28 can be attached (3×28 beads) to contact the T cells. In some cases, cells and beads (for example, DYNABEADS® M-450 CD3/CD28 T paramagnetic beads at a ratio of 1:1) are combined in a buffer, for example, phosphate buffered saline (PBS) (e.g., without divalent cations such as, calcium and magnesium). Any cell concentration may be used. The mixture may be cultured for or for about several hours (e.g., about 3 hours) to or to about 14 days or any hourly integer value in between. In another embodiment, the mixture may be cultured for or for about 21 days or for up to or for up to about 21 days. Conditions appropriate for T cell culture can include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-21, IL-15, TGF beta, and TNF alpha or any other additives for the growth of cells. In other embodiments, subject immune cells are expanded in an appropriate media that includes one or more interleukins that result in at least a 200-fold, 250-fold, 300-fold, or 350-fold increase in cells over a 14-day expansion period, as measured by flow cytometry. Other additives for the growth of cells include, but are not limited to, surfactant, plasmanate, and reducing agents such as N-acetyl-cysteine and 2-mercaptoethanol. Media can include RPMI 1640, A1 M-V, DMEM, MEM, α-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some cases, an 865 mL bottle of RPMI may have 100 mL of human serum, 25 mL of Hepes 1M, 10 mL of Penicillin/streptomycin at 10,000U/mL and 10,000 μg/mL, and 0.2 mL of gentamycin at 50 mg/mL. After addition of additives an RPMI media may be filtered using a 0.2 μm×1 L filter and stored at 4° C. In some embodiments, antibiotics, e.g., penicillin and streptomycin, are included only in experimental cultures but not in cultures of cells that are to be infused into a subject. In some cases, human serum can be thawed in a 37° C. water bath, and then heat inactivated (e.g., at 56° C. for 30 mm for 100 mL bottle). The sera can be filtered through a 0.8 μm and 0.45 μm filter prior to addition of medium.

In some cases, immune cells can be activated or expanded by co-culturing with tissue or cells. A cell can be an antigen presenting cell. An artificial antigen presenting cells (aAPCs) can express ligands for T cell receptor and costimulatory molecules and can activate and expand T cells for transfer, while improving their potency and function in some cases. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof. In some cases, an artificial antigen presenting cell (aAPC) may be used to activate and/or expand a cell population. In some cases, an artificial may not induce allospecificity. An aAPC may not express HLA in some cases. An aAPC may be genetically modified to stably express genes that can be used to activation and/or stimulation. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, II, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83.

In some cases, restimulation of immune cells can be performed with antigen and irradiated, histocompatible antigen presenting cells (APCs), such as feeder PBMCs. In some cases, cells can be grown using non-specific mitogens such as PHA and allogenic feeder cells. Feeder PBMCs can be irradiated at 40Gy. Feeder PBMCs can be irradiated from about 10 Gy to about 15 Gy, from about 15 Gy to about 20 Gy, from about 20Gy to about 25 Gy, from about 25 Gy to about 30 Gy, from about 30 Gy to about 35 Gy, from about 35 Gy to about 40 Gy, from about 40 Gy to about 45 Gy, from about 45 Gy to about 50 Gy. In some cases, a control flask of irradiated feeder cells only can be stimulated with anti-CD3 and IL-2.

An aAPC can be a bead. A spherical polystyrene bead can be coated with antibodies against CD3 and CD28 and be used for T cell activation. A bead can be of any size. In some cases, a bead can be or can be about 3 and 6 micrometers. A bead can be or can be about 4.5 micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a 4, poly(lactic-co-glycolic acid) (PLGA) microsphere, a nonspherical particle, a 5, carbon nanotube bundle, a 6, ellipsoid PLGA microparticle, a 7, nanoworms, a fluidic lipid bilayer-containing system, an 8, 2D-supported lipid bilayer (2D-SLBs), a 9, liposome, a 10, RAFTsomes/microdomain liposome, an 11, SLB particle, or any combination thereof. In some cases, an aAPC can expand CD4 T cells. For example, an aAPC can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells. In some cases, the use of aAPCs can be combined with exogenously introduced cytokines for T cell activation, expansion, or any combination. Cells can also be expanded in vivo, for example in the subject's blood after administration of genomically transplanted cells into a subject.

An immune cell can be transiently or non-transiently transfected with one or more polynucleotides described herein. A cell can be transfected as it naturally occurs in a subject. A cell can be taken or derived from a subject and transfected. A cell can be derived from cells taken from a subject, such as a cell line. In some embodiments, a cell transfected with one or more polynucleotides described herein is used to establish a new cell line comprising one or more polynucleotide-derived sequences.

Expression of a polynucleotide comprising an antigen binding unit provided herein can be controlled by one or more promoters. A promoter can be a ubiquitous, constitutive (unregulated promoter that allows for continual transcription of an associated gene), tissue-specific promoter, or an inducible promoter. Expression of a polynucleotide encoding sequence can be regulated. For example, a polynucleotide encoding sequence can be inserted near or next to a ubiquitous promoter. Some ubiquitous promoters can be a CAGGS promoter, an hCMV promoter, a PGK promoter, an SV40 promoter, or a ROSA26 promoter. expression vectors including, but not limited to, at least one of a SFFV (spleen focus-forming virus) or human elongation factor 11a (EF) promoter, CAG (chicken beta-actin promoter with CMV enhancer) promoter human elongation factor 1a (EF) promoter. Examples of less-strong/lower-expressing promoters utilized may include, but is not limited to, the simian virus 40 (SV40) early promoter, cytomegalovirus (CMV) immediate-early promoter, Ubiquitin C (UBC) promoter, and the phosphoglycerate kinase 1 (PGK) promoter, or a part thereof. Inducible expression of chimeric antigen receptor may be achieved using, for example, a tetracycline responsive promoter, including, but not limited to, TRE3GV (Tet-response element, including all generations and preferably, the 3rd generation), inducible promoter (Clontech Laboratories, Mountain View, Calif.) or a part or a combination thereof. One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1 a (EF-1 a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters, inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metalothionein promoter, a glucocorticoid promoter, a progesterone promoter, and a tetracycline promoter.

Subject polypeptides comprising antigen binding units can be introduced to immune cells. In some cases, a retroviral vector (either gamma-retroviral or lentiviral) can be employed for the introduction of subject polypeptides to immune cells. For example, a polypeptide-encoding sequence comprising an antigen binding unit sequence, for example a CAR or TCR, can be cloned into a retroviral vector and expression can be driven from its endogenous promoter, from the retroviral long terminal repeat, or from a promoter specific for a target cell type of interest. Non-viral vectors may be used as well. Non-viral vector delivery systems can include DNA plasmids, naked nucleic acid, and nucleic acid complexed with a delivery vehicle such as a liposome or poloxamer.

A number of viral based systems have been developed for gene transfer into mammalian cells. For example, retroviruses provide a convenient platform for gene delivery systems. A selected gene can be inserted into a vector and packaged in retroviral particles using techniques known in the art. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells. They also have the added advantage of low immunogenicity. Adenoviral vectors have the advantage that they do not integrate into the genome of the target cell thereby bypassing negative integration-related events.

Non-limiting examples of delivery methods or transformation include, for example, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, and nanoparticle-mediated nucleic acid delivery. Conventional viral and non-viral based gene transfer methods can be used to introduce nucleic acids in mammalian cells or target tissues. Such methods can be used to administer nucleic acids encoding compositions of the disclosure to cells in culture, or in a host organism. Non-viral vector delivery systems can include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell.

In an aspect, an immune cell can be transfected with a polypeptide coding for an antigen binding unit, for example a CAR or TCR. Any concentration of vector comprising an antigen binding unit sequence can be utilized, for example a concentration can be from about 100 picograms to about 50 micrograms. In some cases, the amount of nucleic acid (e.g., ssDNA, dsDNA, RNA) that may be introduced into a cell may be varied to optimize transfection efficiency and/or cell viability. For example, 1 microgram of dsDNA may be added to each cell sample for electroporation. In some cases, the amount of nucleic acid (e.g., dsDNA) required for optimal transfection efficiency and/or cell viability may be specific to the cell type. In some cases, the amount of nucleic acid (e.g., dsDNA) used for each sample may directly correspond to the transfection efficiency and/or cell viability. For example, a range of concentrations of transfections. A transgene encoded by a vector can integrate into a cellular genome. In some cases, integration of a transgene encoded by a vector is in the forward direction. In other cases, integration of a transgene encoded by a vector is in the reverse direction.

Electroporation using, for example, the Neon® Transfection System (ThermoFisher Scientific) or the AMAXA® Nucleofector (AMAXA® Biosystems) can also be used for delivery of subject polynucleotide-encoding sequences into subject immune cells. Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some cases, the electroporation pulse voltage, the electroporation pulse width, number of pulses, cell density, and tip type may be adjusted to optimize transfection efficiency and/or cell viability.

In some cases, an immune cell can be transduced with a virus. RNA or DNA viral based systems can be used to target specific cells in the body and trafficking the viral payload to the nucleus of the cell. Viral vectors can be administered directly (in vivo) or they can be used to treat cells in vitro, and the modified cells can optionally be administered (ex vivo). Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted transgene. High transduction efficiencies can be observed in many different cell types and target tissues. Lentiviral vectors are retroviral vectors that can transduce or infect non-dividing cells and produce high viral titers. Selection of a retroviral gene transfer system can depend on the target tissue. Retroviral vectors can comprise cis-acting long terminal repeats with packaging capacity for up to 6-10 kb of foreign sequence. The minimum cis-acting LTRs can be sufficient for replication and packaging of the vectors, which can be used to integrate the therapeutic gene into the target cell to provide permanent transgene expression. Retroviral vectors can include those based upon murine leukemia virus (MuLV), gibbon ape leukemia virus (GaLV), Simian Immuno deficiency virus (SIV), human immuno deficiency virus (HIV), and combinations thereof.

In some cases, an adenoviral-based viral system can be used. Adenoviral-based systems can lead to transient expression of the transgene. Adenoviral based vectors can have high transduction efficiency in cells and may not require cell division. High titer and levels of expression can be obtained with adenoviral based vectors. Adeno-associated virus (“AAV”) vectors can be used to transduce cells with target nucleic acids, e.g., in the in vitro production of nucleic acids and peptides, and for in vivo and ex vivo gene therapy procedures.

Viral based systems can utilize packaging cells to form virus particles capable of infecting a host cell. Host cells can include 293 cells, (e.g., for packaging adenovirus), and Psi2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. Viral DNA can be packaged in a cell line, which can contain a helper plasmid encoding the other AAV genes, namely rep and cap, while lacking ITR sequences. The cell line can also be infected with adenovirus as a helper. The helper virus can promote replication of the AAV vector and expression of AAV genes from the helper plasmid. Contamination with adenovirus can be reduced by, e.g., heat treatment to which adenovirus is more sensitive than AAV. Additional methods for the delivery of nucleic acids to cells can be used, for example, as described in US20030087817, incorporated herein by reference.

In some cases, transduction parameters can be modulated. For example, the starting cell density for cellular modification, such as viral delivery of a vector encoding an antigen binding unit, for example CAR or TCR, may be varied to optimize transfection efficiency and/or cell viability. In some cases, the starting cell density for transfection or transduction of immune cells with a viral vector may be less than about 1×10⁵ cells. In some cases, the starting cell density for cellular modification with a viral vector may be at least about 1×10⁵ cells to at least about 5×10⁷ cells. In some cases, the starting cell density for optimal transfection efficiency and/or cell viability may be specific to the cell type. For example, a starting cell density of 1.5×10⁶ cells may optimal (e.g., provide the highest viability and/or transfection efficiency) for macrophage cells. In another example, a starting cell density of 5×10⁶ cells may optimal (e.g., provide the highest viability and/or transfection efficiency) for human cells. In some cases, a range of starting cell densities may be optimal for a given cell type. For example, a starting cell density between of 5.6×10⁶ and 5×10⁷ cells may optimal (e.g., provide the highest viability and/or transfection efficiency) for human immune cells such as T cells.

In an aspect, a population of engineered immune cells comprising polypeptide sequences comprising subject antigen binding units can comprise at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% engineered cells. In some cases, detection of a subject antigen binding unit, for example TCR or CAR, on a cellular membrane of an engineered immune cell can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% as measured by flow cytometry.

Expression of a CAR or TCR in an immune cell can be verified by an expression assay, for example, qPCR or by measuring levels of RNA. Expression level can be indicative also of copy number. For example, if expression levels are extremely high, this can indicate that more than one copy of a CAR was integrated in a genome. Alternatively, high expression can indicate that a transgene was integrated in a highly transcribed area, for example, near a highly expressed promoter. Expression can also be verified by measuring protein levels, such as through Western blotting.

In an aspect, subject immune cells can be tested in vitro prior to administering into the subject. Testing may comprise phenotypic analysis, functional analysis, viability analysis, and any combination thereof. A variety of tests including evaluation of specific lysis, cytokine release, metabolomic and bioenergetic studies (using Seahorse), intracellular FACS of cytokine production, ELISA-spot assays, ELISA, and lymphocyte subset analysis may be used to evaluate the functionality of subject immune cells, particularly engineered immune cells. In general, differences of 2 to 3-fold in these assays are indicative of true biologic differences between engineered immune cells and control immune cells.

Subject immune cells can be maintained under conditions necessary to support growth; for example, an appropriate temperature (e.g., 37° C.) and atmosphere (e.g., air plus 5% CO2). In some cases, a soluble monospecific tetrameric antibody against human CD3, CD28, CD2, or any combination thereof may be used in culture.

Cellular compositions described herein comprising immune cells can be cryopreserved. A cryopreservation can be performed in, for example, a Cryostor CS10 at 5% DMSO final concentration. A cryopreservation can be at a freeze density from about 7.5×10⁷ cells/mL to about 1.5×10⁸ cells/mL.

For example, in some cases, an immune cell can be harvested, washed, and re-suspended in a buffer, such as Cryostor buffer. This preparation can be mixed with an equal volume of Cryostore CS10. In some cases, a cellular composition is thawed prior to an introducing into a subject in need thereof.

Any of the treatment methods disclosed herein can be administered alone or in combination or in conjunction with another therapy or another agent. By “combination” it is meant to include (a) formulating a subject composition containing a subject antigen binding unit together with another agent, and (b) using the subject composition separate from the another agent as an overall treatment regimen. By “conjunction” it is meant that the another therapy or agent is administered either simultaneously, concurrently or sequentially with a subject composition comprising an antigen binding unit, with no specific time limits, wherein such conjunctive administration provides an therapeutic effect. Where desired, sequential administration can involve administering an exogenous molecule disclosed herein prior to administering a subject polypeptide comprising an antigen binding unit disclosed herein, including the antigen binding unit specifically binding to an epitope formed by complexing the exogenous molecule with its respective target. Where desired, the exogenous molecule can be administered after death of cancer cells has occurred in the subject, e.g., due to prior administration of a chemotherapy, radiation and/or a cell therapy. For example, the chemotherapy, radiation and/or a cell therapy is administered for a period of time sufficient to effect cell death, before the subject is administered with the exogenous molecule, followed by or concurrent with administering a subject polypeptide (e.g., including the multivalent antigen binding unit), or a cell (including without limitation an immune cell) expressing the polypeptide of the present disclosure.

In some embodiment, a subject treatment method is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, and combinations thereof.

In one embodiment, a subject treatment method involving a subject an antigen binding unit or a cell comprising the same can be used in combination with a chemotherapeutic agent.

Exemplary chemotherapeutic agents include an anthracycline (e.g., doxorubicin (e.g., liposomal doxorubicin)). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludambine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the cellular compositions of the present invention include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In an aspect, compositions provided herein can be administered in combination with radiotherapy such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents, radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-1BB antibody, an anti-OX40 antibody, an anti-ICOS antibody, and combinations thereof. In one embodiment, the agonist costimulatory monoclonal antibody is an anti-4-1 BB antibody. In some embodiments, the checkpoint immune blockade agents are selected from the group consisting of anti-PD-L1 antibodies, anti-CTLA-4 antibodies, anti-PD-1 antibodies, anti-LAG3 antibodies, anti-B7-H3 antibodies, anti-TIM3 antibodies, and combinations thereof. In one embodiment, the checkpoint immune blockade agent is an anti-PD-L1 antibody. In some cases, cellular compositions can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising antigen binding units can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldeskeukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 rU/kg to 500,000 IU/kg, from 500,000 IU/kg to 700,000 IU/kg, from 700,000 IU/kg to about 1,000,000 IU/kg.

In combination therapy, cellular compositions provided herein and other anti-cancer agent(s) may be administered either simultaneously, concurrently or sequentially with no specific time limits, wherein such administration provides therapeutically effective levels of the two compounds in the body of the patient. As disclosed herein, any subject treatment, targeting or labeling methods can be practiced concurrent with, prior to, or subsequent to administering another anti-cancer agent that causes death or apoptosis to, e.g., expose the epitope formed by the exogenous molecule (including but not limited to a covalent inhibitor) with a target of interest.

In a preferred embodiment, the cellular compositions of the present disclosure and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

An embodiment further comprises lymphodepleting a subject prior to administering the subject antigen binding units, for example CAR and/or TCRs, disclosed herein. Examples of lymphodepletion include, but may not be limited to, nonmyeloablative lymphodepleting chemotherapy, myeloablative lymphodepleting chemotherapy, and total body irradiation.

In some cases, an antifungal therapy is administered to a subject receiving modified cells. Antifungals can be drugs that can kill or prevent the growth of fungi. Targets of antifungal agents can include sterol biosynthesis, DNA biosynthesis, and β-glucan biosynthesis. Antifungals can also be folate synthesis inhibitors or nucleic acid cross-linking agents. A folate synthesis inhibitor can be a sulpha based drug. For example, a folate synthesis inhibitor can be an agent that inhibits a fungal synthesis of folate or a competitive inhibitor. A sulpha based drug, or folate synthesis inhibitor, can be methotrexate or sulfamethaxazole. In some cases, an antifungal can be a nucleic acid cross-linking agent. A cross-linking agent may inhibit a DNA or RNA process in fungi. For example, a cross-linking agent can be 5-fluorocytosine, which can be a fluorinated analog of cytosine. 5-fluorocytosine can inhibit both DNA and RNA synthesis via intracytoplasmic conversion to 5-fluorouracil. Other anti-fungal agents can be griseofulvin. Griseofulvin is an antifungal antibiotic produced by Penicillium griseofulvum. Griseofulvin inhibits mitosis in fungi and can be considered a cross linking agent. Additional cross-linking agent can be allylamines (naftifine and terbinafine) inhibit ergosterol synthesis at the level of squalene epoxidase; one morpholene derivative (amorolfine) inhibits at a subsequent step in the ergosterol pathway. In some cases, an antifungal agent can be from a class of polyene, azole, allylamine, or echinocandin. In some embodiments, a polyene antifungal is amphotericin B, candicidin, filipin, hamycin, natamycin, nystatin, or rimocidin. In some cases, an antifungal can be from an azole family. Azole antifungals can inhibit lanosterol 14 α-demethylase. An azole antifungal can be an imidazole such as bifonazole, butoconazole, clotrimazole, econazole, fenticonazole, isoconazole, ketoconazole, luliconazole, miconazole, omoconazole, oxiconazole, sertaconazole, sulcoazole, or tioconazole. An azole antifungal can be a triazole such as albaconazole, efinaconazole, epoxiconazole, fluconazole, isavuvonazole, itraconazole, posaconazole, propiconazole, ravuconazole, terconazole, or voriconazole. In some cases an azole can be a thiazole such as abafungin. An antifungal can be an allylamine such as amorolfin, butenafine, naftifine, or terbinafine. An antifungal can also be an echinocandin such as anidulafungin, caspofungin, or micafungin. Additional agents that can be antifungals can be aurones, benzoic acid, ciclopirox, flucytosine, griseofulvin, haloprogin, tolnaftate, undecylenic acid, cystal violet or balsam of Peru.

An antibiotic can be administered to a subject as part of a therapeutic regime. An antibiotic can be administered at a therapeutically effective dose. An antibiotic can kill or inhibit growth of bacteria. An antibiotic can be a broad spectrum antibiotic that can target a wide range of bacteria. Broad spectrum antibiotics, either a 3^{rd} or 4^{th} generation, can be cephalosporin or a quinolone. An antibiotic can also be a narrow spectrum antibiotic that can target specific types of bacteria. An antibiotic can target a bacterial cell wall such as penicillins and cephalosporins. An antibiotic can target a cellular membrane such as polymyxins. An antibiotic can interfere with essential bacterial enzymes such as antibiotics: rifamycins, lipiarmycins, quinolones, and sulfonamides. An antibiotic can also be a protein synthesis inhibitor such as macrolides, lincosamides, and tetracyclines. An antibiotic can also be a cyclic lipopeptide such as daptomycin, glycylcyclines such as tigecycline, oxazolidiones such as linezolid, and lipiarmycins such as fidaxomicin. In some cases, an antibiotic can be 1^{st} generation, 2^{nd} generation, 3^{rd} generation, 4^{th} generation, or 5^{th} generation. A first-generation antibiotic can have a narrow spectrum. Examples of 1^{st} generation antibiotics can be penicillins (Penicillin G or Penicillin V), Cephalosporins (Cephazolin, Cephalothin, Cephapirin, Cephalethin, Cephradin, or Cephadroxin). In some cases, an antibiotic can be 2^{nd} generation. 2^{nd} generation antibiotics can be a penicillin (Amoxicillin or Ampicillin), Cephalosporin (Cefuroxime, Cephamandole, Cephoxitin, Cephaclor, Cephrozil, Loracarbef). In some cases, an antibiotic can be 3^{rd} generation. A 3^{rd} generation antibiotic can be penicillin (carbenicillin and ticarcillin) or cephalosporin (Cephixime, Cephtriaxone, Cephotaxime, Cephtizoxime, and Cephtazidime). An antibiotic can also be a 4^{th} generation antibiotic. A 4^{th} generation antibiotic can be Cephipime. An antibiotic can also be 5^{th} generation. 5^{th} generation antibiotics can be Cephtaroline or Cephtobiprole.

In some cases, an anti-viral agent may be administered as part of a treatment regime. In some cases, a herpes virus prophylaxis can be administered to a subject as part of a treatment regime. A herpes virus prophylaxis can be valacyclovir (Valtrex). Valtrex can be used orally to prevent the occurrence of herpes virus infections in subjects with positive HSV serology. It can be supplied in 500 mg tablets. Valacyclovir can be administered at a therapeutically effective amount.

Provided herein can also be a method of administering subject immune cells comprising an antigen binding unit. In some instances, the dose of transduced cells given to a subject can be about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single dose. Any number of cells can be infused for therapeutic use. For example, a patient may be infused with a number of cells between 1×10⁶ to 5×10¹² cells/kg inclusive. A patient may be infused with as many cells that can be generated for them. In some cases, cells that are infused into a patient are not all engineered. For example, at least 90% of cells that are infused into a patient can be engineered. In other instances, at least 40%, 50%, 60%, 65%, 70%, 75%, or 80% of cells that are infused into a subject comprise a subject antigen binding unit.

In some cases, a treatment regime may be dosed according to a body weight of a subject. In subjects who are determined obese (BMI>35) a practical weight may need to be utilized. BMI is calculated by: BMI=weight (kg)/[height (m)]².

Body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight−ideal body weight)). In some cases a body surface area may be utilized to calculate a dosage. A body surface area (BSA) may be calculated by: BSA (m2)=√Height (cm)*Weight (kg)/3600.

In some cases, a pharmaceutical composition comprising an antigen binding unit as described herein can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes.

The polypeptides disclosed herein provide an effective tool to locate in vitro and in vivo a cellular target of interest, which is identified by exogenous molecule (a) capable of specifically binding to the cellular target; (b) capable of forming a stable complex (in some instances forming a covalent bond). The resulting antigen binding unit exhibits specific binding to the bound target provides the “GPS” signal indicative of the location, identity and/or expression level of the target in vivo or in vitro, depending on the setting the signal is detected.

In a separate aspect, the present invention provides a method of targeting an intracellular target or an intracellular portion of a target in a subject by utilizing any of the polypeptide comprising an antigen binding unit disclosed herein, including the multivalent antigen binding unit, cells comprising the antigen binding unit. In one embodiment, the method involves: (a) administering to the subject an exogenous molecule that covalently binds to the target or the intracellular portion of a target; and (b) administering to the subject a subject polypeptide disclosed herein (including the multivalent antigen binding unit), wherein an epitope to which the polypeptide or the multivalent antigen binding unit binds is accessible for said binding. In some embodiments, the exogenous molecule utilized in a subject method or a subject composition specifically and covalently binds to an intracellular target or an intracellular portion of a target of interest.

In a related but separate aspect, the present invention provides a method of labeling a tumor cell comprising: (a) contacting the tumor cell with a covalent inhibitor; and (b) contacting the tumor cell with a subject polypeptide disclosed herein (including the multivalent antigen binding unit), wherein an epitope to which the polypeptide or the multivalent antigen binding unit binds is accessible for said binding, thereby labeling said tumor cell.

The subject targeting and labeling methods are particularly useful for diagnosis, prognosis and treatment of diseases associated with the target. Where the antigen binding unit is labeled with a detectable label, a wide range of detection methods are applicable to identify, track or monitor the location and/or expression level of the target.

For example, a subject polypeptide comprising a suitable label or a label to be used in conjunction with a subject polypeptide may be administered to a subject (e.g., a patient) and subsequently detected by an in vivo (i.e., non-invasive) imaging technique. Examples of in vivo imaging techniques include nuclear imaging techniques, such as positron emission tomography (PET) techniques, gamma cameras, SPECT (single-photon emission computed tomography), or nuclear magnetic resonance (NMR) techniques. Examples of NMR techniques include magnetic resonance imaging (MRI) and localized magnetic resonance spectroscopy (MRS). The label may be detected (e.g., imaged) for at least 1, 2, 3, 4, 5, or more time points in the subject. The label may be detected (e.g., imaged) for at most 5, 4, 3, 2, or 1 time point in the subject. Labels may also be detected in a cell culture or in essentially any other milieu on which a detection technique (e.g., nuclear imaging techniques or fluorescence imaging techniques) can be performed, such as tissue explants, organs and tissues removed from a subject (e.g., prior to transplant into a transplant recipient), artificially generated tissues, or various matrices and structures seeded with cells.

### EXAMPLES

### Example 1: Modifying Immune Cells to Express an Antigen Binding Unit

Isolation of Peripheral Blood Mononuclear Cells (PBMCs) from a LeukoPak

Leukopaks collected from normal peripheral blood are used. Blood cells are diluted 3 to 1 with chilled 1×PBS. The diluted blood was added dropwise (e.g., very slowly) over 15 mLs of LYMPHOPREP (Stem Cell Technologies) in a 50 ml conical. Cells are spun at 400×G for 25 minutes with no brake. The buffy coat is slowly removed and placed into a sterile conical. The cells are washed with chilled 1×PBS and spun for 400×G for 10 minutes. The supernatant is removed, cells resuspended in media, counted and viably frozen in freezing media (45 mLs heat inactivated FBS and 5 mLs DMSO).

Isolation of CD3+ T cells

PBMCs are thawed or used fresh and plated for 1-2 hours in culturing media (RPMI-1640 (with no Phenol red), 20% FBS (heat inactivated), and 1× Gluta-MAX). Cells are collected and counted; the cell density is adjusted to 5×10⁷ cells/mL and transferred to sterile 14 mL polystyrene round-bottom tube. Using the EasySep Human CD3 cell Isolation Kit (Stem Cell Technologies), 50 uL/mL of the Isolation Cocktail was added to the cells. The mixture is mixed by pipetting and incubated for 5 minutes at room temperature. After incubation, the RapidSpheres are vortexed for 30 seconds and added at 50 uL/mL to the sample; mixed by pipetting. Mixture is topped off to 5 mLs for samples less than 4 mLs or topped off to 10 mLs for samples more than 4 mLs. The sterile polystyrene tube is added to a “Big Easy” magnet; incubated at room temperature for 3 minutes. The magnet and tube, in one continuous motion, are inverted, pouring off the enriched cell suspension into a new sterile tube.

### Activation and Stimulation of CD3+ T Cells

Isolated CD3+ T cells are counted and plated out at a density of 2×10⁶ cells/mL in a 24 well plate. Dynabeads Human T-Activator CD3/CD28 beads (Gibco, Life Technologies) are added 3:1 (beads: cells) to the cells after being washed with 1×PBS with 0.2% BSA using a dynamagnet. IL-2 (Peprotech) was added at a concentration of 300 IU/mL. Cells are incubated for 48 hours and then the beads are removed using a dynamagnet. Cells are cultured for an additional 6-12 hours before transduction or electroporation.

### Neon Transfection of CD3⁺ T Cells

Unstimulated or stimulated T cells are electroporated using the Neon Transfection System (10 uL Kit, Invitrogen, Life Technologies). Cells are counted and resuspended at a density of 2×10⁵ cells in 10 uL of T buffer. 1 ug of vector comprising an antigen binding unit, CAR or TCR, is added to the cell mixture. Cells are electroporated at 1400 V, 10 ms, 3 pulses. After transfection, cells are plated in a 200 uL culturing media in a 48 well plate.

### Flow Cytometry

Electroporated or transduced T cells are analyzed by flow cytometry 24-48 hours post transfection or transduction for expression of the antigen binding unit, CAR or TCR. Cells are prepped by washing with chilled 1×PBS with 0.5% FBS and stained with anti-TCR and/or anti-CAR, anti-human CD3E (eBiosciences, San Diego), anti-human CD4, and anti-human CD8 and Fixable Viability Dye eFlour 780 (eBiosciences, San Diego). Cells were analyzed using a LSR II (BD Biosciences, San Jose) and FlowJo v.9.

### Example 2. Production of a Model of a Cellular Target

A protein (e.g., an intracellular protein or cell surface protein) or or a mutated variant thereof may serve as a target for an exogenous molecule, e.g., a protein inhibitor, disclosed herein. To raise a subject antigen binding unit, the target of interest is complexed with the exogenous molecule to form a stable complex. The complex is then utilized as an immunogen or part of an immunogen for raising antibodies utilizing any methods known in the art. For example, to raise an antigen binding unit that specifically recognizes the complex of EGFR and its inhibitor such as Osimertinib, EGFR or an intracellular portion thereof containing the binding site of Osimertinib are allowed to form a stable complex. Alternatively, a short fragment of the intracellular part of the EGFR such as a sequence LMPFGCLLDYVREH K can be utilized to conjugate with an EGFR inhibitor (e.g., Osimertinib) via disulfide bond. The conjugate is then used as an immunogen or part of an immunogen for raising the antigen binding unit.

### Example 3. Hybridoma-Based Monoclonal Antibody Production

Adult Balb/c mice may be immunized subcutaneously with the immunogen described in Example 2 (e.g., 100-200 pg) and complete Freund's adjuvant in a 1:1 mixture. After 2-3 weeks, the mice may be injected intraperitoneally or subcutaneously with incomplete Freund's adjuvant and the immunizing conjugate in a 1:1 mixture. The injection may be repeated at 4-6 weeks to enhance the immune response. Sera may be collected from mice 7 days post-third-injection and assayed for immunoreactivity to the EGFR sequence complex with its inhibitor by ELISA and western blotting.

Mice that display a good response to the immunizing conjugate may be boosted by a single intra-spleen injection with 50 μl of the immunizing conjugate mixed 1:1 with Aluminum hydroxide using a 31 gauge extra long needle (Goding, J. W., (1996) Monoclonal Antibodies: Principles and Practices. Third Edition, Academic Press Limited. p. 145). Briefly, mice may be anesthetized with 2.5% avertin, and a 1 centimeter incision may be created on the skin and left oblique body wall. The mixture comprising the immunizing conjugate and Aluminum hydroxide may be administered by inserting the needle from the posterior portion to the anterior portion of the spleen in a longitudinal injection. The body wall may be sutured and the skin may be sealed with two small metal clips. Mice may be monitored for safe recovery. Four days after surgery the mouse spleen may be removed and single cell suspensions may be made for fusion with mouse myeloma cells for the creation of hybridoma cell lines (Spitz, M., (1986) Methods In Enzymology, Volume 121. Eds. John J, Lagone and Helen Van Vunakis. PP. 33-41 (Academic Press, New York, N.Y.)). Resulting hybridomas may be cultured in appropriate media, e.g., Dulbeccos modified media (Gibco) supplemented with 15% fetal calf serum (Hyclone) and hypoxathine, aminopterin, and thymidine.

Screening for positive hybridomas may begin 8 days after the fusion and may continue for 15 days. Hybridomas producing one or more antibodies against the EGFR complexed with its covalent inhibitor may be identified by ELISA on two sets of 96-well plates: (i) one coated the cellular target comprising the purified substrate (or a plurality of polypeptide chains thereof) and the exogenous molecule (i.e., bound sample), and (ii) another one coated with the purified substrate (or a plurality of polypeptide chains thereof) in absence of the exogenous molecule as a negative control (i.e., unbound sample). A counter screen may include the EGFR inhibitor alone in absence of any fragments to which the inhibitor binds. Another counter screen may include the EGFR sequence absent of its inhibitor. A negative control can be a secondary antibody a donkey anti-mouse IgG labeled with horseradish peroxidase (HRP) (Jackson Immunoresearch). Immunoreactivity may be monitored in wells using color development initiated by ABTS tablets dissolved in TBS buffer, pH 7.5. The individual HRP reaction mixtures may be terminated by adding 100 microliters of 1% SDS and absorbance at 405 nm may be measured with a spectrophotometer. Hybridomas producing the one or more antibodies against the cellular target, and not against the 6His tag that is coupled to the cellular target (for purification purposes) may be used for further analysis. Limiting dilutions (0.8 cells per well) may be performed one or more times on positive clones in 96 well plates, with clonality defined as having greater than at least 90% (e.g., 95% or 99%) of the wells with positive reactivity. Isotypes of antibodies may be determined using the iso-strip technology (Roche). To obtain purified antibody for further evaluation, tissue culture supernatants may be affinity purified using a protein A or protein G columns.

In some embodiments, a plurality of monoclonal antibodies (e.g., five monoclonal antibodies) that are immunoreactive to the cellular target may be isolated and compared for their affinities against the cellular target, thus selecting for a monoclonal antibody with desired binding characteristics. Such monoclonal antibody may be deposited with American Type Culture Collection (ATCC), P.O. Box 1549, Manassas, Va., 20108, USA. All animal procedures may be performed in accordance with the guidelines established by the Institutional Animal Care and Use Committee in a USDA and OLAW certified facility.

### Example 4. Llama Immunoglobulin Production

A model of the cellular target of a tumor associated protein (or its mutated variant) such as EGFR may be prepared, as provided in Example 2. Such model may comprise the exogenous molecule bound to the purified substrate of EGFR (or a plurality of polypeptide chains thereof). A llama may be initially immunized subcutaneously with 500 of the model cellular target of EGFR and Complete Freund Adjuvant on day 0 at 8 different sites (62.5 milligram (mg) per site). The llama may be boosted subcutaneously with 500 g of the model cellular target of EGFR and Incomplete Freund Adjuvant on days 15, 29, 57, and 84 at 8 different sites (62.5 mg per site). On day 111, the llama may be boosted with a complex (e.g., a covalently coupled conjugate) between the model cellular target of EGFR and Keyhole limpet hemocyanin (KLH) (i.e., exogenous molecule-substrate-KLH) at the same dose and manner. Production bleeds (500 ml each) may be obtained at days 43, 69, 98, and 125. Serum antibody titers may be determined and PBMC from each bleed may be stored in RNA lysis buffer.

Antisera from each bleed may be tested by ELISA for reactivity and specificity to the model cellular target of EGFR as they were collected. One or more of the antisera may comprise heavy chain antibody (i.e., VHH IgG) against the cellular target of EGFR. Antisera from bleeds prior to immunization of the llama may be included as controls. Two or more independent tests may be performed, each with a different binding condition, to confirm the activity. In an example, one or more llama antibodies against the abovementioned cellular target of the EGFR may be identified by ELISA on two sets of 96-well plates: (i) one coated the cellular target comprising the purified EGFR and Osimertinib (i.e., bound sample), and (ii) another one coated with the purified EGFR in absence of the Osimertinib as a negative control (i.e., unbound sample). In some cases, antiserum titer test may be performed, and the antiserum titer may be positive at greater than 600,000 dilution and absolutely positive at over 10,000 dilution.

Based on the antisera results, PMBC collected on day 125 may be used for VHH library construction. Total RNA may be purified from the lysed cells and used as a template for RT-PCR for construction of a single domain antibody library. The VHH coding DNA may be purified after PCR using specific primers. A phage display vector pADL20c (AbDesign Labs, San Diego) may be used for cloning. A library of at least about 1×10⁸ independent clones may be obtained. A plurality of clones (e.g., 10 clones) may be picked randomly and sequenced. The plurality of clones may comprise VHH inserts in the correct reading frame. The phage display antibody library may be screened with antigen-coated plates (e.g., two sets of ELISA plates, as abovementioned in this Example). After four rounds of panning, 95 positive clones may be randomly picked to select individual positive clones. Over 80% of the clones may be positive. Additionally, positive lysates may be examined for specific binding against other antigens as negative controls. Afterwards, a final positive clone (e.g., lead VHH) may be selected for further analysis. In some embodiments, a second phage display library comprising a plurality of mutations of the lead VHH may be prepared and tested to further optimize the lead VHH and its affinity to the cellular target of EGFR.

CDR-1, CDR-2, and CDR-3 from the lead VHH may be sequenced and grafted into a human immunoglobulin (Ig) VH to generate a humanized monoclonal antibody against the cellular target of EGFR. Alternatively, the lead VHH may be recombinantly fused to a human Fc fragment to form a llama/human chimeric heavy chain-only antibody (i.e., monoclonal HCAb) against the cellular target of EGFR. The resulting antibody may be deposited with American Type Culture Collection (ATCC), P.O. Box 1549, Manassas, Va., 20108, USA. All animal procedures may be performed in accordance with the guidelines established by the Institutional Animal Care and Use Committee in a USDA and OLAW certified facility.

1-24. (canceled)

25. A method of labeling a tumor cell in a tissue, wherein the tumor cell expresses a tumor-associated intracellular target (“TAT”), or an intracellular portion of a membrane bound target (“IPT”), the method comprising:
(a) contacting the tumor cell with a covalent inhibitor that covalently binds to the TAT or IPT to form an epitope that becomes accessible and recognizable by an antigen binding unit upon death of said tumor cell; and
(b) contacting the dead tumor cell with the antigen binding unit, wherein the antigen binding unit exhibits specific binding to said epitope that is formed by covalently binding the inhibitor to an amino acid residue in the TAT or the IPT, thereby labeling said tumor cell in a tissue.

26. The method of claim 25, wherein the covalent inhibitor covalently binds to a TAT selected from the group consisting of KRAS, PI3Kinase, and BTK.

27. The method of claim 25, wherein the covalent inhibitor covalently binds a cysteine or an aspartate residue of a KRAS mutant protein.

28. The method of claim 25, wherein the covalent inhibitor covalently binds cysteine at position 12 of KRAS G12C mutant protein.

29. The method of claim 25, wherein the covalent inhibitor covalently binds aspartate at position 12 of KRAS G12D mutant protein.

30. The method of claim 25, wherein the covalent inhibitor covalently binds an amino acid residue at position 12 of KRAS protein.

31. The method of claim 25, wherein the covalent inhibitor covalently binds to an intracellular portion of a membrane bound target selected from the group consisting of EGFR, FGFR, and Her2.

32. The method of claim 25, wherein the covalent inhibitor is

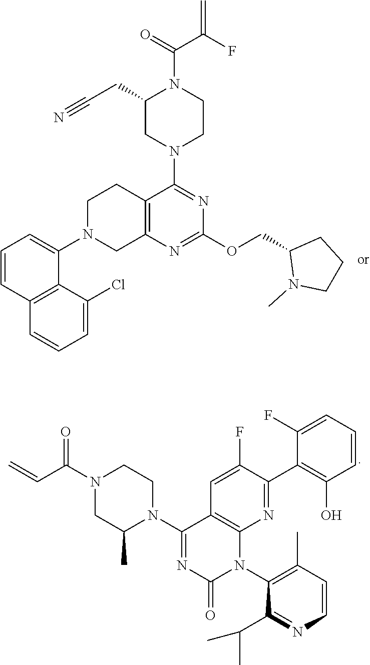

33. The method of claim 25, wherein the covalent inhibitor is a compound selected from the group consisting of 0

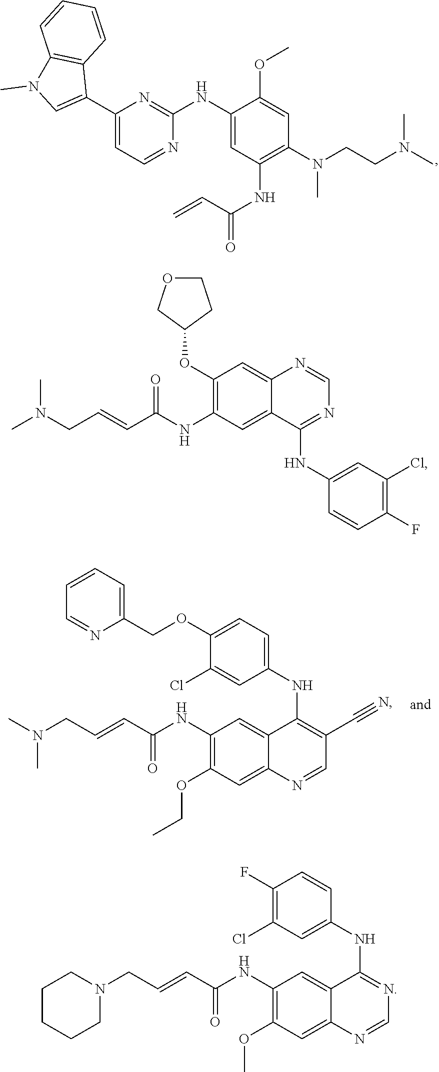

34. The method of claim 25, wherein death of the tumor cell occurs after contacting the tumor cell with the covalent inhibitor.

35. The method of claim 25, further comprising exposing the tumor cell to a chemotherapeutic agent, radiation, cell therapy, or a combination thereof, to induce death of said tumor cell.

36. The method of claim 25, wherein the antigen binding unit comprises a member selected from the group consisting of a Fab, F(ab′)₂, a single chain variable fragment (scFv), a variable fragment (Fv), a single-unit antibody (SdAb), a minibody, a diabody, and a camelid antibody.

37. The method of claim 25, wherein the antigen binding unit is a multivalent antigen binding unit.

38. The method of claim 25, wherein the antigen binding unit comprises a cytokine, a chemokine, a radioisotope, a fluorophore, or a toxin.

39. A method of targeting an intracellular target (“TAT”) or an intracellular portion of a target (“IPT”) expressed by a tumor cell in a tumor, comprising:
(a) contacting the tumor cell with an exogenous molecule that covalently binds to the TAT or IPT to form an epitope that becomes accessible and recognizable by an antigen binding unit upon death of the tumor cell in the tumor; and
(b) contacting the dead tumor cell with the antigen binding unit, wherein the antigen binding unit exhibits specific binding to said epitope formed by covalently binding the inhibitor to an amino acid residue in the TAT or the IPT, thereby targeting said TAT or IPT in the tumor.

40. The method of claim 39, wherein the exogenous molecule covalently binds to an intracellular target selected from the group consisting of KRAS, PI3Kinase, and BTK.

41. The method of claim 39, wherein the exogenous molecule covalently binds a cysteine or an aspartate residue at position 12 of a KRAS mutant protein.

42. The method of claim 39, wherein the exogenous molecule covalently binds an amino acid residue at position 12 of KRAS protein.

43. The method of claim 39, wherein the exogenous molecule covalently binds to an intracellular portion of a membrane bound target selected from the group consisting of EGFR, FGFR, and Her2.

44. The method of claim 39, wherein the exogenous molecule is selected from the group consisting of 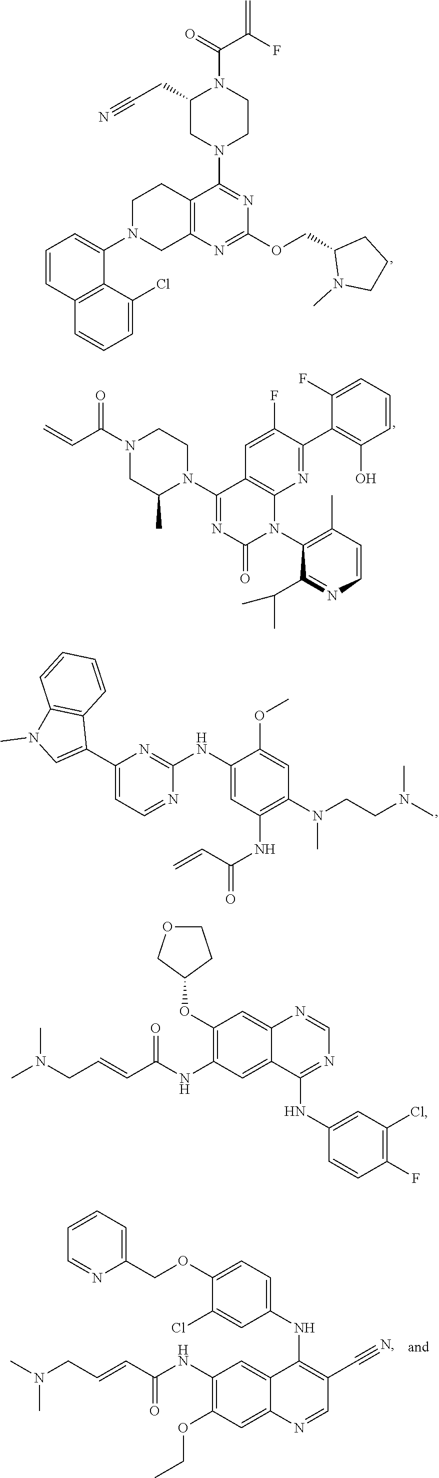 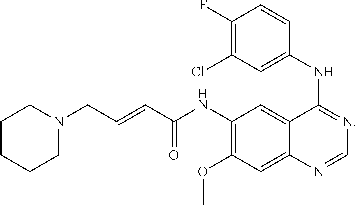 [SMILES: COc1cc2ncnc(Nc3ccc(F)c(Cl)c3)c2cc1NC(=O)/C=C/CN1CCCCC1]
